(19) United States
(12) Patent Application Publication
(10) Pub. No.: US 2003/0096240 A1
(43) Pub. Date: May 22, 2003

(54) Genomic organization of mouse and human sGC

(76) Inventors: Ferid Murad, Houston, TX (US); Iraida G. Sharina, Houston, TX (US); Joshua S. Krumenacker, Houston, TX (US); Emil Martin, Houston, TX (US)

Correspondence Address: Thomas M. Boyce, FULBRIGHT & JAWORSKI L.L.P., Suite 2400, 600 Congress Avenue, Austin, TX, 78701, US

(21) Appl. No.: 09/952,213

(22) Filed: Sep. 12, 2001

Related U.S. Application Data
(60) Provisional application No. 60/233,500, filed on Sep. 19, 2000.

(51) Int. Cl. C12Q 1/68; C07H 21/04; C12N 9/22; C12P 21/02; C12N 5/06

(52) U.S. Cl. 435/6; 435/69.1; 435/199; 435/320.1; 435/325; 536/23.2

(57) ABSTRACT

Murine cDNA encoding the alpha 1 subunit of soluble guanylyl cyclase (sGC) and additional sequence to the known 3′ noncoding part of beta1 subunit of soluble guanylyl cyclase are identified herein. The new genes are further used for expression of encoded proteins. The new part of the beta1 cDNA sequences is further used for screening of regulatory factors associated with modulation of the expression of the beta1 sGC subunit.

The present application claims the benefit of U.S. Provisional Application Serial No. 60/233,500 filed on Sept. 19, 2000, the entire text of which is herein incorporated by reference.

The government owns rights in the present invention pursuant to grant provided by the John S. Dunn Foundation, the Harold and Leila Y. Mathers Foundation and the University of Texas.

### BACKGROUND OF THE INVENTION

1. Field of the Invention

The present invention relates generally to the fields of genomic characterization. More particularly, it concerns nucleic acids and proteinaceous sequence of soluble guanylyl cyclase, and assays for compounds that affect its function in NO-dependent signal transduction.

2. Description of Related Art

NO-dependent signal transduction is associated with a number of important physiological processes, including smooth muscle relaxation (Huang, 2000), platelet aggregation (Severina, 1998), neurotransmission (Dawson et al., 1998), cellular differentiation (Boss, 1989) and apoptotic cell death (Thippeswamy, 1997; Li et al., 1997; Liu, 1999). Soluble guanylyl cyclase (sGC), a NO-stimulated hemoprotein, which converts guanosine triphosphate to cyclic guanosine monophosphate, is a key element in these processes.

Soluble guanylyl cyclase is a heterodimer consisting of α and β subunits (Kamisaki et al., 1986), which are encoded by separate genes (Nakane and Murad, 1994). A heme prosthetic group is crucial for the stimulation of the enzyme by NO (Garbers, 1979; Ignarro et al. 1982). The enzyme has been purified from various animal tissues (Garbers, 1979; Gerzer et al., 1981; Ohlstein et al., 1982) and corresponding cDNAs were cloned from various vertebrate species, including rat (Nakane et al., 1988; Nakane et al., 1990), human (Giuili et al., 1992), bovine (Koesling et al., 1988; Koesling et al., 1990) and fish (Mikami et al., 1998). At least two isoforms for each subunit of the enzyme have been identified in various species, prompting a recent revision of the nomenclature of sGC subunits (Zabel et al., 1998). Although isoforms for both subunits were detected at the MRNA level in various tissues and were found to have an overlapping tissue distribution, until recently only the α₁/β₁ heterodimeric enzyme has been isolated from native sources. However, Russwurm and co-workers described an additional α₂/β₁heterodimer, which was shown previously to be catalytically active in vitro (Russwurm et al., 1998). Alternatively spliced transcripts for both human α (Riutter et al., 2000) and β (Behrends et al., 2000) subunits have also been reported. mRNA for the human α₁subunit undergoes alternative splicing, resulting in several mRNA species that are N-terminally truncated. The α₂₁subunit, an alternatively spliced variant of α₂, has been detected in several mammalian cell lines and tissues at the MRNA level Behrends et al., 1995), and a β₁cDNA splice variant has been detected in humans (Chhajlani et al., 1991).

Evidence that sGC activity is regulated both at the protein and MRNA levels has begun to emerge. Several groups have reported that such treatments as forskolin, dibutyryl-cAMP or 3-isobutyl-1-methyl xanthine (Papapetropoulos et al., 1995), endotoxin and/or IL-1β (Papapetropoulos et al., 1996), NO-donating compounds (Filippov et al., 1997) and nerve growth factor (Liu et al., 1997) affect the sGC mRNA levels in various cell types. There is also evidence that levels of sGC mRNA expression are subject to developmental regulation (Bloch et al., 1997).

Despite the significant role sGC plays in numerous physiological processes, little is known about the genomic organization of the genes for this enzyme in mammalian species. Recently, the genomic organization for the α₁and β₁subunits of sGC in Medaka fish (Mikami et al., 1999) and the β-subunit of sGC in mosquito (Anopheles gambiae) were reported (Caccone et al., 1999). Tandem genomic organization and evidence of directly coordinated transcription for α₁and β₁subunits in Medaka fish indicate the possibility of a similar mechanism of expression of sGC subunts in all vertebrates. Co-expression of both α and β subunits in transfected cells is required for enzyme activity (Nakane et al., 1990). Furthermore, the α₁and β₁sGC genes have been localized to the same chromosome in rat and human (Giuili et al., 1993). Co-expression of both α₁and β₁ subunits is obligatory for enzyme activity in rat lung (8;13) and human cerebral cortex, cerebellum and lung (Zabel et al., 1998). However, the ratio of expression levels for both subunits varies in a tissue dependent manner (Zabel et al., 1998), indicating that the regulation of expression for these subunits is not tightly coordinated as was indicated for Medic fish.

Despite an increasing interest in genetic aspects of sac regulation, relatively little is known about the genes or the promoter regions of mammalian GCS. There remains a need for nucleic and proteinaceous composition of sGC to identify agents that regulate its function, as well as allow the production of animal models with increased or decreased sGC function.

### SUMMARY OF THE INVENTION

The present invention overcomes the deficiencies of the art by providing nucleic acids encoding the alpha1 subunit of soluble guanylyl cyclase (sGC) and additional sequence to the known 3′ noncoding part of beta1 subunit of soluble guanylyl cyclase. The present invention also provides sGC protein and nucleic acid compositions, screening assays for modulators of sGC expression and activity, and animal models comprising sGC with altered expression or activity.

Unless otherwise specified, as used herein, “sGC” may refer to nucleic acids encoding alpha1 subunit and/or beta1 subunit of sGC, or proteinaceous compositions encoded by such nucleic acids.

The invention first provides an isolated nucleic acid comprising a region having a nucleotide sequence that encodes the polypeptide sequences of SEQ ID NO: 2. In certain embodiments, the region is further defined as having the nucleotide sequence of the nucleotide sequence of SEQ ID NO: 1.

The invention provides an isolated and purified polynucleotide comprising a base sequence that is identical or complementary to a segment of at least 15 contiguous bases of SEQ ID NO: 1. In certain embodiments the polynucleotide hybridizes to a polynucleotide that a polypeptide comprising the amino acid residue sequence of SEQ ID NO: 1 or to the complement of such a sequence. In certain embodiments the polynucleotide comprises a base sequence that is identical or complementary to a segment of at least 20 contiguous bases of SEQ ID NO: 1. In certain embodiments the polynucleotide comprises a base sequence that is identical or complementary to a insegment of at least 25 contiguous bases of SEQ ID NO: 2. In certain embodiments the polynucleotide comprises a base sequence that is identical or complementary to a segment of at least 30 contiguous bases of SEQ ID NO: 1. In certain embodiments the polynucleotide comprises a base sequence that is identical or complementary to a segment of at least 35 contiguous bases of SEQ ID NO: 1. In certain embodiments the polynucleotide comprises a base sequence that is identical or complementary to a segment of at least 50 contiguous bases of SEQ ID NO: 1. In certain embodiments the polynucleotide comprises a base sequence that is identical or complementary to a segment of at least 75 contiguous bases of SEQ ID NO: 1. In certain embodiments the polynucleotide comprises a base sequence that is identical or complementary to a segment of at least 100 contiguous bases of SEQ ID NO: 1. In certain embodiments the polynucleotide comprises a base sequence that is identical or complementary to a segment of at least 150 contiguous bases of SEQ ID NO: 1. In certain embodiments the polynucleotide comprises a base sequence that is identical or complementary to a segment of at least 200 contiguous bases of SEQ ID NO: 1. In certain embodiments the polynucleotide comprises a base sequence that is identical or complementary to all contiguous bases of SEQ ID NO: 1.

The invention provides an expression vector comprising a polynucleotide that encodes a polypeptide comprising an amino acid residue sequence of SEQ ID NO: 2. In certain embodiments the polynucleotide comprises the nucleotide base sequence of SEQ ID NO: 1. In certain embodiments the polynucleotide is operatively linked to an enhancer-promoter.

The invention provides a recombinant host cell comprising a polynucleotide that encodes a polypeptide comprising an amino acid residue sequence of SEQ ID NO: 2. In certain embodiments the host cell comprising a polynucleotide that encodes a polypeptide comprising the amino acid residue sequence of SEQ ID NO: 2. In certain embodiments the polynucleotide comprises the nucleotide base sequence of SEQ ID NO: 1. In certain embodiments the polynucleotide is introduced into the cell by transformation of the cell with a vector comprising the polynucleotide. In certain embodiments the host cell expresses the polynucleotide to produce the polypeptide. In certain embodiments the cell is a PC12 cell, a CHO cell or a COS cell. In certain embodiments the cell is an E. coli cell. In certain embodiments the cell is a yeast cell.

The invention provides a process for preparing a cell expressing a polypeptide comprising the steps of: transfecting a cell with a polynucleotide that encodes a polypeptide comprising an amino acid residue sequence of SEQ ID NO: 2 to produce a transformed host cell; and maintaining the transformed host cell under biological conditions sufficient for expression of the polypeptide in the host cell. In certain embodiments the polynucleotide comprises a region having a nucleotide sequence of SEQ ID NO: 1. In certain embodiments the process is further defined as a process for preparing a cell expressing a polypeptide comprising the amino acid residue sequence of SEQ ID NO: 2. In certain embodiments the process further comprising purifying an expressed polypeptide having the amino acid sequence of SEQ ID NO: 2 from the transformed host cell.

The invention provides an isolated nucleic acid comprising a region having a nucleotide sequence of SEQ ID NO: 3.

The invention provides an isolated and purified polynucleotide comprising a base sequence that is identical or complementary to a segment of at least 15 contiguous bases of SEQ ID NO: 3. In certain embodiments the polynucleotide comprises a base sequence that is identical or complementary to a segment of at least 20 contiguous bases of SEQ ID NO: 3. In certain embodiments the polynucleotide comprises a base sequence that is identical or complementary to a segment of at least 25 contiguous bases of SEQ ID NO: 3. In certain embodiments the polynucleotide comprises a base sequence that is identical or complementary to a segment of at least 30 contiguous bases of SEQ ID NO: 3. In certain embodiments the polynucleotide comprises a base sequence that is identical or complementary to a segment of at least 35 contiguous bases of SEQ ID NO: 3. In certain embodiments the polynucleotide comprises a base sequence that is identical or complementary to a segment of at least 50 contiguous bases of SEQ ID NO: 3. In certain embodiments the polynucleotide comprises a base sequence that is identical or complementary to a segment of at least 75 contiguous bases of SEQ ID NO: 3. In certain embodiments the polynucleotide comprises a base sequence that is identical or complementary to a segment of at least 100 contiguous bases of SEQ ID NO: 3. In certain embodiments the polynucleotide comprises a base sequence that is identical or complementary to a segment of at least 150 contiguous bases of SEQ ID NO: 3. In certain embodiments the polynucleotide comprises a base sequence that is identical or complementary to a segment of at least 200 contiguous bases of SEQ ID NO: 3. In certain embodiments the polynucleotide comprises a base sequence that is identical or complementary to all contiguous bases of SEQ ID NO: 3.

The invention provides an expression vector comprising a polynucleotide that comprises the nucleotide base sequence of SEQ ID NO: 3. In certain embodiments the polynucleotide is operatively linked to an enhancer-promoter.

The invention provides recombinant host cell comprising a polynucleotide that comprises the nucleotide base sequence of SEQ ID NO: 3. In certain embodiments the polynucleotide is introduced into the cell by transformation of the cell with a vector comprising the polynucleotide. In certain embodiments the cell is a PC12 cell, a CHO cell or a COS cell. In certain embodiments the cell is an E. coli cell. In certain embodiments the cell is a yeast cell.

The invention provides an isolated nucleic acid molecule comprising a region having a nucleic acid sequence of SEQ ID NO: 4 or a fragment thereof, the region further defined as encoding a murine alpha1 soluble guanylyl cyclase possessing a genomic organization as shown in Table 6.

The invention provides an isolated nucleic acid molecule comprising a region having a nucleic acid sequence of SEQ ID NO: 5 or a fragment thereof, the region further defined as encoding a murine beta1 soluble guanylyl cyclase possessing a genomic organization as shown in Table 6.

The invention provides an isolated nucleic acid molecule comprising a region having a nucleic acid sequence of SEQ ID NO: 6 or a fragment thereof, the region further defined as encoding a murine alpha1 soluble guanylyl cyclase possessing a genomic organization as shown in Table 6.

The invention provides a method of detecting a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these. In certain embodiments the method is further defined as a method utilizing a hybridization technique. In certain embodiments the method is further defined as a method utilizing an amplification technique. In certain embodiments the method is further defined as a method utilizing Southern hybridization. In certain embodiments the method is further defined as a method utilizing Northern hybridization. In certain embodiments the method is further defined as a method utilizing PCR amplification. In certain embodiments the method is further defined as a method utilizing DNA microarray analysis.

The invention provides a method of analyzing protein-nucleic acid interactions utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.

In certain embodiments the method is further defined as a method of analysis of DNA-protein interactions. In certain embodiments the method is defined as a method of RNA-protein interactions. In certain embodiments the method is further defined as a method of analysis of DNA-protein interactions. In certain embodiments the method is further defined as a method of analysis of RNA-protein interactions.

The invention provides a method of analyzing substance-nucleic acid interactions utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these. In certain embodiments the method is further defined as a method of screening for drugs. In certain embodiments the method is further defined as a method of drug development. In certain embodiments the method is further defined as a diagnostic method.

The invention provides a method of producing a transgenic animal utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.

The invention provides an expression vector comprising a nucleic acid having region having a sequence of, or complementary to, SEQ ID NO: 1, SEQ ID NO: 3, SEQ ID NO: 4, SEQ ID NO: 5, SEQ ID NO: 6, or a portion of any of these. In certain embodiments the polynucleotide is operatively linked to an enhancer-promoter.

The invention provides a recombinant host cell comprising nucleic acid having region having a sequence of, or complementary to, SEQ ID NO: 1, SEQ ID NO: 3, SEQ ID NO: 4, SEQ ID NO: 5, SEQ ID NO: 6, or a portion of any of these. In certain embodiments the polynucleotide comprises the nucleotide base sequence of SEQ ID NO: 1. In certain embodiments the polynucleotide is introduced into the cell by transformation of the cell with a vector comprising the polynucleotide. In certain embodiments the host cell expresses the polynucleotide to produce the polypeptide. In certain embodiments the cell is a PC12 cell, a CHO cell or a COS cell. In certain embodiments the cell is an E. coli cell. In certain embodiments the cell is a yeast cell.

The invention provides a process for preparing a cell expressing a polypeptide comprising the steps of: transfecting a cell with a nucleic acid having region having a sequence of, or complementary to, SEQ ID NO: 1, SEQ ID NO: 3, SEQ ID NO: 4, SEQ ID NO: 5, SEQ ID NO: 6, or a portion of any of these to produce a transformed host cell; and maintaining the transformed host cell under biological conditions sufficient for expression of the polypeptide in the host cell. In certain embodiments the polynucleotide comprises a region having a nucleotide sequence of SEQ ID NO: 1. In certain embodiments the process is further defined as a process for preparing a cell expressing a polypeptide comprising the amino acid residue sequence of SEQ ID NO: 2. In certain embodiments the process further comprises purifying an expressed polypeptide from the transformed host cell. In certain embodiments the process is further defined a process of producing an active enzyme. In certain embodiments the active enzyme is employed in biochemical characterization, studies of drug-enzyme interactions, drug discovery, drug development, and/or design.

The invention provides a method for the detection of genetic and/or inherited and/or acquired human diseases utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.

In certain embodiments the method is further defined as a method for detection of chromosomal abnormalities connected with cancer, hypertension, heart failure, stroke, neurodegenerative diseases, Alzheimers disease, Parkinsons disease, endocrinopathy, an inflammatory disorder, shock, sepsis, abnormal gasrointestinal motility, altered muscle disorder, altered movement disorder, ocular disorder, sensory disorder, or dermatological disorder. In certain embodiments the method is further defined as a method for the detection of point mutations, deletions, and/or insertions. In certain embodiments the method is further defined as a method for the detection of aberrations in splicing.

The invention provides a diagnostic kit for the detection of genetic and/or inherited and/or acquired human diseases utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.

In certain embodiments the kit is further defined as a kit for detection of chromosomal abnormalities connected with cancer, hypertension, heart failure, stroke, neurodegenerative diseases, Alzheimers disease, Parkinsons disease, endocrinopathy, an inflammatory disorder, shock, sepsis, abnormal gasrointestinal motility, altered muscle disorder, altered movement disorder, ocular disorder, sensory disorder, or dermatological disorder. In certain embodiments the kit is further defined as a kit for the detection of point mutations, deletions, and/or insertions. In certain embodiments the kit is further defined as a kit for the detection of aberrations in splicing.

The invention provides a method of treating disease comprising utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these. In certain embodiments the method is further defined as a method of gene therapy. In certain embodiments the method is further defined as a method for treating chromosomal abnormalities connected with cancer, hypertension, heart failure, stroke, neurodegenerative diseases, Alzheimers disease, Parkinsons disease, endocrinopathy, an inflammatory disorder, shock, sepsis, abnormal gasrointestinal motility, altered muscle disorder, altered movement disorder, ocular disorder, sensory disorder, or dermatological disorder.

The invention provides a method of screening for drugs, drug design, and/or drug development comprising utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.

Product sGC.

Product sGC for use as a medicament.

Use of sGC for the manufacture or a medicament for the treatment of disease, including but not limited to cancer, hypertension, heart failure, stroke, neurodegenerative diseases, Alzheimers disease, Parkinsons disease, endocrinopathy, an inflammatory disorder, shock, sepsis, abnormal gasrointestinal motility, altered muscle disorder, altered movement disorder, ocular disorder, sensory disorder, or dermatological disorder.

As used herein, “any range derivable therein” means a range selected from the numbers described in the specification, and “any integer derivable therein” means any integer between such a range.

As used herein the specification, “a” or “an” may mean one or more. As used herein in the claim(s), when used in conjunction with the word “comprising” , the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The cDNA cloning, chromosomal localization and structure of the mouse genes for the α₁and β₁subunits of sGC and a comparative analyses with the human sGC genes, using the Human Genome database (NCBI) is provided herein. Organizational and regulatory sequences for human and mouse alpha1 and beta1 soluble guanylyl cyclase genes were characterized and the chromosomal localization were identified. The regulatory sequences are further used in the screening for the structure and identity of regulatory factors and compounds for the modulation of the expression of sGC genes and in studies of associated physiological or pathological pathways. Hormonal regulation of the MRNA levels for α₁and β₁sGC subunits has been identified. The DNA information on the genomic organization and loci of the genes are further used for the diagnosis of genetic abnormalities (polymorpisms and mutations) in sGC genes and association with a potential predisposition to cardiovascular, neurological, genetic, inherited and other diseases for the purpose of diagnosis and treatment.

I. sGC Nucleic Acids

In one embodiment, the present invention discloses a novel nucleic acid sequence and a novel protein encoded by the nucleic acid that has homology to the sGC family of genes and proteins.

A. Genes and DNA Segments

Important aspects of the present invention concern isolated DNA segments and recombinant vectors encoding sGC proteins, polypeptides or peptides, and the creation and use of recombinant host cells through the application of DNA technology, that express a wild-type, polymorphic or mutant sGC, using the sequence of SEQ ID NO: 1, SEQ ID NO: 3, SEQ ID NO: 4, SEQ ID NO: 5 OR SEQ ID NO: 6, and biologically functional equivalents thereof.

The present invention concerns DNA segments, isolatable from mammalian cells, such as mouse or human cells, that are free from total genomic DNA and that are capable of expressing a protein, polypeptide or peptide that has sGC activity. As used herein, the term “DNA segment” refers to a DNA molecule that has been isolated free of total genomic DNA of a particular species. Therefore, a DNA segment encoding sGC refers to a DNA segment that contains wild-type, polymorphic or mutant sGC coding sequences yet is isolated away from, or purified free from, total mammalian genomic DNA. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified sGC gene refers to a DNA segment including sGC protein, polypeptide or peptide coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and engineered segments that express, or may be adapted to express, proteins, polypeptides, domains, peptides, fusion proteins and mutants of sGC encoded sequences.

“Isolated substantially away from other coding sequences” means that the gene of interest, in this case the sGC gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments and recombinant vectors incorporating DNA sequences that encode a sGC protein, polypeptide or peptide that includes within its amino acid sequence a contiguous amino acid sequence in accordance with, or essentially as set forth in, SEQ ID NO: 2, corresponding to the sGC designated “murine sGC”.

The term “a sequence essentially as set forth in SEQ ID NO: 2” means that the sequence substantially corresponds to a portion of SEQ ID NO: 2 and has relatively few amino acids that are not identical to, or a biologically functional equivalent of, the amino acids of SEQ ID NO: 2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO: 2 will be sequences that are “essentially as set forth in SEQ ID NO: 2” , provided the biological activity of the protein is maintained. In particular embodiments, the biological activity of a sGC protein, polypeptide or peptide, or a biologically functional equivalent, comprises binding to one or more proteases, particularly sGC.

In certain other embodiments, the invention concerns isolated DNA segments and recombinant vectors that include within their sequence a nucleic acid sequence essentially as set forth in SEQ ID NO: 1, SEQ ID NO: 3, SEQ ID NO: 4, SEQ ID NO: 5 OR SEQ ID NO: 6. The term “essentially as set forth in SEQ ID NO: 1, SEQ ID NO: 3, SEQ ID NO: 4, SEQ ID NO: 5 OR SEQ ID NO: 6” is used in the same sense as described above and means that the nucleic acid sequence substantially corresponds to a portion of SEQ ID NO: 1, SEQ ID NO: 3, SEQ ID NO: 4, SEQ ID NO: 5 OR SEQ ID NO: 6 and has relatively few codons that are not identical, or functionally equivalent, to the codons of SEQ ID NO: 1, SEQ ID NO: 3, SEQ ID NO: 4, SEQ ID NO: 5 OR SEQ ID NO: 6. Again, DNA segments that encode proteins, polypeptide or peptides exhibiting sGC activity will be most preferred.

The term “functionally equivalent codon” is used herein to refer to codons that encode the same amino acid, such as the six codons for arginine and serine, and also refers to codons that encode biologically equivalent amino acids. For optimization of expression of sGC in human cells, the codons are shown in Table 1 in preference of use from left to right. Thus, the most preferred codon for alanine is thus “GCC”, and the least is “GCG” (see Table 1 below). Codon usage for various organisms and organelles can be found at the website http://www.kazusa.orjp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria or chloroplasts, based on the preferred codon usage as would be known to those of ordinary skill in the art.

TABLE 1
Preferred Human DNA Codons

| | |
|---|---|
| Amino Acids | Codons |

| | | |
|---|---|---|
| Alanine | Ala A | GCC GCT GCA GCG |
| Cysteine | Cys C | TGC TGT |
| Aspartic acid | Asp D | GAC GAT |
| Glutamic acid | Glu E | GAG GAA |
| Phenylalanine | Phe F | TTC TTT |
| Glycine | Gly G | GGC GGG GGA GGT |
| Histidine | His H | CAC CAT |
| Isoleucine | Ile I | ATC ATT ATA |
| Lysine | Lys K | AAG AAA |
| Leucine | Leu L | CTG CTC TTG CTT CTA TTA |
| Methionine | Met M | ATG |
| Asparagine | Asn N | AAC AAT |
| Proline | Pro P | CCC CCT CCA CCG |
| Glutamine | Gln Q | CAG CAA |
| Arginine | Arg R | CGC AGG CGG AGA CGA CGT |
| Serine | Ser S | AGC TCC TCT AGT TCA TCG |
| Threonine | Thr T | ACC ACA ACT ACG |
| Valine | Val V | GTG GTC GTT GTA |
| Tryptophan | Trp W | TGG |
| Tyrosine | Tyr Y | TAC TAT |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

Excepting intronic or flanking regions, and allowing for the degeneracy of the genetic code, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of nucleotides that are identical to the nucleotides of SEQ ID NO: 1, SEQ ID NO: 3, SEQ ID NO: 4, SEQ ID NO: 5 OR SEQ ID NO: 6 will be sequences that are “essentially as set forth in SEQ ID NO: 1, SEQ ID NO: 3, SEQ ID NO: 4, SEQ ID NO: 5 OR SEQ ID NO: 6”.

B. Nucleic Acid Hybridization

The nucleic acid sequences disclosed herein also have a variety of uses, such as for example, utility as probes or primers in nucleic acid hybridization embodiments.

Naturally, the present invention also encompasses DNA segments that are complementary, or essentially complementary, to the sequence set forth in SEQ ID NO: 1, SEQ ID NO: 3, SEQ ID NO: 4, SEQ ID NO: 5 OR SEQ ID NO: 6. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO: 1, SEQ ID NO: 3, SEQ ID NO: 4, SEQ ID NO: 5 OR SEQ ID NO: 6 under stringent conditions such as those described herein.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide base content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Accordingly, the nucleotide sequences of the disclosure may be used for their ability to selectively form duplex molecules with complementary stretches of genes or RNAs or to provide primers for amplification of DNA or RNA from tissues. Depending on the application envisioned, it is preferred to employ varying conditions of hybridization to achieve varying degrees of selectivity of probe towards target sequence.

The nucleic acid segments of the present invention, regardless of the length of the coding sequence itself, may be combined with other DNA sequences, such as promoters, enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

For example, nucleic acid fragments may be prepared that include a contiguous stretch of nucleotides identical to or complementary to SEQ ID NO: 1, SEQ ID NO: 3, SEQ ID NO: 4, SEQ ID NO: 5 OR SEQ ID NO: 6, such as, for example, about 8, about 10 to about 14, or about 15 to about 20 nucleotides, and that are chromosome sized pieces, up to about 1,000,000, about 750,000, about 500,000, about 250,000, about 100,000, about 50,000, about 20,000, or about 10,000, or about 5,000 base pairs in length, with segments of about 3,000 being preferred in certain cases, as well as DNA segments with total lengths of about 1,000, about 500, about 200, about 100 and about 50 base pairs in length (including all intermediate lengths of these lengths listed above, i.e., any range derivable therein and any integer derivable therein such a range) are also contemplated to be useful.

For example, it will be readily understood that “intermediate lengths”, in these contexts, means any length between the quoted ranges, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 130, 140, 150, 160, 170, 180, 190, including all integers through the 200-500; 500-1,000; 1,000-2,000; 2,000-3,000; 3,000-5,000; 5,000-10,000 ranges, up to and including sequences of about 12,001, 12,002, 13,001, 13,002, 15,000, 20,000 and the like.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and/or so on. For a 15-mer, the nucleic acid segments correspond to bases 1 to 15, 2 to 16, 3 to 17 . . . and/or so on. For a 20-mer, the nucleic segments correspond to bases 1 to 20, 2 to 21, 3 to 22 . . . and/or so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

The use of a hybridization probe of between 17 and 100 nucleotides in length, or in some aspect of the invention even up to 1-2 Kb or more in length, allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained. One will generally prefer to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

In general, it is envisioned that the hybridization probes described herein will be useful both as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface to remove non-specifically bound probe molecules, hybridization is detected, or even quantified, by means of the label.

C. Nucleic Acid Amplification

Nucleic acid used as a template for amplification is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to sGC genes are contacted with the isolated nucleic acid under conditions that permit selective hybridization. The term “primer”, as defined herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology).

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, each incorporated herein by reference in entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of MRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641, filed Dec. 21, 1990, incorporated herein by reference. Polymerase chain reaction methodologies are well known in the art.

Another method for amplification is the ligase chain reaction (“LCR”), disclosed in EPA No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, incorporated herein by reference, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Gingeras et al., PCT Application WO 88/10315, incorporated herein by reference). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPA No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990, incorporated herein by reference).

Methods based on ligation of two (or more) oligonucleotides in the presence of nucleic acid having the sequence of the resulting “di-oligonucleotide”, thereby amplifying the di-oligonucleotide, may also be used in the amplification step of the present invention.

D. Nucleic Acid Detection

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means visible to the human eye or spectrophotometrically, to identify specific hybridization with complementary nucleic acid-containing samples.

In embodiments wherein nucleic acids are amplified, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (Sambrook et al., 1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography.

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by Southern blotting and hybridization with a labeled probe. The techniques involved in Southern blotting are well known to those of skill in the art and can be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods for genetic screening to accurately detect mutations in genomic DNA, cDNA or RNA samples may be employed, depending on the specific situation.

Historically, a number of different methods have been used to detect point mutations, including denaturing gradient gel electrophoresis (“DGGE”), restriction enzyme polymorphism analysis, chemical and enzymatic cleavage methods, and others. The more common procedures currently in use include direct sequencing of target regions amplified by PCR™ (see above) and single-strand conformation polymorphism analysis (“SSCP”).

Another method of screening for point mutations is based on RNase cleavage of base pair mismatches in RNA/DNA and RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single and multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. After the RNase cleavage reaction, the RNase is inactivated by proteolytic digestion and organic extraction, and the cleavage products are denatured by heating and analyzed by electrophoresis on denaturing polyacrylamide gels. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Currently available RNase mismatch cleavage assays, including those performed according to U.S. Pat. No. 4,946,773, require the use of radiolabeled RNA probes. Myers and Maniatis in U.S. Pat. No. 4,946,773 describe the detection of base pair mismatches using RNase A. Other investigators have described the use of an E. coli enzyme, RNase I, in mismatch assays. Because it has broader cleavage specificity than RNase A, RNase I would be a desirable enzyme to employ in the detection of base pair mismatches if components can be found to decrease the extent of non-specific cleavage and increase the frequency of cleavage of mismatches. The use of RNase I for mismatch detection is described in literature from Promega Biotech. Promega markets a kit containing RNase I that is shown in their literature to cleave three out of four known mismatches, provided the enzyme level is sufficiently high.

The RNase protection assay was first used to detect and map the ends of specific mRNA targets in solution. The assay relies on being able to easily generate high specific activity radiolabeled RNA probes complementary to the MRNA of interest by in vitro transcription. Originally, the templates for in vitro transcription were recombinant plasmids containing bacteriophage promoters. The probes are mixed with total cellular RNA samples to permit hybridization to their complementary targets, then the mixture is treated with RNase to degrade excess unhybridized probe. Also, as originally intended, the RNase used is specific for single-stranded RNA, so that hybridized double-stranded probe is protected from degradation. After inactivation and removal of the RNase, the protected probe (which is proportional in amount to the amount of target MRNA that was present) is recovered and analyzed on a polyacrylamide gel.

The RNase Protection assay was adapted for detection of single base mutations. In this type of RNase A mismatch cleavage assay, radiolabeled RNA probes transcribed in vitro from wild-type sequences, are hybridized to complementary target regions derived from test samples. The test target generally comprises DNA (either genomic DNA or DNA amplified by cloning in plasmids or by PCR™), although RNA targets (endogenous mRNA) have occasionally been used. If single nucleotide (or greater) sequence differences occur between the hybridized probe and target, the resulting disruption in Watson-Crick hydrogen bonding at that position (“mismatch”) can be recognized and cleaved in some cases by single-strand specific ribonuclease. To date, RNase A has been used almost exclusively for cleavage of single-base mismatches, although RNase I has recently been shown as useful also for mismatch cleavage. There are recent descriptions of using the MutS protein and other DNA-repair enzymes for detection of single-base mismatches.

E. Cloning sGC Genes

The present invention contemplates cloning sGC genes or cDNAs from animal (e.g., mammalian) organisms. A technique often employed by those skilled in the art of protein production today is to obtain a so-called “recombinant” version of the protein, to express it in a recombinant cell and to obtain the protein, polypeptide or peptide from such cells. These techniques are based upon the “cloning” of a DNA molecule encoding the protein from a DNA library, i.e., on obtaining a specific DNA molecule distinct from other portions of DNA. This can be achieved by, for example, cloning a cDNA molecule, or cloning a genomic-like DNA molecule.

The first step in such cloning procedures is the screening of an appropriate DNA library, such as, for example, from a mouse, rat, monkey or human. The screening protocol may utilize nucleotide segments or probes that are designed to hybridize to cDNA or genomic sequences of sGCs. Additionally, antibodies designed to bind to the expressed sGC proteins, polypeptides, or peptides may be used as probes to screen an appropriate mammalian DNA expression library. Alternatively, activity assays may be employed. The operation of such screening protocols are well known to those of skill in the art and are described in detail in the scientific literature, for example, in Sambrook et al. (1989), incorporated herein by reference. Moreover, as the present invention encompasses the cloning of genomic segments as well as CDNA molecules, it is contemplated that suitable genomic cloning methods, as known to those in the art, may also be used.

As used herein “designed to hybridize” means a sequence selected for its likely ability to hybridize to a mammalian sGC gene, for example due to the expected high degree of homology between the human sGC gene and the sGC genes from other mammals. Also included are segments or probes altered to enhance their ability to hybridize to or bind to a mammalian sGC gene. Additionally, these regions of homology also include amino acid sequences of 4 or more consecutive amino acids selected and/or altered to increase conservation of the amino acid sequences in comparison to the same or similar region of residues in the same or related genes in one or more species. Such amino acid sequences may derived from amino acid sequences encoded by the sGC gene, and more particularly from the isolated sequences of SEQ ID NO: 2.

General methods for screening a mammalian DNA library are exemplified by, but not limited to, the methods detailed in Example 1 herein below. Nucleotide probes may derived from nucleotide sequences from the human sGC sequence, and more particularly from the isolated sequences of SEQ ID NO: 1, SEQ ID NO: 3, SEQ ID NO: 4, SEQ ID NO: 5 OR SEQ ID NO: 6. Such sequences may be used as probes for hybridization or oligonucleotide primers for PCR™. Designing such sequences may involve selection of regions of highly conserved nucleotide sequences between various species for a particular gene or related genes, relative to the general conservation of nucleotides of the gene or related genes in one or more species. Comparison of the amino acid sequences conserved between one or more species for a particular gene may also be used to determine a group of 4 or more consecutive amino acids that are conserved relative to the protein encoded by the gene or related genes. The nucleotide probe or primers may then be designed from the region of the gene that encodes the conserved sequence of amino acids.

One may also prepare fusion proteins, polypeptides and peptides, e.g., where the sGC proteinaceous material coding regions are aligned within the same expression unit with other proteins, polypeptides or peptides having desired functions, such as for purification or immunodetection purposes (e.g., proteinaceous compostions that may be purified by affinity chromatography and enzyme label coding regions, respectively).

Encompassed by the invention are DNA segments encoding relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and more preferably, of from about 15 to about 30 amino acids in length; as set forth in SEQ ID NO: 2 and also larger polypeptides up to and including proteins corresponding to the full-length sequences set forth in SEQ ID NO: 2, and any range derivable therein and any integer derivable therein such a range.

In addition to the “standard” DNA and RNA nucleotide bases, modified bases are also contemplated for use in particular applications of the present invention. A table of exemplary, but not limiting, modified bases is provided herein below.

TABLE 2
Purine and Pyrmidine Derivatives or Analogs

| | Modified base | | Modified base |
|---|---|---|---|
| Abbr. | description | Abbr. | description |
| ac4c | 4-acetylcytidine | Mam5s2u | 5-methoxyaminomethyl- |
| | | | 2-thiouridine |
| chm5u | 5-(carboxyhydroxyl- | Man q | Beta,D- |
| | methyl)uridine | | mannosylqueosine |
| Cm | 2′-O-methylcytidine | Mcm5s2u | 5-methoxycarbonyl- |
| | | | methyl-2-thiouridine |
| Cmnm5s2u | 5-carboxymethyl- | Mcm5u | 5-methoxycarbonyl- |
| | aminomethyl-2-thio- | | methyluridine |
| | ridine | | |
| Cmnm5u | 5-carboxymethyl- | Mo5u | 5-methoxyuridine |
| | aminomethyluridine | | |
| D | Dihydrouridine | Ms2i6a | 2-methylthio-N6- |
| | | | isopentenyladenosine |
| Fm | 2′-O-methylpseudo- | Ms2t6a | N-((9-beta-D- |
| | uridine | | ribofuranosyl-2- |
| | | | methylthiopurine-6- |
| | | | yl)carbamoyl)threonine |
| gal q | beta,D-galac- | Mt6a | N-((9-beta-D- |
| | tosylqueosine | | ribofuranosylpurine-6- |
| | | | yl)N-methyl- |
| | | | carbamoyl)threonine |
| Gm | 2′-O-methyl- | Mv | Uridine-5-oxyacetic acid |
| | guanosine | | methylester |
| I | Inosine | o5u | Uridine-5-oxyacetic acid |
| | | | (v) |
| I6a | N6-isopentenyl- | Osyw | Wybutoxosine |
| | adenosine | | |
| m1a | 1-methyladenosine | P | Pseudouridine |
| m1f | 1-methylpseudo- | Q | Queosine |
| | uridine | | |
| m1g | 1-methylguanosine | s2c | 2-thiocytidine |
| m1I | 1-methylinosine | s2t | 5-methyl-2-thiouridine |
| m22g | 2,2-dimethyl- | s2u | 2-thiouridine |
| | guanosine | | |
| m2a | 2-methyladenosine | s4u | 4-thiouridine |
| m2g | 2-methylguanosine | T | 5-methyluridine |
| m3c | 3-methylcytidine | t6a | N-((9-beta-D- |
| | | | ribofuranosylpurine-6- |
| | | | yl)carbamoyl)threonine |
| m5c | 5-methylcytidine | Tm | 2′-O-methyl-5-methyl- |
| | | | uridine |
| m6a | N6-methyladenosine | Um | 2′-O-methyluridine |
| m7g | 7-methylguanosine | Yw | Wybutosine |
| Mam5u | 5-methylamino- | X | 3-(3-amino-3- |
| | methyluridine | | carboxypropyl)uridine, |
| | | | (acp3)u |

II. Mutagenesis, Peptidomimetics and Rational Drug Design

It will also be understood that this invention is not limited to the particular nucleic acid and amino acid sequences of SEQ ID NO: 1, SEQ ID NO: 2, SEQ ID NO: 3, SEQ ID 5 NO: 4, SEQ ID NO: 5 OR SEQ ID NO: 6. Recombinant vectors and isolated DNA segments may therefore variously include these coding regions themselves, coding regions bearing selected alterations or modifications in the basic coding region, or they may encode larger polypeptides that nevertheless include such coding regions or may encode biologically functional equivalent proteins, polypeptides or peptides that have variant amino acids sequences.

The DNA segments of the present invention encompass biologically functional equivalent sGC proteins, polypeptides, and peptides. Such sequences may arise as a consequence of codon redundancy and functional equivalency that are known to occur naturally within nucleic acid sequences and the proteinaceous compositions thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements to the antigenicity of the proteinaceous composition or to test mutants in order to examine sGC activity at the molecular level.

Site-specific mutagenesis is a technique useful in the preparation of individual peptides, or biologically functional equivalent proteins, polypeptides or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired proteinaceous molecule. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

As modifications and changes may be made in the structure of the sGC genes, nucleic acids (e.g., nucleic acid segments) and proteinaceous molecules of the present invention, and still obtain molecules having like or otherwise desirable characteristics, such biologically functional equivalents are also encompassed within the present invention.

For example, certain amino acids may be substituted for other amino acids in a proteinaceous structure without appreciable loss of interactive binding capacity with structures such as, for example, antigen-binding regions of antibodies, binding sites on substrate molecules or receptors, or such like. Since it is the interactive capacity and nature of a proteinaceous molecule that defines that proteinaceous molecule's biological functional activity, certain amino acid sequence substitutions can be made in a proteinaceous molecule sequence (or, of course, its underlying DNA coding sequence) and nevertheless obtain a proteinaceous molecule with like (agonistic) properties. It is thus contemplated that various changes may be made in the sequence of sGC proteins, polypeptides or peptides, or the underlying nucleic acids, without appreciable loss of their biological utility or activity.

Equally, the same considerations may be employed to create a protein, polypeptide or peptide with countervailing, e.g., antagonistic properties. This is relevant to the present invention in which sGC mutants or analogues may be generated. For example, a sGC mutant may be generated and tested for sGC activity to identify those residues important for sGC activity. sGC mutants may also be synthesized to reflect a sGC mutant that occurs in the human population and that is linked to the development of cancer. Such mutant proteinaceous molecules are particularly contemplated for use in generating mutant-specific antibodies and such mutant DNA segments may be used as mutant-specific probes and primers.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. A table of amino acids and their codons is presented herein above for use in such embodiments, as well as for other uses, such as in the design of probes and primers and the like.

In terms of functional equivalents, it is well understood by the skilled artisan that, inherent in the definition of a “biologically functional equivalent” protein, polypeptide, peptide, gene or nucleic acid, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted.

In particular, where shorter length peptides are concerned, it is contemplated that fewer amino acids changes should be made within the given peptide. Longer domains may have an intermediate number of changes. The full length protein will have the most tolerance for a larger number of changes. Of course, a plurality of distinct proteins/polypeptide/peptides with different substitutions may easily be made and used in accordance with the invention.

It is also well understood that where certain residues are shown to be particularly important to the biological or structural properties of a protein, polypeptide or peptide, e.g., residues in binding regions or active sites, such residues may not generally be exchanged. In this manner, functional equivalents are defined herein as those peptides which maintain a substantial amount of their native biological activity.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a proteinaceous molecule is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biological functional equivalent protein, polypeptide or peptide thereby created is intended for use in immunological embodiments, as in certain embodiments of the present invention. U.S. Pat. No. 4,554,101, incorporated herein by reference, states that the greatest local average hydrophilicity of a proteinaceous molecule, as governed by the hydrophilicity of its adjacent amino acids, correlates with its immunogenicity and antigenicity, i.e., with a biological property of the proteinaceous molecule.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within±1 are particularly preferred, and those within ±0.5 are even more particularly preferred.

In addition to the sGC peptidyl compounds described herein, it is contemplated that other sterically similar compounds may be formulated to mimic the key portions of the peptide structure. Such compounds, which may be termed peptidomimetics, may be used in the same manner as the peptides of the invention and hence are also functional equivalents.

Certain mimetics that mimic elements of proteinaceous molecule's secondary structure are described in Johnson et al. (1993). The underlying rationale behind the use of peptide mimetics is that the peptide backbone of proteinaceous molecules exists chiefly to orientate amino acid side chains in such a way as to facilitate molecular interactions, such as those of antibody and antigen. A peptide mimetic is thus designed to permit molecular interactions similar to the natural molecule.

Some successful applications of the peptide mimetic concept have focused on mimetics of β-turns within proteinaceous molecules, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

The generation of further structural equivalents or mimetics may be achieved by the techniques of modeling and chemical design known to those of skill in the art. The art of receptor modeling is now well known, and by such methods a chemical that binds sGC can be designed and then synthesized. It will be understood that all such sterically designed constructs fall within the scope of the present invention.

In addition to the 20 “standard” amino acids provided through the genetic code, modified or unusual amino acids are also contemplated for use in the present invention. A table of exemplary, but not limiting, modified or unusual amino acids is provided herein below.

TABLE 3
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | Beta-alanine, beta-Amino- | aHyl | Allo-Hydroxylysine |
| | propionic acid | | |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | aIle | Allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In one aspect, an compound may be designed by rational drug design to function as a sGC in inhibition of sGC. The goal of rational drug design is to produce structural analogs of biologically active compounds. By creating such analogs, it is possible to fashion drugs which are more active or stable than the natural molecules, which have different susceptibility to alteration or which may affect the function of various other molecules. In one approach, one would generate a three-dimensional structure for the sGC protein of the invention or a fragment thereof. This could be accomplished by X-ray crystallography, computer modeling or by a combination of both approaches. An alternative approach, involves the random replacement of functional groups throughout the sGC protein, polypeptides or peptides, and the resulting affect on function determined.

It also is possible to isolate a sGC protein, polypeptide or peptide specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have enhanced and improved, or reduced, biological activity, for example, NO-dependent signal transduction, relative to a starting sGC proteinaceous sequences. By virtue of the ability to recombinatly produce sufficient amounts of the sGC proteins, polypeptides or peptides, crystallographic studies may be preformed to determine the most likely sites for mutagenesis and chemical mimicry. In addition, knowledge of the chemical characteristics of these compounds permits computer employed predictions of structure-function relationships. Computer models of various polypeptide and peptide structures are also available in the literature or computer databases. In a non-limiting example, the Entrez database (http://www.ncbi.nlm.nih.gov/Entrez/) may be used by one of ordinary skill in the art to identify target sequences and regions for mutagenesis.

III. Recombinant Vectors, Host Cells and Expression

Recombinant vectors form an important further aspect of the present invention. The term “expression vector or construct” means any type of genetic construct containing a nucleic acid coding for a gene product in which part or all of the nucleic acid encoding sequence is capable of being transcribed. The transcript may be translated into a proteinaceous molecule, but it need not be. Thus, in certain embodiments, expression includes both transcription of a gene and translation of a RNA into a gene product. In other embodiments, expression only includes transcription of the nucleic acid, for example, to generate antisense constructs.

Particularly useful vectors are contemplated to be those vectors in which the coding portion of the DNA segment, whether encoding a full length protein or smaller polypeptide or peptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned”, “under control” or “under transcriptional control” means that the promoter is in the correct location and orientation in relation to the nucleic acid to control RNA polymerase initiation and expression of the gene.

The promoter may be in the form of the promoter that is naturally associated with an sGC gene, as may be obtained by isolating the 5′ non-coding sequences located upstream of the coding segment or exon, for example, using recombinant cloning and/or PCR technology, in connection with the compositions disclosed herein (PCR™ technology is disclosed in U.S. Pat. No. 4,683,202 and U.S. Pat. No. 4,682,195, each incorporated herein by reference).

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with an sGC gene in its natural environment. Such promoters may include promoters normally associated with other genes, and/or promoters isolated from any other bacterial, viral, eukaryotic, protist, or mammalian cell, and/or promoters made by the hand of man that are not “naturally occurring”, i.e., containing difference elements from different promoters, or mutations that increase, decrease, or alter expression.

Naturally, it will be important to employ a promoter that effectively directs the expression of the DNA segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high level expression of the introduced DNA segment, such as is advantageous in the large-scale production of recombinant proteins, polypeptides or peptides.

At least one module in a promoter generally functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase promoter, the spacing between promoter elements can be increased to 50 basepairs apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various other embodiments, the human cytomegalovirus (CMV) immediate early gene promoter, the SV40 early promoter and the Rous sarcoma virus long terminal repeat can be used to obtain high-level expression of the instant nucleic acids. The use of other viral or mammalian cellular or bacterial phage promoters which are well-known in the art to achieve expression are contemplated as well, provided that the levels of expression are sufficient for a given purpose. Tables 4 and 5 below list several elements/promoters which may be employed, in the context of the present invention, to regulate the expression of an sGC gene. This list is not intended to be exhaustive of all the possible elements involved in the promotion of expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 4
Promoter and Enhancer Elements

| Promoter/Enhancer | References |
|---|---|
| Immunoglobulin Heavy Chain | Banerji et al., 1983; Gilles et al., |
| | 1983; Grosschedl and Baltimore, |
| | 1985; Atchinson and Perry, 1986, |
| | 1987; Imler et al., 1987; Weinberger |
| | et al., 1984; Kiledjian et al., 1988; |
| | Porton et al.; 1990 |
| Immunoglobulin Light Chain | Queen and Baltimore, 1983; |
| | Picard and Schaffner, 1984 |
| T-Cell Receptor | Luria et al., 1987; Winoto and |
| | Baltimore, 1989; Redondo et al.; |
| | 1990 |
| HLA DQ a and DQ β | Sullivan and Peterlin, 1987 |
| β-Interferon | Goodbourn et al., 1986; Fujita et al., |
| | 1987; Goodbourn and Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; |
| | 1989 |
| Muscle Creatine Kinase | Jaynes et al., 1988; Horlick and |
| | Benfield, 1989; Johnson et al., 1989 |
| Prealbumin (Transthyretin) | Costa et al., 1988 |
| Elastase I | Ornitz et al., 1987 |
| Metallothionein | Karin et al., 1987; Culotta and |
| | Hamer, 1989 |
| Collagenase | Pinkert et al., 1987; Angel et al., |
| | 1987 |
| Albumin Gene | Pinkert et al., 1987; Tronche et al., |
| | 1989, 1990 |
| α-Fetoprotein | Godbout et al., 1988; Campere and |
| | Tilghman, 1989 |
| t-Globin | Bodine and Ley, 1987; Perez- |
| | Stable and Constantini, 1990 |
| β-Globin | Trudel and Constantini, 1987 |
| e-fos | |
| c-HA-ras | Deschamps et al., 1985 |
| Insulin | Edlund et al., 1985 |
| Neural Cell Adhesion Molecule | Hirsh et al., 1990 |
| (NCAM) | |
| α_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., |
| | 1981; Sleigh and Lockett, 1985; |
| | Firak and Subramanian, 1986; |
| | Herr and Clarke, 1986; Imbra and |
| | Karin, 1986; Kadesch and Berg, |
| | 1986; Wang and Calame, 1986; |
| | Ondek et al., 1987; Kuhl et al., |
| | 1987; Schaffner et al., 1988 |
| Polyoma | Swartzendruber and Lehman, 1975; |
| | Vasseur et al., 1980; Katinka et al., |
| | 1980, 1981; Tyndell et al., 1981; |
| | Dandolo et al., 1983; de Villiers et |
| | al., 1984; Hen et al., 1986; Satake et |
| | al., 1988; Campbell and Villarreal, |
| | 1988 |
| Retroviruses | Kriegler and Botchan, 1982, 1983; |
| | Levinson et al., 1982; Kriegler et |
| | al., 1983, 1984a, b, 1988; Bosze et |
| | al., 1986; Miksicek et al., 1986; |
| | Celander and Haseltine, 1987; |
| | Thiesen et al., 1988; Celander et al., |
| | 1988; Choi et al., 1988; Reisman and |
| | Rotter, 1989 |
| Papilloma Virus | Campo et al., 1983; Lusky et al., |
| | 1983; Spandidos and Wilkie, 1983; |
| | Spalholz et al., 1985; Lusky and |
| | Botchan, 1986; Cripe et al., 1987; |
| | Gloss et al., 1987; Hirochika et al., |
| | 1987; Stephens and Hentschel, 1987 |
| Hepatitis B Virus | Bulla and Siddiqui, 1986; Jameel and |
| | Siddiqui, 1986; Shaul and Ben-Levy, |
| | 1987; Spandau and Lee, 1988; |
| | Vannice and Levinson, 1988 |
| Human Immunodeficiency Virus | Muesing et al., 1987; Hauber and |
| | Cullan, 1988; Jakobovits et al., |
| | 1988; Feng and Holland, 1988; |
| | Takebe et al., 1988; Rosen et al., |
| | 1988; Berkhout et al., 1989; Laspia |
| | et al., 1989; Sharp and Marciniak, |
| | 1989; Braddock et al., 1989 |
| Cytomegalovirus | Weber et al., 1984; Boshart et al., |
| | 1985; Foecking and Hofstetter, 1986 |
| Gibbon Ape Leukemia Virus | Holbrook et al., 1987; Quinn et al., |
| | 1989 |

TABLE 5
Inducible Elements

| Element | Inducer | References |
|---|---|---|
| MT II | Phorbol Ester (TFA) | Palmiter et al., 1982; |
| | Heavy metals | Haslinger and Karin, 1985; |
| | | Searle et al., 1985; |
| | | Stuart et al., 1985; |
| | | Imagawa et al., 1987, |
| | | Karin et al., 1987; |
| | | Angel et al., 1987b; |
| | | McNeall et al., 1989 |
| MMTV (mouse | Glucocorticoids | Huang et al., 1981; |
| mammary tumor | | Lee et al., 1981; Majors and |
| virus) | | Varmus, 1983; |
| | | Chandler et al., 1983; |
| | | Lee et al., 1984; |
| | | Ponta et al., 1985; |
| | | Sakai et al., 1988 |
| β-Interferon | Poly(rI)x | Tavernier et al., 1983 |
| | Poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | |
| | Newcastle | |
| | Disease Virus | |
| GRP78 Gene | A23187 | Resendez et al., 1988 |
| α-2-Macroglobu- | IL-6 | Kunz et al., 1989 |
| lin | | |
| Vimentin | Serum | Rittling et al., 1989 |
| MHC Class I | Interferon | Blanar et al., 1989 |
| Gene H-2κb | | |
| HSP70 | Ela, SV40 Large T | Taylor et al., 1989; Taylor and |
| | Antigen | Kingston, 1990a, b |
| Proliferin | Phorbol Ester-TPA | Mordacq and Linzer, 1989 |
| Tumor Necrosis | FMA | Hensel et al., 1989 |
| Factor | | |
| Thyroid Stimu- | Thyroid Hormone | Chatterjee et al., 1989 |
| lating Hormone a | | |
| Gene | | |

Turning to the expression of the sGC proteinaceous molecules of the present invention, once a suitable clone or clones have been obtained, whether they be cDNA based or genomic, one may proceed to prepare an expression system. The engineering of DNA segment(s) for expression in a prokaryotic or eukaryotic system may be performed by techniques generally known to those of skill in recombinant expression. It is believed that virtually any expression system may be employed in the expression of the proteinaceous molecules of the present invention.

Both cDNA and genomic sequences are suitable for eukaryotic expression, as the host cell will generally process the genomic transcripts to yield functional mRNA for translation into proteinaceous molecules. Generally speaking, it may be more convenient to employ as the recombinant gene a cDNA version of the gene. It is believed that the use of a CDNA version will provide advantages in that the size of the gene will generally be much smaller and more readily employed to transfect the targeted cell than will a genomic gene, which will typically be up to an order of magnitude or more larger than the cDNA gene. However, it is contemplated that a genomic version of a particular gene may be employed where desired.

In expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

The term “antisense nucleic acid” is intended to refer to the oligonucleotides complementary to the base sequences of DNA and RNA. Antisense oligonucleotides, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

Antisense constructs may be designed to bind to the promoter and other control regions, exons, introns or even exon-intron boundaries of a gene. Antisense RNA constructs, or DNA encoding such antisense RNAs, may be employed to inhibit gene transcription or translation or both within a host cell, either in vitro or in vivo, such as within a host animal, including a human subject. Nucleic acid sequences comprising “complementary nucleotides” are those which are capable of base-pairing according to the standard Watson-Crick complementary rules. That is, that the larger purines will base pair with the smaller pyrimidines to form only combinations of guanine paired with cytosine (G:C) and adenine paired with either thymine (A:T), in the case of DNA, or adenine paired with uracil (A:U) in the case of RNA.

As used herein, the terms “complementary” or “antisense sequences” mean nucleic acid sequences that are substantially complementary over their entire length and have very few base mismatches. For example, nucleic acid sequences of fifteen bases in length may be termed complementary when they have a complementary nucleotide at thirteen or fourteen positions with only single or double mismatches. Naturally, nucleic acid sequences which are “completely complementary” will be nucleic acid sequences which are entirely complementary throughout their entire length and have no base mismatches.

While all or part of the gene sequence may be employed in the context of antisense construction, statistically, any sequence 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting of the corresponding host cell gene simply by testing the constructs in vitro to determine whether the endogenous gene's function is affected or whether the expression of related genes having complementary sequences is affected.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

As an alternative to targeted antisense delivery, targeted ribozymes may be used. The term “ribozyme” refers to an RNA-based enzyme capable of targeting and cleaving particular base sequences in oncogene DNA and RNA. Ribozymes either can be targeted directly to cells, in the form of RNA oligo-nucleotides incorporating ribozyme sequences, or introduced into the cell as an expression construct encoding the desired ribozymal RNA. Ribozymes may be used and applied in much the same way as described for antisense nucleic acids.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

It is proposed that sGC proteins, polypeptides or peptides may be co-expressed with other selected proteinaceous molecules, wherein the proteinaceous molecules may be co-expressed in the same cell or sGC gene may be provided to a cell that already has another selected proteinaceous molecule. Co-expression may be achieved by co-transfecting the cell with two distinct recombinant vectors, each bearing a copy of either of the respective DNA. Alternatively, a single recombinant vector may be constructed to include the coding regions for both of the proteinaceous molecules, which could then be expressed in cells transfected with the single vector. In either event, the term “co-expression” herein refers to the expression of both the sGC gene and the other selected proteinaceous molecules in the same recombinant cell.

As used herein, the terms “engineered” and “recombinant” cells or host cells are intended to refer to a cell into which an exogenous DNA segment or gene, such as a cDNA or gene encoding a sGC protein, polypeptide or peptide has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic gene, and also include genes positioned adjacent to a promoter not naturally associated with the particular introduced gene.

To express a recombinant sGC protein, polypeptide or peptide, whether mutant or wild-type, in accordance with the present invention one would prepare an expression vector that comprises a wild-type, or mutant sGC proteinaceous molecule-encoding nucleic acid under the control of one or more promoters. To bring a coding sequence “under the control of” a promoter, one positions the 5′ end of the transcription initiation site of the transcriptional reading frame generally between about 1 and about 50 nucleotides “downstream” of (i.e., 3′ of) the chosen promoter. The ” upstream” promoter stimulates transcription of the DNA and promotes expression of the encoded recombinant protein, polypeptide or peptide. This is the meaning of “recombinant expression” in this context.

Many standard techniques are available to construct expression vectors containing the appropriate nucleic acids and transcriptional/translational control sequences in order to achieve protein, polypeptide or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

The following details concerning recombinant protein production in bacterial cells, such as E. coli, are provided by way of exemplary information on recombinant protein production in general, the adaptation of which to a particular recombinant expression system will be known to those of skill in the art.

Bacterial cells, for example, E. coli, containing the expression vector are grown in any of a number of suitable media, for example, LB. The expression of the recombinant proteinaceous molecule may be induced, e.g., by adding IPTG to the media or by switching incubation to a higher temperature. After culturing the bacteria for a further period, generally of between 2 and 24 hours, the cells are collected by centrifugation and washed to remove residual media.

The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed.

If the recombinant proteinaceous molecule is expressed in the inclusion bodies, as is the case in many instances, these can be washed in any of several solutions to remove some of the contaminating host proteins, then solubilized in solutions containing high concentrations of urea (e.g., 8M) or chaotropic agents such as guanidine hydrochloride in the presence of reducing agents, such as β-mercaptoethanol or DTT (dithiothreitol).

Under some circumstances, it may be advantageous to incubate the proteinaceous molecule for several hours under conditions suitable for the proteinaceous molecule to undergo a refolding process into a conformation which more closely resembles that of the native proteinaceous molecule. Such conditions generally include low proteinaceous molecule concentrations, less than 500 mg/ml, low levels of reducing agent, concentrations of urea less than 2 M and often the presence of reagents such as a mixture of reduced and oxidized glutathione which facilitate the interchange of disulfide bonds within the proteinaceous molecule.

The refolding process can be monitored, for example, by SDS-PAGE, or with antibodies specific for the native molecule (which can be obtained from animals vaccinated with the native molecule or smaller quantities of recombinant proteinaceous molecule). Following refolding, the proteinaceous molecule can then be purified further and separated from the refolding mixture by chromatography on any of several supports including ion exchange resins, gel permeation resins or on a variety of affinity columns.

For expression in Saccharomyces, the plasmid YRp7, for example, is commonly used. This plasmid already contains the trp1 gene which provides a selection marker for a mutant strain of yeast lacking the ability to grow in tryptophan, for example ATCC No. 44076 or PEP4-1. The presence of the trp1 lesion as a characteristic of the yeast host cell genome then provides an effective enviromuent for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the MRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more sGC protein, polypeptide or peptide coding sequences.

In a useful insect system, Autograph californica nuclear polyhedrosis virus (AcNPV) is used as a vector to express foreign genes. The virus grows in Spodoptera frugiperda cells. The sGC protein, polypeptide or peptide coding sequences are cloned into non-essential regions (for example the polyhedrin gene) of the virus and placed under control of an AcNPV promoter (for example the polyhedrin promoter). Successful insertion of the coding sequences results in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., U.S. Pat. No. 4,215,051, Smith, incorporated herein by reference).

Examples of useful mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation) and processing (e.g., cleavage) of proteinaceous products may be important for the function of the proteinaceous molecule.

Different host cells have characteristic and specific mechanisms for the post-translational processing and modification of proteinaceous molecules. Appropriate cells lines or host systems can be chosen to ensure the correct modification and processing of the foreign proteinaceous molecule expressed.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the sGC gene, provided such control sequences are compatible with the host cell systems.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the Bg1 I site located in the viral origin of replication.

In cases where an adenovirus is used as an expression vector, the coding sequences may be ligated to an adenovirus transcription/ translation control complex, e.g., the late promoter and tripartite leader sequence. This chimeric gene may then be inserted in the adenovirus genome by in vitro or in vivo recombination. Insertion in a non-essential region of the viral genome (e.g., region E1, E3, or E4) will result in a recombinant virus that is viable and capable of expressing sGC proteins, polypeptides or peptides in infected hosts.

Specific initiation signals may also be required for efficient translation of sGC protein, polypeptide or peptide coding sequences. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements and transcription terminators.

In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the proteinaceous molecule at a position prior to transcription termination.

For long-term, high-yield production of a recombinant sGC protein, polypeptide or peptide, stable expression is preferred. For example, cell lines that stably express constructs encoding an sGC protein, polypeptide or peptide may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including, but not limited to, the herpes simplex virus thymidine kinase (tk), hypoxanthine-guanine phosphoribosyltransferase (hgprt) and adenine phosphoribosyltransferase (aprt) genes, in tk⁻, hgprt⁻ or aprt⁻ cells, respectively. Also, antimetabolite resistance can be used as the basis of selection for dihydrofolate reductase (dhfr), that confers resistance to methotrexate; gpt, that confers resistance to mycophenolic acid; neomycin (neo), that confers resistance to the aminoglycoside G-418; and hygromycin (hygro), that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower proteinaceous molecule production than adherent cells.

Large scale suspension culture of mammalian cells in stirred tanks is a common method for production of recombinant proteinaceous molecules. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

It is contemplated that the sGC proteins, polypeptides or peptides of the invention may be “overexpressed”, i.e., expressed in increased levels relative to its natural expression in cells. Such overexpression may be assessed by a variety of methods, including radio-labeling and/or proteinaceous molecule purification. However, simple and direct methods are preferred, for example, those involving SDS/PAGE and proteinaceous composition staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific proteinaceous molecule in relation to the other proteins produced by the host cell and, e.g., visible on a gel.

IV. Methods of Gene Transfer

In order to mediate the effect of transgene expression in a cell, it will be necessary to transfer the expression constructs (e.g., a therapeutic construct) of the present invention into a cell. Such transfer may employ viral or non-viral methods of gene transfer. This section provides a discussion of methods and compositions of gene or nucleic acid transfer.

1. Viral Vector-Mediated Transfer

The mammalian sGC nucleic acids are incorporated into an adenoviral infectious particle to mediate gene transfer to a cell. Additional expression constructs encoding other therapeutic agents as described herein may also be transferred via viral transduction using infectious viral particles, for example, by transformation with an adenovirus vector of the present invention as described herein below. Alternatively, retroviral or bovine papilloma virus may be employed, both of which permit permanent transformation of a host cell with a gene(s) of interest. Thus, in one example, viral infection of cells is used in order to deliver therapeutically significant genes to a cell. Typically, the virus simply will be exposed to the appropriate host cell under physiologic conditions, permitting uptake of the virus. Though adenovirus is exemplified, the present methods may be advantageously employed with other viral vectors, as discussed below.

Adenovirus. Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The El region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al, 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

Retrovirus. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins.

The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and ψ components is constructed (Mann et al., 1983). When a recombinant plasmid containing a human cDNA, together with the retroviral LTR and ψ sequences is introduced into this cell line (by calcium phosphate precipitation for example), the ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types.

However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

Adeno-associated Virus. AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Chatteijee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996).

AAV-mediated efficient gene transfer and expression in the lung has led to clinical trials for the treatment of cystic fibrosis (Carter and Flotte, 1996; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al, 1997; McCown et al., 1996; Ping et al., 1996; Xiao et al., 1996).

Other Viral Vectors. Other viral vectors may be employed as expression constructs in the present invention. Vectors derived from viruses such as vaccinia virus (Ridgeway, 1988; Baichwal and Sugden, 1986; Coupar et al., 1988) canary pox virus, and herpes viruses may be employed. These viruses offer several features for use in gene transfer into various mammalian cells.

2. Non-viral Transfer

DNA constructs of the present invention are generally delivered to a cell, in certain situations, the nucleic acid to be transferred is non-infectious, and can be transferred using non-viral methods.

Several non-viral methods for the transfer of expression constructs into cultured mammalian cells are contemplated by the present invention. Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Once the construct has been delivered into the cell the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the P-lactamase gene, Wong et al. (1980) demonstrated the feasibility of liposome-mediated delivery and expression of foreign DNA in cultured chick embryo, HeLa, and hepatoma cells. Nicolau et al. (1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell zmembrane and promote cell entry of liposome-encapsulated DNA (Kaneda et al., 1989). In other embodiments, the liposome may be complexed or employed in conjunction with nuclear nonhistone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferring (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as prostate, epithelial or tumor cells, by any number of receptor-ligand systems with or without liposomes. For example, the human prostate-specific antigen (Watt et al., 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM may also be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

3. Methods of Making Transgenic Animals

As noted above, a particular embodiment of the present invention provides transgenic animals that contain an inactive SGC.

Although the present discussion refers to transgenic mice, it is understood that mice are merely exemplary model animal, and any other mammalian animal routinely used as model animal (e.g., rat, guinea pig, rabbit, cats, dogs, pigs and the like) may be generated using the technology described herein. In a general aspect, a transgenic animal is produced by the integration of a given transgene into the genome in a manner that permits the expression of the transgene. The terms “animal” and “non-human animal”, as used herein, include all vertebrate animals, except humans. It also includes individual animals in all stages of development, including embryonic and fetal stages. A “transgenic animal” is any animal containing one or more cells bearing genetic information received, directly or indirectly, by deliberate genetic manipulation at the subcellular level. The genetic manipulation can be performed by any method of introducing genetic material to a cell, including, but not limited to, microinjection, infection with a recombinant virus, particle bombardment or electroporation. The term is not intended to encompass classical cross-breeding or in vitro fertilization, but rather is meant to encompass animals in which one or more cells receive a recombinant DNA molecule. This molecule may be integrated within a chromosome, or it may be extrachromosomally replicating DNA. The genetic information may be foreign to the species of animal to which the recipient belongs, foreign only to the individual recipient, or genetic information already possessed by the recipient expressed at a different level, a different time, or in a different location than the native gene.

Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a mutant form of the SGC polypeptide which lacks the carboxy-terminal domain of wild-type SGC.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); in The Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

Female mice are induced to superovulate, e.g., by using an injection of pregnant mare serum gonadotropin (PMSG; Sigma) followed, 48 hours later, by an injection of human chronic gonadotropin (hCG; Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

25 μg of a Sa1I-linearized SGC targeting vector is electroporated into 1×10⁷ embryonic stem (ES) cells. After a suitable period of incubation, e.g., 36 hr, the transfected cells are then selected using G418 and FIAU. The G418-FIAU-resistant ES colonies are picked into 96-well plates (Ramirez-Solis et al., 1993). Positive ES clones are injected into C57BL/6 blastocysts and transferred into pseudopregnant ICR female recipients. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

The resulting male chimeras are bred with C57BL/6 females. Germline transmission can be screened by using a phenotype, such as coat color and confirmed by Southern analysis. To obtain the targeted SGC allele in an inbred 129/Sv background, a male chimera is directly bred with 129/Sv female mice.

As noted above, transgenic animals and cell lines derived from such animals may find use in certain testing experiments. In this regard, transgenic animals and cell lines capable of expressing a mutant SGC may be exposed to test substances. These test substances can be screened for the ability to restore TGFβ signaling, and alter the growth of the cell lines and/or the colorectal, neurofibrosarcoma, glioma, astrocytoma, lung cancer or pancreatic tumors in the transgenic animals. Compounds identified by such procedures will be useful in the treatment of colorectal or other cancers involving an aberrant TGFβ-signal caused by altered or dysfunctional SGC expression and/or activity. Thus the compounds identified may be used to prevent, treat, ameliorate tumor growth, cell proliferation, decrease tumor size, or otherwise have a beneficial effect against colorectal cancer or other cancers modeled by the animal or cell lines.

### a. ES Cells

ES cells are obtained from pre-implantation embryos cultured in vitro (Evans et al. 1981; Bradley et al 1984; Gossler et al. 1986; Robertson et al. (1986). Transgenes are introduced into ES cells using a number of means well known to those of skill in the art. The transformed ES cells can thereafter be combined with blastocysts from a non-human animal. The ES cells thereafter colonize the embryo and contribute to the germ line of the resulting chimeric animal (for a review see Jaenisch, 1988).

Once the DNA is introduced, e.g., by electroporation (Troneguzzo et al., 1988; Quillet et al., 1988; Machy et al., 1988), the cells are cultured under conventional conditions well known in the art. In order to facilitate the recovery of those cells which have received the DNA molecule containing the desired gene sequence, it is preferable to introduce the DNA containing the desired gene sequence in combination with a second gene sequence which would contain a detectable marker gene sequence. For the purposes of the present invention, any gene sequence whose presence in a cell permits one to recognize and clonally isolate the cell may be employed as a detectable (selectable) marker gene sequence. The presence of the detectable (selectable) marker sequence in a recipient cell may be recognized by PCR, by detection of radiolabeled nucleotides, or by other assays of detection which do not require the expression of the detectable marker sequence. Typically, the detectable marker gene sequence will be expressed in the recipient cell, and will result in a selectable phenotype. Selectable markers are well known to those of skill in the art. Some examples include the hprt gene (Littlefield, 1964), the neo gene, the tk (thyroidinc kinase) gene of herpes simplex virus (Giphart-Gassler et al., 1989), or other genes which confer resistance to amino acid or nucleoside analogues, or antibiotics, etc.

Any ES cell may be used in accordance with the present invention. It is, however, preferred to use primary isolates of ES cells. Such isolates may be obtained directly from embryos such as the CCE cell line disclosed by Robertson (1989), or from the clonal isolation of ES cells from the CCE cell line (Schwartzberg et al., 1989). Such clonal isolation may be accomplished according to the method of Robertson (1987). The purpose of such clonal propagation is to obtain ES cells which have a greater efficiency for differentiating into an animal. Clonally selected ES cells are approximately 10-fold more effective in producing transgenic animals than the progenitor cell line CCE.

### b. Homologous recombination

Homologous recombination (Koller and Smithies, 1992), directs the insertion of the transgene to a specific location. This technique allows the precise modification of existing genes, and overcomes the problems of positional effects and insertional inactivation observed with transgenic animals generated by pronuclear injection or use of viral vectors. Additionally, it allows the inactivation of specific genes as well as the replacement of one gene for another. In particular embodiments, the DNA segment comprises two selected DNA regions that flank the SGC coding region, thereby directing the homologous recombination of the coding region into the genomic DNA of a non-human animal species.

Thus, a preferred method for the delivery of transgenic constructs involves the use of homologous recombination, or “knock-out technology”. Homologous recombination relies, like antisense, on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination is used as follows. First, the target gene is selected within the host cell. Sequences homologous to the target gene are then included in a genetic construct, along with some mutation that will render the target gene inactive (stop codon, interruption, and the like). The homologous sequences flanking the inactivating mutation are said to “flank” the mutation. Flanking, in this context, simply means that target homologous sequences are located both upstream (5′) and downstream (3′) of the mutation. These sequences should correspond to some sequences upstream and downstream of the target gene. The construct is then introduced into the cell, thus permitting recombination between the cellular sequences and the construct.

As a practical matter, the genetic construct will normally act as far more than a vehicle to interrupt the gene. For example, it is important to be able to select for recombinants and, therefore, it is common to include within the construct a selectable marker gene. This gene permits selection of cells that have integrated the construct into their genomic DNA by conferring resistance to various biostatic and biocidal drugs. In addition, a heterologous gene that is to be expressed in the cell also may advantageously be included within the construct. The arrangement might be as follows:

vector·5′-flanking sequence·heterologous gene·selectable marker gene·flanking sequence-3·-vector

Thus, using this kind of construct, it is possible, in a single recombinatorial event, to (i) “knock out” an endogenous gene, (ii) provide a selectable marker for identifying such an event and (iii) introduce a transgene for expression.

Another refinement of the homologous recombination approach involves the use of a “negative” selectable marker. This marker, unlike the selectable marker, causes death of cells which express the marker. Thus, it is used to identify undesirable recombination events. When seeking to select homologous recombinants using a selectable marker, it is difficult in the initial screening step to identify proper homologous recombinants from recombinants generated from random, non-sequence specific events. These recombinants also may contain the selectable marker gene and may express the heterologous protein of interest, but will, in all likelihood, not have the desired “knock out” phenotype. By attaching a negative selectable marker to the construct, but outside of the flanking regions, one can select against many random recombination events that will incorporate the negative selectable marker. Homologous recombination should not introduce the negative selectable marker, as it is outside of the flanking sequences. Examples of processes that use negative selection to enrich for homologous recombination include the disruption of targeted genes in embryonic stem cells or transformed cell lines (Mortensen, 1993; Willnow and Herz, 1994) and the production of recombinant virus such as adenovirus (Imler et al., 1995).

Since the frequency of gene targeting is heavily influenced by the origin of the DNA being used for targeting, it is beneficial to obtain DNA that is as similar (isogenic) to the cells being targeted as possible. One way to accomplish this is by isolation of the region of interest from genomic DNA from a single colony by long range PCR. Using long range PCR it is possible to isolate fragments of 7-12 kb from small amounts of starting DNA.

Gene trapping is a useful technique suitable for use with the present invention. This refers to the utilization of the endogenous regulatory regions present in the chromosomal DNA to activate the incoming transgene. In this way expression of the transgene is absent or minimized when the transgene inserts in a random location. However, when homologous recombination occurs the endogenous regulatory region are placed in apposition to the incoming transgene, which results in expression of the transgene.

### C. Site Specific Recombination

Members of the integrase family are proteins that bind to a DNA recognition sequence, and are involved in DNA recognition, synapsis, cleavage, strand exchange, and religation. Currently, the family of integrases includes 28 proteins from bacteria, phage, and yeast which have a common invariant His-Arg-Tyr triad (Abremski and Hoess, 1992). Four of the most widely used site-specific recombination systems for eukaryotic applications include: Cre-loxP from bacteriophage P1 (Austin et al., 1981); FLP-FRT from the 2μ plasmid of Saccharomyces cerevisiae (Andrews et al., 1985); R-RS from Zygosaccharomyces rouxii (Maeser and Kahmann, 1991) and gin-gix from bacteriophage Mu (Onouchi et al., 1995). The Cre-loxP and FLP-FRT systems have been developed to a greater extent than the latter two systems. The R-RS system, like the Cre-loxP and FLP-FRT systems, requires only the protein and its recognition site. The Gin recombinase selectively mediates DNA inversion between two inversely oriented recombination sites (gix) and requires the assistance of three additional factors: negative supercoiling, an enhancer sequence and its binding protein Fis.

The present invention contemplates the use of the Cre/Lox site-specific recombination system (Sauer, 1993, available through Gibco/BRL, Inc., Gaithersburg, Md.) to rescue specific genes out of a genome, and to excise specific transgenic constructs from the genome. The Cre (causes recombination)-lox P (locus of crossing-over(x)) recombination system, isolated from bacteriophage P1, requires only the Cre enzyme and its loxP recognition site on both partner molecules (Stemberg and Hamilton, 1981). The loxp site consists of two symmetrical 13 bp protein binding regions separated by an 8 bp spacer region, which is recognized by the Cre recombinase, a 35 kDa protein. Nucleic acid sequences for loxP (Hoess et al., 1982) and Cre (Stemberg et al., 1986) are known. If the two lox P sites are cis to each other, an excision reaction occurs; however, if the two sites are trans to one another, an integration event occurs. The Cre protein catalyzes a site-specific recombination event. This event is bidirectional, i.e., Cre will catalyze the insertion of sequences at a LoxP site or excise sequences that lie between two LoxP sites. Thus, if a construct for insertion also has flanking LoxP sites, introduction of the Cre protein, or a polynucleotide encoding the Cre protein, into the cell will catalyze the removal of the construct DNA. This technology is enabled in U.S. Pat. No. 4,959,317, which is hereby incorporated by reference in its entirety.

An initial in vivo study in bacteria showed that the Cre excises loxP-flanked DNA extrachromosomally in cells expressing the recombinase (Abremski et al., 1983). A major question regarding this system was whether site-specific recombination in eukaryotes could be promoted by a bacterial protein. However, Sauer (1987) showed that the system excises DNA in S. cerevisiae with the same level of efficiency as in bacteria.

Further studies with the Cre-loxP system, in particular the ES cells system in mice, has demonstrated the usefulness of the excision reaction for the generation of unique transgenic animals. Homologous recombination followed by Cre-mediated deletion of a loxP-flanked neo-tk cassette was used to introduce mutations into ES cells. This strategy was repeated for a total of 4 rounds in the same line to alter both alleles of the rep-3 and mMsh2 loci, genes involved in DNA mismatch repair (Abuin and Bradley, 1996). Similarly, a transgene which consists of the 35S promoter/luciferase gene/loxP/35S promoter/hpt gene/loxP (luc⁺hyg⁺) was introduced into tobacco. Subsequent treatment with Cre causes the deletion of the hyg gene (luc⁺hyg^{S}) at 50% efficiency (Dale and Ow, 1991). Transgenic mice which have the Ig light chain κ constant region targeted with a loxP-flanked neo gene were bred to Cre-producing mice to remove the selectable marker from the early embryo (Lakso et al., 1996). This general approach for removal of markers stems from issues raised by regulatory groups and consumers concerned about the introduction of new genes into a population.

An analogous system contemplated for use in the present invention is the FLP/FRT system. This system was used to target the histone 4 gene in mouse ES cells with a FRT-flanked neo cassette followed by deletion of the marker by FLP-mediated recombination. The FLP protein could be obtained from an inducible promoter driving the FLP or by using the protein itself (Wigley et al., 1994).

The present invention also contemplates the use of recombination activating genes (RAG) 1 and 2 to excise specific transgenic constructs from the genome, as well as to rescue specific genes from the genome. RAG-1 (GenBank accession number M29475) and RAG-2 (GenBank accession numbers M64796 and M33828) recognize specific recombination signal sequences (RSSs) and catalyze V(D)J recombination required for the assembly of immunoglobulin and T cell receptor genes (Schatz et al., 1989; Oettinger et al., 1990; Cumo and Oettinger, 1994). Transgenic expression of RAG-1 and RAG-2 proteins in non-lymphoid cells supports V(D)J recombination of reporter substrates (Oettinger et al., 1990). For use in the present invention, the transforming construct of interest is engineered to contain flanking RSSs. Following transformation, the transforming construct that is internal to the RSSs can be deleted from the genome by the transient expression of RAG-1 and RAG-2 in the transformed cell.

V. sGC Proteins, Polypeptides, and Peptides

The present invention also provides purified, and in preferred embodiments, substantially purified mammalian sGC proteins, polypeptides, or peptides. The term “purified mammalian sGC proteins, polypeptides, or peptides” as used herein, is intended to refer to an sGC proteinaceous composition, isolatable from mammalian cells or recombinant host cells, wherein the sGC protein, polypeptide, or peptide is purified to any degree relative to its naturally-obtainable state, i.e., relative to its purity within a cellular extract. A purified sGC protein, polypeptide, or peptide therefore also refers to a wild-type or mutant sGC protein, polypeptide, or peptide free from the environment in which it naturally occurs.

The sGC proteins may be full length proteins, such as being 826 amino acids in length. The sGC proteins, polypeptides and peptides may also be less then full length proteins, such as individual polypeptide, domains, regions or even epitopic peptides. Where less than full length sGC proteins are concerned the most preferred will be those containing predicted immunogenic sites and those containing the functional domains identified herein.

Encompassed by the invention are proteinaceous segments of relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and more preferably, of from about 15 to about 30 amino acids in length; as set forth in SEQ ID NO: 2, and also larger polypeptides of from about 51, about 52, about 53, about 54, about 55, about 56, about 57, about 58, about 59, about 60, about 65, about 70, about 75, about 80, about 85, about 90, about 95, about 100, about 110, about 120, about 130, about 140, about 150, about 160, about 170, about 180, about 190, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, up to and including proteins corresponding to the full-length sequences set forth in SEQ ID NO: 2.

Generally, “purified” will refer to an sGC protein, polypeptide, or peptide composition that has been subjected to fractionation to remove various non-sGC protein, polypeptide, or peptide, and which composition substantially retains its sGC activity, as may be assessed, assays described herein or would be known to one of skill in the art.

Where the term “substantially purified” is used, this will refer to a composition in which the sGC protein, polypeptide, or peptide forms the major component of the composition, such as constituting about 50% of the proteinaceous molecules in the composition or more. In preferred embodiments, a substantially purified proteinaceous molecule will constitute more than 60%, 70%, 80%, 90%, 95%, 99% or even more of the proteinaceous molecules in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of sGC proteins, polypeptides, or peptides will be known to those of skill in the art in light of the present disclosure. These include, for example, determnining the specific sGC proteinaceous molecule's activity of a fraction, or assessing the number of proteins, polypeptides and peptides within a fraction by gel electrophoresis. Assessing the number of proteinaceous molecules within a fraction by SDS/PAGE analysis will often be preferred in the context of the present invention as this is straightforward.

To purify an sGC protein, polypeptide, or peptide a natural or recombinant composition comprising at least some sGC proteins, polypeptides, or peptides will be subjected to fractionation to remove various non-sGC components from the composition. In addition to those techniques described in detail herein below, various other techniques suitable for use in proteinaceous molecule purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of an sGC fusion protein using a specific binding partner. Such purification methods are routine in the art. As the present invention provides DNA sequences for sGC proteins, any fusion protein purification method can now be practiced. This is exemplified by the generation of an sGC-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography.

The exemplary purification methods disclosed herein represent exemplary methods to prepare a substantially purified sGC protein, polypeptide, or polypeptide. These methods are preferred as they result in the substantial purification of the sGC protein, polypeptide or peptide in yields sufficient for further characterization and use. However, given the DNA and proteinaceous molecules provided by the present invention, any purification method can now be employed.

Although preferred for use in certain embodiments, there is no general requirement that the sGC protein, polypeptide, or peptide always be provided in their most purified state. Indeed, it is contemplated that less substantially purified sGC protein, polypeptide or peptide, which are nonetheless enriched in sGC proteinaceous compositions, relative to the natural state, will have utility in certain embodiments. These include, for example, antibody generation where subsequent screening assays using purified sGC proteinaceous molecules are conducted.

Methods exhibiting a lower degree of relative purification may have advantages in total recovery of proteinaceous molecule product, or in maintaining the activity of an expressed proteinaceous molecule. Inactive products also have utility in certain embodiments, such as, e.g., in antibody generation.

VI. Antibodies to sGC Proteins

A. Epitopic Core Sequences

Peptides corresponding to one or more antigenic determinants, or “epitopic core regions”, of the sGC proteins of the present invention can also be prepared. Such peptides should generally be at least five or six amino acid residues in length, will preferably be about 10, 15, 20, 25 or about 30 amino acid residues in length, and may contain up to about 35 to about 50 residues or so.

Synthetic peptides will generally be about 35 residues long, which is the approximate upper length limit of automated peptide synthesis machines, such as those available from Applied Biosystems (Foster City, Calif.). Longer peptides may also be prepared, e.g., by recombinant means.

U.S. Pat. No. 4,554,101, (Hopp) incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the sGC sequence disclosed herein in SEQ ID NO: 2.

Numerous scientific publications have also been devoted to the prediction of secondary structure, and to the identification of epitopes, from analyses of amino acid sequences (Chou & Fasman, 1974a,b; 1978a,b, 1979). Any of these may be used, if desired, to supplement the teachings of Hopp in U.S. Pat. No. 4,554,101.

Moreover, computer programs are currently available to assist with predicting antigenic portions and epitopic core regions of proteinaceous molecules. Examples include those programs based upon the Jameson-Wolf analysis (Jameson & Wolf, 1988; Wolf et al., 1988), the program PepPlot® (Brutlag et al., 1990; Weinberger et al., 1985), and other new programs for proteinaceous molecule tertiary structure prediction (Fetrow and Bryant, 1993). Another commercially available software program capable of carrying out such analyses is MacVector (IBI, New Haven, Conn.).

In further embodiments, major antigenic determinants of a polypeptide may be identified by an empirical approach in which portions of the gene encoding the polypeptide are expressed in a recombinant host, and the resulting proteinaceous molecules tested for their ability to elicit an immune response. For example, PCR™ can be used to prepare a range of peptides lacking successively longer fragments of the C-terminus of the proteinaceous molecule. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs™ system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants can also be constructed and inserted into expression vectors by standard methods, for example, using PCR™ cloning methodology.

The use of such small peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

B. Antibody Generation

In certain embodiments, the present invention provides antibodies that bind with high specificity to the sGC proteinaceous molecules provided herein. Thus, antibodies that bind to the proteinaceous products of the isolated nucleic acid sequences of SEQ ID NO: 1, SEQ ID NO: 3, SEQ ID NO: 4, SEQ ID NO: 5 OR SEQ ID NO: 6 are provided. As detailed above, in addition to antibodies generated against the full length proteins, antibodies may also be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

The methods for generating monoclonal antibodies (MAbs) generally begin along the same lines as those for preparing polyclonal antibodies. Briefly, a polyclonal antibody is prepared by immunizing an animal with an immunogenic sGC proteinaceous composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, Pa.); low-dose Cyclophosphamide (CYP; 300 mg/m²) (Johnson/ Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified sGC protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, 1984). For example, where the immunized animal is a mouse, one may use P3-X63/Ag8, X63-Ag8.653, NS1/1.Ag 4 1, Sp2 10-Ag14, FO, NSO/U, MPC-11, MPC11-X45-GTG 1.7 and S194/5XX0 Bull for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1: 1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

C. Antibody Conjugates

The present invention further provides antibodies against sGC proteinaceous molecules, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins” (described in U.S. Pat. Nos. 5,686,072, 5,578,706, 4,792,447, 5,045,451, 4,664,911 and 5,767,072, each incorporated herein by reference).

Antibody conjugates are thus preferred for use as diagnostic agents. Antibody diagnostics generally fall within two classes, those for use in vitro diagnostics, such as in a variety of immunoassays, and those for use in vivo diagnostic protocols, generally known as “antibody-directed imaging”. Again, antibody-directed imaging is less preferred for use with this invention.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or period. Conjugates with fluorescein markers are prepared in the presence of these coupling agents or by reaction with an isothiocyanate.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷,¹⁵²Eu, galium⁶⁷ ,³hydrogen, iodine ^{123,} , iodine¹²⁵, iodine¹³¹, indium¹¹¹,⁵⁹iron, ³²phosphorus, rhenium¹⁸⁶, rhenium ¹⁸⁸, ⁷⁵selenium, ³⁵sulphur, technicium^{99m} and yttrium⁹⁰. ¹²⁵I is often being preferred for use in certain embodiments, and technicium⁹⁹m and indium¹¹¹are also often preferred due to their low energy and suitability for long range detection.

Radioactively labeled monoclonal antibodies of the present invention may be produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium-^{99m} by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) and ethylene diaminetetracetic acid (EDTA). Also contemplated for use are fluorescent labels, including rhodamine, fluorescein isothiocyanate and renographin.

The much preferred antibody conjugates of the present invention are those intended primarily for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. No. 3,817,837; 3,850,752; 3,939,350; 3,996,345; 4,277,437; 4,275,149 and 4,366,241; each incorporated herein by reference.

D. Immunodetection Methods

In still further embodiments, the present invention concerns immunodetection methods for binding, purifying, removing, quantifying or otherwise generally detecting biological components such as sGC proteinaceous components. The sGC antibodies prepared in accordance with the present invention may be employed to detect wild-type or mutant sGC proteins, polypeptides or peptides. As described throughout the present application, the use of wild-type or mutant sGC specific antibodies is contemplated. The steps of various useful immunodetection methods have been described in the scientific literature, such as, e.g., Nakamura et al. (1987), incorporated herein by reference.

In general, the immunobinding methods include obtaining a sample suspected of containing an sGC protein, polypeptide or peptide, and contacting the sample with a first anti-sGC antibody in accordance with the present invention, as the case may be, under conditions effective to allow the formation of immunocomplexes.

These methods include methods for purifying wild-type or mutant sGC proteins, polypeptides or peptides as may be employed in purifying wild-type or mutant sGC proteins, polypeptides or peptides from patients′ samples or for purifying recombinantly expressed wild-type or mutant sGC proteins, polypeptides or peptides. In these instances, the antibody removes the antigenic wild-type or mutant sGC protein, polypeptide or peptide component from a sample. The antibody will preferably be linked to a solid support, such as in the form of a column matrix, and the sample suspected of containing the wild-type or mutant sGC protein antigenic component will be applied to the immobilized antibody. The unwanted components will be washed from the column, leaving the antigen immunocomplexed to the immobilized antibody, which wild-type or mutant sGC protein, polypeptide or peptide antigen is then collected by removing the wild-type or mutant sGC protein, polypeptide or peptide from the column.

The immunobinding methods also include methods for detecting or quantifying the amount of a wild-type or mutant sGC proteinaceous reactive component in a sample, which methods require the detection or quantification of any immune complexes formed during the binding process. Here, one would obtain a sample suspected of containing a wild-type or mutant sGC protein, polypeptide or peptide, and contact the sample with an antibody against wild-type or mutant sGC, and then detect or quantify the amount of immune complexes formed under the specific conditions.

In terms of antigen detection, the biological sample analyzed may be any sample that is suspected of containing a wild-type or mutant sGC proteinaceous molecule-specific antigen, such as a diseased urogenital tract tissue section, secretion or specimen, separated or purified forms of any of the above wild-type or mutant sGC proteinaceous-containing compositions.

Contacting the chosen biological sample with the antibody under conditions effective and for a period of time sufficient to allow the formation of immune complexes (primary immune complexes) is generally a matter of simply adding the antibody composition to the sample and incubating the mixture for a period of time lone enough for the antibodies to form immune complexes with, i.e., to bind to, any sGC antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological or enzymatic tags. U.S. Pat. Nos. concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The sGC antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

1. ELISAs

As detailed above, immunoassays, in their most simple and direct sense, are binding assays. Certain preferred inumunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the anti-sGC antibodies of the invention are immobilized onto a selected surface exhibiting protein affinity, such as a well in a polystyrene microtiter plate. Then, a test composition suspected of containing the wild-type or mutant sGC antigen, such as a clinical sample, is added to the wells. After binding and washing to remove non-specifically bound immune complexes, the bound wild-type or mutant sGC protein, polypeptide or peptide antigen may be detected. Detection is generally achieved by the addition of another anti-sGC antibody that is linked to a detectable label. This type of ELISA is a simple “sandwich ELISA”. Detection may also be achieved by the addition of a second anti-sGC antibody, followed by the addition of a third antibody that has binding affinity for the second antibody, with the third antibody being linked to a detectable label.

In another exemplary ELISA, the samples suspected of containing the wild-type or mutant sGC antigen are immobilized onto the well surface and then contacted with the anti-sGC antibodies of the invention. After binding and washing to remove non-specifically bound immune complexes, the bound anti-sGC antibodies are detected. Where the initial anti-sGC antibodies are linked to a detectable label, the immune complexes may be detected directly. Again, the immune complexes may be detected using a second antibody that has binding affinity for the first anti-sGC antibody, with the second antibody being linked to a detectable label.

Another ELISA in which the wild-type or mutant sGC proteins, polypeptides or peptides are immobilized, involves the use of antibody competition in the detection. In this ELISA, labeled antibodies against wild-type or mutant sGC protein, polypeptide or peptides are added to the wells, allowed to bind, and detected by means of their label. The amount of wild-type or mutant sGC antigen in an unknown sample is then determined by mixing the sample with the labeled antibodies against wild-type or mutant sGC before or during incubation with coated wells. The presence of wild-type or mutant sGC proteinaceous molecule in the sample acts to reduce the amount of antibody against wild-type or mutant sGC proteinaceous molecule available for binding to the well and thus reduces the ultimate signal. This is also appropriate for detecting antibodies against wild-type or mutant sGC protein, polypeptide or peptide in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a proteinaceous molecule or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid (ABTS) and H₂O₂, in the case of peroxidase as the enzyme label. Quantification is then achieved by measuring the degree of color generation, e.g., using a visible spectra spectrophotometer.

2. Immunohistochemistry

The antibodies of the present invention may also be used in conjunction with both fresh-frozen and formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown etaL., 1990; Abbondanzo et al., 1990; Allred et al., 1990).

Briefly, frozen-sections may be prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

VII. Diagnostics and Screens for Mammalian sGC

A. Diagnostics

As with the therapeutic methods of the present invention, the diagnostic methods are based upon the novel gene encoding sGC, which encode a protein that is predicted to have sGC activity. The diagnostic methods of the present invention generally involve determining either the type or the amount of a wild-type or mutant sGC proteinaceous molecule present within a biological sample from a patient suspected of having a disease associated with aberrant sGC activity. Irrespective of the actual role of sGC in the etiology of disease, it will be understood that the detection of a mutant form of sGC is likely to be diagnostic of a disease, such as those described herein, and that the detection of altered amounts of sGC, either at the mRNA or protein level, is also likely to have diagnostic implications, particularly where there is a reasonably significant difference in amounts.

The finding of a decreased amount of sGC in one, or preferably more, cancerous samples, in comparison to the amount within a sample from a control sample, will be indicative of the role of sGC in a particular disease. Following which, disease in others would be similarly diagnosed by detecting a decreased amount of sGC in a sample. The finding of a increased amount of sGC in one, or preferably more, patients, in comparison to the amount within a sample from a control subject, will be indicative of the role of the sGC in a particular disease. Following which, disease in others would be similarly diagnosed by detecting a increased amount of sGC in a sample.

The type or amount of sGC proteinaceous molecule present within a biological sample, such as a tissue sample, secretion, or body fluid, may be determined by means of a molecular biological assay to determine the level of a nucleic acid that encodes such an sGC proteinaceous molecule, or by means of an immunoassay to determine the level of the protein, polypeptide or peptide itself. Any of the foregoing nucleic acid detection methods or immunodetection methods may be employed as a diagnostic methods in the context of the present invention.

B. Modulators and Screening Assays

The present invention further comprises methods for identifying modulators of the function of sGC. These assays may comprise random screening of large libraries of candidate substances; alternatively, the assays may be used to focus on particular classes of compounds selected with an eye towards structural attributes that are believed to make them more likely to modulate the function of sGC.

To identify a sGC modulator, one generally will determine the function of sGC in the presence and absence of the candidate substance, a modulator defined as any substance that alters function. For example, a method generally comprises:

(a) providing a candidate modulator;

(b) admixing the candidate modulator with an isolated compound or cell, or a suitable experimental animal;

(c) measuring one or more characteristics of the compound, cell or animal in step (c); and

(d) comparing the characteristic measured in step (c) with the characteristic of the compound, cell or animal in the absence of said candidate modulator,

wherein a difference between the measured characteristics indicates that said candidate modulator is, indeed, a modulator of the compound, cell or animal.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit or enhance sGC activity. The candidate substance may be a protein or fragment thereof, a small molecule, or even a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on sGC. Regardless of the type of inhibitor or activator identified by the present screening methods, the effect of the inhibition or activator by such a compound results in altering sGC activity or expression as compared to that observed in the absence of the added candidate substance.

2. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

3. In cyto Assays

The present invention also contemplates the screening of compounds for their ability to modulate sGC in cells. Various cell lines can be utilized for such screening assays, including cells specifically engineered for this purpose.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

4. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that alter sGC activity or expression. In these embodiments, the present invention is directed to a method for determining the ability of a candidate substance to alter sGC activity or expression, generally including the steps of: administering a candidate substance to the animal; and determining the ability of the candidate substance to reduce one or more characteristics of sGC.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

VIII. Pharmaceutical Compositions

Pharmaceutical compositions of the present invention comprise an effective amount of one or more sGC proteinaceous sequence, nucleic acid or antibody or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one sGC proteinaceous sequence, nucleic acid or antibody or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The sGC proteinaceous sequence, nucleic acid or antibody may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0. 1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The sGC proteinaceous sequence, nucleic acid or antibody may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the sGC proteinaceous sequence, nucleic acid or antibody is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fingi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

IX. Kits

Certain embodiments of the present invention concerns diagnostic or therapeutic kits. The components of the various kits may be stored in suitable container means. The container means will generally include at least one vial, test tube, flask, bottle, syringe or other container means, into which the mammalian sGC proteinaceous molecule, nucleic acid, antibody or inhibitory formulation are placed, preferably, suitably allocated. The kits may also comprise a second container means for containing a sterile, pharmaceutically acceptable buffer or other diluent. The kits of the present invention will also typically include a means for containing the vials in close confinement for commercial sale, such as, e.g., injection or blow-molded plastic containers into which the desired vials are retained.

In one embodiment, a diagnostic kit may comprising sGC probes or primers for use with the nucleic acid detection methods. All the essential materials and reagents required for detecting sGC nucleic acid markers in a biological sample may be assembled together in a kit. This generally will comprise preselected primers for specific markers. Also included may be enzymes suitable for amplifying nucleic acids including various polymerases (RT, Taq, etc.), deoxynucleotides and buffers to provide the necessary reaction mixture for amplification.

Such kits generally will comprise, in suitable means, distinct containers for each individual reagent and enzyme as well as for each marker primer pair. Preferred pairs of primers for amplifying nucleic acids are selected to amplify the sequences specified in SEQ ID NO: 1, SEQ ID NO: 3, SEQ ID NO: 4, SEQ ID NO: 5 OR SEQ ID NO: 6, or a complement thereof.

In another embodiment, such kits will comprise hybridization probes specific for sGC corresponding to the sequences specified in SEQ ID NO: 1, SEQ ID NO: 3, SEQ ID NO: 4, SEQ ID NO: 5 OR SEQ ID NO: 6, or the complement thereof. Such kits generally will comprise, in suitable means, distinct containers for each individual reagent and enzyme as well as for each hybridization probe.

In other embodiments, the present invention concerns immunodetection kits for use with the immunodetection methods described above. As the sGC antibodies are generally used to detect wild-type or mutant sGC proteins, polypeptides or peptides, the antibodies will preferably be included in the kit. The immunodetection kits will thus comprise, in suitable container means, a first antibody that binds to a wild-type or mutant sGC protein, polypeptide or peptide, and optionally, an immunodetection reagent and further optionally, a wild-type or mutant sGC protein, polypeptide or peptide.

In preferred embodiments, monoclonal antibodies will be used. In certain embodiments, the first antibody that binds to the wild-type or mutant sGC protein, polypeptide or peptide may be pre-bound to a solid support, such as a column matrix or well of a microtitre plate.

The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

The kits may further comprise a suitably aliquoted composition of the wild-type or mutant sGC protein, polypeptide or polypeptide, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

Therapeutic kits of the present invention are kits comprising an sGC protein, polypeptide, peptide, biological functional equivalent, immunological fragment, domain, inhibitor, gene, vector, probe, primer, polynucleotide, nucleic acid, complement, antibody, or other sGC effector. Such kits will generally contain, in suitable container means, a pharmaceutically acceptable formulation of an sGC protein, polypeptide, peptide, biological functional equivalent, immunological fragment, domain, inhibitor, antibody, gene, polynucleotide, nucleic acid, complement, or vector expressing any of the foregoing in a pharmaceutically acceptable formulation. The kit may have a single container means, or it may have distinct container means for each compound.

When the components of the kit are provided in one or more liquid solutions, the liquid solution is an aqueous solution, with a sterile aqueous solution being particularly preferred. The sGC compositions may also be formulated into a syringeable composition. In which case, the container means may itself be a syringe, pipette, or other such like apparatus, from which the formulation may be applied to an infected area of the body, injected into an animal, or even applied to and mixed with the other components of the kit.

However, the components of the kit may be provided as dried powder(s). When reagents or components are provided as a dry powder, the powder can be reconstituted by the addition of a suitable solvent. It is envisioned that the solvent may also be provided in another container means.

The container means of the kits will generally include at least one vial, test tube, flask, bottle, syringe or other container means, into which the antibody may be placed, and preferably, suitably aliquoted. Where wild-type or mutant sGC protein, polypeptide or peptide, or a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

Irrespective of the number or type of containers, the kits of the invention may also comprise, or be packaged with, an instrument for assisting with the injection/administration or placement of the ultimate sGC proteinaceous molecule or nucleic acid composition within the body of an animal. Such an instrument may be a syringe, pipette, forceps, or any such medically approved delivery vehicle.

X. Examples

The following examples are included to demonstrate preferred embodiments of the invention. It should be appreciated by those of skill in the art that the techniques disclosed in the examples which follow represent techniques discovered to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The structures of the genes encoding the α₁ and β₁ subunits of murine soluble guanylyl cyclase (sGC) were determined. Full-length cDNA's isolated from mouse lungs encoding the α₁, (2.5-kb) and β₁(3.3-kb) subunits are presented. The α₁. sGC gene is approximately 26.4 kb and contains 9 exons, while the β₁sGC gene spans 22 kb and has 14 exons. The positions of exon/intron boundaries and the sizes of introns for both genes are described. Comparison of mouse genomic organization with the Human Genome database predicted the exon/intron boundaries of the human genes and revealed that human and mouse α₁and β₁sGC genes have similar structures.

Both mouse genes are localized on the third chromosome, band 3E3-F1, and are separated by a fragment that is 2% of the chromosomal length. The 5′ untranscribed regions of α₁and β₁subunit genes were subcloned into luciferase reporter constructs and the functional analysis of promoter activity was performed in murine neuroblastoma NiE-115 cells. Results indicate that the 5′ untranscribed regions for both genes possess independent promoter activities and, together with the data on chromosomal localization, indicate independent regulation of both genes.

Abbreviations: sGC, soluble guanylyl cyclase; bp, base pairs; NO, nitric oxide; cAMP, cyclic adenosine monophosphate; FISH, fluorescence in situ hybridization; DAPI, 4′, 6-diamidino-2-phenylindole; CMV, cytomegalovirus.

Isolation of a eDNA clone for mouse sGCα₁subunit A mouse lung λ Triplex cDNA library (Clontech) was screened by hybridization using a 1.3-kb rat sGCOα₁cDNA fragment obtained by PCR using Taq polymerase (Gibco) and the oligonucleotide primers 5′-⁹¹TGCACTTCAGAGAACCTTG-3′ and 5′-⁵²⁰CTCCACCTTGTAGACATCCA-3′ (superscript indicates position of codon at which the primers start). Six positive clones were identified from approximately 1×10⁶ independent phage plaques. Positive clones were subsequently purified, sequenced bidirectionally for positive clone identification, and analyzed using DNASTAR software (DNASTAR, Inc., Madison, Wis.). Following analysis, the clone was defined as mouse (XI sGC and submitted to the NCBI database. This clone was used in all subsequent experiments and alignments described herein.

Isolation of genomic clones for mouse sGCα₁and β₁subunits. A bacterial artificial chromosomal (BAC) high-density membrane mouse library was purchased from Genome Systems (St. Louis, Mo.). The hybridization was performed overnight at 46° C. in standard hybridization solution (Sambrook et al.,1989). A random primer-labeled α³²P-dCTP-labeled cDNA fragment (0.9 Kb) for the β₁sGC probe was generated by RT-PCR from a total RNA preparation from murine neuroblastoma N1E-115 cells, using the oligonucleotides 5 ′-−3GACACCATGTACGGTTTCGTG-3′ and 5′-²⁴³CCCTTCCTTGCTTCTCAGTAC-3′ (superscript indicates the base pairs upstream of the start codon or the position of the codon at which the primers start).

The membranes were then re-hybridized with an α₁sGC cDNA probe (1.3 kb containing coding sequence) using the same conditions. Positive BAC clones were identified using the manufacturer's procedure and purchased from Genome Systems (St. Louis, Mo.). BAC plasmid purification kit (Clontech) was used for BAC DNA isolation from bacterial culture. BAC DNA was subjected to restriction and Southern blot hybridization analysis ((Sambrook et al.,1989)) using the same hybridization probes to confirm isolation of positive clones (data not shown).

Determination of boundaries and sizes of introns. Based on the α₁and β₁ sGC cDNA sequences, sequencing oligonucleotide primers were designed to determine the genomic structure of each subunit. All sequencing analyses were performed at the Molecular Core Sequencing Facility at the University of Texas-Houston Medical School on an ABI Prism 377 DNA sequencer with the DigDye Terminator cycle sequencing kit (Applied Biosystems, Calif.). Primers positioned in the exons of both subunits were used to determine the intron sizes by PCR with Pfu-Turbo DNA polymerase (Stratagene) from BAC DNA templates. PCR conditions were: melting step at 95° C. for 1 min, primer annealing at 55° C. for 1 min, extension step at 72° C. for 3 min, repeated for 35 cycles. PCR products were separated by electrophoresis on 1% agarose gels.

3′- Rapid amplification of cDNA end (3′-RACE) of mouse sGC,β₁subunit. Poly (A)⁺ RNA was purified from lung tissue of CD57 mice using an MRNA extraction kit (Dynal). Determination of the 3′ end of β₁sGC MRNA was performed using a SMART RACE cDNA Amplification kit (Clontech). In brief, the first-strand cDNA synthesis was achieved by incubating the poly(A)⁺ RNA with a 3′cDNA-specific primer and Superscript Reverse Transcriptase (Gibco) for 1.5 hrs at 42° C. Next a “touchdown” PCR reaction to amplify the fragment was executed using an oligonucleotide

5′-²⁵⁸CTGCTACAAGCATTGCCTAGACGGACG-3′ (superscript indicates the base pairs downstream of the stop codon where the primer starts), specific to the 3′ end of the published β₁sGC sequence, and the universal primer mixture that recognizes the modified 3′-end of cDNA. PCR conditions were as suggested by the manufacturer (Clonthech). PCR products were subdloned into pCR 2.1-Topo vector (Invitrogen) and both strands were sequenced for verification.

Chromosomal localization of mouse sGCα₁and β_{1 subunits. Chromosomal localization of α}₁and β₁sGC genes was performed by Genome Systems (St. Louis, Mo.) using fluorescence in situ hybridization (FISH). Briefly, purified BAC DNA for each clone, containing the genomic sequence of α₁and β₁sGC, was labeled with digoxigenin dUTP by nick translation. The labeled probe was combined with sheared mouse DNA and hybridized to normal metaphase chromosomes derived from mouse embryo fibroblast cells in a solution containing 50% formamide, 10 % dextran sulfate, and 2×SSC. The hybridization was detected using fluorescent antidigoxigenin antibodies followed by counterstaining with DAPI. In addition, a probe specific for the telomeric region of chromosome 3 was co-hybridized with each clone to verify specific labeling of the telomere to chromosome 3. Specific measurements identifying the hybridization signal between the heterochromatic-euchromatic boundary to the telomere of chromosome 3 indicated the band location of each clone on mouse chromosome 3.

Cloning of luciferase plasmid constructs. In order to create the plasmid constructs containing the 5′-upstream regions of α₁and β₁sGC extended to the first identified exon for each gene, DNA fragments were obtained by PCR using the specific genomic clones as templates and Pfu Turbo DNA Polymerase (Stratagene). Positive strand oligonucleotide primers for each construct were:

1.6 kb -α₁5′-¹⁹⁰¹GTCAGTGTCAGACCTGAAGATGCTG-3′ and

1.4 kb -β₁5 ′-⁻¹⁵²⁸CTCTCTGTGTGTGAGAGAGAG-3′ (superscript indicates the base pairs upstream of the start codon). Each of these positive strand oligonucleotide primers contained a Kpn I restriction site linker sequence at the 5′ end. The negative strand primers were:

5′-⁻¹⁰⁴CATGATGCGATCACAGGAGGC-3′for the α₁, construct and

5′-⁻¹⁰⁵CGCCCGGAGCCTAGGAAGCAG-3′ for the β₁construct (superscript indicates the base pairs upstream of the start codon). Each of the negative strand primers contained a Bgl II restriction site linker sequence at the 5′ end. After restriction digestion of the ends, the PCR fragments were directionally cloned into the luciferase reporter vector pGL3-Basic (Promega) between the Kpn I and Bgl II restriction sites upstream of the luciferase gene.

Transfection and detection of luciferase activity. N1E-115 mouse neuroblastoma cells were maintained in DMEM with 4mM L-glutamine, 4.5 g/L glucose, 1% penicylin-streptomicyn mixture and 10% fetal bovine serum (Hyclone). Cells were transiently transfected with each (α₁and β₁ sGC) luciferase plasmid construct using Fugene-6 transfection reagent (Roche). Cultures were incubated in the presence of Fugene and DNA (1 μg) for 48 hr, and assayed for luciferase activity using a luciferase reporter assay (Promega). Cells were co-transfected with a β-galactosidase construct (CMV-β-gal, ⅕ of the concentration of sGC constructs) and assayed for β-gal activity in the N1E cell lysates to normalize the transfection efficiency between cell groups (not shown).

### RESULTS:

Cloning of the mouse α₁ sGC subunit cDNA. The cDNA for mouse α₁ subunit of sGC was not previously isolated and reported. Described herein, 6 clones were isolated by screening a mouse cDNA library (Clontech) using a rat cDNA sequence as a probe. The clone containing the longest insertion was sequenced and analyzed for the presence of the open reading frame (ORF) encoding the α₁ sGC subunit. The sequence comparison of isolated cDNA demonstrated 93.3% and 83.9% homology with rat and human α₁ cDNA, respectively, confirming that the isolated clone indeed encodes mouse α₁ sGC. The sequence was submitted to the NCBI database.

Isolation of 3′ endfragment of β₁ sGC cDNA. The NCBI database contains a 2.3-kb cDNA sequence for mouse β₁ sGC (accession N AF020339). Northern analysis of mouse lung total RNA performed in our laboratory showed a 4-kb transcript for β₁ sGC (data not shown). To find the missing portion of the mRNA for β₁ sGC, a 3′-RACE analysis was performed on the mouse lung mRNA prepararation. The first cDNA strand was generated using a primer located upstream of the known 3′ end of mouse cDNA and the oligo-dT adaptor primer. A 1-kb fragment was successfully isolated. Sequence analysis of this fragment indicated that it contained the expected 70-bp region identical to the known mouse 3′ end of β₁ cDNA followed by a 956-bp novel sequence containing a conservative consensus for the polyadenylation signal and polyA stretch. The sequence was highly homologous to the rat 3′ end of β₁ cDNA (data not shown). This allowed us to conclude that the complete 3′-UTR for β₁ sGC cDNA had been isolated. The full CDNA sequence of mouse β₁ sGC subunit was submited to NCBI database.

Genomic organization of mouse α₁ and β₁ sGC genes. Three overlapping BAC clones were isolated for each of the sGC genes by separate screening of a BAC mouse genomic library (Genome Systems, Inc.) utilizing probes containing a 1.3-kb fragment of the coding sequence for mouse α₁ sGC and a 0.9-kb N-terminal fragment of the coding mouse β₁ sGC sequence. Southern analysis of isolated clones with probes specific for the 5′ and 3′ cDNA fragments for both sGC subunits confirmed that at least two out of three clones for each subunit contained a genomic fragment that hybridized with both 5′ and 3′ probes from the α₁ and β₁ cDNA's ( data not shown ). Genomic sequences isolated during screening have 99% and 100% homology in coding regions with the murine α₁ and β₁ cDNAs, respectively, confirming successful isolation of the genes for these two isoforms and subunits. Comparison of the coding sequences for the α₁ sGC subunit gene with previously cloned cDNA revealed seven mismatches in codons 49 (TAC→GAC), 52 (GAG→GAA), 319 (AAC→AGC), 343 (AAC→AAT), 445 (GAA→GAG), 487 (ATC→ACC) and 690 (GTA→ATA). Out of the seven codons, four of the replacements (49, 319, 487, 690) introduce different amino acid residues in the final protein sequence. The source for the BAC genomic library (Genomic Systems, Inc.) utilized in the analyses differed from that of the cDNA library (Clontech), indicating that the inconsistency in these sequences reflect DNA polymorphism between different strains of mice (i.e., 129/SvJ I vs. 200 BALB/c, respectively).

The positions of the exon/intron boundaries were identified by sequencing using oligonucletide primers located within the coding sequences of each gene (Table 6).

TABLE 6
Exon-intron splice junctions
of the α1 and β1 sGC genes.

| | | |
|---|---|---|
| | | splice |
| splice donor* | size of intron (kb) | acceptor |

α1 sGC

| | | |
|---|---|---|
| INTRON 1 | CAT⁻¹⁰³G/GTGGGTTCGCTCAGC | >2.0 |
| | TCCACTGCTCATAG/GT GCT | |
| INTRON 2 | CCA⁸⁵GAG/GTGAGTGTTCTCCCT | 5.5 |
| | CTTTTTCTTTCCAG/TGT GAG | |
| INTRON 3 | AAC¹⁰⁶AG/GTAAGCTAAGTTACC | 2.2 |
| | TTAATTATTCCCAG/G AAA | |
| INTRON 4 | GCA¹²⁶G/GTAATAAATAAAACT | 1.9 |
| | CTGTGTGCTTGCAG/GTG CCC | |
| INTRON 5 | TCA³⁶²AGG/GTAAGGAAAATGTAA | 3.0 |
| | CCTTTCCTTTGCAG/GTT ATG | |
| INTRON 6 | TAG⁵²⁴AAG/GTAGGGAAGGTGGAA | 4.2 |
| | TATATTGTATGTAG/GTG GAG | |
| INTRON 7 | ATC⁵⁷²AAG/GTA AGGCCGTGACT | 4.4 |
| | TTGTTTTGCCTTCAG/ATG CGA | |
| INTRON 8 | TAG⁶²⁴AG/GTATGGATGGCACTA | 2.8 |
| | TAAATTGTTCTCAG/G TTA | |

β1 sGC

| | | |
|---|---|---|
| Intron 1 | acc¹atg/GTGAGTGCTGTCAG | 0.5 |
| | TCTCTGCCCTTCAG/tac ggt | |
| Intron 2 | atc²⁶aa/GTAAGTGAACAGCC | 2.5 |
| | TCCATTTTCTTTCAG/a aaa | |
| Intron 3 | ctc⁶⁰a/GTAGGTTGAAAAC | 2.4 |
| | CATCTACAAAACAG/ac ctc | |
| Intron 4 | tttg⁹⁸cag/GTGAGATGTTCGAG | 0.8 |
| | CTGCTGCACTACAG/aac ctc | |
| Intron 5 | atg¹⁶⁴aag/GTAGTGTTCACCCG | 1.1 |
| | CCATTGACATCTAG/gtg att | |
| Intron 6 | ccc²⁴¹cag/GTAAAATGCACAG | 1.1 |
| | TTTCTGTGTCTTAG/ctc cag | |
| Intron 7 | agc²⁸⁰aag/GTAAGCAAGAACC | 1.0 |
| | CTTTCCTGTTTAAG/gaa ggg | |
| Intron 8 | cca³²⁵ag/GTAACAACTTTTAA | 3.0 |
| | CTCTGTGTGACAG/t gtg | |
| Intron 9 | gac³⁹¹ac/GTAAGCAAGGGAG | 1.5 |
| | CTAATTCCCACAG/a ttg | |
| Intron 10 | tac⁴⁷⁰aag/GCAAGTCTTCATGG | 2.5 |
| | TGTGTCACCCTAG/gtg gaa | |
| Intron 11 | gtt⁵¹⁷cag/GTGAGTAAATAAAT | 0.4 |
| | CTTTGCTTCTGCAG/ata aca | |
| Intron 12 | tac⁵⁶⁹ag/GTGAGGAGGGAAAT | 0.3 |
| | CTCATGACTTTCAG/g tgt | |
| Intron 13 | acg⁶¹¹gag/GTATGGCTCATTAG | 1.1 |
| | TCGACCCATTTAAG/gaa aca | |

*The positions of codons at which the introns interrupt the coding sequence are indicated, except for the first intron of α1 sGC gene, where it indicates the base pairs upstream of the start codon.

Intron sizes were estimated using PCR (see Table 6) and for introns 1, 2, 10, 11, 12 of β₁ sGC by complete sequencing. While the size of intron 1 for sGCα₁ was not determined, partial sequencing indicates it is more than 2 Kb.

The α₁ sGC gene encompasses at least 26.4 kb and includes 9 exons and 8 introns, while the β₁ sGC gene contains 14 exons and 13 introns and spans 22 kb. Start codons are positioned in the second and first exons of the α₁ and β₁ genes, respectively. The GT/AG donor/acceptor consensus was maintained in all introns for both genes, except for intron 10 in β₁ sGC, where the donor site was GC. The sequences that flank the exon/intron boundaries in the α₁ and β₁ sGC genes are presented in Table 1.

Chromosomal localization. BAC clones containing α₁ and β₁ genes were used for chromosomal localization by FISH analysis. DNA from BAC clones containing genomic regions of α₁ and β₁ sGC genes was labeled with digoxigenin dUTP and hybridized to normal metaphase chromosomes derived from mouse embryo fibroblast cells. A total of 80 metaphase cells were analysed for each genomic clone with 71 and 72 chromosomes exhibiting specific labeling for α₁ and β₁ sGC genes, respectively. Both genes co-localized to mouse chromosome 3. The α₁ sGC gene positioned at 44% and PI sGC at 46% of the distance from the heterochromatic-euchromatic boundary to the telomere of chromosome 3, which corresponds to band 3E3-F1.

Analysis of the promoter activity of 5′ regions for α₁ and β₁ sGC genes in the N1E-115 cell line. Murine neuroblastoma N1E-115 cells were selected for promoter analysis as host cells since expression of sGC was shown in this cell line (34). 1.6-kb and 1.4-kb of the 5′-flanking regions extended until the first identified exons of the α₁ and β₁ sGC subunit genes were subdloned upstream of the luciferase gene of the pGL3-Basic luciferase reporter vector (Promega). The constructs were transiently transfected in N1E-115 cells. Luciferase activity generated by each of the constructs was compared with the activity of the promotorless pGL3-basic plasmid. The upstream regions of the α₁ and β₁ sGC genes demonstrated different transcriptional activity in N1E-115 cells. Values were normalized using β-galactosidase construct (CMV-β-gal ) cotransfections and the total protein concentration of each group. The values obtained represented the normalized means ±S.D. of three different experiments. The relative level of luciferase activity for the β₁ sGC construct was 4.6-fold higher when compared with the control, while the α₁ sGC construct activity was only 2-fold higher than the control promoterless plasmid.

Prediction of the organization of the human sGC genes. cDNA's for mouse and human α₁ and β₁ subunits were compared with the Human Genome Database. A clone was identified containing putative genomic regions for both human α₁ and β₁ subunits (clone AC021433), which is ascribed to chromosome 2. Exons 3-9 of the human α₁ gene and all exons of the human β₁ gene were identified in this genomic fragment. The comparison of predicted exon-intron boundaries and donor/acceptor sites from the human genes with those of the mouse genes revealed that they are identical in both species with the exception of intron 4 (donor and acceptor sites) and intron 9 (acceptor site) of the β₁ subunit.

### DISCUSSION

The mouse α₁ and β₁ sGC genes map to the third chromosome. However, they are separated from each other by an extended region comprising 2% of the total chromosomal length. This finding excludes the possibility of tandem organization and directly coordinated transcription as proposed for the fish genes. This conclusion is supported by the independent ability of the 5′-flanking regions of the α₁ and β₁ sGC genes to drive transcription. However, the trans-coordinated transcriptional regulation by the same factors is possible.

The rat α₁ andβ₁ genes map to chromosome 2 and are closely linked to the particulate guanylyl cyclase isoform locus (GC-A) and quantitative trait locus (QTL) which have been associated with salt-sensitive hypertension in Dahl rats (Azam, et al., 1998). Thus far, no direct connection of the genomic loci identified for α₁ and β₁ sGC to hypertension in mouse or human has been demonstrated (Danziger, et al., 2000). Here it is shown that both mouse genes co-localize on chromosome 3 in the area corresponding to band 3E3-F1. The α₁ and β₁ subunits are co-localized with Muc1, Pk1r, Ntrk1, CD1 and Fcfr1 loci located at 44.8-46.5 positions of chromosome 3. It is contemplated that mutations in these genes contributes to these diseases.

Probing of the Human Genome database with the mouse and human cDNA sequences identified the clone AC021433 which contains 8 exons of the human α₁ gene and 14 exons of the human β₁ gene. This fragment is ascribed to human chromosome two. Although this fragment is missing the first two exons of the α₁ gene this is the most probable candidate for the locus of human the α₁ and β₁ genes. In a previous report (Giuili, et al., 1993), α₃ and β₃ subunits of human sGC (later confirmed to be α₁ and β₁ sGC genes (Zabel, et al., 1998) were colocalized to human chromosome 4 at q31.1 -q33, what represents some discrepancy with recent results. However, previously reported mapping of the chromosomal position for α₁ and β₁ of sGC were made using cDNA for corresponding isoforms as probes for the localization (Giuili, et al., 1993). Considering the high level of homology between coding regions of both sGC subunits in various isoforms and, in addition, the existence of regions of extensive homologies (in the catalytic domain, for example) to the particulate guanylyl cyclase family, the use of cDNA for chromosomal localization could result in ambiguities.

The transcriptional regulation of the expression of sGC has not been previously examined. Recently, evidence to support altered expression of mRNA of sGC subunits has emerged. In primary rat pulmonary artery smooth muscle cells, prolonged NO treatment leads to decreased NO-stimulated sGC activity and mRNA levels (Filippov et al. 1997). sGC levels rise in unborn rat pulmonary artery, beginning at approximately 20 days of gestation and mRNA, protein, and activity remain elevated at least 8 days following birth (Bloch et al., 1997). Decreased rates of sGC transcription have also been indicated in other models following NO treatment and administration of cAMP-elevating agents (24;25). Furthermore, nerve growth factor administration to rat PC-12 cells results in decreased steady state levels of sGC α₁ and β₁ mRNA.

It was found by the inventors that estrogen treatment decreases α₁ and β₁ sGC mRNA levels in rat uterus. The precise mechanisms underlying these effects on sGC in specific tissues are largely unknown. The activity of putative promoter regions demonstrate different transcriptional activity for both subunits, demonstrating the potential for finely tuned regulation.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Abremski and Hoess, Protein Eng; 5(1):87-91, 1992.

Abremski et al., Cell; 32(4):1301-1311, 1983.

Abuin and Bradley, Mol. Cell. Biol., 16:1851-1856, 1996.

Alt et al., J Biol. Chem., 253(5):1357-1370, 1978.

Angerer et al. in Genetic Engineering: Principles and Methods Setlow and Hollaender, Eds. Vol 7, pgs 43-65 plenum Press, New York, 1985.

Arai et al., Cancer Lett., 122(1-2): 157-163, 1998.

Austin et al., Cell, 25:729-736, 1981.

Azam, M., Gupta, G., Chen, W., Wellington, S., Warburton, D. & Danziger, R. S. Hypertension 32:149-54, 1998.

Baichwal and Sugden, In: Gene Transfer, Kucherlapati R, ed., New York, Plenum Press, pp. 117-148, 1986.

Baker, and Harland, Gene Dev., 10:1880-1889, 1996.

Ballester et al., Cell 63:851-859 1990.

Basu et al., Nature 356:713-715 1992.

Batterson and Roizrnan, J. Virol., 46:371-377, 1983.

Behrends, S., Harteneck, C., Schultz, G. & Koesling, D. ( ) J Biol Chem 270: 21109-13, 1995.

Behrends, S., Steenpass, A., Porst, H. & Scholz, H. Biochem Pharmacol 59:713-7, 2000.

Bellon et al., de Ses Filiales,190(1):109-142, 1996.

Bellus, J. Macromol. Sci. Pure Appl. Chem, A311:1355-1376, 1994.

Benvenisty and Neshif, Proc. Nat'/. Acad. Sci. USA, 83:9551-9555, 1986.

Bemardis et al., Digestion 60:82-85, 1999.

Bems and Bohenzky, Adv. Virus Res., 32:243-307, 1987.

Berns and Giraud, Curr. Top. Microbiol. Immunol., 218:1-23, 1996.

Bems, Microbiol. Rev., 54:316-329, 1990.

Bertran et al., J. Virol., 70(10):6759-6766, 1996.

Bloch, K. D., Filippov, G., Sanchez, L. S., Nakane, M. & de la Monte, S. M. Am J Physiol 272:L400-6, 1997.

Bollag et al., Nat Genet 12:144-148 1996.

Boss, G. R. Proc Natl Acad Sci U S A 86:7174-8, 1989.

Bradley et al. Nature, 309:255-258, 1984

Brannan et al., Genes & Development 8:1019-1029 1994.

Brannan, C I et al., Gene Devel., 8:1019-1029, 1994.

Bresalier and Kim In: Sleisenger and Fordtran's gastrointestinal and liver disease, M. Feldman, B. Scharschmidt and M. Sleisenger, eds., W. B. Saunders and Company, pp. 1906-1942, 1998.

Bronner et al., Nature, 368:258-261, 1994.

Brown et al., J. Neurochem., 40:299-308, 1983.

Buchberg, Cleveland, Jenkins, Copeland, Nature 347:291-294, 1990.

Caccone, A., Garcia, B. A., Mathiopoulos, K. D., Min, G. S., Moriyama, E. N. & Powell, J. R. Insect Mol Biol 8:23-30, 1999.

Chalfie et al., Science, 263(5148):802-805, 1994.

Chen and Okayama, Mol. Cell. Biol., 7:2745-2752, 1987.

Chhajlani, V., Frandberg, P. A., Ahlner, J., Axelsson, K. L. & Wikberg, J. E. FEBS Lett 290:157-8, 1991.

Chomczynski and Sacchi, Anal. Biochem., 162:156-159, 1987.

Clarke et al., Nature, 359:328-330, 1992.

Coffin, In: Virology, ed., New York: Raven Press, pp. 1437-1500, 1990.

Cohen et al., In: Cancer: principles and practice of oncology, V. Devita, S. Hellman and S. Rosenberg, eds., Philadelphia: Lippincott-Raven publishers, pp. 1144-1197, 1997.

Colberre-Garapin et al., Dev Biol Stand., 50:323-326, 1981.

Copeland et al., Science 262:57-66 1993.

Cotten et al., Proc Nat'l Acad Sci U S A., 89(13):6094-6098, 1992.

Couch et al., Am. Rev. Resp. Dis., 88:394-403, 1963.

Culver et al, Science, 256:1550-1552, 1992.

Cumo and Oettinger, Nuc. Acids Res., 22(10):1810-1814, 1994.

Curiel, Ann. N Y Acad Sci. 716:36-56. 1994.

Dale and Ow, Proc. Nat'l. Acad. Sci. USA, 88:10558-10562, 1991.

Danziger, R. S., Pappas, C., Barnitz, C., Varvil, T., Hunt, S. C. & Leppert, M. F. J Hypertens 18:263-6, 2000.

Davey et al., EPO No. 329 822.

Dawson, V. L. & Dawson, T. M. Prog Brain Res 118:215-29, 1998.

DeClue et al., Cell 69:265-273, 1992.

DeLuca et al., J. Virol., 56:558-570, 1985.

Deng, C. et al., Cell 82:675-684, 1995.

Derynck and Zhang, Curr. Biol., 6:1226-1229, 1996.

Derynck, “TGF-β-receptor-mediated signaling,” Trends Biol. Sci., 19:548-553, 1994.

Dickson et al., Development, 121:1845-1854, 1995.

Dietrich et al., Cell, 75:631-639, 1993.

Donehower et al., Nature, 356:215-221, 1992.

Donehower, L A et al., Nature, 356:348-352, 1992.

Dubensky et al., Proc. nat'l. acad. sci. USA, 81:7529-7533, 1984.

Eliyahu, Raz, Gruss, D. Givol, M. Oren, Nature 312:646-649, 1984.

Elroy-Stein et al., Proc. Nat'l Acad. Sci. USA, 86(16):6126-6130, 1989.

Elshami et al., Gene Therapy, 7(2):141-148, 1996.

Eppert et al., Cell, 88:543-552, 1996.

Evans et al. Nature, 292:154-156,1981.

Fechheimner et al., Proc. Nat'l. Acad. Sci. USA, 84:8463-8467, 1987.

Fenoglio-Preiser et al., In: Gastrointestinal Pathology, New York: Raven Press, 1989.

Ferkol et al., FASEB J., 7:1081-1091, 1993.

Fero, Randel, Gurley, Roberts, Kemp, Nature 396:177-180, 1998.

Filippov, G., Bloch, D. B. & Bloch, K. D. J Clin Invest 100:942-8, 1997.

Fodor et al, Science, 251:767-773, 1991.

Fraley et al., Proc. Nat'l. Acad. Sci. USA, 76:3348-3352, 1979.

Freifelder, Physical Biochemistry Applications to Biochemistry and Molecular Biology, 2nd ed. Wm. Freeman and Co., New York, N.Y., 1982.

Frohman, M. A., In: Pcr Protocols: A Guide To Methods And Applications, Academic Press, N.Y., 1990.

Gall et al. Meth. Enzymol., 21:470-480, 1981.

Garbers, D. L. J Biol Chem 254: 240-3, 1979.

Gerzer, R., Bohme, E., Hofinann, F. & Schultz, G. FEBS Lett 132: 71-4, 1981.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Ghosh-Choudhury et al., EMBO J, 6:1733-1739, 1987.

Gingeras et al., PCT Application WO 88/10315

Ginsberg et al., Proc. Nat'l Acad. of Sci. USA, 88(5):1651-1655, 1991.

Giphart-Gassler et al. Mutat., Res., 214:223-232, 1989.

Giuili, G., Scholl, U., Bulle, F. & Guellaen, G. FEBS Lett 304:83-8, 1992.

Giuili, G., Roechel, N., Scholl, U., Mattei, M. G. & Guellaen, G. Hum Genet 91:257-60, 1993.

Glorioso et al., Ann. Rev. Microbiol. 49:675-710, 1995.

Gomez-Foix et al., J. Biol. Chem., 267:25129-25134, 1992.

Gopal,Mol. Cell Biol., 5:1188-1190,1985.

Gossen and Bujard, Proc. Nat'l. Acad. Sci. USA, 89:5547-5551, 1992.

Gossen et al., Science, 268:1766-1769, 1995.

Gossler et al. Proc. Nat'l. Acad. Sci USA 83:9065-9069, 1986.

Graff et al., Cell, 79:169-179, 1994.

Graff et al., Cell, 85:479-487, 1996.

Graham and Prevec, Biotechnology, 20:363-390, 1992.

Graham and Prevec, In: Methods in Molecular Biology: Gene Transfer and Expression Protocol, E. J. Murray, ed., Humana Press, Clifton, N.J., 7:109-128, 1991.

Graham and van der Eb, Virology, 52:456-467, 1973.

Graham et al., J. Gen. Virol., 36:59-72, 1977.

Greenblatt, Bennett, Hollstein, Harris, Cancer Res 54:4855-4878 1994.

Groden et al., Cell, 66:589-600, 1991.

Grunhaus and Horwitz, Seminar in Virology, 3:237-252, 1992.

Guha et al., Oncogene 12:507-513, 1996.

Gutmann, Saporito-Irwin, DeClue, Wienecke, Guha, Oncogene 15, 1611-1616, 1997.

Hacia et al., Nature Genetics, 14:441-447, 1996.

Halling et al., Am J Clin Pathol 106(3):282-288, 1996.

Harland and Weintraub, J Cell. Biol., 101:1094-1099, 1985.

Harlow et al., Antibodies: A Laboratory Manual, Cold Spring Harbor, N.Y., 1988

Heldin et al., Nature, 390:465-471, 1997.

Hersdorffer et al., DNA Cell Biol., 9:713-723, 1990.

Herz and Gerard, Proc. Nat'l. Acad. Sci. USA, 90:2812-2816, 1993.

Hinds and Weinberg, Curr Opin Genet Dev 4:135-141, 1994.

Hoess et al., Proc. Nat'l. Acad. Sci. USA, 79:3398-3402, 1982.

Hogan et al. Manipulating the Mouse Embryo Cold Spring Harbor Laboratory, Cold Spring Harbor, N.Y., 1986.

Hogan et al., In: Manipulating the mouse embryo, New York: Cold Spring Harbor Press, 1994.

Holland et al., Virology, 101:10-18,1980.

Hollstein, M et al., Science, 253:49-53, 1991.

Honess and Roizman, J. Virol., 14:8-19, 1974.

Honess and Roizman, J. Virol., 16:1308-1326, 1975.

Huang, P. L. Semin Perinatol 24:87-90, 2000.

Ignarro, L. J., Degnan, J. N., Baricos, W. H., Kadowitz, P. J. & Wolin, M. S. Biochim Biophys Acta 718:49-59, 1982

Imler et al., Gene Ther; 2(4): 263-268, 1995.

Innis et al., PCR Protocols, Academic Press, Inc., San Diego Calif., 1990.

Ionov et al., Nature, 363:558-561, 1993.

Ishizaki et al., Surgery 111:706-710, 1992.

Jacks et al., Nature Genetics, 7:353-361, 1994.

Jacks et al., Nature, 359:295-300, 1992.

Jacks et al., Curr Biol 4:1-7, 1994.

Jacks, Annu Rev Genet 30:603-636, 1996.

Jacks, T et al., Current biology, 4:1-7, 1994.

Jacks, T. et al., Nature Genet., 7:353-362, 1994.

Jaenich, Proc. Nat'l. Acad. Sci. USA 73:1260-1264, 1976.

Jaenisch, Science, 240:1468-1474, 1988.

Jahner et al. Nature, 298:623-628, 1982.

Jahner et al., Proc. Nat'l. Acad. Sci. USA 82:6927-6931, 1985.

Jhanwar, Chen, Li, Brennan, Woodruff, Cancer Genet Cytogenet 78:138-44, 1994.

Jirciny, Trends in Genetics, 10:164-168, 1994.

Johnson, Look, DeClue, Valentine, Lowy, Proceedings of the National Academy of Sciences of the United States of America 90:5539-5543 1993.

Jones and Shenk, Cell, 13:181-188, 1978.

Jones, Hancock, Vogel, Donehower, Bradley, Proc Natl Acad Sci USA 95:15608-15612, 1998.

Kaartinen et al., Nature Genetics, 11:415-421, 1995.

Kamijo, Bodner, van de Kamp, Randle, Sherr, Cancer Res 59:2217-2222, 1999.

Kamijo, T. et al., Cancer Res. 59:2217-2222, 1999.

Kamijo, T. et al., Cell, 91:649-659, 1997.

Kamisaki, Y., Saheki, S., Nakane, M., Palmieri, J. A., Kuno, T., Chang, B. Y., Waldman, S. A. & Murad, F. J Biol Chem 261:7236-41, 1986.

Kaneda et al., Science, 243:375-378, 1989.

Karlsson et al., EMBO J., 5:2377-2385, 1986.

Kato et al., J. Biol. Chem., 266:3361-3364, 1991.

Kaufinan Methods Enzymol. 185:537-566, 1990.

Kearns et al., Gene Ther., 3:748-755, 1996.

Kelleher and Vos, Biotechniques. 17(6):1110-1117, 1994.

Kingsley, Genes Dev., 8:133-146, 1994.

Kinzler and Vogelstein, Cell, 87:159-170, 1996.

Kioussi, Gruss, Trends Genet 12:84-86, 1996.

Klein et al., Nature, 327:70-73, 1987.

Knudson, Jr., Annu Rev Genet 20:231-251, 1986.

Koesling, D., Herz, J., Gausepohl, H., Niroomand, F., Hinsch, K. D., Mulsch, A., Bohme, E., Schultz, G. & Frank, R. FEBS Lett 239:29-34, 1988.

Koesling, D., Harteneck, C., Humbert, P., Bosserhoff, A., Frank, R., Schultz, G. & Bohme, E. FEBS Lett 266:128-32, 1990.

Koller and Smithies, Ann. Rev. Immun., 10:705-730, 1992.

Kotin and Berns, Virol., 170:460-467, 1989.

Kotin et al., Genomics, 10:831-834, 1991.

Kotin et al., Proc. Nat'l. Acad. Sci. USA, 87:2211-2215, 1990.

Kretzschmar, Doody, Timokhina, Massague, Genes Dev 13:804-816, 1999.

Krull et al., Curr. Biol., 7:571-580, 1997.

Kulkarni et al., Proc. Nat'l. Acad. Sci. USA, 90:770-774, 1993.

Kwoh et al., Proc. Nat'l. Acad. Sci. USA, 86:1173, 1989.

Lakso et al., Proc. Nat'l. Acad. Sci. USA, 93:5860-5865, 1996.

Largaespada, Brannan, Shaughnessy, Jenkins, Copeland, Current Topics in Microbiology & Immunology 211:233-239, 1996.

Leach et al., Cell, 75:1215-1225, 1993.

Lee et al., Nature, 359:288-294, 1992.

Legius, Marchuk, Collins, Glover, Nat Genet 3:122-126, 1993.

Levrero et al., Gene, 101: 195-202, 1991.

Li, Y., Maher, P. & Schubert, D. J Cell Biol 139:1317-24, 1997.

Littlefield, Science, 145:709-710, 1964.

Liu et al., Nature, 381:620-623, 1996.

Liu et al., Proc. Nat'l. Acad. Sci. USA, 94:10669-10674, 1997.

Liu, H., Force, T. & Bloch, K. D. J Biol Chem 272:6038-43, 1997.

Liu, L. & Stamler, J. S. ( ) Cell Death Differ 6:937-42, 1999.

Lu et al., Nature Genet., 19:17-18, 1998.

Machy et al. Proc. Nat'l Acad. Sci. USA, 85:8027-8031, 1988.

Maeser and Kahmann, Mol. Gen. Genetics, 230:170-176, 1991.

Malkin, D. Cancer Genet. Cytogenet., 66:83-92, 1993.

Mann et al., Cell, 33:153-159, 1983.

Marchuk et al., Genomics 13:672-680 1992.

Markowitz et al., J. Virol., 62:1120-1124, 1988.

Markowitz et al., Science, 268:1336-1338, 1995.

Martin et al., Cell 63:843-849, 1990.

Massague, Cell, 85:947-950, 1996.

McClatchey et al., Genes Dev 12:1121-1133, 1998.

Menon et al., Proc Natl Acad Sci USA 87:5435-5439, 1990.

Mikami, T., Kusakabe, T. & Suzuki, N. Eur J Biochem 253:42-8, 1998.

Mikami, T., Kusakabe, T. & Suzuki, N. J Biol Chem 274:18567-73, 1999.

Miller et al., PCT Application WO 89/06700

Mizukami et al., Virology, 217:124-130, 1996.

Morrison, White, Zock, Anderson, Cell 96:737-749, 1999.

Mortensen, Hypertension; 22(4):646-651, 1993.

Mulligan, Science, 260:926-932, 1993.

Myers, EPO 0273085

Nakane, M., Saheki, S., Kuno, T., Ishii, K. & Murad, F. Biochem Biophys Res Commun 157:1139-47, 1988.

Nakane, M., Arai, K., Saheki, S., Kuno, T., Buechler, W. & Murad, F. J Biol Chem 265:16841-5, 1990.

Nakane, M. & Murad, F. Adv Pharmacol 26:7-18, 1994.

Nakao et al, EMBO J, 16:5353-5362, 1997.

Nakao et al., J. Biol. Chem., 272:2896-2900, 1997.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Nieto, Sechrist, Wilkinson, Bronner-Fraser, EMBO Journal 14:1697-1710 1995.

Nigro et al., Nature 342:705-708, 1989.

Nishisho et al., Science, 253:665-669, 1991.

Oettinger et al., Science, 248:1517-1523, 1990.

Ogawa, Neuropathologica, 77(3):244-253, 1989.

Ohara et al., Proc. Nat'l Acad. Sci. USA, 86: 5673-5677, 1989.

Ohlstein, E. H., Wood, K. S. & Ignarro, L. J. Arch Biochem Biophys 218:187-98, 1982.

Onouchi et al., Mol. Cell. Biol., 247:653-660, 1995.

Orlow et al., Int J Oncol 15:17-24, 1999.

Oshima et al, Cancer Res., 57:1644-1649, 1997.

Oshima et al., Dev. Bio., 179:297-302, 1996.

Ostrove et al., Virology, 113:532-533, 1981.

Palmiter et al. in Sambrook et al. Molecular Cloning: A Laboratory Manual Cold Spring Harbor Laboratory, Cold Spring Harbor, N.Y., 1989.

Palmiter et al. in The Qiagenologist, Application Protocols, 3rd edition, published by Qiagen, Inc., Chatsworth, Calif., 1982;

Palmiter et al. Nature, 300:611, 1982.

Papadopoulos et al., Science, 263(5153):1625-1629, 1994.

Papapetropoulos, A., Marczin, N., Mora, G., Milici, A., Murad, F. & Catravas, J. D. Hypertension 26:696-704, 1995.

Papapetropoulos, A., Abou-Mohamed, G., Marczin, N., Murad, F., Caldwell, R. W. & Catravas, J. D. Br J Pharmacol 118:1359-66, 1996.

Parada, Land, Weinberg, Wolf, Rotter, Nature 312.649-651 1984.

Parsons et al., Cell, 75:1227-1236, 1993.

Paskind et al., Virology, 67:242-248, 1975.

Pathogenesis. (Johns Hopkins University, Baltimore, 1992).

Pease et al., Proc. Nat'l. Acad. Sci. USA, 91:5022-5026, 1994.

Perales et al., Proc. Nat'l. Acad. Sci. 91:4086-4090, 1994.

Pignon et al., Hum. Mutat., 3:126-132, 1994.

Pinkel et al., Proc Nat'l Acad Sci USA. 83(9):2934-2938, 1986.

Polyak, Biochem. Biophys. Acta, 1242:185-199, 1996.

Ponnazhagan et al., Hum. Gene Ther., 8:275-284, 1997a.

Ponnazhagan et al., J. Gen. Virol., 77:1111-1122, 1996.

Post et al., Cell, 24:555-565, 1981.

Potter et al., Proc. Nat'l Acad. Sci. USA, 81:7161-7165, 1984.

Prolla et al., Nat Genet 18:276-279 1998.

Quillet et al. J. Immunol., 141:17-20, 1988.

Racher et al., Biotechnology Techniques, 9:169-174, 1995.

Radler et al., Science, 275:810-814, 1997.

Ragot et al., Nature, 361:647-650, 1993.

Ramirez-Solis et al., Methods in Enzymology, Acad. Press, 225:855-878, 1993.

Renan, Radiother. Oncol., 19:197-218, 1990.

Reynolds et al., Hum Genet 90:450-456, 1992.

Riccardi, V M. Neurofibromatosis: Phenotype, Natural History and

Riccardi, Womack, Jacks, Am J Pathol 145:994-1000, 1994.

Rich et al., Hum. Gene Ther., 4:461-476, 1993.

Ridgeway, In: Vectors: A survey of molecular cloning vectors and their uses, Rodriguez R L, Denhardt D T, ed., Stoneham:Butterworth, pp. 467-492, 1988.

Riggins et al., Cancer Res., 57:2578-2580, 1997.

Rippe et al., Mol. Cell Biol., 10:689-695, 1990.

Ritter, D., Taylor, J. F., Hoffinann, J. W., Camaghi, L., Giddings, S. J., Zakeri, H. & Kwok, P. Y. Biochem J346(Pt 3):811-6, 2000.

Robertson In: Teratocarcinomas and Embryonic Stem Cells: A Practical Approach, E. J. Robertson, Ed., IRL Press, Oxford, 1987.

Robertson et al. Nature, 322:445-448, 1986.

Robertson, In: Current Communications in Molecular Biology, Capecchi, M. R. (ed.), Cold Spring Harbor Press, Cold Spring Harbor, N.Y. pp. 39-44, 1989.

Roizrnan and Sears, In Fields' Virology, 3rd Edition, eds. Fields et al. (Raven Press, New York, N.Y.), pp. 2231-2295, 1995.

Rosenfeld et al., Cell, 68:143-155, 1992.

Rosenfeld et al., Science, 252:431-434, 1991.

Roux et al., Proc. Nat'l Acad. Sci. USA, 86:9079-9083, 1989.

Rudolph et al., Cell 96:701-712, 1999.

Russwurm, M., Behrends, S., Harteneck, C. & Koesling, D. Biochem J335:125-30, 1998. Sambrook et al., In: Molecular Cloning: A Laboratory Manual, 2d Ed., Cold Spring Harbor Laboratory Press, Cold Spring Harbor, N.Y., 1989.

Samulski et al., EMBO J., 10:3941-3950, 1991.

Sanford et al., Dev., 124:2659-2670, 1997.

Santerre et al, Gene. 30(1-3):147-156, 1984.

Sauer, Methods in Enzymology, 225:890-900, 1993.

Sauer, Mol. Cell. Biol., 7:2087-2096, 1987.

Schatz et al., Cell 59(6):1035-1048, 1989.

Schutte et al., Cancer Res., 56:2527-2530, 1996.

Schwartzberg et al. Science 212:799-803, 1989.

Serrano, Lin, McCurrach, Beach, Lowe, Cell 88:593-602, 1997. Severina, I. S. Biochemistry (Mosc) 63:794-801, 1998.

Shah, Groves, Anderson, Cell 85:331-343, 1996.

Shenk, Cell 13(4):791-798, 1978.

Sherman, Daston, Ratner, in Neurofibromatosis Type 1:From gentype to Phenotype M. a. C. Upadhaya, D. N., Ed. (BIOS Scientific Publishers, Oxford, 1998).

Sherr, C J. Gene Dev., 12:2984-2991, 1998.

Shiraishi et al., Transplant International, 10(3):202-206, 1997.

Shoemaker et al., Nature Genetics 14:450-456, 1996.

Shull et al., Nature, 359:693-699, 1992.

Sieber-Blum, Zhang, J Anat 191:493-499 1997.

Sirard et al., Gene Dev., 12:107-119, 1998.

Srivastava et al., J. ViroI., 45:555-564, 1983.

Srivastava, Zou, Pirollo, Blattner, Chang, Nature 348:747-749, 1990.

Stemberg and Hamilton, J. Mol. Biol., 150:467-486, 1981.

Stemberg et al., J. Mol. Bio., 187:197-212, 1986.

Stewart et al., EMBO J., 6: 383-388, 1987.

Stratford-Perricaudet and Perricaudet, In: Human Gene Transfer, Eds, O. Cohen-Haguenauer and M. Boiron, Editions John Libbey Eurotext, France, pp. 51-61, 1991.

Stratford-Perricaudet et al., Hum. Gene Ther., 1:241-256, 1990.

Takaku et al., Cell, 92:645-656, 1998.

Tanaka, Omura, Watanabe, Oda, Nakanishi, J Surg Oncol 57:57-64, 1994.

Tate-Ostroff et al., Proc. Nat'l. Acad. Sci., 86:745-749, 1989.

Temin, In: Gene Transfer, Kucherlapati (ed.), New York: Plenum Press, pp. 149-188, 1986.

Thiagalingam et al., Nature Gene., 13:343-346, 1996.

Thippeswamy, T. & Morris, R. Brain Res 774:116-22, 1997.

Tomlinson et al., Cancer Metast. Rev., 16:67-79, 1997.

Top et al., J. Infect. Dis., 124:155-160, 1971.

Troneguzzo et al., Nucleic Acids Res., 16: 5515-5532, 1988.

Tur-Kaspa et al., Mol. Cell Biol., 6:716-718, 1986.

U.S. Pat. No. 4,367,110

U.S. Pat. No. 4,430,434

U.S. Pat. No. 4,452,901

U.S. Pat. No. 4,559,302

U.S. Pat. No. 4,668,621

U.S. Pat. No. 4,683,195

U.S. Pat. No. 4,683,195

U.S. Pat. No. 4,683,202

U.S. Pat. No. 4,683,202

U.S. Pat. No. 4,800,159

U.S. Pat. No. 4,800,159

U.S. Pat. No. 4,873,191

U.S. Pat. No. 4,883,750

U.S. Pat. No. 4,959,317

U.S. Pat. No. 4,960,704

U.S. Pat. No. 5,252,479

U.S. Pat. No. 5,633,365

U.S. Pat. No. 5,633,365

U.S. Pat. No. 5,665,549

U.S. Pat. No. 5,665,549

U.S. Pat. No. 5,672,344

U.S. Pat. No. 4,727,028

Van der Putten et al. Proc. Nat'l. Acad. Sci. USA 82:6148-6152, 1985.

Varmus et al., Cell, 25:23-36, 1981.

Venkatachalam et al., Embo J 17:4657-4667, 1998.

Vogel, Brannan, Jenkins, Copeland, Parada, Cell 82:733-742, 1995.

Vogel, Parada, Mol Cell Neurosci 11(1-2):19-28,1998.

Wagner et al., Proc. Nat'l. Acad. Sci. 87, 9:3410-3414, 1990.

Waldrip et al., Cell, 92:797-808, 1998.

Walker et al., Proc. Nat'l Acad. Sci. USA, 89:392-396, 1992.

Wang and Anderson, Neuron, 18:383-396, 1997.

Wang et al., J. Biol. Chem., 270:22044-22049, 1995.

Watanabe et al., Exp Cell Res. 230(1):76-83, 1997.

Weinberg, Science 254, 1138-1146 1991.

Weiss, Histological Typing of Soft Tissue Tumors (Springer Verlag, ed. (2nd Ed), 1994).

Werthman et al., Journal of Urology, 155(2):753-756, 1996.

White, Cell, 92:591-592, 1998.

Wicketal., Oncogene, 12:973-978, 1996.

Wigley et al., Reprod. Fertil. Dev., 6:585-588, 1994.

Williams et al., Nat Genet 7, 480-484, 1994.

Willnow and Herz, Methods Cell Biol., 43Pt A:305-334, 1994.

WO 90/07641 filed Dec. 21, 1990.

Wong et al., Gene, 10:87-94, 1980.

Wrana and Attisano, Trends Gene., 12:493-496, 1996.

Wu and Wu, Adv. Drug Delivery Rev., 12:159-167, 1993.

Wu and Wu, J. Biol. Chem., 262:4429-4432, 1987.

Wu and Wu, Biochem., 27:887-892, 1988.

Wu et al., Genomics, 4:560, 1989.

Xu et al., Cell 62:599-608, 1990.

Xu et al., Genes Chromosomes Cancer 4:337-342, 1992.

Yan, Y. et al., Gene Dev. 11:973-983, 1997.

Yang et al., Proc. Nat'l. Acad. Sci. USA, 87:9568-9572, 1990.

Yang et al., Proc. Nat'l. Acad. Sci. USA, 95:3667-3672, 1998.

Zabel, U., Weeger, M., La, M. & Schmidt, H. H. Biochem J335:51-7, 1998.

Zambetti, Levine, Faseb J7, 855-865 1993.

Zhang et al., Nature, 383:168-172, 1996.

Zhang, P. et al., Nature 387:151-158, 1997.

Zhu, Richardson, Parada, Graff, Cell 94:703-714, 1998.

What is claimed is:

1. An isolated nucleic acid comprising a region having a nucleotide sequence that encodes the polypeptide sequences of SEQ ID NO: 2.

2. The nucleic acid of claim 1, wherein the region is further defined as having the nucleotide sequence of the nucleotide sequence of SEQ ID NO: 1.

3. An isolated and purified polynucleotide comprising a base sequence that is identical or complementary to a segment of at least 15 contiguous bases of SEQ ID NO: 1.

4. The polynucleotide of claim 3, wherein said polynucleotide hybridizes to a polynucleotide that encodes a polypeptide comprising the amino acid residue sequence of SEQ ID NO: 1 or to the complement of such a sequence.

5. The polynucleotide of claim 3 wherein said polynucleotide comprises a base sequence that is identical or complementary to a segment of at least 20 contiguous bases of SEQ ID NO: 1.

6. The polynucleotide of claim 3 wherein said polynucleotide comprises a base sequence that is identical or complementary to a segment of at least 30 contiguous bases of SEQ ID NO: 1.

7. The polynucleotide of claim 3 wherein said polynucleotide comprises a base sequence that is identical or complementary to a segment of at least 50 contiguous bases of SEQ ID NO: 1.

8. The polynucleotide of claim 3 wherein said polynucleotide comprises a base sequence that is identical or complementary to a segment of at least 100 contiguous bases of SEQ ID NO: 1.

9. The polynucleotide of claim 3, wherein said polynucleotide comprises a base sequence that is identical or complementary to all contiguous bases of SEQ ID NO: 1.

10. An expression vector comprising a polynucleotide that encodes a polypeptide comprising an amino acid residue sequence of SEQ ID NO: 2.

11. The expression vector of claim 11, wherein said polynucleotide comprises the nucleotide base sequence of SEQ ID NO: 1.

12. The expression vector of claim 10, wherein said polynucleotide is operatively linked to an enhancer-promoter.

13. A recombinant host cell comprising a polynucleotide that encodes a polypeptide comprising an amino acid residue sequence of SEQ ID NO: 2.

14. The recombinant host cell of claim 13, comprising a polynucleotide that encodes a polypeptide comprising the amino acid residue sequence of SEQ ID NO: 2.

15. The recombinant host cell of claim 14, wherein said polynucleotide comprises the nucleotide base sequence of SEQ ID NO: 1.

16. The recombinant host cell by claim 13, wherein said polynucleotide is introduced into said cell by transformation of said cell with a vector comprising said polynucleotide.

17. The recombinant host cell of claim 13, wherein said host cell expresses said polynucleotide to produce the polypeptide.

18. A process for preparing a cell expressing a polypeptide comprising the steps of
(a) transfecting a cell with a polynucleotide that encodes a polypeptide comprising an amino acid residue sequence of SEQ ID NO: 2 to produce a transformed host cell; and
(b) maintaining the transformed host cell under biological conditions sufficient for expression of said polypeptide in the host cell.

19. The method of claim 18, wherein the polynucleotide comprises a region having a nucleotide sequence of SEQ ID NO: 1.

20. The process of claim 18, further defined as a process for preparing a cell expressing a polypeptide comprising the amino acid residue sequence of SEQ ID NO: 2.

21. The process of claim 18, further comprising purifying an expressed polypetide having the amino acid sequence of SEQ ID NO: 2 from the transformed host cell.

22. An isolated nucleic acid comprising a region having a nucleotide sequence of SEQ ID NO: 3.

23. An isolated and purified polynucleotide comprising a base sequence that is identical or complementary to a segment of at least 15 contiguous bases of SEQ ID NO: 3.

24. The polynucleotide of claim 23 wherein said polynucleotide comprises a base sequence that is identical or complementary to a segment of at least 20 contiguous bases of SEQ ID NO: 3.

25. The polynucleotide of claim 23 wherein said polynucleotide comprises a base sequence that is identical or complementary to a segment of at least 50 contiguous bases of SEQ ID NO: 3.

26. The polynucleotide of claim 23 wherein said polynucleotide comprises a base sequence that is identical or complementary to a segment of at least 100 contiguous bases of SEQ ID NO: 3.

27. The polynucleotide of claim 23, wherein said polynucleotide comprises a base sequence that is identical or complementary to all contiguous bases of SEQ ID NO: 3.

28. An expression vector comprising a polynucleotide that comprises the nucleotide base sequence of SEQ ID NO: 3.

29. The expression vector of claim 28, wherein said polynucleotide is operatively linked to an enhancer-promoter.

30. A recombinant host cell comprising a polynucleotide that comprises the nucleotide base sequence of SEQ ID NO: 3.

31. An isolated nucleic acid molecule comprising a region having a nucleic acid sequence of SEQ ID NO: 4, SEQ ID NO: 5, SEQ ID NO: 6 or a fragment thereof, the region further defined as encoding a murine alpha1 soluble guanylyl cyclase possessing a genomic organization as shown in Table 6.

32. An expression vector comprising a nucleic acid having region having a sequence of, or complementary to, SEQ ID NO: 1, SEQ ID NO: 3, SEQ ID NO: 4, SEQ ID NO: 5, SEQ ID NO: 6, or a portion of any of these.

33. The expression vector of claim 32, wherein said polynucleotide is operatively linked to an enhancer-promoter.

34. A recombinant host cell comprising nucleic acid having region having a sequence of, or complementary to, SEQ ID NO: 1, SEQ ID NO: 3, SEQ ID NO: 4, SEQ ID NO: 5, SEQ ID NO: 6, or a portion of any of these.

35. The recombinant host cell of claim 34, wherein said polynucleotide comprises the nucleotide base sequence of SEQ ID NO: 1.

36. The recombinant host cell by claim 34, wherein said polynucleotide is introduced into said cell by transformation of said cell with a vector comprising said polynucleotide.

37. The recombinant host cell of claim 34, wherein said host cell expresses said polynucleotide to produce the polypeptide.

38. A process for preparing a cell expressing a polypeptide comprising the steps of
(a) transfecting a cell with a nucleic acid having region having a sequence of, or complementary to, SEQ ID NO: 1, SEQ ID NO: 3, SEQ ID NO: 4, SEQ ID NO: 5, SEQ ID NO: 6, or a portion of any of theseto produce a transformed host cell; and
(b) maintaining the transformed host cell under biological conditions sufficient for expression of said polypeptide in the host cell.

39. The method of claim 38, wherein the polynucleotide comprises a region having a nucleotide sequence of SEQ ID NO: 1.

40. The process of claim 38, further defined as a process for preparing a cell expressing a polypeptide comprising the amino acid residue sequence of SEQ ID NO: 2.

41. The process of claim 38, further comprising purifying an expressed polypetide from the transformed host cell.

42. The process of claim 38, further defined a process of producing an active enzyme.

43. A method for the detection of genetic and/or inherited and /or acquired human diseases utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.

44. A diagnostic kit for the detection of genetic and/or inherited and /or acquired human diseases utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.

45. A method of treating disease comprising utilizing a nucleic acid comprising a region having a sequence of, or complementary to, SEQ ID NO: 1, SEQ ID NO: 3, SEQ 20 ID NO: 4, SEQ ID NO: 5, SEQ ID NO: 6, or a portion of any of these.

46. A method of screening for drugs, drug design, and/or drug development comprising utilizing a nucleic acid comprising a region having a sequence of, or complementary to, SEQ ID NO: 1, SEQ ID NO: 3, SEQ ID NO: 4, SEQ ID NO: 5, SEQ ID NO: 6, or a portion of any of these.


SEQUENCE LISTING (15 sequences)

SEQ ID NO: 1 - DNA, 2430 bp, Mus musculus
  CDS (286)..(2361)
gagaagtgggagggactcagagccgcgggtttctcacacaccgccttctaggcagccctcctccagtgcctgccagccggaccggaccccaaggcgaagagcagcagtgcacagcctggggagccagcggagcaaagacacctttggcccgatgcccctggcctcctgtgatcgcatcatggtgctgggtcactcctgtccttgagtcagtagaagcagatcttcatcagtccacatcaacaccagctagtcagaaggaagccactgccaagctccaggaacaccatgttctgcaggaagttcaaggatctcaagatcacaggggagtgtcctttctccttactggcccctggtcaggttcctaaggagccaacagaggaggtggctggaggctctgagggctgccaggctactctgcccatctgccagtactttcctgagaagaatgcagaagggagtctcccccaaagaaagacaagccgcaacagagtctacctgcacaccctggcagagagtatttgcaagctcatcttcccagagtgtgagcgactgaaccttgcacttcagagaaccttggcaaagcataaaatagaagaaaacaggaaatcttcagaaaaagaagaccttgaaaaaataatcgcagaagaagcaattgcagcaggtgccccagtggaggcgctcaaagactctctgggcgaggagctgttcaagatctgctatgaggaagatgagcacattttgggcgtggttggcggcaccctgaaggactttctaaatagcttcagcacgctcctcaagcagagcagccactgccaagaggcggagaggcggggacgactggaagatgcctccatcttatgcctggacaaggaccaggactttctaaatgtttactacttcttcccgaagagaaccacagccctgcttctccctggtatcattaaagcggctgctcgcatactgtacgaaagccacgtggaggtgtccctgatgcctccctgcttccgaagtgactgtaccgagtttgtgaaccagccctatttgctctactccgttcatgtgaagagcaccaagccgtccctgtccccaggcaagccccagtcctcgctagtgatccccgcttcgctcttctgcaagactttcccgttccatttcatgctggaccgagacctggccatcctgcagctgggtaacggcatcagaaggctggtgaacaagagggacttccaagggaagcccaactttgaagagttctttgaaattctaactcccaaaatcaaccagacatttagtggcatcatgacaatgttgaacatgcagtttgtcatccgggtgaggagatgggataactcggtgaagaaatcgtcaagggttatggatctcaaaggtcaaatgatctacatcgttgaatccagtgccatcttgttcttagggtcaccatgtgtggacaggctggaagatttcacaggacgggggctctatctgtccgacatcccaattcataacgccctgagggatgttgtcttgataggggagcaggcacgggctcaagatggcctcaagaagaggttggggaagctgaaggcaaccctggagcatgcccaccaagccctggaggaagagaagaagaggacagtggatctgctgtgctctatcttcccctctgaggttgctcagcagctgtggcaaggacaaattgtgcaagccaagaaattcagcgaggtcaccatgcttttctcagatatcgtagggttcactgctatctgctctcagtgttcacctctgcaggtcatcacgatgctcaacgctctctacactcgctttgaccagcagtgtggagagctggatgtctacaaggtggagaccatcggggatgcatattgtgtggcaggtggattgcacagagagagtgacacccatgctgtccagatagcactgatggccctgaagatgatggagctctccaatgaggtcatgtctccccacggagaacctatcaagatgcgaattggactacattctggatcagtgtttgctggagttgtcggagtgaagatgccccggtattgcctgtttggaaacaatgtcactctggctaacaaatttgaatcctgcagtgtgcctcggaaaatcaatgtcagccccaccacatacaggttactcaaagactgtcctggctttgtgttcaccccgagatcaagggaggagcttccaccaaacttccctagtgacattcctgggatctgtcactttctggatgcttatcaccatcaaggacctaattccaaaccatggttccaggataaagatgtggaagatggaaacgccaacttcttaggcaaagcgtcaggggtagattagtgagccacatgctcttatgtttgatgcctttgaaggtgtgcagaacctctgtgttgaccttaggattac

SEQ ID NO: 2 - protein, 691 aa, Mus musculus
MFCRKFKDLKITGECPFSLLAPGQVPKEPTEEVAGGSEGCQATLPICQYFPEKNAEGSLPQRKTSRNRVYLHTLAESICKLIFPECERLNLALQRTLAKHKIEENRKSSEKEDLEKIIAEEAIAAGAPVEALKDSLGEELFKICYEEDEHILGVVGGTLKDFLNSFSTLLKQSSHCQEAERRGRLEDASILCLDKDQDFLNVYYFFPKRTTALLLPGIIKAAARILYESHVEVSLMPPCFRSDCTEFVNQPYLLYSVHVKSTKPSLSPGKPQSSLVIPASLFCKTFPFHFMLDRDLAILQLGNGIRRLVNKRDFQGKPNFEEFFEILTPKINQTFSGIMTMLNMQFVIRVRRWDNSVKKSSRVMDLKGQMIYIVESSAILFLGSPCVDRLEDFTGRGLYLSDIPIHNALRDVVLIGEQARAQDGLKKRLGKLKATLEHAHQALEEEKKRTVDLLCSIFPSEVAQQLWQGQIVQAKKFSEVTMLFSDIVGFTAICSQCSPLQVITMLNALYTRFDQQCGELDVYKVETIGDAYCVAGGLHRESDTHAVQIALMALKMMELSNEVMSPHGEPIKMRIGLHSGSVFAGVVGVKMPRYCLFGNNVTLANKFESCSVPRKINVSPTTYRLLKDCPGFVFTPRSREELPPNFPSDIPGICHFLDAYHHQGPNSKPWFQDKDVEDGNANFLGKASGVD

SEQ ID NO: 3 - DNA, 955 bp, Mus musculus
ctgctacaagcattgcctagacggacgttctaaaagtgataagcacccactgtgttaagtttgttaaatctgatagaacgagacttaatagtatctggccatgcgtgtatatatcatggctcagtagatttgttttatgctccatgtatatgtgtgtgtatatgtatattttaatgactataccataaaacaaagtttatatcatgttggtgcatggcattctagaaaccattttgtacacgagtgaatctaagttttagggaaaaaaggcaatttatttgtagacttctgaagtaagaattagtatgctatattaggaaaaggagtgactattttgaagtatgtcaattcccttctgggactctattattgcaaaaattggttgctcattcaaattttatgccaattacattttatctaacatctacattgccctaatttgtactgaagtccttgtatattgtgtttggttgactactggtagctgtgatgggggctgcattgtttcacactgaagggttacatttgctttagcaagtgtttggggtcaaactatgtccaggaacaaggctgggaatacatagctaggacactgctgttggaggccccaccccagcccccgaacctgcccctggcctcgctccaggcttgagcttctttattagcttagaaggatgtcaacttatccaggatatcatattcagaatattcatcaaaatcattttaattctagcatagaatggactgaattcctgttggtatatttggcctatcatcctttaaatgtctctgataatttattgatatctatctttataaaatagaaaaaaagtacttttgtgtaaagatatttgtctttaaatttagtatttcatatcagcacatcaatgtatgtataaatgttacatgttaattgtgtaaaagattctacaataaattatttttaccacttgcc

SEQ ID NO: 4 - DNA, 7697 bp, Mus musculus
  modified_base (605)..(6955): N = A, C, T/U OR G
ccaaatagaggcaagaccttactcaaaaaaaaaaatattcttgtgtccacaaaattacctttgaaatgagtagagtagcctcaaacctaaaagccaggccaccaaagccacccaaggagaaagaaagttccccaccaggtttcagattcaggaaactacagtggttctgcaccagcttactagagaaaatgtttttagttttaatgtgccaacttttcaacttttcttagagtctctttttttctcttccttgtccccttccctgctatgtgtgtatgtatgtgtgtgtgtgtgtgtgtgtgtgtgtgtgtgtgtgtgtgtgtgtgtgtgtctgcctttggtggtttggaggatggcgactgggcgggagcagactcagttctctagctgagcctgggagaagtgggagggactcagagccgcgggtttctcacacaccgccttctaggcagccctcctccagtgcctgccagccggaccggaccccaaggcgaagagcagcagtgcacagcctggggagccagcggagcaaagacacctttggcccgatgcccctggcctcctgtgatcgcatcatgggggttcgctcagctgtttgcttttcnnnnnnatgcatgaaatacagtagttagagcattagtgaagcaataatgaagatcatttgcggggtggaaaagatggaaattgatagtttgtgctaaggacagtaagagaaaaatgtgatttatctttaattgatactctgttggcaatttttttgtgtgtgcacctagctatctgttgtgtgtaaagtcatctatcctttctcttctgactttccactgctcataggtgctgggtcactcctgtccttgagtcagtagaagcagatcttcatcagtccacatcaacaccagctagtcagaaggaagccactgccaagctccaggaacaccatgttctgcaggaagttcaaggatctcaagatcacaggggagtgtcctttctccttactggcccctggtcaggttcctaaggagccaacagaggaggtggctggaggctctgagggctgccaggctactctgcccatctgccagkactttcctgaraagaatgcagaagggagtctcccccaaagaaagacaagccgcaacagagtctacctgcacaccctggcagagagtatttgcaagctcatcttcccagaggtgagtgttctccctttagcaatgatagtggtatttcaaaattggaaatgctaggggtttcaaaagaaaatatttagaaattaagtttctcacttttaagagcacggtaaacagaatgtccttgaaacatatgaaaactgcatttttaaataacagtattctaaattgtgtgcaatcttcaaaagtttcacatcttgaattccttccaaaagatcattagactctgaaaaattcctgtctttgcttcgtctgtcagcctctgcttactttacctgtaaagtggagttgtattacacctacctctgtgtgttagtatacactgagtggggatggagcttggtatggggtgtctaatgaagcgaagactgaaaatgaaactccannnnnntgtgagcgactgaaccttgcacttcagagaaccttggcaaagcataaaatagaagaaaacagnnnnnaagtagcccattgttaccgctatatttctttagaagtactacttttcctagcctaaaatttctcagttttcatgcccatactctgtaaaaggctcaacagcagtctctatctctttcccctgaggctaattattaacgtgagaactctgccagactatattcccgtgattgcctccagcctttgaaaacactcctcaatttgccttattgagcagaatttttcatggacaaatgagaaagacatagtttggtttgaaaccaaagtctgtgactgtgaaagtctgggaatcagcttcgcacccaggtttatctaactctcagcaaattaacagcaggaaaggaggaatgcttgaggccacgtgtagtatagaaggggttactctgcattaacacacacagagttcaaggacttcaggctgggagtcactaacaaacaaatgcaaacatgttgagagaaatattagttattcagggatgtggtgtgcttcctaattccctgttctcacagttttatttattacctgctgcaattgcttcttctgcgattattttttcaaggtcttctttttctgaagatttcctgggaataattaaaggtcttaaattaaaagacagattaatgagtgttgaaaatttggatcatatacaacatataatgataggtgagaattcttttccagaagcaaatcttgacataccatataagatacaatctgaaagtcagtgtcttcatacatcaactgcatggctttgtcattcatgtctgcctacatggcttttaaaaccttatcattatagtgactgattctgttgaaatttgccaagttattctagcactttaagggtataaaagggtatttaattgcaaggttcaacttgccattagctgagccaagtttaaatgcaggtatacttcttctgttttaaatagcattttaataaaacacagtataatttgagaatttttaaaaggagactgttttaagaaggaaaattggccagaaaaaaactttgttgcatagaaatatgatgcttataagtatataaagtactaagtaggaatattttatccaagtgggctggggtatcatattatagctatatactcaatttggatatggagagtcacaatccttttttggataattcatcacagtaccagannnnnnnctcttttgagctgacccacttgatggccgacttgtgctgtgtgcttgcaggtgccccagtggaggcgctcaaagactctctgggcgaggagctgttcaagatctgctatgaggaagatgagcacattttgggcgtggttggcggcaccctgaaggactttctaaatagcttcagcacgctcctcaagcagagcagccactgccaagaggcggagaggcggggacgactggaagatgcctccatcttatgcctggacaaggaccaggactttctaaatgtttactacttcttcccgaagagaaccacagccctgcttctccctggtatcattaaagcggctgctcgcatactgtacgaaagccacgtggaggtgtccctgatgcctccctgcttccgaagtgactgtaccgagtttgtgaaccagccctatttgctctactccgttcatgtgaagagcaccaagccstccctgtccccaggcaagccccagtcctcgctagtgatccccgcttcgctcttctgcaaagactttcccgttscatttcatgctggaccgagacctggccatcctgcagctgggtaacggcatcagaaggctggtgaacaagagggacttccaagggaagcccarctttgaagagttctttgaaattctaactcccaaaatcaaccagacatttagtggcatcatgacaatgttgaayatgcagtttgtcatccgggtgaggagatgggataactcggtgaagaaatcgtcaagggtaaggaaaatgtaacgcggattcaaaataaaaccaattgtttcatactgaagggaaagaaatcacatgacaaatgagcagacgctatttggctaacaaatctgtctaaaattctaaaatgatttaacaagtgaatttcttcctacatccgtttttgctgcctacttaattgattgcaagtatttattgaatacaatttgcctctttaaaattgcagtgggtattgtggacacgcacatctcataggtagaatgcttgcctgacctgcaggtaggcctaagggtttgatccccacatcctgtaaaactggattgggttatatcacatattttttaattctnnnnnnnnacttgtttctttcatgctcagagcagctcatcatttctgtgtagcatatcctagtatattcttcatgctcaagagcagctcatcatttctgtgtagcatatcctagtatattcttcaaatgtctgcatgaatttcagaactgagaccaaactgaagtgattaaaaagtctattcttctttagctagttggaaacaaaatggatgatctcagcacccacttctaatgccagactcaaaaagattaccccagtagccatttcctctctggtttcggaaaggattgggaaacttgactaatgcatggtaacaggttctcctttcctttgcaggttatggatctcaaaggtcaaatgatctacatcgttgaatccagtgccatcttgttcttagggtcaccatgtgtggacaggctggaagatttcacaggacgggggctctatctgtccgacatcccaattcataacgccctgagggatgttsycttgataggggcagcaggcacgggctcaagatggcctcaagaagaggttggggaagctgaaggcaaccctggagcatgcccaccaagccctggaggargagaagaagaggacagtggatctgctgtgctctatcttcccctctgaggttgctcagcagctgtggcaaggacaaattgtgcaagccaagaaattcagcgaggtcaccatgcttttctcagatatygtagggttcactgctatctgctctcagtgttcacctctgcaggtcatcacgatgctcaacgctctctacactcgctttgaccagcagtgtggagagctggatgtctacaaggtagggaaggtggaaaaagaacagtttagcaggcctaaactgtgaccttggaaaggcccagcacccgagtgaacaccagtgctgatgaatagctctctgcatttgggtcacacaggctaaatccggggtcaggcatagtgcattaattatgtgggtgttctccagatggaaatagctctgtacaaactaattgttcccggagaggtctgccacgttgtcttctaaaaatgagcatagaatgaagcgagattagggtgaatggattattcttacagtgagaaggtagannnnnnnnctcatcttcttggttgacttggaagcattctatctcatttaaactcatggtgcacaaaatcagtatacatggctatggatttctgctagtaaacgttggggagtgtgtgacacagcaatatataaatggtctaacagctgaatttgaaaatgttgtgaaggcttaaacacacagctttctctaagcttatagaaggccaatgctaaacctgtctttactatagctttcttcttaagttttgttgactttcttaaaactagatattaaaaagttgagtgtatagaagtctgggttatttcaatggcccattgctgaaaagtaataaagtgaaaagtgttctgtaagattaagtagtaaatttactataagataaaactctggtcaaagcaccagatttttgacaaccagctaatgtgcaacaagtcacggccttaccttgataggttctccgtggggagacatgacctcattggagagctccatcatcttcagggccatcagtgctatctggacagcatgggtgtcactctctctgtgcaatccacctgccacacaatatgcatccccgatggtctccacctagatacaatataatttgccccgtataaataaaagtttagaagaagacagcataacagaggcatacaagagaaaccgcagtgctcatgccctaaggagaagcagggtaagtggaggtgtcagagcaactctttatttcataaaatgcataaggtgccaacttctgactttagggcagtgaactgaaagacatcacaatttgcaaaaactctatcagatcatcagatggcctctattgcagatttactactaatcatgtgttcttttcttaaggcaaaacaacaacaaaaactatataaaatggtttttatatagacaaatggatttgtttttttagtttatttttattcatgattttatatttatgtatatgtgtatttgtaactcatgtatgtgtgagtgcctacaggtgccagaaaagggcattcgagttccttnnnnnnngtaccaaaaggatgtcagttcacatctgaatacccactgatagactgagaaggttaacacacattctgagtaaaaagttgtggaagaaaatgcaatgtctgttaagaattcatgccgcaattgtctcccaaggacagtgaaagccttaaagcacattaaagtaggaatgctaaagatgttcattccattctcccagaagatcgccaagagagaaaacagggatggaggatggactaagatgacaagtttgtcaagtgagatgcatgcatttcaggtccttagtgccatccatacctgtatgtggtggggctgacattgattttccgaggcacactgcaggattcaaatttgttagccagagtgacattgtttccaaacaggcaataccggggcatcttcactccgacaactccagcaaacactgatccagaatgtagtccaattcgcatctgaaggcaaaacagcagtgtcccaggtcactgcaaagtcacctctcacctgacagttttccttgagtgtcaatgctagatgttcctaagcagtaactaataacaccacaccctttgaaagccccagtgagccacctttgcccatgcaaggccacattcggagagcagagtcagcgggcacacctgctcactctgctccaccaggtggtcagctttaccccacctgtctttaacatctggaataaatatgttctcaggcctctctggatcactgcagcagaataacaaagaatctataggctgctcccaggaacctgtgttgtcctttatatgaaagaaagatgtccttttatattctagaaggaaattcttgaaacaaagaccaagaaggacatacctgtgcttccagatattcagaaaggctaaaagggagtaactatnnnnnnnngtttctcaaattgcattaaaatgataagtgcttgtttattattaaaaccctaggcaattttgtgtttaggaattctactattacataaatcacaccatccccaattatcatataatcaatctgtcttttaatctgagtcaaattatgagtgagtagaattttccatgaaatttactcattgtagtatttcttttatgagactattgacttatggaaatcctttaagacctaataagactagtattaatattcatttatttctgttgtctttttgtcctgaattgccaagtgtttctgccagcattaagcttccattttatttaaattgttctcaggttactcaaagactgtcctggctttgtgttcaccccgagatcaagggaggagcttccaccaaacttccctagtgacattcctgggatctgtcactttctggatgcttatcaccatcaaggacctaattccaaaccatggttccaggataaagatgtggaagatggaaacgccaacttcttaggcaaagcgtcagggrtagattagwgagccacatgctcttatgtttgatgcctttgaaggtgtgcagaacctctgtgttgaccttaggattacagaarawaaaaaaaawcagtgttaaaattacagaggctaaacacaggtttcctctgtctcccatttaagatggaaaagaaaagtgttcacttcagcgcttcaaccttcttctatttaagaaacagacctcaaa

SEQ ID NO: 5 - DNA, 15093 bp, Mus musculus
  modified_base (1213)..(8441): N = A, C, G, or T/U
acttactttaatgtcttccccaacacmwmrrrwysakrrwtkcsssmwmrcmagcwscymmrrggsmatgggttccacgaaaccsgtamtkaagggmarararrrarrrmcsgtggtcaagggacccggcgccattgcaaacccgggttccttccacccrgkgcctytgaccttgtcattcgccttcaaggataagctttygggkaccctattcttaaaagggggggactctgcgggatcttttgaattctcgtcgtccctttcccctcccccatcttcttacccctgctcacctgtcaaccgtggtgcaacaggggtagctcaggaacctgagccctgagcttcccacaccaccctgcccgtggaacccagaatccccagaagttggaggctggggttgagtggtggggacccaaccagacccagagactcggagcactccagcagatcatatgtccccacctcccaccaaaagcgctgttcctcgcggccctccgctctgtgtgccctggggcttgtttctgcatctcagccagctctctagccagtcaaggtccacccgctcaggtgtgatgttccgcttgaccatcctgacagcactcaccatggtgtcccgcgcccggagcctaggaagcagcttccaaggcaagagtaccgcggaagggacccagacagcgactacaagcggcgacagtgctyccagacccagcacaraagctgaaagctaagggcatcartcttcggccaactctgattgccacctctttcarccaaygacgagtggagccgcaggaggctgggcgctgtttcttcccatatcttagccaatcagtgacagaggagggaattcagatgggctcagagctactgtgctcaaacattttcccgccccagtctggtgacaagtcatttcttttgaggccttaaggcagatagaacagaaccagctgtgctccttccagcatggatggggtattgtcattgtcatcctcgtacatctgttctaatgtcctagagcttgggcgtgtgtgtgtgtgtgtgtgtgtgtgtgtgtgtgtgtgtgtgtgtgttgcgcgcgcgcgcccactgktkattaacattgcyactgktttttgmaattmtgcacawaaacatttcctctggagctttacctaacaacagcgcaaactaggtactcctcaaaattaacttttttctttttgtccattaagtnnnnnnnnnnnnnnnnnnnnntacggtttcgtgaaccatgccctggagctgctggtgatccgcaattatggtcccgaggtgtgggaagacatcaagtaagtgaacagccccgctcccagggccgcgcgttggcaccgagcccgcgcccgcccgcttccctggctgctcccagcagggtcgtggtgcgcatcctccggtcycctctcccacgctccgaagccgggaccaggagaggagggtaggccggggcgtggccgcktgatcaagccccttggggtcgctgcggcctyttcacccggactgagcgtgggaacacccagacaggcagagcactgtgactcccaggccaggttcagagggttggctagctcccatgcaaggggacctgggggcttcctgggcctctgcaaggtcttaggatcagaggcaggaaatgaacagaagattcgctttcttttgcgatttgcacaccaggaacacgtaatatggttaaatcatttggttatggtatagtacactcaaagattatttctcagtaagtcatgagaaacccgatatgtaataaagggacattttccctttaacttgcccaaatcacccttgctttgctgtgttttcttctggcaaccctagcagggcattctattttgtagagcttttgagtctggtactcagataaagacgagaaaatnnnnnnnnnnnnnnnnnnnnncattagtactcaaacagctttactccatgaaaacccgttttgaattatatttttttagccatgggtctgatctgtgaacttcatttgtcagtggattgcctctctctttttgccagtgcttcacatagcttattactataacacatttgaagaataatgtttcctgcacactacatttgagtgcttactcttcgggtgatgctgaagtattggcagtagtttttttgtttgtttgtttgtttgtttttccctccatgcaagaccttgtacagggatagggatgggggcaggagatttccactttcgattttctgtttacccaacagcttatgctgaaaaggtaggatttagtatagccacagtctagcatcatccatcaacccatcacgactgtacaatgaaaacctgcataatggtacactgtagtcttggagcacacattcctttatggtcctatgtgtagctaacacagagcaggaaagttgttttcaacctactgaggactttgcttgcagcagccaccaggtcatatgttttggaatcatcgtatattattctgacaagaaactggccttcttcatccagctgtgcctcttttytgaaagaaaatggaagagtgatggagagcaatgttgaaataagagcatctgtcaaattcatgatggtgtctatttctgaaacaggatttctggaggcaaaattgcttaagatgacgctgtgtacctaacaacccagtgatttcagaggaattgccacatgcttctgcccaaattaaaacaatacatgcaatacattttagtactctcatgaataacacactgcaatgtagctatgtttaaactgggaaatagtaaatatatattgactccctataggtgtgaggcactgtactgggaactggagatgcaggctttagccaaacaaactggaatcctcttattttggaacaaatacactctagggggagcggcatgttcggctaagaaggcagggagtggatcaagagagccagcagggtgtgtgactgtttgggtagatagcacagcctactgaaactggcctgagggagggtagagtctctgcatgacaannnnnnnaagtaggttaaaatatttcatatttaaaaaaaaaaaagtttttttgccacaaaaggttcttacatctttatttttgtgtgtgtgggggggaagaaggttgtgtgttgctcgtgctcgtgcacatgtgtgcatgctatacagatatttatgaccaccatcatgtgtatgggggcagaggagaacttgagaatcagttctttcaaccatgtggaccttgggcatcaaagtctggccactaagcttcatagagtcattttaccaatgctgtgtccttgatttgaataaatgtttttttaaagaaacaatacaataaatttctaataaaaactattaaacataagtgtgttagtatataaattgagaaaaatggttatatccctaatagtgtgatttacaccattaactcgttagtatagtcttttaagatttcatagtagttcgttggtcagagattctatgttcacactgtacaataattgtgaatattttatatcatttccatatacacttgtgctaattcttctcattgagagtcttctgcctgttctttctttttcatctacaaaacagacctcaatgctggcgaaatcctgcagatgtttgggamsatgtttttygtcttctgccagsagtctggctatgataccatcttgcgtgtcctgggatctaatgtcagagagtttttgcaggtgagatgttcgaggtcctgagagtgaaagctctcagtgtctgttattaaatttgagctctgaaatacttctatttagccagctggacaagagtgtcttgctgctggcaaggaaccctagtgaccattctcacataaaatttaattattagatgattctacacatatttcagaattttcagtcatgtgttttcatagtttctcattgttgattctgaatatgcaagccttcaattagtattctcttaaagtccacagggtctatcatgacctgtccaattgcgtttgaggaagaattactcatgctaatggtgtggtcttttgttttcaatatgtaatctggtttattttttgtaacatacttctaaatataagctctcattggacagtgtatgagtatttaagtaaacagtttctgggctccttattaggagaaagcagcaggcgcttcaaggtatccacttttggagaacttaattcatccgcatttggaggttacaaatcagttaatctaacccagaaaggaaaaggagattttgagggaaaattccaaaaggttgatttccgattaaaaaaaaataaccnnnnnnnnngttgtaaaacggcgggccaagtgaattgtaatacggactcactatagggcgaattcgagctcggtacccggggatcctctagagtcgacctgcaggcatgcaagcttacaaaaaattaggaagtttaattttctaagcctaatacccattcagatgggtattatagtgcgagcagaaaagaatgtggttgatttacacaatgttctctcctgaatactacagtgtgcaacaacaaacaatcttagatgcctgcttttcatcttaagtatgctcttgagacttgccgttaacaatcacatagattcgtcccaactattctgctctcttttcattctctctgtggtacttacctcagagccatctcaggggcgccatgaaggatccccctaggtaaacgtttacactagtgatctgcccagcaaacccagatgtgaaccttctctctgctgtttgcatgcctatgacctcatagacattcctagtcactatgcttccgttttcactacagaacctcgatgccctgcatgaccacctcgccaccatttacccagggatgcgcgcgccttccttcaggtgcaccgatgcggagaaaggcaaagggctcatcctgcactactactcggaaagagaggggcttcaggacatcgtgatcggsmttatcaagtactgttgctcaacagatacacggcactgagatagacatgaaggtagtgttcacccgctaagatgttccgaatattggaacatttgaaatgatacctcactgattgctgcttgtgggctacattagcggtatctgtgagccaaaatggcgaaaaccccagaagtcttctctttttggccttgaaatgcattatactgggcgatttcccccttgcaacagattctctttctatttatatgttactcaaggcaaaaagaaattcaattcctctccgtgaatacacgccagctggaactagggtgggccatgtgacatgcgagttatcagattgtggagagtgaaatgtaaagaacattaaattagccgattttacaaatcacttattccatgtgaattctcttctctttcctaaaacgtaccaagaacatttttatcatgtcaaccaaaagtctttggttctttcttgattttgctttttaatcaagttgtgctggtgaacttagccaagccagctcagtctttttctgatgctattgataaccactctttgctatttctgagtggcaagttttwaaggtgtcaaatmcagaagagccatgccaatggaaaaaaaannnnnnnnnnnnnnnnnnnnnnactccaagaaaagtcctttatttatgcccacatattaataatttccagaaattgctgtcttacaaagatgctcattttgaaagtacaaaaatatagaaattagcacattactcatcctctgatctaatgtcataagaagttcagagccagattaagatagcacataaagaaagcagggtttttttagaaatgtccaatacaatcactcctgttataaagaattaataatgaggctttgaaaatgcagtttatacttcagcaaaaataagcatcgtggaccaaaatactgttggattaagagagggtgtatttggtccattcactccccatgcatgagttgaaatatcacgccaatctcagtttatatgtacaaccagcacatattgatcaacgatgtttactgaagttccactggatctgtttttaacttatgcggattacatttaagttaaaagttgttaccttggtgaacagaggaagaggatgctatctgcttctggtaagtagatcatttggcctttgagacgtaagcagctaatctctgctccagtcagttcatcctcacactcaagtttctccacatccagcaacccttccttaaacaggaaagacaaaacgtcagcttctgcctgacaaagcagctggagggatcggaacaacttcatttctcaccagccctaagagagggaaacgcatcccttagaacaggagagggggcattgctccaggtccaaggcattgttagaagcacagtgcctgatacccttctgtgcactcaggaaggcagcagctaagctgctgtgcctgcctgcatagtagcttctgtgtttttccactctggccacaacatgcattactgaatggtacaaaactaagggaagctagccctgtattcacaagacatcaaaaagcagcaagtcagcctcaagcatataaaacacatgtagatgtatctttgaaaaaagttattgaagaaatatgaaaatgtgtggatgctcggggaaaattggtttgctawaatttcccaagagcgcaatcgaggggatatggaaaccatcttaactccaaactacatgaggtttatgagctttcaatccttttttwtttcccaagaaaaaaaaatggttagccggaattatwaaccaatttacggaaaaagtaaaccacagctgacattatcaaagacttcttgttaggggaaagaagcattggaacttcaagggtacttgacgttacaacgttggttttattaagtgagactgagatgttcccagccatacacttgctccaactttttgttcactcattctcatcagctgacagaggtccgtgtccccagtcaaacactgctctcttgaaagttgtattttcactttcttttatttttatctcagagactatgctcagagtcgatgtcccctctctgttgttgcaggtggacaaaatgccttgcttacatttattcaaagggttcttgcttaccttgcttctcagtacaaagrctgtattgatgtgtgaaagaatcccatggaaactgatgtcgatgtgagggcggaccagagagaagacagacagaaggctgcagttcccaggctggagctaagacacagaaaaagagcaaagagcatgcagctaggttgacttacgattagccattggtcttgcaattaagctagaccagttataagtgaaaaatttaatcgtaagtattgtgtgggagattttccctgactgtggcctccaattttgctatatgcaatggagtatttaagcaaatgccaaatacttgcaggaacaaggaaggattcaggcgactactatgtagatataaatataaagctcatgaaaataaaataagaaagagaaaaaaggagatttatcttctaaactgaagatgcctgtggtatcaaatgccaggaagtcaaaacaccggtaaatacttccttacaaaacatccaaggagagtcaatattaatagaagtgctaacacaaagtgcccactgatgatgaagcaagtaactgactgaagggacctcctgnnnnnnnnnnnnngagaaaatgaaagccccaaaagaaagaaccagcataaaaggctttaggcagaaaaaaagggatcccaaggaggggtggctagacctcgggaaacccaatcactgtctacagtagcattgcactgataagattcttaaaagtcataaagcaaaacgaagtgttttctcaatagaagcatcattactcctgacacaagcatggagataaatttttcttctggtttttatgataaagaagaacaaggtaagtttgttaaatgcactagccttgtattaatatctaaggcaatttcttgggagcagctttctgcatattctttcagtgaaggtagatgcctttcaaaagtacaactcaacatgtatttcaagtttgctgattgctagtgggggtttagatacactgaaagcaaagccatgttaggtcagcttctccttgggtttcctcaacctgatcttctgaatagtgaagttggagaaagagaaagtgagatgccacttgctgtggctgtggctggggctgaccacgcaacatggacaggtggtgctgattttaactcaagctctgtgtgacagtgtgatgaacctggacgacctaacaagaagaggcctgtatctgagtgacatccctctccacgatgctacccgagacctggttcttttgggaraacagttccgggaggagtacaaactgacacaagagctggaaatcctcaccgacaggctgcagctcacactgagagccttggaggatgagaagaaaaagacagacacnnnnnnnnnnnnnnnnnaaataaattttattccctgtcatgtgagcaatgtttcttctttattaccactattggtgcaaacattttaagatgctcatactgacaacatcaagcctactgctgtcataagaacagggttagaagctaggactacaaatgaacatggcgaagtcctattaatggccttgtgctttttcccacctgttcatttgtttggctgattttgagttggtttgaaatggttcaggattggggaatatctgtgaccacggcaggcaaatggttagtatgtttttttgttttgcacgtagctaacaattgatgctggttttaaagatgtcttactaaaacccagcacaatggcactgatacagtaaagaggcccagccagagaggtaaaagctcaccagcatcttacagtactgtccactaattcccacagattgctgtattctgtccttcctccatctgttgccaatgagctgagacacaagcgcccagtgcctgccaaaagatacgacaatgtgaccatcctcttcagcggcattgtgggcttcaatgctttctgtagcaagcatgcatctggagaakgggccmtswagattgtcawkctcstcarcgryckcyaswsycgatktgayayactgacwgaattcaggcgaaaaaacccatttgtttacaaggcaagtcttcatsggagcctgttgtagtaagtgcagtacagtttgtgggcttccgagactcccctgaagaagcccttggtcactgtattggattgtagctgacttgctagcatgttaagagtcactgcagaccactggcttaaataatttcctcactgcaaagaagtgtatttgccgggcgtggtggcacacacctttagtcccagcactcgggaggcagaggcagacagatttctgagtttgaggccagcctggtctacaaagtgagttctgggacagccagggctatacagagaaaccctgtctcaaaaaaccaaaaaaaaaacaaaaaacaaaaaacaaaaaacaaaaaacccaaaaaccaaaacaaaacagtgtattgtttgataatttcatacatgtagttaatgcacttgatcatattcataccacacaagtcttctcttacctccctgccctctcaatcttcctgagaagtgcccctcccactttcatgtcttttgtttctgttttttgtttgttttgttttgttttgttttgtgatcaattcaaattgtgccagggctgcttgcacaracatgggtaaggagcttgtctactgaggtatagacaacacaccagtwgccacaaccactggaaaaaccatgactccccattttcctcagaaacaatcccacatctataatttaatgttgacttgcccaagccttgggcaggccctgggaaggcaccagaagctacaatatgatcttgagtataatagccacatcattcccagaaggcaaaatgtcagggaattctcctgcatcatcagcgtttccattttcttctgtgccctctttgcagatgtgcactgagatgatacagatgtttcatctagtgctgggcatgccataatctttcccagccccttagccagccatgagtctcattgctcactgcagaggggcactctactgagcaaggctgagggcagcacttgtctatggtataaacttcaacctggagtagttatcacttaggaaactttcttttctttgcagccatgatgaggtttcatggagcaatctctggctgacacaaagaatcccgcagtttctctgcacttaggttaacgagtctgtgcagattgtttattacatttagtataaacacagcattcattttttttttttttactcagccaaagaccttttttttttttttcctaaaatacataaatgctttgcactggaagaatccatggagcttgagaagggtcctgggcctcaggtttacttttagataccatcaacttgaagataaacatttacaagacatctctgccctttgttttaagtagaaacaaatataatattggaaaaatgaagtcagwggaatattgctttataaaggcaagaatgttactgagwgtctctaggctcctggggtactgdgacttcataagaaccatttatcttgtggttcttttctgagccatagkgctacacttggtgagcactcagaacaggccatggtttagccaactttgcttattttgaaacaaaaaaagtgggtggtgcctgaggaatgatacccaagcctcttttatgttcacagagatgcatacctatatatgaatacacaaaataaaccatcacatttaaaagttaaaagtataaataaatgaaaaattaaaatctgggtagaagccaggagtagatcaatgaacttcagtcamcagctcctatgttcttggactgctgccacttcagggagaagggaagtaatagaaggttttcctgaggcctgtgcaagcgggaggaccctagtctgcaaaagctaggatggccatgcacttctgtagccttggtgatgggagagatggaggcagatggatcctgaggagttgctggccagtcaagctgaactcagctctaggttctgaaagagactctgcctcaaaaataaggtctctcactgkctctctctctttgkgtctctctctgtctctgtccctctgtctctgtgtctttctcwcwcwcatwcwcwcwmagmwwawtggcawmwmttaagggagagagagaaagagaaagagagagacmgagacakmgwgagkgwgwgwgwgagwgwgwgwgagaaaggagagagagagagagagagagagagagagagagagagagagagagagcctctatcaacctgtagcctatattacttaggaaagcaatctattacccacacgcatggaatcacttgaacatgcatgagtaaggacatttttacatgtatctgcatgcacacataratattctactgagttataagaggatttcagatgaagtgatcaagacccaagctgccggaaaaaaatgtaaattacaaataattagaagattccaacctgctagagctcacaatgctaaagaggatgatgatgatggtggtggtggtggttatgatgatgatgatgataaactgtcactttatgtcaaccactgaacatgtaaatgtgtgtgtcaccctaggtggaaacagttggtgacaagkatatkwcagtgagtggcttgccagaaccttgtatccaccatgmayggtccatttgcacacctggckkymgwyaykagtggawmykagctcggtaccaagcttsawgcatagmtkgwgwatctstwcasgygwstavmtaaatggaagactkggstgctaatagctcagttgataactagttcatattccttctgagggaagggttaacatgcacaaagaagagcattttgaacacaaagagccgattgattgcatttcattctcccatagacatggtaaatgaactagctcatctgctctgtggacagcatgtcctgtggatgaggaaagctcccaatatttctgtttttattgttccatcatggaagttacccactccttctcattgccattttcaacttcaggaacattgctcttcacggagagatatttgaatgaaggcgtttgagagagtctataaaacacatatgttaaccatcaaggctattgaagcaaaaactttgcttctgcagataacaatcgggatccataccggggaggtggtgacaggtgtgatttggacagcgggatgccctcggtatttgtcttcttttggggaaataccgtcaacctyacaaagcaggacagaaaccacaggagaaaagggaaagataaatgtttccgaatatacatacaggtgaggagggaaatgtcgcactatttgcctggtaccttgcgagtggactgtgtgcttttgctacctttaaattattgctacctccatgtctcccctctgctaccacaattacttttcactggagaagagtgtaccactctttccaccagaagccatcttacctcaccctgaaggtttccaataaagcaccatcactaaaatcctaactgcttcaaactacaactgcagtgttggagtcaggctgtcttattgtctatgaaacctttatcaagttccccctctgtttcatctcatgactttcaggtgtctcatgtctccagaaaactcggatccactgttccatttggagcacaagaggcccagtgtctatgaagggcaagaaggaaccaatgcaagtctggttcctatccaggaaaaatacaggcacggaggtatggctcattakagcagtgcttttttattwttatttttatttccctgagagagatgataaaagtgggagagttcaaggcagaaacactggaaattgcacttggaatgaaaagagttcttgcttgcagaaaggaactcagtaaaatgagacatcactgtcacactgatgagccagttagaacaagatctgctttagaaagtgctgtaaggtctccagcctgggtcttgtgatgaagtagaaagcaaagcctcaggcagaggacaactggggtgtccttcctcacagtccaagstccaraatgccaggtccaaatggkggggtccccccttgctggcaccccttatragatytcaagcaagtctstaggaatggccatattggcctggcagggtttwtcaatcaggaagtcaaattatccatttaaggaccgcaraaactccagtgggcatgtttgaagagactcttggatgacctggtctcttcttgagttcctccctgaaaagatgcctagttttgattttttcagaatcttaaataactccttaagggaagatgtaggktcctgagtgctragattcccactttagggataacccmtgtccagggcttgttactgtgccaagccattgtytggccttttcacaagaggtcaaattagaagcccaagttctgtttactccatctttgatgcatgcactcctcagagggtgtgagatagcttgaacacatatcattccctaaaactgttttcctagaatataagcaatattagagggaatagaacgaccacttttaaatagttaaattcttttggttgttttcactctgctaaataacttgcttgtgaaacatgttcatgggtgcccttcagaagggccaaactgcctggtgtcattttcttagtcataagaccctaagaataatttcctctcaacctggggtgwagatgttttatagaaactgtatataaatgcatatcctattgtcagagacagggamattgtttgccagacattgtaggctcattaagactgatggttaatatttttckacccatttaaggaaacaaatgaggaggatgaaaactgagtgtcaggttgtggaacaaagagaacgtcggttgggttcgggtgacagtctaatgtgtgtcaagcaaggagcacttctttccctgtggatagcaatttctacttcgtgttttagtggcccaaggctttctcctagttacacagatctcacactatggtttatttgattttagctctgctttcgattacttttaaggtctcagtatattttccaaagtttgggtttttgatgtggatgacttgagctgtttcttaaattctgctacaagcattgcctagacggacgttctaaaagtgataagcacccactgtgttaagtttgttaaatctgatagaacgagacttaatagtatctggccatgcgtgtatatatcatggctcagtagatttgttttatgctccatgtatatgtgtgtgtatatgtatattttaatgactataccataaaacaaagtttatatcatgtttggtgcatggcattctagaaaccattttgtacacgagtgaatctaagttttagggaaaaaaggcaatttatttgtagacttctgaagtaagaattagtatgctatattaggaaaaggagtgactatttttgaaagtatgtcaattcccttctgggactctattattgcaaaaattggttgctcattcaaattttatgccaattacattttatctaacatctacattgccctaatttgtactgaagtccttgtatattgtgtttggttgactactggtagctgtgatgggggctgcattgttcacactgaagggttacatttgctttagcaagtgtttggggtcaaaactatgtccaggaacaaggctgggaatacatagctaggacactgctgttggaggccccaccccagcccccgaacctgcccctggcctcgctccaggcttgagcttctttattagcttagaaggatgtcaacttatccaggatatcatattcagaatattcatcaaaatcattttaattctagcatagaatggactgaattcctgttggtatatttggcctatcatcctttaaatgtctctgataatttattgatatctatctttataaaatagaaaaaaagtacttttgtgtaaagatatttgtctttaaatttagtatttcatatcagcacatcaatgtatgtataaatgttacatgttaattgtgtaaaagattctacaataaattatttttaccacttgtctacagtgcgcattaatttaataaaatggtattgacaccaaaaaa

SEQ ID NO: 6 - DNA, 177556 bp, Mus musculus
  modified_base (2293..144567): N = A, C, T/U OR G
tcacctggcaaaggcggcagggaaatatctcttactgacaggtaaaaaaaaaaaaatgtggatagagggtgccacttctctttggctgcagtgtgggatgagaagagacttcagagttcccgcctatctcccatctttagtttaagggagaataaaccgagttctggactatcagttcttgcaatctggttacctcccaccctccatttccataccaatggtagcttcttcccaggaggagttttgcactaagagcaggagttttgcactaagagcaggagtttggccatgcagttgctaaaagaagctcattactgtcacagctggaaataatctgtgtgtaatttccttatatttgattcctgcttaggaatctcagggagttccctgcacattgggtgccatttacttttcaactttgagatcaggtaagagaaaatcagagtactatgaattagacttcacctggaaaccagacagtggacctctaaatgtggcccacctgcttgagagtcattggagggagggagcacttgttaaagtgcagactcccggaaacaacaacttatttatcaattaaaacagcagagttgggttccagagatctgcattttaaagaaatgccccaagagatatttatcagcattttcaatcccaggagactattgcagtacttccctaagggttggggtaactctattagtatttgcccaccggatggcaagctctactaatagctaacatttccataactttgtggattgttagataagcatgtgaattcataatttccttacttcctggaccatggacatatttagggaggcagagaaacaccaagcttcctagaaccccttctattttgtttgttcctgtctcctaggctcaataggttagagcgacgagtatgcatgattcaggacactgaggtaagaacagaagagatagatatcccctaaaatgccagaccatacaccacagggttacatctgggaccaaggagaaaggatctttattaagaatctgtgccaggaaatagtagctaagccagaagatgaataccagaagatgaatgatagaattgggatctagaaaagtcaatcaattggctcatagtggttttactgcactgatactcaagtgtaagcactatgttcagtgtaatttatgataagtaacagtattgattttcattcatgggttacaaatgaggtgtgactcatcttctccggattgtttttccctgccccagagatgaacactaccatgtgccccttcacttctctttcccagccttacggtttcacggatggctctttcaatgctcattctcacctcccaggatgaactgcttccgtctacacattcctgatgttcaaatgtaacgaaaacatggaaaggttaattcatgttgggaaacagatcaaggactttgatggtagtagaaggaggttctctacctgtgagaaggaaggtaggaaaaaaactttagaccctataatgcacctaccagcacaaaaagcaaagatacttttttaactccccaaatcaaaaacgattaaataatgctatctccttgtaaaaagtcaccccaaatcgtgatacttatgtaatataggaagtacaactaacccaaatcaatgacattttgcattcccttttatggaggaaatgagtagaatttacaaatagtttatgtttctctcaccatctcttcagtcatctttatgatattgtgagctatccaatatttatgtttcttttccctttaagtcagctatagctggtttctgttgctggtgatgcacaactctggctaatggagaaaccaataatttccctaatgattaatcctcgggaaatgtaccttcttttaaagaaagctgtggctatctgtgagaggggaatcctaccttatatataagcagcaccagtattgccattttctcagtgaaacacagcatggccaacagtagaatatgagattgacacacacagtactaggatatatgtagaagtagagtgggagactttccactattattgatatttctgaaagattaacatagaaatgtaacttctaggtctgctttgtcagctcagactgctataacaaaataccatagactgggtggcttaaacaacacaaatcaatttcttacagttctggaaactgagaagtccaagatcaagatacaggcagatccagttcttggtaaagtcccgcttcctgacttgcagacggttatctttttgatgtatcctcacatggtagagaggaagctctggtctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgacttacagtgtctccacatgatagttcccccatgtgacaaaaaacatcaaaatatcaacctaaaatatggaaacaaaatacttaaactggtttggccacatttaaggaaccattaatgatacttcacttgagaagcatcaataccctattgaaaaaattggaaagtaaagcaatatttgttccagtggaatgattctaatgctttgtagttaaaacaaaaacaaaaaacaggagaccttacctctaaataacctttgtggtttaaaagtactttcacaggggttattccttatggttttctatcaggtagatagtatgttgctgtgtttgcacatgaggacctgcagcccagagcagtgaggggctcacacattaatcagggtcttcacttgcctcattgacccattgcagatcacctaacccaaacccacaactctttcctgaagatatcattttttttttcctttacgtgtcacgagaatcaacagttggtgttttttgttataacacctagtagagtggaaagaaattagaagataaatatctgcaacattatagatatgtaaattgggagaattcacatcatttccctaaactttacttttcttgtctgtaaaaataaggtaatacaaatagcaacttatatagggttttgtagggtttttggtaaggatcaaaagagttaacacttacgaaagcatttcatgtatagtaatatataatgtacatttaattttctgtctttattatcaggttctctatttgaattatatgaattttaaacattcttaaaataagaaaataccacaaaggagataagagcatgagtggtactcataatggtattctagcttttggccctgctgtggacaaattgctgaacgtctctaaaccttagttttctctcctgaaaataaagcaattacagagattaaatgtgatgatatctataaaggacatattacagtgattgatacatggtgatatggtttggctgtgtctccacacaaatctcgtcttgaattgcagctcccataattcccacctgttgtgggagggaccctgtgggagataattgaatcacgggggcagtttcccccatactgttctcatggtagtgagtaagtctcaccagatctgatggttttataaggggaaacccctttggcttggttcttattctttcttttgtctgctgccatgtcagatgtgccttttgccttttgccataattgtgaggcctcctcagccgtgtggcactgtgagtccattaagccttctttctctttataaattacccagtcttgagtatgtctttatcagcagtgtaaaaacggatgaacatacaccgtaagtgtgacttttatcattcatttaagcatcctgtttataaaaatgtaaataatccattttcacatatgctgtgttttcgataaaataacttgcttaaatccaatcctggcatcatagataatttaagccatggttttctgatcccgtcctcttttgtgactccagtcccacagacaggaaccaaagccagcagaacaaagcagccccagatgtgttctactctattatctgaagtaaatgaaaatcaaccctgaggtatgtttttatattctgtctaccaaaataatgtgcattctgtttattgaactgctcttatttcttccttgcaatctgttactttcattttcattcctgccaagagagaaataaaaaagttgctcataatttctaaggcgatttagacctacttttttggcctgtaatggaaatctgcggtttctttagtctgatcctaggtaaagctgattttagtgaagccaggggtttttacacattaaaaagaaaaaaatggaatggtggaataaactgagttagggagtcctgaggtttatgtggattctcttacctatgtaaccgctaggcatgccatggtggatagaataattcccacccccacacgtcttccctgacaagatgtccatatcctaatcctcagagcctgtgactatgtcaccttataaagcaaaaggtactttgcatatgtgattaagtcaaggacattgatatgcaaagattatcctgaattctgagtgggcccaatttaatcacaacagttcttataagggagagtcaggagtgacaaagtaagagaagcaggtctgatgatggaagccgaggtcacaatgacgccaagaagaggccatgagccaaggaaggcagagggcctctggatgctgcaaaaggaaaggaaatagacctcttggaacctctagaaggaaagcagccatgctgacacttagatatttggaattctgatctctagatctataagctaaaaaaattctgtttttttttaagtcactaagattataacaatttgtcacagcagcagtaggatactaattcagtatgtcaagtagctttcctaacccaaattctgagacatctcagggacctgggtatggactaaatgtttgcgtctttctaaaattctatgttggaagctctaacctcccctccaaggtttttagggcctttgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcttgactacatattgtcaaattgctccctgacataactgtaccaatttatacaatcaatagtagcacataaaaattcagaaatcccccattatcttgtgataaagatttttccaaactaatttgtgagcattgattttttgctgtaagagctcagtaggcattgaagggcagatggaatttgagatgtcctagagaacaggaaatcatggaaaagtaggaagaagctgcagctgaaagatgactgggtatctgtgtaattttgattggattcagaagatagcaaacctgctacagaaattgtgatgccaaaagagtcaacagggtgaatgaacagtcacacttatattttatcacctgtaacatgatggagggtatattgtatgactttcaggtaggtattggccaatctacctaatatagatattgaccagcttctcaggttgttttgttgtggtgtgaaaagataatcgcagtaaatgtcatagttactagggaaagagtattctaaataagaatagatttcagataagtgttggtgaatgaattgtattttaactatttttctataaatagcgatcctcagaggtttatgcaattttttttatcgtgaacaaagaacaaatatttgaaatcattcagaaaacaaataaatgtatgtagtcagcaagaaataaatggccagctttagaaaagatgcctaatgtaaggactgttgtatttactttgttgcttatcttactgagtcattccattactaacattctgaaagcctagtagagcttcactgccttcatctgaaggcagagtgagtgttggcatgtaagacattacctttagatttaaggtgataggaagtttccttcgaagtctcaagacttagcaagaatttaaagggtctcaatggaatgcctatatccaaaatcagagttaaggatgagaaatgggaggaagagaagtcagatgagaagggatttgtatctcaaattttcactagaatcaactgagataaattttaccagagttggaagtaggagtagggaacaaataagaaaaaacattaaacactaccaaagtgcccaacaatttccataactggtccagtaattattcctataatagtgtccggcaacttttatgccaatccattatttcatcagatcgtacacatgtgcactaattaaatgcctgcagaaagaagactgtaagttaattttaaaaaataactcgaaggattcaaatgtcaattttaaatgacatttagaaattcataggctaacaaaccacagcatacaattcagttatacaggtttctgtcagttaccactaagatgtcttgaataaattaatatatttttctaaatcactggcagagtttctgccattattaatgttgtagaaaaaaaccgagtccttgtcacatgaccatgaaaagttagacacacagacactttgaaggatgaggggggaacaaaatttattggggggaaaggagaaaacactctcagcaaagcgtgaagggttcccattaataggcccccatctcacagattgaatcccagtcactacccaggaataagagaagacagtctcctcccctctgcaaagggcgctcaaacgtcctgaggctccactctgtcctcctagtgcttaggtgggcattaatcagaaagaatcagttggaaaaggacgggcctcatgcccgaccctgcagtctggtttttcagccttcaggctgttttaggcttgaaggcagggtttctctgggggattcttggctacctgctctctctgtcattaaggacaagtcaaagtcaccttcaggttatgtcccttgtcctgctatgccctttactcaaagggtaattgcttgcaacagaaatgtactatatccttgtttctttctcttgaattctagttgggaaacaagccctcacattaatttttttttttacaaagaagaaagaaaagaaaaagaaaaacaatacatgatttattttcagactgtttattgtaaaaattagttcagagaagataaagagaaataattactaaagtttttgagtttctcaaaacagttgaatcctgcagactggattgttagaacttgggtaaataaagagaaggaagcttgaaagcaaatgccagcagcagcaaatttgcatttacatagtgtgtttgcaaaacagcaagaagattagcttagaagaattggaaggggaataaaattgagtaatcagaagtccttttggatcaaaagagaatgaatagattatggggatttttgaattcttatccaagatcttcaccctaccacaaattaagaaagagctactgcagtttcttaaatgaggtggtcagattgcagttacggccagaaaggaaagtaggcagtatagtgccaaatgttggcaaggaatataggaatatgagaattctcatgcactagtgatggtaatgaccacaggtacagcaattctggagatcactgcgacagtacttaaggcaaattaaatatacacataattaggacccagcaattttactctcggagaatgtgtgcatgtgggtatgagtgtgtgtgagtgtgtgtaccacgtatgtatgtatataaaatttctctagcaggaattctcacataaaaacatgaagagacacatacatcacaacattgggagttgggcacattctgggatccattagtaaaaaatattactttgtagattgttatattgatgacccccatacctcccagtattcatacgctttggtaatcttcttccacattgggcttggcattgtgtttcattttgaccaaatgggtcaaatgttatgtagaggcttattaagagcttgcatttaggggcttgtccctttgaactacttggatccagctgccatgtttaggtcacatactgttgaaaaggtcctgtggaggaatattgagatacgtaaaaaacggctaaagtcttcttggacagttgccagctaaatatagctgaatgagtgactctagccaaggctatacagaacagaagcagcacccagatgaatatgctattttaaaccacaaaatttatattaaaataacatagagatatgaaaatatacagcaaacaagggaaggataacaacaacaacaaacaagagaaagagagagagataaagagagagaaagagaagaaggaaggaagaaaaccaagtggctgctgctacattggaaattgcatgattgttgtcttgtgaatgattctcttaaaggtgtctataagcactcattattaacatgatttagagaagggaacgacacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaacaatattaagccaaggaagaaaattttttgtgagaaatattgttttgcataaaacaaaagtgttttgcactgagtttattttcaattttattttattttttgagacagagtctcaatttgttgcccaggttggagtgcagaggcacaaacatggttcattgcagccttgaccttctgggctcaagcaatcctcccacttcagccccccacatagctggaactacaggtgtacaccaccatgcccagctaatttttgtattttttgtagagacagagttttaccatgttgcctggcctggtctcaaactcctgagctcaagcaatctgcctaccttggcctcccaaagtgctgggattataggcactagccactgtgtctggcttatttttaattttaaattgaacttcaaaactcttctaagttttcctttcaaaaaccttgcttagctagaatttgacttactctaatttaacacaactggtaagagcctagtagaagaaaataacaccaaaaatatattaatttgtttaaacaatttaacatctgctaaccagttattagatatgtctttcttatgtatatcaatggttctaaatgtacttctccaagttatatgacccaaatgagtaagtttcatctaaaatccaagttgatgcctgactcaggccttatttgagtcctgccaaaacagatccatttctcgtacattttacagatgattttatgaagaaggaaaatgaggaataatgtctgccttttgtattcttctttgagcaaattttaatgtcttaccattctttaattaactaatgaaacctttgttgtgtctttatatttgtatgagaagcaatgattttacctttggttaaaaatgcttgagcaattttttcttgcatcaaacaaatttgaaataaacaaagaatcacagtgattataaacatgaagacccaatatacgagttactcataaacaatggatcaaatagcatcagtagtcactacatccaattattatttctgattttagtggaagaaaataaaattctgaagtttttaccaaatgaatataaatttttgatgaattttgttataaagatattttagtgaaaattttagacacaaagacatttgaaaagcactagaattgataccaaagaaccaagtcacaaacagtattacaatttctggaattttttgtgaaatgtgactttttatgaatttctgccaattgtattcttaggagtttgtctttctgtttgtataaccattgcttcatgtgaaagaaacttttgaaaattaaaattaataagtattctttaatcaattacaagtaaaactagactaatatggctgtgctatggaacatcaatacatggataaagttaattttgatgaagtcgttgacaaatattcataaagaggctaaaaaatggaaatacaatgctattattcatttctgtgtaaaactaaagaaaatgtctgaggggagccccaatcattttagaggtttattttgctaaggttgaggatgaacctggggaaaagaaacaaaatcacagaaatatctgtgatccatgctttttcctaagagggtttggagacacaatatttaaagcggaaagcgtgggcagtaggggaaaaaggaaagaacaaaaaggggagggtggataataaaggcaagcagttgcattcttttgaagctttgatcaatttcactgaatttacattttacctgtgaaaggagtgggtagtagaatagtcaactatgtgtttgtctggtgctcagtgaatcttcatttttatataggataaagtaaacatagaccagaggaagaagtcaaatacgcatttgcctcaggtgagtagagggatgacttctagttctatatttgtcctgtacctgtgaagataagctgttgatttatattctcagggtgaaattcagtacaacttcattttacagtaaggatcttggggcccgcaggagattttctgtgagaaaattgtaagagagggcccctgagaaggtatgtgccttctatctttgcagttatctatttaggaacaaaatgggaagcagttgtgtgtgacgcagttcccaagcttaacttttccctttggcatcgtgagttgggggtcctgagattttattttactttcacaactggaacagattttaaaaactttaaaaaacaaaaatatgtcagtgtccaaaaagtattaatgcattactttttcttatttttgtattgtttattttattttttaacatttttattgacatgaatatataggccattttcattatttgtttgatttcatgactattaccaaaaaaattttgtcatatagaggagggaagtattaaaaagggatctctgacatacacttggtatagggtcctggtaccaattgtgaaaagtaaaccctttcttataatccaaagtcacttggcaaagtttcttcatctcaggtgtccattttctactcacaaggtggttaaacaaaaaaagtatagaaattgggatctatcagcagacctgtccagtttctaacttggtaaactctaattaaattacagtgttaagctttacttttttttcaaaagaattagagaaaatgcaacttagaacaatgggcttcaaactagaatgtgcctatgtctgagtgttctcctgcacagagaattttaaggaaataaatgtttccatcctctttttacaatggttcttctcccatatatagaaaagtattaacttgtcacccatcacaaatcttaacacagaatgcatactggtgtgtaacaacctcagggctcctaacataggaatacaaattgtattttgcttcaggggaagactcctttaaaaattaatcaaagctcctaaaatcttgtcttggcttgtcatacacatatttgtagctagagtgaagttctgcgttagacacagggtatttggcccttgcttaagactgaattgacaaagtcttaagtagaataatgaaagttgttattttaaatgtaactagactatttgttaaggcttccacattttgggttatttgaatataatccacagataaaaatttaaatgatttctactggtgtattttagtcaataaagcagttaaaatttcatgaaaatatatgaaatgtttattcctattacagtaaatccttatcctattttatctcataaatataatgatatataatacttatgtttgtgataatataatttgatataaaatacaatatttttcacctcctaaaaataataaataaatatacttagggttatggctaaaaatgtagacatatgcaagttaaatacacagtttttcaaattttttatatagcacattaactaaaatatttgaagatcattggctctgagaactagaaaatagcttgagtacttttcctgatctgtcattaatttgctgagcaatgctgagtataatgtttaaagtgggaatccaaattatctttctgtaaaattatactttgggagctggagatacctttcatgcctgtaaactgatattgtcttaattttttgttccctaaggaagaataattttcttataattaagaattttcctgttctatcattatagcctctaaaatattaaatcttcttttgttggtctggatacttatatctaactgaactaggatatattatctttttttcaatgtcatctaacacaaaagctgtaatcattgatttgagatttgagctatgcaaaatggattaggtttccatacccaaaattataaaagtacttagatcataggctattaagtatttttagacttctttttctttttaatttagctgtttacagttacaactttccctataagcactgttttagctgcatctcaaaagtttggtatgttgtgtcttcattttcattcatctcaaagtattttctattttttttgtgatttcttctttgacttctttgttatttaggtgtgcattgtttaatatttacatatttgtgaatatcccacattaattttgattattgacctctaattttattctattgtgattgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttatttagaggtaaggtctcctgttttttgtttttgttttaactacaaagcattagaatcattccactggaacaaatattgctttactttccaattttttcaatagggtattgatgcttctcaagtgaagtatcattaatggttccttaaatgtggccaaaccagtttaagtatttttgtttccatattttaggttgatattttgatgttttttgtcacatgggggaactatcatgtggagacactgtaagtcatagttaaagatccttcaaaatatattctaaatggcattttattacagatgtatgttttataagcataccagtaaatacactcaatgcaattcttttgcttcaaatgtcaataaaacttagtaagattttaatacattagcataagctaattttaactttttatgttgctgatgaagcacccattcaggaaaaaaaaagctcatgctttatgtaaacatattaatggacaatttatctccttttcattatttttactcatgctatatttgttagaaaactgataatatagttgtatgttatgaatctagttagatttggattttattttcaacaacaacaacaacaacaaaaccagtaatctgaacatgaaaagtcgatgtaggcaagagattttcttgcagattgtcagttgtcactcaatgtttcatatgtgagtccttggcttattgttactaactccaaacaatcggtaacagagttatgtagtcctttaaattttacaagaagctttcaaagatgtctcatttgatcctcttagtatcttagttgagcaaggtgggccaggcaatgctatcaccattataaaatgggaagttccaggctcagacaggttgaattgccaagcaagtaactgacaagggtgatctagatcttttagtttgacatggtgcagctcttttcactatgcattcactgtcagtggacatattacatttatataaggtggacaaaaggggtttagtttcattaatcattgacttcaaacacagcaataacaaccaccacctcaaaaacaatattaactaacagtttgtgaggacttaacattttgcaatcattgctttaagcatcacacttgcaaacctgtggggtaggtaatatttttaaccttctcagcactggagtatattgatctgttatttttcctgttttacaagtgaggaaactgtagttcaaatacattaagtaatttgtccaatggttgaaaacaggcagtctgactaaagaatctgtacaattaactcctacactaaactgtccccatagctaatttttgttttttaaatcaaataaagttacaaaaagaagcaaccaaatattttaattagtggctcttattccctgctattttctatttatatctataataataagtagtttcaacatagactagagaagaacattattttaatatagtatctagaggcttttgagagttatggacagcttttgaaatctagattccaacttccactgggcgagactatgggaaagaaagtatatttagctctactttgatgggaatcttaatatcctccacactttctgaggacaacaattcataactttactaattatacttcttggctaagattttaagttttcactgttccccaacaagttcataaagagatttccagatataggaaaatatcattgactaaatgacctccaaaatacctttcagtgctcaaagtcaatcaatctttgacataatcatgttagatctatccttactaaactaaaaaaaaagattaagataatctctcccagccatgtacaagcttaggctgtaaagaagatgaaaacaagttagtttttattttaatcaatttgacttcagaagcaaaactttatagctcaatctggtattcataagcttttcttgaaaatgaaacatatttttatttaatttcatatactcccagtatgttatagtgatgtgttgatttaacttaatgcttttacttattataacattgatactaagcatcatctttaattctcttttaatttcttttaattttcatacataattgtttaaaccttataaataaatggatatacatatatatgtatgcatgactaatgttttttgtttataagttctgatggcaaggtaaatattaaaagaatttgatgtagaaaaaagtaatttaaaaagtgcttcttttaatattcaatactacctttttcatatgacaaaacttacagttcattggcaagttaacaataaatctgaatgtcacttctttaggaatctgaattcttggatctctctttgttctattgatctagaatcacatcaccgaatgtgtcatttagaatttgcttttctctttgcttgtaagtattctgaattccttgcttgaagtattgagtgactatattagcaaaaataatgacaacagatgtgattattgtgcgactaaccaagaccagcagcagaagcttcacaaagaatcaatattcggctcttagccacagagattaaagaggcacatgaatataaagcaaatcattgtataagaaatagttgaaatgaaatgacatgctgctccataaaatgatctaaaaatggaagccacagaaaaaactggcaaaacaccagagctttgacaaacacatgcacagacctgggaacacccccaaatatatatgtatcttcctttatccttaatcaaaggtttctaattttcacattaactagcaaagccttgaaaacaatgattacaacactttatttatttatttatttttgagacggagtctcgctctgtcactcaggctggagtgcagtggcgcaatctgggctcactgcaagctccgcctcccaggttcacgccattcttctgcctcagcctcccaagtagctgggactacaggagaccgccaccacgcccagccaattttttgttatttttagtagagacggggtttcaccgtgttagcctggatggtctcgatctcctgacctcgtgatccacccgcctcggcctcccaaagtgctgggattacaggcgtgagccaccgcgcccggccgattacaatacttttaatacttaacttaccaaagtaatttatttttattcgctgaaaatgtgagtcacttcagctgacatttctgaaagccacatattgcctttttacttattgttcattcattagactgaacttcatgttgattttagggctggctaagccattccctaagctcttctttttcatctttaactgatcctcctcagcttggggcatacatacaagaatgcaatgcattgttcagagaatatagcataatctacttcctgatatgattcctatgctacttggccagcagtgatgacaacaggtagggagcttttatagaagaatggtgacccctcttttgagatcctctacagctggcaggcatccttctgtccctgaacatcacagcctgtgtaatatcttgtaggccaaacaaaagtcagtaaatgtttagtgtctagaacttcttttagattcagaatgaatataagacaattggaaaataaaacagtaaaaacattcacaaatgcaaaaatgtgttggacaaaaacaacaacaagtgcaaaaagttattggacaaaagggaaagtgagggtagaagaactgaaatagagagaaagagacagagagaaagggacattggcagaaatgctcattattcatttcctctcatatctttctctaagtgagctttcctcatttttcattcccagagaacaattcaatctccttatcttttgcctatgtaaagtttaaggtggaggacaccacaacacaccgtaaaccagagagcatgggtccattgctgctcaaaggtgaacagataagagacatagcttttttttttaagtttttttagcaagttaaaagttattttatgtatattattaaaaagtcaaataacaacagattctggcaaggttatagagaaaagggaactcttatatagtgctggtgagaatgtaaattagttcagccattgtggaaagcagtttggtgatttcgcaaagaacttaaaacagaaacatcatttgacccagcaatctcattattgagtatatacccaaaggagtataaatcattctaccataaaggcacatgcatgcatatacgttcatcacagcactagtcacatggaatgaaataaactattcacaaaatgaatacttgtgatatatgtgaattaatatgtgaattcactatatgcaaagacatggactcaatctagatgtccatcagtggtagactggatagagaatgtggtacatatataccatggaatactatgcagacatcaaaaagaatgagatcatgtcctttgcatcaacatggacggagctggagactattatcctatgtaaattaacagaaacagaaaaccaaataccacatgttctcacttgcaagtgggagctaaacacggagtacacatgaacacaaagaagggaacaatgaccactggggcctacttgaggatggaaggtgggaagagggtgaggatcaaaaaactatttgttgggtaccatgcttagtgcttgggtgacaaagtaatctgtacaccaaacccccaagacatgtaattggcctatataacaaacctgcatatgtacccctgaaactaaaataaaagttaaaaaaaattcagttatgtaacagtaaagaaaattcatctgtgtagaaaaataatcaaaagatacagaaagttataaaaagtaagagtcatggccattccagccactggttctaattacagacgatagccatatcaacacttcctgtgtttcctcccaaaccattattcttacagcaacatgtatgtgcctgtgttattacccacatagcaccttactgtatactaactctaaaaaatggaaaatgattacattcttgtggtgttttgtaaacctgacgatacttttctgcatcatcattttctagtcaccctcctgctttttcatggttgtttaatgtgccattttaatggttcaaaatcagtggttaattgttgttaaataatgattaatgttttaatagttgtttaaaacaaggtgttagattgtatgaaaggctgtaatctatttactcattccaccaataatggaaatttttaaagtaatttgttgttttgtttgtttgctacatgccactatttctgtatcagtttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactttgacaatagaaaatccctatactatgggaaacattttttgaaagcccaacttatagatgggttcttctgtcctctcaatgattctgtaagccatttaataccctataataaatttcttccaatgtaaacaagttagagaagattgtgttctctatatagattttgctttctctctctctctcaactaacccccaactgatacaccatggttttggtaatcaactatacgcatttgctcttgtgacatcttttggacaatatatcatctgccttaccttgataggttctccatggggagacataacttcatcagagagctccatcatcttcagggccatcagcgctatctgaacagcatgagtatcactctctttgtgtaatcccccagctacacaataggcatcgccaatggtctccacctagacataatatattttccatactataaataaatgctcaggaagaatcacagcctacatcggatagaagaaattaagtcatatagcagaattctcattgctcaggccttctggaggatcagggttaatcagatacgcccagaaatggctttctttttcaggaaatgcgcaaggtttagacccattacattgaggcactgcactgagaggcagtacagtcctatgaacacagtaacagttgacttctactcccagatttgcacactgactcatcatatcatttcatctcttttttttcaccatataagatagtttttgtagaaacaatatatttttgaattataataaattgtttaaagatgttgggatcatttactcgttttatatcctcctcatcataaaatagtattttgcctcaagtcactactcaataaatgtcacatcaatgcagtaaaattgtgcaatgtaaaaccaagatattaatagtcataagagtaaagagaaacggttcttctaaaggacccttggggtcctcatagcaattacatatctaatggatactaatcacaattataaaaaccaagatgttgaaccagcaggtaaaaacaacggtggtgtttgaaagaccactggaaatcaagctattatcaaaaatctaagtattaaccttatcattatcatcatcattattgttgtcgttacaggcaatttttatctgtctgtgatgctggctgcaaaaagcagatagttaattttagtccagaaataaccccctaattctaagagcttcatttttcttcttctccaaatgtttattgtaactataagaccctattattcttacacaaagcctcaattgtgaaaggatgagggtgagaaaggaaaaaagaaaggaggcacaaatcttcagaaattcccctcagagtagatgctgatacccttattatggctccagatttcttctttacctgtctttctcctaatctctgactttatcttcttattctccatattgctcacttagttccaatgtcactgccttccattatcctttaagcacatcaaacatgctcccacttgctattcctttctcctggagcaatctctctgcagataaccacgtggttgtccctcctttccttcagtccctgctaaaacctcaccttattagaaaagctgattcctaccaccctctaagaaatagcaatcaccccttacccactgcccttcttcttccccttaacctactttatttttctttcacattattatactgacttaaatactataaatttactcatttgtttattgtctttgtttcccctaatcatatgtaagccctttgagtttatctagtttattgactcctacattacctgtgcccagtaatgaatgaaccaggtggcaatgccacccataagctttttctcagcaatgagtacataagaagtgctcatcatgatggccatcacaggtgctcaataaataggctaattagaagaagtcctacacccacccctacctgatgttaggggcctaccatatctgcttttgcagccctttgtgcatactgcatcccttcgtcacgtcatatggaaatctggcattggtccattgtgccctcttctccctctccatatgtgaactttcctcacagggattgtaccctctagctctgtagccactgcgcacccatgctggcaaatgctggataaccagtacatattcgctgaaacaatgggtaaatgaacacacagagaaacagaaaaatggccaaaaacccaagaaagacagagagaaaataaaattaaataaagccctagtataatccagtcttggaataaataacgctgactattctttcaaagccattggcactcatagggtaggctacgattatacattttgtgtatctgaaacccttctatatttattttcagctatctcgaagtaatctcaagcaaaatcagaagtgaaaaaaaagtagaaatgagggctggcatagaaggtaatttactatctagttacaactctagttagattaatcagggaactctgtggctgttgtccttataccttacagtgaggagcatagtttcaaaatgcttgtcaggatacttaaaaactataccctatatccttgatagaaataaccaaaagtagaagagggctacttctgttagcaattacagcagtttgcaaggaagagagacttccttctcacaattctaagacaggagaccgtctcaagatagaaaatggctacaaataaatatagtaaattccacaagaggccatcaaacttaacgtaactgaagccaattattttttctgtaagtatacatggacctctttttccccaagggaatcatggctctgtttggactttcctgttaggaatttttaaaagtaaggttttacttctggcatctagaataaaacaagacaagagcttatgtattatcatgaataatattcgtttcttccacaataatttatttatttcctgttgggttcagctctgaaatgtcagattgttatgtggcaagttcagaagatgattcaagccatacaaatgagaaatacgctttattcttagtgccaatgtctaaaagaaagcaaagtaatttaaatcagtaaaaacttttaattctaaagaagcaggcagaacttgaaatctcttcattagctctcttactaatagtagttacgtttaaaaagaaaaggtatcagggatggtcactgctgtatccccagtgcctacaatgatgcctggctcatcctgtatgactgatgaatatcatgtattacctaacataatctttataacaatcctgttcatgaagattatgggagataatgtgtgttattcatgcatataaactctgtgagtgaatattattttctctgtcttagaagtgatgagaaaggttttttaacttggtcagtttacaaagcaagtaaggcagagaacaagatttgaagataggtctttctttttcaaatgtctctgcccttggccaagctgcaaaaatgccttaagtaggtgattttgatttctgttaactggccatgaaaagtatcatgttaactctatattgaagtaatctatttaaaggcaatttttataatcctcatgtgtctttactcttacagatacaaaggataatgtttccttgtataagccatactttgaattatctcacacttaataattcaactcggtttccacagtgaagtggcagattcactattaaacttctcctattcacaggtcctacaccccatcctccattctttgagcaccctatcctccctccattctctcctcattctttgagaggacagtgtggcagtcctcccagaacaattatctctgtttctgggcagattcagagctattgctggctggaaaacacccacataatgaatgcaccatacctgctcccaggactgggcttgcaagaccctacagaggtggcctttcagttgcattataattcaggtcctaggcacagtgcttgttccccagtgagtttgctggtaaaacaattatttccctggtccactcctaccttgtagacatccagctctccacactgctggtcgaagcgagtgtacagtgcattgagcatggtgatgacctgcagcggtgagcactgggagcagatggcagtgaacccaacgatgtctgagaagagcatggtgacattactgaacttcttggcttgcacaacttgcccttgccacagctgctgagcaacctcacagggaaatatggagcacagaaggtctactgtctttttcttctcctcctccagggcttggtgggcttgctcaagggtagccttcagcttccccagcctcttcttcaggccatcttgagctcgggcttgttcccctattaagaccacatccctcagtgcattgtgaattgggatgtctgagaggtagagccctcgtcctgtaaaatcttctaatctgtccacacagggtgaccccaaaaacaagattgcactggattcaacaatgtagatcatttggcctttgaggtccataacctatgaaggaaaggataaccaattgtggtttattcaagtttcccacatctttctgagacaaaagaaggggaagtagatacagtaataaaccctttgtgtttaaacctgctgaaaaggaacaatttgctttcttatagttcctattttctatgagttctaaaatgcgtgcagatgtttgaagagtatattagagtatactctacacagaaatcaagatttacaatgaacaatgcaaaacgctcttgcttcaaggctttctcactaagcagcatgctgatgcttttgctactatattcttcttaatggtctcacttgattaatctgaagttctattttttgtataatatggtagtcaggaaaatgtgtcctttctttcttccttcttccctccctcttacttttttctttgttctatttttttggcttccttctcttttagcatgtttctcttctcttccccacttctcccataagtacttgctccatctggcaggtatggaagtgaaaaagcctccaaaggtaagatttttatcttaatttttttttctgtcgtgagagagatggggcttcaagtgatcatcctggaccacaagaaggtttacttgcacatgtgtggacaaagatgtgcatgtgtgttcttgcagcaaaaagccaaaagcaacccaaatatccttcagtagggtactagttaaataaagtagagcttaggagaacgaggcggatggatcacccaaggtcaggagttcaagaccagcctggccaacatggcgaaactttgtctgtataaaaaatacaaaaattagccggatgtggtggtgcaagcctgtaatcccagctactcgagaggctgaagcggggagaactgcttgaaccccggagttggaggttgcagtgagctgagaacacgccactgcactccagcctgggcgacagagcaagacgcagtctaaataaataaaataaaatagagcacaaccatatgatgggataccactaagactccaagaaagccaaatagacctatatctaatgacatgggaggatgcacaaaatacactgtttcatgataaaagcagttgtaaaatagcatttttttgttttaaacaaaaaatgtatataattagtttttcatttttttgctacaataaacacctttcaatattactcattttttaaacatctcaatatattttattatttttatgatagaaaagcaataataaagctgttttccttttggggaacaaatggtgcttctgttaaagttatacatgtgtgaaacttctctcttagctttgcttgtattatcaactatgagttagcaaactttctttcctgagactaaataattaccaaagctctctaccatgagatgacagaatatgttttaacttccacattcggtaacaattaatcaaataaattatttgtttttttcatgtctattaaatgttgattaaaaatccatttagttgcctcctacaagaaacaagtgacttttgtttgttgtaaaacaatacctcttattattcaacctaagtcagctatttgataattgaaaacatttttatataaacatttatcaaatttcattataaaacattccataaagatattaaaaggcagactgcattcaataaatatttttggagtaatgaatcaagtaagcaaacaaatgcagaaagaattttgttttcagtttattacagctttacaattcttttaccattgttttacatatgttttatcaaacatttgaagtgatgcatatatttttacccttttgtttcttgctcttaaatatgaaatagctttcatattcaagatagtattatgttttccttacccttgaagattttttcacagagttgtcccatctcctcactcgtacaacaaactgcatattcaacatagtcatgatcccgctaaacgtctggttgatttttggagtcagaatttcaaagtattcttcaaaattaggctttccttgaaagtctctcctgttcatcagccttctgatgccattgccaaattgcagaattgtcatatctttgtcaaacatgaaatggaatggaaatgtcttgcagaatagcgatgtgggaatcaccagcgaggactggggtttgctgggggacagggatggcttggtgcttttcatgtgaacggagtacaacaagtagggctgattcacaaactcgctgcaatcattatggaagcagggaggcattaacgacacttccacttccgtttcatataatacgtgagcagctgcctttatgatgccgggaagaatcagggaggtggttctcttagggaagaagtagtaaacatgtagaaaatcatcctccttatccaggcatagaatggaggcgtcctcaagcctgcccctttttcctgcttcttggcaatggctgctctgtttcagaagggtactgaagctgtttaaaaaatctttaagggtgcctccaaccaccccaaggatgttttcatcttcctcgtaacatattttaaaaacctcttcaccaagagattctttgataacctccactggaactcctgaaatcacataatacatgttctgccatatcaaatatgccaccttggtaaagctgtgccattattcctattttcaacttgcgttatttgctgatttagctccctttatatctgctaataacatttccaaccaaaatgaaacccctatatccaaattatctacctaggttagcttctaaaattctagtaactgaagaaaaatcccaaactagttcattaaaaaagaatgaccggttgagtataatacacaactttttatctccctgaaacctaactacaaaagaataatttgaattccagtattaagtacattttggcaaaagtcctccaatctttctgttaacaacaaaagcaatccatcctcatgaagaggatcttcctctatgagggtttccagcagcaaaagggtcagacacggaagttaatttaaaattactggcatgaaactcagttttgtctagaaatgattacaaagcattttttcctgaatcaaatcgcattcttgttgtttagtgttagctgaatgaatgtctgcttggagtatataatttactttctatagcattttcacgagtgctataaggttagaaataagcctctgtatgtctgtgtctttttacaatgttaggcttttattaatattattttttatcataaaagccatgagttacatgaagtttttaaggaggcaattttttttttcttctttgagacagagtctcgctctgtcgcccaggctggagtgcagtggtgcgatctcggctcactgtaagctctgcctcccaggtttaggtgattcttatgcctcacctccagagtagctgggactacaggtgcacacgagcatgcctgcctaatttttgtacttttagtagagacgggttttcaccatgttggccaggctggtcttgaactcctgacctcaggtgatctgcccacctcggcctcctaaagtgctgggattataggcgtgaaccaccatgccttgccaagaggcagtttttttagtactttctgtttacttgtttcttagagctgcaggcacacaggctcaagtcgctccacaaatcagttagtattgtaaacgatacataatagtatgcttaattaatatagaaaatataaatgttataggttaaatattttgtaacaaagtaacacttaacatcaaaaggaaaaagagataggagaaagaattaacaaaggagggtgggtatggtgaagaagacaaaaggagtcttggtttgggtcaggccgtcttataagaaagactctttgagatggcagagcctttggtggcagatgtccagtttttatcacgagtgaatgtaagaaggtgtcaggacgaccgttttgagttgttgaaggtttaattttttatagttacagagtcctctggtgagaactgatagtggaaaagtgtgtttgtgtttttatcttgttgtatgtagttttcatttttttaaatttgtttattaaataaaacatgctatttttgttggcaaaatgccctacaaaatacaaaatggagtcttttttaaagatggagttagttatgtcaagggtgctctgtatgatcttccctttctataatctcctttaatctttacactctgagtaccacattttttcttatctgtaatgtgggaaaaagaatagtgccctcttcatagcactgttgtggggcttatattcagaatagggccttgctcctaggggggcctcaatatacgttagtcattattattattatcaatatcttatacttttagaaagatgcaacctcgtggttcatgtactgaaactttggaaaggacagcaagacaaagtcacatatcaattttttctctcaaactaatcattaatgtgagaatactgttaacttttttcccatgactgctcctaacctttaaaagcattctttattttaccttgttgagtagaatttttgatgtataaatgaggaacagttagttaggttcaagatcaaagcctgtgaatatctgtgagacagcttcccatctagccttatctactcctcagcaagtaaacataaacagagaaaatgttttaggtcaaacaaaaacatatagtatcaaaaagagttactctttacgttaacagaatacataatattgaacacttcatactgggagtcaccaacaaataaatatgaatatttttggagaaaatgtgagttctacaggtatatggcatactttctaattacataaacaattctattacctgctgcaactgcttgctctgcaattgttttttcaaagtcttctctttccaaagatttcctggaaatatttcaatattttaagttataaacttataaatacatgctaaaagttcagattataatatacagaaacaatgatagaaaatagtttaaattaggccaggcacagcggctcacgcctgtaatcccagcactttgggaggccgaggtgggtggatcacaaggtcaggagttcaagagcagcctggccaatatggtgaaaccccatctctgctaaaaatacaaaaattagctgggcatggtggcgtgtgcctataatcccagctacttgggaggctgaggcagaagaattacttgaaccgggacctggcggggcagaggtagcagtgagccgagatcagccaccactgcactctagcctggccaacacagggaaactgtctcaaaaaaaaaagaaagaaagaaagaaagaaaatagtttaaattcccttttctgtagaagcaaatcttgactatcaattttatgagaaaggagcaatgacatataccatccagaacaatgttggaaaaccccaaaatatatatgacatagaatgttttgttattcttcataagaaatatttacctttaaagtgaactttacatagactaatacagacagtctcagacttaggattttttttttttttacctttatatcggtgcaaaagctgtaagtattcagtggaaactgtacttcaaagtttgaattttgattttctcccatgctacaggtatggagtacaatgctctcatgtgatgctgggcagtggcagtgagccacagctctcatgtggccatgaaattgtgagggtaaacaaccggtactctacagtgcactgtgttgccagatgattttgcctaactctatgctactttaagttgctgaggacatgtaaggtaggcaaggctaagctattcagtaggttaagcgtgttaaatgcattttccacttagggtgttttcaagttacgatgggtttatctggacgtaactccactgtaggtcaaggagactctgtactcaggagaaccttccctggctagcaacctcaaatgtgaagtggccaagaagaggatggtaattctgataaccaagaaaaacctacatacagatagaattcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaccccgtctctactaaaaatactaaaaaaatagccaggcgtggtggcgggcgcctataatcccagctactcgggaggctgaggcagaagaatcgcttgaacccgggaggcacaggttgcagtgagccgaaatttcgccactgcactccagtctggttgacagggtgagactccattatcaaaaaaatagaaaaaaaaaaaaaaaataaagcaaaacaaaacgaaaaacaaaaaaaaacacagcatctatactgagtgaacgtatatgcatattgtatgcactgttggttttatttttatttaaccagactgagaaagaggtaggttcatagaaacttcagaaagaaacacaaattaggacacacttggaaactactattactgtggtattagaatctttggcatgggagattgatggctttagcaaactgtatgaaattcaagtatttacttaattatatggttcactttataaaatatttaaattctcttctgaccatttattcaagtgcttcaatatgaacttctgaaatatttgcattttataggtatattttatgtgtttaattttttcaaaagcactgttttgacttctcgagggattctcacattaagtttatcaggtccagagataccagacaatttgaaccacatggttaatactagatcatgtcataaagtgatataccaatgagaagttaaaaatatgattagaaaagctatttgttttgccagtttgatcaaagcaaaaaaattagaattgaataattaattcataagtttaggatacatataattcaatttctctagttaaatagaaacagagtagaacatcaaaagtaggcctgtcatttaaggttgactctcttgccttgtctatgagttagggaaatcaaccttaatgtggtaaaattttcaaattataaataaggatactaatatttaaccagtatagtaagttattctgacagtgaggtagtaacatcccagttttgctacaaaatatatctaaaagaatatatacaatagcatctatagttattgtgatttgtttttaaaaattgcagagaatgcgacagggtactcctgaaaaaaaaaaaagaaaagaaaaatgaacaacattttgtttttatttagctgtaactgaagaaatgttgtcccaaactttttctgttttgggtctggttgcagttgagatcgagttttccctttgtaagagggctcattgcttgcaatttcttaacccaaaaagaagaggaaaatctatactttaataatgtatttgcctgtgaataatactttagaaaagtaggatttttttacattatagatattagctcacaattacgattacttctttagaatgaattcctagatttgcaattaaatcaaagaatccacaaaattttaaggaatttttatgcctattgtcaatttatccttcagaaagattaatcaaaataccccctcatcagtggtatttgagatgcctctttttcagtaccttacccaatttaatattgtaacttttttttctttttttgagacagagtctcgttctgtcgcccaggctggaatgcaatggcatgatctcggctcactacaacctctgcctccccaggttcaagcaattctactgcctcagcttcccgagtagctgggattataggcacccaccactgcgcctggctaatttttgtatttttagtagagatggggttttgccatgttggccaggctggtttcaaactcctgacctcaggtgacctgtccgccccaccctcccaaagtgctaggattacaggcgtgagccactgcacctggcctggtattgtaactttatgtatttgatagttgatggttaaaaatgacattgcatttatttttgaatatgaatctctatcattttatttcccattgttttaaaatagtattgtaatcttatttgatagtgctagcttgcttttagctggctttcaggttgttcactatacaagtgtgcttggctaagaaggcagagcccacaagtgctttttttttctaattctaccaaagagaccatataggatactggggaactgataccagatttgtaattttacaatgattattcaatcttcaaattttatagatcaaagtccagaaatattttcctattttagacatatggagtatttgcatatatgttttgaatttttgtaattaatctagtaaatgcagtattcataactcattttttatatttaggtatttttattaaactgaaatgaagacaattcatgccatttccccctttgatatccaggttattcagcaaagaaatgaagaatgtgatcatactcaatttttaattgaagaaaaagagtcaaaagaagaggatttttatgaagatcttgacagatttgaagaaaatggtacccaggaatcacgcatcagcccatatacattctgcaaagcttttccttttcatataatatttgaccgggacctagtggtcactcagtgtggcaatgctatatacagagttctcccccaggtaaaatgacagcatacttccttggggcctgagacaaaagcccatagaaatactattgttacaggcagccatccattcatttaaccctctgactatgtattaggtaatatgcagtttattgtaagttacacaaattgtttaattgtgaactaagtgtatttgagcataccacaactttccctaggacaactatttttttataataatcacatacagaaacagtaggaatgttatgtaagaatagtatacttgttacagtgaaaaaaaatgggtaactttttagtaacagtcacctttcacatgtttatttattcactgaataaatattcattggatatctaccatctgtaaatagctttaaaaatcaactattgatggtgttaagggaaaaggggagattttaaaaagaactaaaacacaaaatgatagagatgatgatggattcagtagaaaaagcatcaaaggaagcagttgggatgatgatgagaaactgctgttgacttctatgacagatccattggtaaaatcctttatgcttactctttttttcttaaatactaacttttggagttgtatatggcatacgtttagagatactttttgcttgtaattaaagagacttgaaaattcaggattagcccagttggatcaggtgaaatggaagcagagtagtgtgctgtcctggctctggtattgactctacctgtcccttactgtatgaccttgaacttattacttcacttctctcagcctgtttctctttgttctggcatgagaatagaaagcatatgaaaaatacttagttcattgtaagcaatcaatcagtgttacctattgttttcacttttagccctctagataaatattaagagagggtttgctcatgtttttggtattttaatttcatttcaagccatacacatttaacataacactgtacattttaaaagataaattttcattttttctccttctgaaaatgcattgtaaatttatgctagcttacatttgaatattagtcatctgaatccatatcagatttcatgttcttgtaactatttaatgtccatttaatcactgagttgtatagattgagatttgagttgatagttcagctaagtttcccttgaattataataattataattttctagttttaaatataactgagctgatttaatcaagcgattaatatcaaagtataaaaactattttaactgatgtgatactctttgctctctatctatattttagctccagcctgggaattgcagccttctgtctgtcttctcgctggttcgtcctcatattgatattagtttccatgggatcctttctcacatcaatactgtttttgtattgagaagcaaggtaatcaagatattatttcattaaatgtgagaaaggtatgtcacaaattagaagtattcaggggggaaaattatcacattctctgagaaagtatagagagataaaacccagacctcaaaggaagatctatttaaaaggcaataacattgatttttggtttgcctgaggagcagtggaactctatcaactgattaaaattaacagaggtaaattttagagactgtctttctgagacggccttgtttcacatcaaaaaaccaacatcttttactccctgtacagctacagtgccttattttagaaatcagaactaaaaaggatttttttttttgaacttcactcaaaaccacttatttattctttcttaaattgtctattgttcttagtagcgtttttgtgggtgaataaaagtgatgaaatattcacacagttgataatttagatagtgctgggatctcacggtttctgttcttgggatagatacagcaaaagtcattttcctagagtacccattgaatcctttctgtatttggaactgactgtttgcagctgctctatgaatacagggtgggagaatggggaggcttggcttattctttgagagtctggtaatgtatgttggaaccataccggaaaatcagggggataaaaaagattcacaaaagcttgaaataattatgtgaagagtagcacagttactgtcctgaattcagacgggacagtcacttttctgcagtgttgcaaggcagccactaggcttattacaggcagaactctgacagaaggccctctcctgttctaatgatgcacttattctgctttgtggtttggtgggaaatgagatatgcgtgtccttgttgctgaattcttaggtagggagaaaggcattcattaatggttgtatcttgccttttcaaggaaggattgttggatgtggagaaattagaatgtgaggatgaactgactgggactgagatcagctgcttacgtctcaagggtcaaatgatctacttacctgaagcagatagcatactttttctatgttcaccaaggtaatcatttttagattaattatagtggctatcagtacctatctttagctaacaaaggaatgccacaatattttattccattaaatttacatattctctgaggtgtaattagattttacagcccttgttgtgtattactttataacaaggataatcttatttaatatttgctaataaactcagaagaaatcacatactgtatgttatttgccatgtcttaatacatttgtggagtgtttatgccattacgccatgggacaaataatctgcattaagctaattctatagttttggaccttcaaaataggcattatgtagaaaagtgattttttaaagcagctaataggatggcataataaatacattggtatatttgacatcaaaaattatcttttctatagtatgttagcaaaaaatctaaaaggtctctttctgggtttgaactattttaaattaaaaatatttacctactctaatccaaatacactgttccaaatatattattcaattaaaatgtatgtaaaatgtgatttttaataaacgtcagagaaaaataaaactatagattgttgttttccaatttaggaaagcattttactaggagatacatgggaattcagaaatataaaaattatgtctaaatttgcttcagaacaatagaaaagataactgtggggattattgtgagatgatattctgtgtattcataattttgtataaacaaagtgtaggtgcattatgtagtaacacagtcaagaaggcatgttttggtgagctattggttaaaaatttaatctgaattccaccatgatcacttttcacttaaaaatgtggtcttaaaatttctgaaaaatgtaatacactttgtaacaacctcctgttatattattactattgtattatacattacataacaataatatataaggccaaataagaataatatattgttggcacagtagttcacacctgtaatcccagcactttcggaggccgaggtgggtggatcacaaggtcaggagttcaagatcagcctggcaaacatggtgaaatcccatctctactaaaaatacaaaaattagccaggagtggtggtgggcacctgtaattgcagctactagggaggctgaggcaggagagtcgcttgaacctgggaggcagaggttgcagtgagccgagatcatgccactgcactcctgggcaacagagcaagacactgtctcaaaataataataataataataataataataatatatcgttaatttttaagtacatttaagattaattaaatattatttaactatgacatataagaataatatatcagataatataaggatatggatgtcttttccctgagaaataaatatcaggaaaataaaagaaacaaaactaagaaacataaacgctttttaaaaaaattattaagctacattgatctaaagatgtcccatctgctatcagtaagtgttttcttcttctgcctcttcacaggttgagcttaggagaatgtcagcttctcattaggggcaatttctatttatggaaagaaaggttttcttcagaaataaaagcagaaaatttggagtttatttgttaattcccatatgcagacattggtcacccagtgagaaaattgcagattgtcctatgagacatgctcaggtgtgtaggaagatttgtactcacactacataaacaaagatcaagtaaaagtagttttacttgtaaaataattctaagatgattagtttgaatttagaaaaaacttaatggttcattaaaataatgatatagggcatttttagaaccacgagaccgttatgatttaagttttgtaactcagtatgagcatttttgaccattcatttttgaacgtacaatcatgttttttctctgctcatgggcaggtctcgtatacctttggccgatgatctggcttctttctagatgtgtctttttctgcaagtggatttacatatccattttaacgaaattaaaagacaccgtgtatttcattagtaaaatctttgattgttttattttctttataggtatgctaatttttgttttgctttatgaacattgctgctgagaatatattgcattaatccaaaattctgcaaagtcaccccttttaaagaattttggataggattaattcaaaattcttcaaattattaatcccagattctccaaagacataactaaattttttccttttagagtaattattattcagagtggccagaagtaatgactttgcattaaaaaaattcaagccaaagatataaccttatgccattttaaccaattaggttttgttgtcgttgttgttgttgtttgagatggagtctcgctctgtcacccagactggaatgcagtggcatgatctcagctcactgcagcctcctgctcccgggttcaagcaattctcctgcctcagcctcccgagtagctgggactacaggcgctagccaccacacccagctcatttttgtatttttggtagagacgcgtttcaccatgttagccaggctgatcttgaactcctggcctcaagtggtctgcccacctcgcctcccaaagtcctaggattacagatgtgagccactgtgcccagcctcaaagagtacatttttaaaaagcagttaactttttacttgtcagattatcactaaaataataaacattattatgttgttggaaaaatgacacaagttacaatgatgacgcataaaattcagcaggattgaataatgtttcaaataaatgagaaaaatttgtcaaagtaaaaagaagacatgagagccagaagtaagaatgacacatagttttagatacataaattacatgctgaggctgggtcataaacgtggttgggaatttttactcattgtttccaaaagggaaaaataagcagttacaagattcatagaatacagaaagcaaaatgaaagtttctcaaaagaagcattattattcttgagagagagaataaagatagatttttttcctctccttatgatgagaaaaaatttgagaataataaattacagcctgtgttaaatacctcaacgtttatcatagagatgttttctacatgatcgtttcaatttagactgatggcaccaaaaagtgcaacctggcaaaaatgcatctcaagttcactgctttttggtagatgcttaaatatctaagagaataaactcacaatcttcgttcaattctccataattctattgtcttcagcctgatgttttgaatggtaggaggtggcaggatcagattccttgacagaaatcgggattgcattgtcatgtatataagttgagactgatcttgcctcatttctccatatgacagtgtcatgaacctggacgatttgacaaggagagggctgtatctaagtgacatccctctgcatgatgccacgcgcgatcttgttcttttgggagaacaatttagagaggaatacaaactcacccaagaactggaaatcctcactgacaggctacagctcacgttaagagccctggaagatgaaaagaaaaagacagacacgtaagaatgtaacgcttggagcactactgttattcataacataatgtgactctactatatttaagtttgagaaccagactaaaaagccatgtgacctgtaatagctctggtgtagtaaataaaatctttcacattgcttttaaaaagaaattatgcttaaggaaataaaccagtcttaatggtagtggaaatatgtctgctatttttagaagccaagttggaataatataggccaaaatatcttttggaaaatcatgagaataactcaaattcaaatttagttcaaaaattgaagtttaactatcatttattcctgcattctttcatacaagagttatatattgaccatcttcagtatgctgggggctgccgcgtgcagagagctgctctgcacaatgaacttaaccactggccattacatgtttaaaaatttattttctgggaccttgaatttaggattagaaagaaatgaatctgacaaattgtaatattatatgtaatgacaacaattacatctatagtgacatacattagcaaaaatatcaaaatataaaacatttctatgagtggtggattcaattatgaaatattgagcaaggacatgaaagataatacttgattttcaagaaatgtatttaagtaagtacatagcatgcatactacttggccttgctttcatatgctaattaaacgcacatcactgacatttataagctattttacccaggttcttatgtgttcgttttttaatgtttatataaacatttggcggagcttctaagaccttaggactaacgttcaaagtcacatgggtttgtaggtttgagttactcaatattctgttctggctgtttgtattagaaaatcatctaggtttcattttttttgtattgtgactcaatttgctaatatctacattcatgataaatagataaataaccagcactataaataacatagcctttaaattatgacatatgcagatattctgtactgaaaaagctcttaattcacttcttttcctaaattgtcagcttaagcagagtattagcttctcttcctagaccaggagtccaaaactgggagttgctgggagttaaacagcagccttgtctttaatacgagtagctgataagtacttattacagaactacttctttgtttggattccccaaggtgggtttcacattcaggaactatttgcaccatttcgagttactgtgcttcaaaaatccttatattattttttagattattagatttagaatttggtttttgtagatatttgggtttcaaggtgatgtcattagaaaccatttactattaattttctttaaagctaagtagttttactgttcatacttactattcaaatatagctttaatatttcataaaacattcaatgtttaaaagttgaaaatgaaagaccggagaaattctactatgactaggaaaaacattgagagctcagctccattttgcatatttaaacttcccacatatgcccttttgctttagagcaaaaggcaggaagaaaaaggcaggagggcttcatttgttcctcttcagtcttccaatccagagcccataggtctagtttctgcttctttccctctaaattcaccagctgtcatcctcctcctgtgtgctcacatttgtcccttagcatgatcccatagtagtctcagttgtacctaaagaaatacaataatttattttctagaccaacttcctctggttttatcagtttcttctatggaggagtgaggcatgaatgagactggagagaaattctgcttcttttaatattacttaccaaaaatagagacatatttcttattctaagaggcaaacctgtttccattattgtgctattcataccatgttttaggtgctggtaatgacagtcttcatactgtgactatcgtaatagcagggtcagaatacaggactacaaatgaaaactttgattttctattaccagtctttttgttttgtctgtttgcttggcttattttgatttggtttgaactagtttgggcttgcggatgtcttatgtgattaggatgaacaaataatttatgttttctgtaaatccttgcttataaatacagctaatatttgatgctaattttagaggatgtcctgctagaatccaggcctttaaagtacaaacgacactgatgctgtgtgaaaaggacagcagaagcactaaaggctttcccagtatttcttacagtggctttctgctgatcccactgaacagattgctgtattctgtccttcctccgtctgttgccaatgagctgcggcacaagcgtccagtgcctgccaaaagatatgacaatgtgaccatcctctttagtggcattgtgggcttcaatgctttctgtagcaagcatgcatctggagaaggagccatgaagatcgtcaacctcctcaacgacctctacaccagatttgacacactgactgattcccggaaaaacccatttgtttataaggcaagtgttctttatcgctgactgcagagctatccagaggctggcgttctgagactcccctccagaggccatgtcatcacagctctctgactccagcactgcagccttgagtacagtgagcctccatgtattcactctttaccatgttcttaaataattgcctcttgttataaaactgtctcttccttgtaaccacaatgaatgtttcatgaagtgggtgattccctggttaaaatgaaatgttcaccatcttatttgcacttaggctaacaaatctggacaggctgtttatcacatgtagtataaacatagcattcattttagtcctctgcaagagacatttttactgagatatataaatgctttgtacagtaaagaagtcatgaagcatgagaaggactcagatttgcgtttagataacatcaacttgaagaagataaacatttataaggcgtttttgccctttgtttcatttagaaataaatataatattggaaaaaagaagtcagtggaatattgctacataaaggcaagaatgttaagtaaaccatctctagatatctggggtaatataactacacaaggactatttattttatagtcattttctgaggtacactctagcatgtggtgagcacatcatacaaacaaatgattaatgaccgtgataaatcactgaacttgaaatggtgggtgtcgctgccggcaagccatcactgttttaggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcttaaagttgctggtagacttgaaaaaatatgtttttgttagggtaataaatgaaacagtcttttttatgctaaccgtgaacatctaaatatatgtactggtaggtggagactgttggtgacaagtatatgacagtgagtggtttaccagagccatgcattcaccatgcacgatccatctgccacctggccttggacatgatggaaattgctggccaggttcaagtagatggtgaatctgttcaggttagtaaatgaagtagatattgtaataatggtatgtaaacctttttggacaattgattcatatcgttgtccggaaaatcattaacgtgtataaagaagggcatcttgacaacaaagactatagcatttcatccagcccactgtactacattctttcatagacatggagatttatctacctgtttgaaattatctagtccgtttcatgtactgaaagtcatggggatgagaaaaaaacctcttgtaccttctagtttattttttaaaacctctttcatgtctcatgtaagttactagattcacatctttgaaatcttcagacttcaaatgtttctcttgagataggatatttccctggggagatagagatttcatggagggcttttgaagttgtccataatatgcttgaaattcttatagacatctcttacgatgttatatcatgactctacattattctgtaatataaagtaagcaaaaattcatgtgttacaggaacagaaaaccaaacaccacatgttctcactcataagttggagctgaacaatgagaacacatggacacaaggaggggaacatcacacaccgggacctgttgggagtttgggggcaaggggagggagagcattaggacaaatatctaatacgtgcggggcttaaaacctagataatgggttgataggtgcagcaaaccaccatggcacatatatacctatgtaacaaacctgcaccttctgcacctgtatgccagaacttaaagtaaaataaaaaaaaaaaaattcatgtgttagtgatcaaaatgattgaagcaaagctttcttttttgcagataacaatagggatacacactggagaggtagttacaggtgtcataggacagcggatgcctcgatactgtctttttgggaatactgtcaacctcacaagccgaacagaaaccacaggagaaaagggaaaaataaatgtgtctgaatatacatacaggtgagagaaaatgtcttggtatttactgatttgcaaagaaaatgtgtctttgcatgtggtttaattctctgagagattttgttgctttaacagagttatcaccttcactcttccccactgcttgtagagttgtttactttttatttgagaagattgtgtcattatttccattttatatgatttcacatcatcttaggaaatcctgatatgatcattgtttctaaagctttgagttacctttcagtacaacttcagcatttgttcttgccatcttggtttataacatgatacatgtcaccttctctataaaacttgtgtatacttctctctctactccccttcccttgtcttttagatgtcttatgtctccagaaaattcagatccacaattccacttggagcacagaggcccagtgtccatgaagggcaaaaaagaaccaatgcaagtttggtttctatccagaaaaaatacaggaacagaggtatgactaatcaaagtgtaatttgcgtacttaagacagagtataagtatggaaaactaaagcaaaatcactgaaaatgtatttcatttgaaaagaattcttgctaacagaaattaacttctaagtaaagcagtggaaataattgaacattaatgagccaattacaatcataagtactttttaggggatactttagtgatttttgtcctctaatgaaatgaaaaccaacagactggattgagaaggctgctttttttcttgctgttgtggttgttttgctcttcctctgtagtctggctcaaaggtaagtgggctgtgaggggctcccctgacccctttcctcagctctctaagttggctctgagatcaccacagataagggctgtggttcatgggaggtttcttaaccatgaagcttaagatccttttaaggggcaggctatctttctatggagtgattggggaattttggaaatcctagtccctcatgtgtttctcctagatagatgagtgatttttaacttgctagaatctgattaattccttagggaagatttaagttctttctactactatggttatactccaggcagagcccatgtgctagaatggacttcagaacagtggcacatccaaagtggaatcacacctctcatagaggccttgcacacactaagatcatggctagcctcttcctagccttttgtaaggagtggcatcagaagcaagtttgatactttgttaccctgttgatgtgttgtttcctcatgggggataagactgcatgctcatatttctcttttagtgtgtataaactctctaaagatgcttttagggcataacaacttctaaagggaatagaatgccagggtttgaaatatttatcttctttcacctggtttaacattaaatcattttcctatgaggtggacatgcttattgatgttcttctgataaactaagccgtaattacctcaaacattggtcatcttagtcgtgcggtcctgtttttcccagaattatggtgggaatgatttacagaaactgtagatgtgaacgtggatcacactctcataaagagaaaaatatatttaattgatattataaacctcactatcactgtaaatcaatccctcttttcttctgcctatttaaggaaacaaagcaggatgatgactgaatcttggattatggggtgaagaggagtacagactaggttccagttttctcctaacacgtgccaagcccaggagcagttcttccctatggatacagattttcttttgtccttgtccattaccccaagactttcttctagatatatctctcactatccgttattcaaccttagctctgctttctattactttttaggctttagtatattatctaaagtttggcttttgatgtggatgatgtgagcttcatgtgtcttaaaatctactacaagcattacctaacatggtgatctgcaagtagtaggcacccaataaatatttgttgaatttagttaaatgaaactgaacagtgtttggccatgtgtatatttatatcatgtttaccaaatctgtttagtgttccacatatatgtatatgtatattttaatgactataatgtaataaagtttatatcatgttggtgtatatcattatagaaatcattttctaaaggagtgaattctaagttttaggggaaaaaatgcaatttattttcagactcccaaagtaagaattaacatatcatgctaagaaaatagtgactattttgaagtatgctacttccctttcagaaatatagaatacacgtttctgttattaaagtatttgattactaattcaaatcatatggcaattataattcttctaaaatgctatcatttgtaactgtatcccctgtattaaatctcattaaccacaggcagctgttacagaaagctgcattgtttcatttttagctgttacattagttcaggctaaatgttgggagctccaaccacatccaagaataaatctggaaacacactgctgggatactgctgttagagcccttcttggccttgtattcccagaaatgagctccctttccttagcttagaagaatgtgattatatccaggacatcatgttcagaaaacttagtttactttcagcatagaatgcattactgttggaataattggcctctagctcttaaatgtctctgataacttattaatatctatctttataaaatagagtgcaactacttttgtgtaaaaatgtttgcctttaaatttagtatttcatatcagcacatcgatatatgtataaatgttccatgttaatgtgtaaaagagtctgtaataaattatttttttcacgtgtctctatacagtttttatttcaataaaaatattaacattatttttcattttattaatgccattattttgtaaatacattttacaattttgcctatttgctaccattataaattttcactgactcctcatagacagcatattcttaaatgctatatttctttttaataccaacagagtgacaggaaataaagactgggcatggaaaggggaaacagttaaatattgaatgtcatcagcttatttaaagagctctgaatcatttagggatgatagacatgtatacaagtaatgaaaatatgcgatattcataatgaagaaatatattcaaatgcagaaataatacttagagaaagagtaacttgaaagaaaagaggtgaagtggggatagttgttatgcttattttggggggagagttaggaatactcagaaactacattgacactgtcatcttcccagaatcctgcatatatttatttagcatctactttgggtaggtactgctcaagatatttgagaaatgtcattgaacaaaacatctctgcctttgaatacctcatattccaatttttacatcttgcatgtttttaaatctctactttattccgccaacactcttcgaagtccaggcactcatcatgtgtaacttgaacaattgcaagatcttccctataaatctgtctatttcctacctttgccacctccattccacactctcaaattgctgccagagtggtcttttacaaatccaaattgctctcctgcgtacaatcacttaaatgctactcattgtctgtacaccttagcaaagcatgtaaacattgtcaccttaaccagccttatctgctagttgctacctttgtaattctatgctctgctctgtctgaataccctctacccatactctgctgcaaattcatttcttgctaagactcagctcaatatcctcttgttggaaatttccctgttaggaaagtttagcttcccctattgtcccaggatctttacaatggttcacatattatctgtttatatataggattccttattaggctttaagtttcttatggctaaattagttcctcttacaccagatattccttgaaccttccacaaaataggcacttgacaaatatttgttgaacaaaggaatggagggagcagcctcttcgagcagtggggctagaagccagatttaaagggaaggaggaataagaaatgagatagattggagaaaagaggaggcacaaaaatggcactgttaaacttgtgcttagagaaaatcatgctgacattggtgcaggggctaatttagagggaaagagcctggtttaaggttggcattagggggctactgtacttcatgcataggcctgctttaaggtgatggcaggacaggaggctaattgaagagcagtcaagattcacattaaaagtgcataataacgcacaggccaaaggggatgagaaggttagggccaagtagaaagcttctggcttgcttagtgggtagttgatggttccattacacgtggtggaggatgtagaaaggtaactgagcttagaggaaaggtgcctaatattaggcatggctttgaagttctggtgaatacatttttaaaaaacaatgattttcaggtagcattaatgcgaggagagaatagaacttttttttccaagttgggccaaactgctttgaatttccaaacaacctgttactagctccatgatattggtaaagtcatttaacctttgtgtaacctggttaggtcattgtaaagacagagattataataaccactcactagagttgttggcagatcaaactgaaatacgaagaagtcagaggagtgggacttttggcttgccatttcagaaagattaaaaggtatttaagaaatgaaaacttccaaatctttggtaacagatattgttatagttgaatcatggacagcacatgtcatgaaaaaacaggagggtcaacaagaaaaatcaataaacacatttatctttgccaattattgaaacaaaccccagtggaaaccaccaaaaaattagaaccaaatgttgtgaaacgataacagtttatcagtttgaactgaattgttttaaactttaacaattaaataccattttaaaggtatgtatttttaattatataagttatatatttattatattatagattacatatattatatgtattttagcactaattcactatttcattcagtttaaatctgtatttgattcaccaaatgatatgttctatttcctttgtcattgaaaggaaaaataatttgcttacctccctcataggttttctttcttttttttttgtaccaccctcactaatggtttatagaacctctagagggcagcagagaatgctgtttgtaattgaaatctgaaaaagaactgaaaggagacattctctcacaggacagagttcatcttgtctcctagctgtcacttcctgtgtgcaaccttgaattcactccttccactctttctcaagttcaaaggaatataataaaaataattatttctactcattaagaccatttgtatagcatgaaatctatccacacgtatcttgctctgaaatgtcaccaaaatatttcctgctaattctgcaggaaggtaaaaacatttgctaaaacaagcaatttgtaataggaacaatagttttgtacaatatttttatgagtaaactaacttgcttgtagctcatgttaaaagttattaaatagaaataggatactatttacttataaggataaaattggaaaaaaatttattttacattaatagaaaatgcttttgtgattcttccttgctacttataccgtatcaaagagaagaaatatagtgtgagttaacaaaacaaaatttaacaaaaaacaaagcaccgtacagagaatcaagaacctgagtccaattctggtgaggaaatcttgtggcctttacaagtcacttaactttacaactcagttacctcatctgaaaaaaaggagctggttaaggtgatatccaaactaaattattgctccaaaattccatcatttcgttggtaacaattccagggtttttgtatcagaagcattctaaccaaagtgactccatctttacagaggctgggtaaaatgaggctgagacctgctgggctgcgttcccaggacgttgggcattcttagtcacagaatgtctacagttaagggaataggttaataatgtttaccaaacagacccaggacttaacagacccaggaaatgtccaggtgtcccaatatcttaggaataaatgtattcttaatttaagaatacgttttgctttaaagataataatacagattattgtagaacagtagttaagcaaatactcagctgataaaacaccatgcagtaaagaagcaggctgaaacccaccaaaaccaagatggcagtgaaagccacctctggtcatcttcactgctcattatatcccaattataataaattagcagtttaaaagatgctcctactagcgccatggcagtttacagatgccatggcaacatcagaagttactctatctggtctgaaagggggagtaactctcagtcctgggaattctccactcctttcctggaaaactcatgaatattccacccctcgtttagcatttaatcaagaaataatcataagtttactcagtccagcagccatgccactgctctatggggtagccattcttttactcctttactttcctaataaagttgttttcactgtactctgtggattcacctcagattctttcttggatgagatccaagaactctctcttgtggtctggatcagtacccttttccagtaactcttgtgctaggattttggcctgaaacttttgggaatacaatctaaagttttgacaatctagtagtgttgaatagatcaaccgaaagactgaaagctaacttccaagaaataaacaaatctgaggtaaacacttggtgaggacttttgactccaagctaaaatcaaaatgactaaggtcctgattagtgtatatgagtattaatgttatgtatgtctactaaaaatgtgccctataatatttaattatgttagctaaatttaagtgtttaataaacagtcacattcttgatgacctgaaaacaaggagagaatgtatgtctctatttcaattatatgtactcacacaattgctgtggtacagtctatcccaaatagggcttaaatataggggccagtcatggtggctcacgcctgtaaaccagcactttgggaggccaaggcaggtgaatcacttgaggtcgggagtttgagaccagcctagtcaacatggtgaaaatgtctctacagaaaatacaaaaattagccggatgttgtgatgggcacctgtaatcccagatactcgggaggctgaggcagcacaatcacttgagcctgggaggcggaggttgcagtgggctgagatcactactgcactccagcctgggcgtcagagcaagaatctgtctcaaaaaaaattacaggtaaaaaatatcagtgagtattacatataggttggggtttttaaaatgttattcttatgaaatatatttcccaattatgtaaagtagtgaccacagttgcctaccaaacagttttagatcattagggggaaaggagtcacataaaattaaagtattaacttagaaaacacttttaaactgttgtaaatactgtcccttttgtggttttctgcatcacatgaataaatgtcaatatattaataatttttatatacccatgaatataataccatcatattgttctggacaacaacagtacctaaatctgacactgtgactacatacagttttagatgtactagtacaaataacgtaatcaaagatattttgtttatttttgtgtaaaaatcattacagtctaaggcaggcatgggtgaaatcagcgactgagtcctctgctcccgggcaactggggctctgaagagtgggggtcaggggagtcccctgagaagtttgtgtcctggaatccaagacaaggtgagggcagggcctgaagaaaatacaagtctcaggaaatatgtagatttcaaattgatctgacaaaaaaccctagtggtctgtccaaagaagccagtagccagaagaagcattacaggcaggaagcattatgaagttaagtgtgtctggtgttggaagccttttgcacagtgctagtttgtgtgtgcagtggtgattttggcacctgtttaataattgtatatcctcaatcgcaagtattggacaggatattgaataatgggaggttgcaatttcttaatgtgttagtataactattttttcctctaaagcacagtgtttttaagtctgtttagaaaacttccaggcgcggtggctcatgcctgtaatcccagcactttgggaggccgaggtgggcggatcacgagatcaagagtttgataccagcctgaccaacatgttgaaaccccgtctctactaaaaatacaaaaattagctgagcgtggtgtcacgtgcctgtaatccaagctactcaggaggctgaggcaggataatggcttgaacccaggaggaggagattgcagtgagctgggatagcgctactgcactccagcttgggcgacagagcgaaactccatctcaaaaaaaaaaaaagaacactgcaagggtaatgtctaacagcatggtttaatagtaatacaatgtgaggcccaaatataagtcacatatgacatggatttttaaatttaaaaatttctagtagccacacttcaaaaggtagaaaccagtgaaattgactttaataatatgttttatttaactcagtataagcaaaatatgatcatttcaacatgtaatacatttaaaatcattaattaaatatttaaaatcagtgtgtattttaaaatcagtgtgtatttatacactttaaaatcagtgcgtatttatacactttaaaatcagtgcgtatttatacactaaaatcagtgcgtatttctacactttaaaatcagtgtgtatttatacactaaaatcagtgcgtatttatacattttaaaatcagtgtgtatttatacactttaaaatcagtgtgtatttatacactaaaatcagtgtgtatttatacactttaaaatcagtgtgtatttatacactaaaatcagtgtgtatttatacactttaaaatcagtgtgtatttatacactaaaatcagtgtgtatttatacactttaaaatcagtgtgtatttatacactttaaaatcagtgcatatttatacactaaaatcagtgcgtatttctacactttaaaatcagtgtgtatttatacactaaaatcagtgcgtatttatacattttaaaatcagtgtgtatttatacactttaaaatcagtgtgtatttatacactaaaatcagtgtgtattttacattttgcagcacatctaatttcagaccagccacttttcaagtgctcagtggtctcatgaagctagtggctatcatattgaacagtgcaggtctacatatacaggttattgattggatcttttatccaaaacaatcatcttagatacttgattgttttgtttcctccttaccagaagattggatcaaccttgttatttataaaaacaaaacaagtagctgagtagttgaaggggtcattaaattaagaaaaaagagtataggaaggtttatactttttattctgatttataagatgtagttttgaatagagtgtttcttttgaactctaacattctatatactacaacataatataaatggtgataaaaagagtaataaaggaaaatgagttctatatgaggtatttctcaaacaacatttgcagaaaagaaagtgtactattttataagttgctaacattagcaacttcatgatttgagatgataaaggatcttacatgttcacctgacaattcttttgcagtcttttaataagaagcaattataaagcactaagaagcacctattatcttttgacaaagttaaggctctttataaagatctagttcatcagtcagttaccacccacatcatatttctttttttcatttgtattcctgttaaaggttctttccctgtcaacccaacttactctttttgcacttgacagtaatattaccaaagtattaactgaaagagaattatgcaaaagttgactaccaaaagatttgtacccttgaatacacaggaacaattttcacttgatttgtgctcagtaaagtgtatcagaaagttcagaacccttcagacctcaggaggagcaaatgttcctgtcacactgaccatatgtctttttgtgaatttaccaaaagttcttcctgattcatgaatgtttgaaattcaaaactcattgtatacagcttgttggatttcttgacatgtaaagcacttttaaaaagagttcttttaaagatagtgttaatactataataaaagttataaaatttgcttttaaaatatatatttctttaaatattgtgctgtattataactgaccatcaaacaacagacatgagagttgagataagtggtgaccagatccctgctgccctcaggctcacaactaccagcatggaaatcagggagcaatcctgcagtcactgcaagttcaatggtgatttgaacgttttacatgtttaatccttgcagcaatttattaagtatatactcttattatcgactccattttactcatgaggaaactggagcacataaagtttggggaactgcccaaatgttaaacagagagtgagtgggtaaagccagtatccatacccagacaaatggattccagagcccaagcttccaactaggatgctcggatgcatctcatagcaaaataagcaagcggctggcaagtggcgtacatctggtgggcgttttaacaccagatgagtgtgtgtcaggtaacaaagttctagtcaggcaagcaggcaaggagtgtgaagaagatatggcaggtggtattggccctaaactttacgactaactctgaacaacaggatgtggaaggctggggcacaagaagtgggggctgaggcagggaaggcaaacatgttcccctaaagatggatcacaagacagcgattataaaggctagaccgggagaacctatatgttgtgcaggaggaaaaacagcctgatgctaattcgcccctctgtgtataaaaggcttacgttcttctgcctgatacccagattggttctagagttttcagatttggggactgatgaactccaacagcaaaccctgatgagctgtacaagtagggaaagcatgtccttgactgtatcctcccattaaggcatctgctaagagagcagtcttgctggatgtggaaggaatcctccataaaggtagtcagccttagtcttcctcactctgccaccatgactagcatttcaatcttatttgtatgggcttctgacaaaggaaatttggggcagggagggggctctttctaaggcttataacaggttcttccagaaatatagaagaaatttttcagctttcccaatctcagttaggtctgtgcttccatagcatctgagtaaaagaccattgctattcttctatctgttacaattttaatgatttactgtcttctggtcaaatagactgtgggagaaattaggtcagacactgcacatccactcattggctccagtagccagttccagagccttgtcacagagtaggggttcagaattgtcaggtatccttcctccaaatactggagaagagttggtgaagaagaatgtcgtttgtacaacttgtaaagtatctgtgttttccacttgatggtctgttttgtgcattggctgtagatttatttactggaacggccttctcatcatatacatgtgcacatccatgactgatgccatgctgtagtttttaacaaagcattgttgaacatttgaaggataatatcagaagggaatagctgcgcagaatataaaacactcaagattaaaaattcttctgcctgcaatcccagcactttgggaggctgaggtgggcagatcacaaggtcaaaaaatggagaccatcctggccaaatggtgaaaccccatctctactaaaaacacaaaaattagctgggcatggtggctggtgcctgtagtcccagctacttgagaggctgaggtaggagaatcacttgaacccgggaggcagaggttgcagtgagctgagatcgcgcctctacactccagcctggcaacagagtgcagtctcaaaaaaaaaaaaaaaataaatgcttcttctcaagtttctaaattcccgtggctatattcaatcacatggcgtcccagtgccagccaaaggacacctcagcgaggaatctttctgtcttcactcttcaccaagtcccagtgcctctaaatctctcttggcctacctaaagttatcgtccgttttcttgcattattgatgtctagaacttatcacaatttattatgaagttaagggaataatttttaaattgttagcaattatccttaccaccagataataaaacattgttgatatatatgaagacatcccagcattcaggcataatgtatgcactttcttctaaatggtgccgtaaaagagaacgtcatttgcagatatttgccataagcaacaaccactctaagtgaagagaaaaagtaccttgccccctctggttttcacttagtaatttaggaattacacgttatttatatgggattgtatatgtttatccacattttaatgactagatttaaattgattttaaattactatcaatatattatgataatcattaaaacatcattacatcattaaacaatcatttagaaagttttgctccttttaaaactaatgcatgaattaagctgaataacaacaaaaaagtatcaatattttaccatcactaacaccattgaataatactgttttcatgaccacaaagtaccgttttgccaagcataagttatttaggctgccaaattatactgaaaacaaatatcaaatgtcaccaaaatataaaatcttcatccatttttctatatcagattgcaagagaagaaagaacctgatttgctcagtgattcatatgaatctgatttgaattaataatgtaaaattattaatgacagtattggattatatcccatataattaagaaaacttcctgaattcatatttatgtaagtaaatgacttaaataataaataaatgagggataaaggtcaattcttccttacagaatgatttcaataaaaataattatgaacagatcagtacttggggcaagggaaggagagaatttggagagttggagtaatggatctaacgtggagtgaaattaatcaacaggttaaactattctatatctttgtgaaattccctttaccataaaaagtggttcgtgtatttaaaaatggttctctgttacaccatccttagtataatagaaaagtttttttttctcaatgtattctcagggaataaccccatgcttttagttacagtattttccccagtttattttttttttgtaaaagattacacaatatcaagtccttttttcgctcaatatcatagttaagcatttttccacatagagtgagtatttaatgttcacataaaatttcatcccgtagctggaccatgattttctcaaccattttagcatagccagacattgacactcagaaactgaggatggattgagtagtgaggtacagcccagttttcttgtgcatttcagttgcctacatggtacagccctaatacactgaagcaagacctacttgttcattcagcacatattaattgagctcccacaacatgccagactctcgtctaagagcataggttagattgatggaaaataaacagagctccttgccctcagagggcttaccttctagcagagggggacagaaaatacaccataaatatattaggttgggtcaaaagtaattgtgtttttgccattaaaaataatttgcaccaacctaataatataaccataaattaaatgatagaggacgatgtattctatggggaaaaaattaagcacagaggaaaggagatcaggagtgcaaggatggggtggggctacttgcaatttggaaattgagagttggggctggcctcaatgaagggcaaagatgatgagtgagcaaacactggaatgctgtgtggtaggtattgcaactatttgaaagaaaagcagctcagggagagactgccagtatttgggctcatgacaggttcaagttacttatgttccagaaaccaaaaggagagtgtaccaggagcaatcaagccttttagtacctatgagtttaattataagaaagaaaatgtatgtcccagggaagttgagacagttcataattatccatcaatccaatgtaataaggagatatttgaaatatcctgcatgcattgttattctcactcatgaaacctttttatgcctttggcagttgtctgctccctgcttcctggtaatggtttttgatgactatgtattatttcatcatatagcttatccacaagtttttcacaactcttcttttgttggatatttggattaattccaattttcaattattataagcaatgctacagtgatatccttgcttatataaattatttttaaagaatataaaaataataaccggccagatgtgatgactcatgcctgtaatcccagcaatttggaaggctggggcacagggtcccttgagcccagaagttcaaggctgcagtgagctatgatcacaccactgcactccagcctggctgacagaaagagactctgtctctaaataaaaccaaaaactctatttgcagttgactagttgacaatacaggaaaatggacatactcctaaactactactttgtatttaactgtgtgaagggtaatttggaaatacataaaaaacacattaaatgttatcaagaaattcttgaaatttattctctgaaaataatatgaaatgggcagaacattttgtgattaagaatgttcattgaattatgatttgtaataattaaaaatgaaaacaaatttaacttataaaaaacttaatgatatatctatatattgaattatatggaaaaaatctcataggtagtacattgtatatacaatattgtatacacattgtatattatgattttatggcatatttgtattttttggtgtctggtggtgggatgtttgcttttataaacactttgaaacacatgtgtaataaagatataagtgttctgttaatgtacacagtattttttgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattctttaaagtacctaatgctttctttaggagagaggttatatcaagttaaaagttctcaaggaaagaactggtctcagaatttttgccatagccaccagccacaggtggctattgagcacatgaaatgtggcaggttcaaatcgagatgtgagtgtaaaatatacaccagatttcaaacagtaagcactaaaacagatggaaaatagctcctgaacaattcttatattgattgtttggtcaaatgaaatctattcttaaaattaaattttaaggccaggcgtggtggctcacacctgtaatcctagcactttgggaggcagaggcaggcggatcacctgacgtcacgattgtgagatcaacctggccaacatagtgacactccaacttcattaaaaatacaaaaattagccgggcatggtggtgcacatctgtagtcccagctacttgggaggctgaggcaagagaaacgcttgaacaagggaggtggaggttgcagtgagccgagattgtaccactgcaccccagcctgggcagtgagccgagatcacaccactgtaccccagcctgggctatagagtaagactccgtctcaaaaaaaaaaaaaaaaaaaactaaattttgcttatttctttctacatttttacaggaaaatccaaaattatatatgtgtctcgaactatatttctactggatgtctccatcctggacttttctacaacaaataactgtcactctttataattttaactccctgcataccgttttctgcccaccatctttctgtttacagcttacttgttttaccatcctgacctcagcaatctttctgggatctctgatgcactgaactaactaacgtttctctgtccttcaccccttgagtgtgcttgccccactctcaccttgttgcacatggcgattaatgattagtgatttcagtcactccttgccatttactctcagttctcttgttctctctcacttggtcacacttctagagccaaaattctatcctggataaattgcactctccagtgatttggtgcctgctctctggcaactgaatgtggctggagaaaaaaaaataaacaaaaactcaaaaccaaatttactagattcagtttaaaatcataaccatgaatgtcaagtggactctccacattgctaggacatcacgcgatacattttgagatcattcttttgtttttgtttttgtgttagttttgagacagcatcacatctctctctgtcacacagtctggagtacagtggcacaataatgggtcactgcaatctctgcctcccaagttcaagcgattcttgtgcctcagcatcctgagtagctgcaactacaggtgcatgccaccacatgtggataatttttgtatttttattaaaggtggggttttgccatgttggccaggctgatctcaaactcctggcctcaagtgacccacctgccttgtcctcccaaactgttaggattacaggtgtgagccactgtggctggcctactagaccattgattttatcaattttcacaccttagcctctctctcaaacctctatttctactttcagctgattactctgatatctttttcactaagaaatttcaaataatcagaggaatattaccattttgatatctatagcctcctagtatgtattcccatataatctgaattcctatgttcatatcacaaatgaactatctattgcaaactctctatttgggtgttaaatcatgattcatcgaactcctcaagaattgtgctctgacaaataggccttcttcctcccacatatttactttttagggcatttatacttacgaatttataaatttataaaaatggtattatgtctctaatttatgaaaaaacttttattggttacattttccgtgtcggcaccattttattgttttgcttagtagtaaaactcctcaagacagtggtttgttatttgcttctaccacttttctaacactgccacattgctgaaatcaatggtcaatattcaggcctcattgtgcttgacccatcagcaacatttgacacagctgatcactatgttcattaccctttccagcttctggtagcttttagcattcctgagcatgcggcagcattaacttcacctctgcttcacctccatcttcacacagctttcctttctgcacttttctgtgtccaagtattcttctctttttggtcttttatgaagacaccagttattcggatttaagaaccaccttaagtccaggatcttgagatccttaaataattatatttataaaaatcttggccgggcacagtggctcacgcctgaatcccaccactttgggaggttgaggtgggcagatcacgaggtcaagagatcgagaccatcctggccaacatggtgaaaccccatgtctactaaaaatacaaaaattagctgggtgttgtggcatgcaactgtagtcccagctactcaggaggctgaggcaggagaattgcttgaacccgggaggcagaggttgcagtgagctgagatcatgccactgcactccagcctggcaatagagtgagactctgtctcaaaacaaaacaaaacaaacaaacaaaaaattatttccaaagaaagtcacattctgagattctagatggacatggattttgggaaatactgttttactctttgcaggaccttggtatccttaaataactataactataaagatcctatttccaaacaaggtcataatttaaagttctggatggacatgaattttgggaaatactattttactacagtgtgtaaaatctacatgagaaaaaattgacaatcatttatgttatattatcacccaggaattgtgccttttcaatgttctattgtttattttgctatctttgagttatttttgttttattttgatctatttttttagaaaatgcatttaccacacaactaataaccttctactagaatagtgcccaaagttatggaaatgatgagaaatcaatatttttgttctaaataattgacagtcaggtaaatgtctccctgatgttctagtacacagagataagagatcagagttgagaaaacgaaacttaaaactgcagggagaatatttgaagaaaccctgtccaagtcaccttgacccatgaaatcatgtactgtgtggacacataatttggcaagctaatgggcctacaagttaagcattagtggagtccttccccatctcttgaccaataataggaatactttcaatatttatggcaaaccatggagttgttttaaactgaagctgtcccactaatggtgtggctgcagctgagtgacaaacagaaacctcccacaaactgaaccagtaactgcccaagaaactggagaactgaaataagaaactcatcaacaggttcccaaaagccttttgttttcaaaagactgaacataccagctaggtatgggctttttaaaaagtacaaatgaggctgagcacggtggctcacgcctgcaatcctagcactttgggaggccgaggcaggtggatcacgaggtcaggagttcaagaccagcctgaccaatatggtgaaaccccatctctactaaaaatacaaaaattagcccggtgtggtgtcacacgcctataatcccagctactcaggaggttgaggcaggagaatcacctgaacccaggaggcagacgttgcagtgagcggagatggtgccactgcactccagcctgggcaacagagtgagattccatctcaaaaaaaaaaaaaagtacaaatgattaggatatatccatggcacatttaataaatgtttgttgataatgatatcacaggcatgtagtatttacttatgtatattactcctgtgcatattatttttaaaaagagttgcttcctatatttaaagatctaaggatgaacatttcatgcaatcagtacatgaaagtaaaaatcaaaagaattgaacaagttcaacatcttgctagctacggtcatgtacaaaaaatggaataaagaaataagcagtatgtgattcacttctgttttgacccaagaagtttaggattttgactggtagtatatatgatagaaaagtataaaataaatataacataagtaaattcttatttaaaaagagcatccagaagctagtatttctatatgttgccttaagtctctcttcataaccactcaaattatcgagagctataatggtcaggaactctatctccttttctacagctaaacatgttggtgctttaaattgggaaacccaattttaaagcattacaagtttgagttattaaattagcaatgtttttttcttccaaaatatatttacattatttacttttaatagataattgtggaaaatgagtgatatggttttgcttggtccctacccaaatctcatcttgaattgtagctcctgtgatttccacatgttgggggagggacccagtagcaggtaactgaatcatgggagcaggtctttcccttgctgttctcatgatagtgaataagtatcacgagatctgatggttttattaaggggagctcccttgcacatgtcctcttgcctgctgccatgtaagatgggactttgtcatttgccttctgccatgattgtaagacatccccagccatgtggaactgagtccattaaacctctttcctttataaattacctagtctcaggtatgtcattattagcagcgtgagaacagactaatacagtaaattggtaccggtagagtggggagctgcgataaagatacccaaaaacatggaagtgactttggaactgggtaacaggcagagcttggaacagtttggaaggctcagaagaagacagggagatgtgggaaagtttggaacattctagagaattgttgaataaaatgctgatagtgatatgaacaataaagtccaagctgaggtagtctcagatgggcatgagaaacttgttggaaactagagcaaaggtgactcttgctatgttttagcagagactggtggcattttgcccctgccctggaggtctgtggaactttgaacctgagagtgatgaattaggacatctggtgcaagaaatatttctaaggagaaaagtgttcaagaggttacttgggtactattaaaagtattgagttttatgtatgcacaacaatatggtttggaattggaacttatgtttaaaagggaagcagagcataaaagttcagaaaatttgcagcctgatgatgccatagaaaagaaaaactcattttctgaggagaaattcaagtgagctgtagaaatttgcataagtaatgaggagtcaaatgttaattgccaagacaatgggaaaaatgtctccatggcatgtcagagatcttcatggcagcccctctcatcacaagcagggaggcctaggaggaataaatggttttatgggccaggcccagggccttgctgctttgtgcagtctcaggacgagccggtgggtacacagaaatcatggatgaaattggaaatcatcattctcagtaaactatcgcaagaacaaaaaaccaaacactgcatattctgactcataggtgggagttgaacaatgagaacacatggacacagcaaggggaacatcacactctggggactgttgtggggtggggggagggggagggatagcattgggagatatacctaatgctagatgacgagttagttggtgcagcgcaccagcatggcacatgtatacatatgtaactaacctgcacattgtgcacatgtaccctaaaacttaaagtataataataataaataaataaataaataaataaataaataaataaaaagaattgagttttgggaacttccgcctagatttcagaggatgtatggaaatgcctggatatccaggcagaggtgtgctgcaggggcagagtcctcatggagaacctctgctagggcagtgcagaagggaaatgaggggttccagctcccacagagtccccactggagtattgcctagtggagttgtgagaagagggccaaaattctccagacccctgaatggtagatccactgacagcttgcactgtgtacctgaaaaagctgcagacactcaatgccagcctgtaaaagcagccaggaaggggacagcccctgcaaagccacaggggcagaggtgccccaagaccatgggaaccggcctcttgcatcagtacaacgtgactgtgagacatgagtcaaaggagatcattttggagctttacaatttgactgccttgctggattttggacttgcatggggcctgtagccccttcattttgaccaatttctcccatggaatagttgtattaactgaatgcctgtacccctattgtatctaggaagtaactagtttgtttttgatcttacaggctcataggtgaaaggaacttgccttgtctcagactgagactttggactgtggacttttgagttaatcctgaaatgagttaagactttgggggactgttgggaaggtgtgattgatttggaaatgtgaggacatgagatttaggaggggagaggggcagaataatatgatttggctgtgtccccacccaaatctcatcttgaattgtagctcccaaatgtcatgatgatagagacaggaggcagccaaaggttccgctacccttcataacccacctaccccagccccctccacccctgtgaaaccctaccttcaagtctaaaacagcctgaaggctgaaaaaccagactgccaatctggatgaagcccgccctttcctgattgactctgaataatgcccagctgtgcactggaagaatgggacgtagcctcaggaaatgcatgtcatttgtggggggtggagcctggcttctcctgttcctgggtggggacctgggattcagtttgtgaggtgggaaacctgctcacaggactctttcctgctttgctgagagttagttttcctttttgcctaataaaataaattttgttccccttcaacctttaacgtgtctgtgtgcctaacttttcctggttgtgtgacaagaacccagtttttttctacaacatttttggtggccaacattcggcttgaggaagggtgagtaccgtgcaaaccaacacatcttttttccttttgcttctaagcctttttctcctcagacctcttctgaggggagaggaaattgtgcactaccccaccctgatggctgcaggcatacacaggatgggcaaataaatggtgggttcctcgctcccctccctgccagggctgggctgcatggcccaagggtgcccaacagcaggctggtcaatgctccctgccatgcaaccatggagccttcccctcccccggccaaaaattgtactctgattgactgcaattaaatatatctcccttgtggaggaaactatttgcataagaataagaggttcttccacaggtatcttttcttttctccaccctgtcaacaggtaacacagccctgcatttaaacgtcttttccttttctccactgggtcagcagttgacttaagcaagggtttttgttttttgttttttttccttttggaagatgttttgctaggccaggagtgatggggatcattgtttatattttctgtagagttttaattgtgacaaattctttatgaggttggttttaagctgtagccaatctggtatgctttgcatgattttcttttttttttttaaagatggagtgtcactctgtcacccaggctggagtgcagtggcaccatctcagctcagtgcaacttccacttctctctctcaagtgatcatcctgccttaccctctcaaatagctgtgattacaggcatgcatgcaccaccacacccagctaatttttgtatttttagtagagaccggatttcaccatgttggccaggctggtcgcaaactcccgacctcaggtggttcacctgccttggcctcccaaagtgctgggattataggtatgagccacaactccaggcctttgcatgactttctgtatggtcagcagtgaactttgctgcaggcctccatcttggtttatggacttgggggcatgacgtgtaactccatggcaatgttttgcttagcctctgcacaacccaggttcagtcatggcttagcaactgagtcctttcaggtttgatatctgtgtaacttttctatttgttgattctcttctcctccatgaaccatcttggattttcctttctctgaggctttagtaaagtttgaaaggctgaaatactggcttcttggtatggctaaagtcaggtaataggagatttaaaaggattttcttaaggagtgctcaactaaattaaagatgaatatctaagttacaggtatatttaaaaggcctttttgttttattttatttttttctcttctgggatcttgctttgctggaaaacggctttttctcagttggctgtattatttttctccattctgctttgccaattttaatgcacacaagagaggggagagatctctgtcttcctcattgaaccccaggaattaaaagtggatagatccctctcaaaatctcttttcgcctcccagtaatgcctgcctattaggctctaaaagctgcttgttttcctagccctccctcttaaagggaccaataatccaaatagaagatcagaaaatgaaaaatcgtatggctactgggttttcttcttcctgtctgtgtagttatatatgtgttgggtgtgtaatgtctatttaaaaaaaaggtctaattaattggcctaaaagaagataagtgcttggatcaaatattttttaaagggtaaaataaaagctgtggtacctttcagttcatatgactttcatcttcaaaaaatttaaacagcctaaaagattattggtaaagtgcagatgtcatcaaaatataaataggtggactaaattatgcaggtcaactgctaggtttgctaaatgttttaaggtcataaactgctttttgggttttgagaactatttgtcttgcctgctccacaattggtaaggcctggggacatatagaaataaccacgcccttaattatgctggaattagtcaaaccttggatgcacctagcacataatcaaaacaacttaccaagttttacattaaagttaaaaattgctaggagtaaccattataacatgtaattgaaactactggaaatagatttacatgcaagctgtataagaacagtaggatgtgtttttagtaaaagattataagaaggtgtggaaatgtaagttcttgcttaggtttaaaagattgttttgaatttgataagataaagctaaaagtccaaacaagttgtaaaggaattgtacaaattaatcttgcaaaaattcaatatgtgaacatattgactaaattcaaaaggatattatatggttttcttgtaaattgagcattaaagtaaaagcaaaacaaggttctcttaaggcactaatctgctctttagcaaaatttttaaggggttataaaaagttttttgttttttaaatttctgattcatcattttggcaaaataaataacaaggtaatctggagttctatttcataatatcaagtgttttaaaccgctaacacatttaacaggcttctgaaaatcaaattcagtttcaaaattttctttcctgatgcctggctttttgatgcttcagagagcccttggagtatccaaaagagaggaaaacaggattatttgacatatttaggtatatgagattaccaaaatggtgttcaatattctttaggttatattttggtaaataatactaatatgtgtttcaaatttgtatgggattttaaaaattctaatgtctgagtatacgctatcataattaaggtttttatgttaaataattgtaaatgacagagataaccaagcttctttgtcaattgtgtttctaactgtaactaacctggacattttgttattcacagacaattttcttgttttaattcttttcaaaagatggcttataataagctgtagaactctgacaggtgctctcaaatacaggtttctgataactttggaataaagggaaaacacacagaactcatgaagagctaaaatattcacaaacccataaaaaaactgaggcaatctttttgacttttgcttggaatattgctgatccttgtttttttttcagagtcaaggaaacttattttgaactatttatgacttttaataattgagtaaggtatactcctgtcaacaaaatacagagcatgtttgtctctctgcctggcttctccagaatttgcaaactagttgtgaatattcttaacttatggcaatataaatgtttgcatcagtgcaataagaatccattttcttttgcaagagggtgcaattgataaactagtttttttaccaaggtgttgactggaagggtatgcttccttttaaggagtcaagctcaacttgcagagccgataagagtttcttgagaaaactggcctcatacccttgtctacacagtccctctacagggtttctgacctgtggttagtaaagaatgtcactttcttacaggctcaggagctccaagtttatcttgggaccttaagaggagacgattacccaactcacaggtatttgagcatataaactgatggctgggcttggctttaaaaagtcttatctgagattcctcgtggaacagatttccatcaaaaccaatgtaaaaggcctatgtagaaatagttattccttctgcactttatgcaaacactcagcccaagtgtaagattaaagtctattttacaaacaactcatccctatcatgattttttttaaacaaaattgaggattggagagagagaaattatgtttcaaaacttatcacacatttgttattaaattctagactcatcagttgtttttaagtttttgcctacattttagagtaaccctgcttgttcctgtgaaccaaccagtaatctccaactaaagctcagaaggagtaaaagggatgggtaatgtcaaaattttggatcaacattctagttctgagcaattagcctgcaaatcctgccaggtgatgggaataaataggatgcccatcacctggaggtttcctttttgggaaagtaagaccaagggagctaaccaaagccaagccccatgcacccaaatcttagcaaagataactatagccaccagttatctgggcatgtcacaagacgccctcttccttgttggaggaggactcaattccacagcctcacctaagcatttggcttataataagaaatccatgctagcctctgagacacatttttgtcccaaactcaattctaagcttcacatcaaagccctgggggcagggggaactggatctgaaggacccagatgcagatgataatgcaagttaaaaggcacaatgcagatgagtgtgactgattcctgctgaataagccaagcttcccatttcatgaataaaggtcacactagtatccatggcataaatgaggtctggagaatccaaaggctatggacagcaggggagatagggtatacaagggtaaaagcgaatactctcacccccagacccccctgttaacacaagtgaagaccactttgacacccaccctgtcacagtatctgggacttggggatacaaggaaggaggaatctgctcccctttttgtagatgagtagccattcatcattagtctgtatacctttctttctttttttttttttttttttttttgagacagagtttcactcttgttgcccaggctagagtgcagcagcgtgatcttgcctcactacaacctccacctcctgggttccagcaattctcttgcctcagcctctcgagtagctgggattacaggcatgcaccaccatgcttggctaatgtttgtatttttagtagagacagggtttcaccatgttggccaggctggtctcaaactcctgaccttaggtgatctgcttgcctcgacctctcaaagtgctgagattacaggcatgagccactgcacccggccatctgtacccctttcaaatgcatcctgagtttctaggacccctttgaaaaaaaagacccttcttttttcgtgtttctcctctgtcctctcttcacagataggtaattgtgtttccgtactatgggacacctcacccagatgcattctccaaactggggagagttaatttctcaaactttaacctagtttgcttaggattgggctcaggggaagggaacccagaagcctgacatgctggctaaagggtaaaagtttttttttttttttttaccagttaggtttttggcctccctctccctgtgcaaactggtaaaaggcctcagaattttttagctgtcctcaaccccacccccattttgttttgatacatgttttctataacctggtttatttctcaccttcaggcaatcaaactccaaacgttcatgcaactggagacttggatgagggccccttttgccagggaccctttgataggcttctgagggagctctgactgccgttttccccaagcagtgccccctgtcagcagaaagcagttcagatgagtctttgtccttatccttattccaacagcagttaggtatacttctttagagggggaaatgatagagacaggaggcagccaagggtcccccagtgaaaccctgccttcaagcctaagatagcctgaaggctgaagaaccacactgctggtccgggatgaagcctgccctttcctgactgattctttctgaatagtgcccacctgtgcactgggaggatgggatggaaccttggaagtgcatgttgtttgcagtggggaggagcctggcctctcctgttcctgggtgggaacttgggattcaatctgtgagatgggagacctgcaaacaggactctatcttgctttggtgagaattagttttccttttcatccaataaattccattccccctcacccttcaaagtgtctgtgtgcttaacgttttctggtcctgtgacaagaacccggttttttgttttttgtttctaaaacaatgggtggaacctgacgggaggtaattgaatcatgggggtgggtctttcccatgctgttctcatgacagtgaataagtctcacaagacctgacgattttataaaggagagttcccctgcacacgtcttcttgcctgctctcatgtaagatggtactttgtcctcattcatcttctgccatgattatgaggcctccccaggcatgtggaactgtgcatcaattaaacctctttcctttataaattacctagtatcaggtatgtctttattagcagtgtgagaacagactaatacaatgagtaaactcattttaattagtctttctagaaaaggtgccaatctgaagaatttcttaaatgagtaaccacagcataggttataatgcataaaatattgaaataatccatgagtccacaaatacatttaaaaacatttaaaaattaggaacatcagaagatgacgttcttttatacaaatgagtgccaacaaataaatatagaaagaatgctagaaactgaaaagtcatcatcttgaaacatcataaaagtaattatttcagacaggattcattgatttatgctaataacattgtgtgaaagatcgggaacaaaatactctaacctaaaataactgcctctacagattacttgttagttacaaaaagaaaaaggtacatttacaaagtagaaattttgcagacatctccttaatcaagagatcaaaattactgtcaccaaatataggagaacctgagatcacatgcttcctgatatgtacacgcactgtgaaggatacaatatcacctatgcattattcctattgtaggtatttgacctgaacttaatcatgaagaagaacccaactcttaactgagaggcattttgcaaagcctctgagatggatactttgaaaatagcaatgtaatgataagacaaaaaaataaaaaaattaattaatgtctagggaaattttctggattaagggagactaatagacatgcaaactaaatgtcagctgtgattcttgattgtgttctggattaggaaaattaaacatcataaatttaatttgaggaagaattttgaaaatttgaatatgaattgcatattacataaagtgctgtataaaagttaagttttctaaatgagatcactatattgtatttatgcaggaggatatcctgcttaagagatgggaagttcatacatttaataatgaagatacagcaaatgtgggaaaatgttaaaaatcattgaattaaagtaaagtatatgtgggtattccttgtacaatttttgtaactttgccataagtttgtactttttaaaataaaatgtttgaaaaattacaataattttcatcataatatgcaataaaaaggacaaacttctttttttgaaaaaattttttttgaggctgaatcttgctctgtcacctagtctggagtgcagtggtctgattccagcttactgtaacctctgcctcctgggttcaagtgattctcatgcctcagcctcctgagtagctgggattacaggtgtgcaccacaaggctgagctaatttttgtatttttagtagagataaggtttccccatgttttccaggctggtctcgaactcctcacctcacgtgatctccccactttggcctcccaaagtgctcagattacaagtgtgagctactacgcccagcccagggacggacttactcactcattgagcatcatttttctttgcagttggatagctaaacaaacaaaacaggcaaaaagtccttcctttgtagaacttccattctttctccttatttctgagcttccattgttaaatagatgagggtggggaatgataataagtactggatttgatattttgtagtctctttcctcactacatggaattatttcagatgcatgaatttgtttgctttgaatagatataccgtggctaatttcttccacctagagctctataaaaatattgtaataaaaacactaaccaacaacacttatgcatagtccataacagaaatggccctattagttctgtcttttgtattattacagacccaaagcaagacaccactgaactatttattactttaaaaacattctaagctgtgaggttgctttggtttttccttctgagattaagaataaaatatctatttcagatttttaaaaaagcattccttagattgtctttgtttgcaggccttgtaggcactatcttgtttgtctaaatctaaaaagattaattatatcattattaatggtcatgtatcaccaacagaaaataaaatataaaattttaatttaaagctaaattaaaaagtattagtcaaagattttttatacctttagatatatgccaatttaacagaatcaaaatttgtattaaaacacataagactttttaaaaatagaattaacaggaaatatttgactaacacatatttatgtgtctatataactggtaatgggtcagaaagaatatactcctctagacactgcatattgtttttaatgttgcttaggctctaatttcaaatgcagggtcagataaaagttggttgcattgtttttctatgcatgctttttaatttcatcctttgcatttattaacactaataggtagtttttttttcttgttattgttgatgctacctgttcagaggtattggataataattattcagaactctagattttcactgggttttgttttgtcatgtaattacctaccttatgtgtgtaggtatttgggtggtatgggagagaggaagaaaatttgaaatctatctttttaaatttttttcttatttttttttacaagtccctctcaatgaaaatctacctttgtcaaacacatttgctattcaaaacaactttaatgaccagatgaagtagaaggaaggaaagtgggcacttccatatttgtttttaatgagatatcttagaaagttaaggaacttgaggtcacaaaggagaagcaaggtataactgtgaagaggatactggctttgatttcaattaggggagatgaaggaaaatggaaggagatttcctaactcaacagagaagatggaatttaaatttaccaaaatcagtaataaggtagaagtgagaatgaagtactttcaaagctaaagacagaaggagaaaagtgtttaaaagctaattttaaagtaaaagagagatgaggagttaccagatttgtgtgagaaagagcaaatacatttactgtttttaaatatcatagaagagaggaacaaatttgagtaaataagtttaagaagaagcttgctttccccttattatttgttaatcagcaagatagtggggaaaatgtcttcaggacatgtcagagacctttatggcagcccctcccatcacaggcccagaggcctaggagaaaaaaatggttttgtgggccaggaccagggtccccctgctatgagcagcttaggatcttggtgtcctgtttcccagctgttccagccatggctaaaagggccaaggtacaactcaggccatggcttcagagggtgcaagcccaaagccttggcagcttccatgtgatgttgagcatgtaggtgcacagaagtcaagaatttagatttgggaaccttcacctagatttcagatgatatatggaaacacctggatgttcaggcagaagagtgctacaggggcagggccctcatggagaacctctgctagggcagtgcaggagggaaatgtggggttggagcccccacaaagagtgagaagagggctgtgagaagagggccactgttctccagaccccagaatgttagctccatcaatagcttgcaccatgcacctggaaaagctccagacactcaatgccaacccatgaaagcatccaggaggggggctataccctgcaaagccacagggtttggagctgcccaaggccatgggagcccacctcttgcatcagcatgacctggatgtgagacatggagtcaaaggagatcattttggaactttaaagcttaatgactgccctattggattttagacttgcatgggccccatagcccttttatttgtgccaatttctctcatttggaaatgagtgtatttacctaatacctgtactccgttgtatctaggaagtaactaacttgcttttgattttacacactcatagacagaagggacttgccttgtctcaggtaagtctttggactacaaaattttgagttaatgctgaaatgggttaagacttggggcacttttgggaagacatgattggctttgaacaatgtgaagacatgagatttggcaggggctaggggcagaatgaaatgatttgactgtgtccccacccaaatctcatctttaattatagcttccataattcccatgttctgtgggagggaccctgtgagagttaattgaatcatggaggcgggtctttcccatgctgttctcatgagagtgaaacagtctcaagagatctgatagttttatgaagggaagttcccctacacaaactctcttgcctgcctccatgttagatgtaactttgctcctctttcaccttccaccatgattgcgtggtcttcccagctatgtgaaactgtgagtcaattaaacctctttctaaattacccagtcttgaatatgtctttatcagcagtgtgagaacagaataatacataatttaaccctaaagaagagctctttaatggagaaattcatatatcatgaaatttatcctattcctgaaatctttactgtgataactgaggacctctagccagattccttacttacatcacttggaattagtttgcctccgtataagactagtttgaacttatagtgctgagtttacaagtttccaaatatatgcaaaatatacgtaatcatactgatgattaatggtgagatgcttataaaatggtgagtctaacaagctctattaggtacagatacatgcaatcactgaattctgtcaagagatgatgccctttatgcacaataatgcacattcttccatttattattcaatataataaattctaacaattttccaaaagtgttcatataaaaaataatcagactttatgttttagtaagttgtttgttttcagtaatcatttattcttgagcagatattttttggtgttttattttgtcatgtttattggctattatttctgttagaaacagcttctgttactaagaaagaaacaccgacaagggaagacacctttcccataaaaaaaaatttattccagctgcgttttgatttgaagttgaataaagacaaaggtaaaaagaaaagctaagaagacgttcccgggtgagtcattggagtgtcaactagcctggtcaggggctgctgcttaactacacatatttgataggaagtgtccctttaactgtgagaaaatggtttcctaatgatactttttgcttttcactcatgaaatcactctgggttagcttctcatcagtccataaatgcccatacctggttctgtcaacatttttggttttcagattatgtgctttatgaggcaaaacatagcaacacaaacatttaaattgcttccatgttttacaacctcaactgctgtattttatataaagggcacaaaatgaatactgttattgatgaaaattgggttaccctgaggactcttgcttaactgaaatcacaaatggagcagacagaaaaatcaaaagtatatgctgagaacggtgagtaaagagtgaactcctcagagcagtaatatttttcatttctagaggccagaagtaagaaaagagagattttttttcttcatttgggaaatttgatttccaatgttacttgaacacaatataagtaatataagttttaaaaatgaatagttttacttagaaaaaaagttatttgtatgagggactcaaaaatagttttaaaatattactatatgccagaatctgattttgtttctttgttttgttttatttcttttggtttctatttatcctgggcctgcaggttttattttttaaatgtaacaaagctgatttgatgttcctcaaaatgcttctatgttgatcagcactcaatgcaaggtctagttgtcataatgacactgtatatactccagtcaggagagtaagaagtttctgctttgcaaactagtgggccatttattcaaccaattttaatcttcctgacagtcatacatttacctaaaacaataataaaaaaaactctattaaaacactagaaaatagttggtattactccagaaaggccaaaccaattgtcaaggattagatgtatattaaatgggctgatttttgacaggcaaacttgaaagttaaatgttttggggtaatgcttgcaactagccacactttgcttgtcagtaactagttactacattagtcttccccttctaatagttttgatccctattctaggaatggtacaagttatccagagactgagttgttcttttatcagagtaaattcttcgcatatgtttgcaagtcatgactaaagaatacacaaaagagcatatttaaatatattttgctaattagtaacaataacttttttcttgtttctgatttttctctgggattatgccagttacttgatacatgttcattaattttatactaaaaacaacattaataacggtgctattctaagattttagtccaacttcaagctaatcatattaatacaagctatcctgcttagtgaataataatcataatataatttgtaaaataataataatataatttgtaagtgaatgaaatggactcccttagaaaatgttaatagacacctttttatagcatatgtcctataactgaataaaaagcttagctaattgattggttatttatgttgtattaaataatgatttaatagtaaatgaagcttgaaacatcttgtaagtaaattaatgctggatagttgaatacctatattccaaaagtattattttggcaaaggatataaaagttaattcacctaattatgaacatatataaagccattaagaactaattttttaagatacttttaaagctttttaaaattcctgaatattttactatttaccacatcaccactacattagcagaataataaaaatagctaatacccttgagtacttattatgatctagataatgtgctaagctctctccaggtattatcaacaaatttaatacaacaaattcataaggcagataccattgctgccatttgacagaaaaggtataagactaagaaacttggctaaagttgtgcaggttataagaggcgaggaatggaccggaagcctgatcttccattgtgaagcccatctcatgctcttaactatattgaacctttccctccacaaagggattcattgcctcttctcctatgaacaaatggaaagaaatttaaccaaaataaaattgtcaacatatcatatattgccaaaaaaatttagtgttaccttccctggttcttaccaatgaatatcaataaatattattgattgattcctgatagacttgaaatgatgaattaattccatgaagaggtagaaaatagattacttaactaatactacaactgaacactgaacattctgactaaacaaccaacaaaatataaaatagttgtacattaaagaatacgagaatgaaagttacatttaccataaattataccaccataggacaaacattagttaagaacagattacttttagccatacttaggactgtcagaggaatcagcctcattttgttcatgacttctaaaggactttcagagagaaagagcattttaaaaataagagttaatatttaaagtttatgaattgttgtaaccaacttggttttaaagctggaagtcacctattaataccataccactaacacatttacattaataatagccgttctatgaatagcattagcatcttctcaataaacccatgtagccaacacttacacttcttgattcctgaataaatcatgcaacaaaagatgaatttttagtttctatgaattacaagtaaaaagagtcacctctttaaattatgtgagttgtgtttttttacggatagcattaaggtttcctaattacttttttttgtttaatcattttatgggcaaaaccttaaggactcttagaaaagataaagctttgcctttcaaagaaaccactgccatctcccactgagcgaaagaagtttgaccatgactttgccatctccacttcctttcatgggatacacaatattgttcagaaccggagcaaaattcgcagggtgctctggttggtggtggttctgggctcagtctcacttgtgacatggcagatctacattcgcttgctcaactacttcacatggccaaccacaacgtccattgaggttcaatatgtggaaaagatggagttcccagctgtgacattttgtaatttgaacaggtaaaaattacttttttaaaaataattagttcctataaatttgctagtataatctttcttggatgactgacaaagagttttctcatgtggaacttatcatatgtaaaaccactgaattaaattgcacttaaattgcactcaaaagcacaaatttaagggagcaaagaattcattaatttcttctttcacttattttccatcttttcttccttccttccacaatcatattctaggtaacatcccaaatcctatcattaaatagatgtaaaagagatgggggtttcactacattgcccagactattctcaaactcctaggctgaagcgatccacctgcctcagcctcccaaagtgctgaaattatagatgtgagccactgtgcccagccagatgtaaagtcttgatccctgcatattagaagttcattaattagcagaaaagagaaacatcaacaatcatagtgcaatatggtaattgcacaacaaaataaaacaatgtgcaagggctctgaaatcccacagaacagattaatagtctccatcttgggtgttatggaaatctgggtattaaaagatgattacagctttctaagtggagatgaagaagaaatggaagagggaactactttgcaaacacataacgatataaaatgttctggtgtacatatgatgagctacttaatgtagtagaggcaaggccagctccacgggtctgtgatcagtgcggctgcaaagagccctgagcttagaaaagggttacacttgattcaatgctctactgccagtgtcttgagtccttgactgcaactcattccagaaacctgcagtccattgactgtacaccaaatctgctaagaggaggacaactttccaccactaggcttccagacaatgctttgtaattgcctgtggtgaagcatgaaataccatacataggatttttgctgaggaagggagggcttcttaagactcaccacttagctattagggaagtcactcagccttcttagtgttaattttcatatttataaaatgagataatatcatctacttcacatatttgttgttacattagataaggtaataagtactccattgtgtttcattcctgggggacttctagaacaggtgttcaatatataatagatgttatagcccgtttgtatgattgtcaatatttcaagaactcaagatatgtatcaagggtcctaacagtcgaaacaaatcaatcctttccccccaaaaaatgattttcaatactattgcttcatactgttttaagtagcatgtgttctaacaaaaacagagattggttgcagttatccttgactgaaataaaatgtagatattacttccagaatggcaaagtgaggaagtccataaactctatccccttcaaaaccaaccataactggtgaaaaaatatttatcttaaaaggcaaacttttcttttaaaaaagaaacatttaaatagtatctcctcaaattcaagtctactggaacctcagaatgatcatattttaaaattgggtcattacaaatataattacataagttgaggttatactgcactaggatgagccttaaatccaatgactgaagttcttataaggagaagagagaacacacagagatacagcagggaaaattcatgtgaaatagaagcagaaattgataaggttttgccacaagccaaggtatacttaggacaaaaggcataagacatagagaaaacaaaaagtaaaacggcagatgcaaatctaactatattactaatgaaattaaattagaatggattagacaatccaattgaaaggcaaaggttatcagaatggatttttttagaaagatccagctatatgttgtgtgcaggagacacaatttagatttgaagatgcaaatgggttaaaagtaaaagaatggaaaaaggtatgtcaaccttcagaaagctagagtagatatcctaatctcagacaacatagactttaaagtaatatatgttacctgatataaaaaattgtcattttatgttgaataaatgatcaatctatcaggagatataagttaacctatatagttagatttttcaataccccacttttagtaatcaatagaacaacttgacagagaataaacaaggaaatagaagacacagaagacttaaacatctttataaacctactagactttgcaaacatccttagaacactctactcaacaatagcaaaatatgcattcttctcatgtgcacatggcacattctcttagaatggaccatatacaaggccatatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccccacctgaaccactcaggtttataataggtagagcatgtattgctctcaactcaggtgtatttcatctttacctgtagctaaaggattctagctctgattcaccaggctcagtaattgatggacacaatccatggagcactatcagggttttcttgatttgggagcacgaagaggggacttcagactctccccatgcaggctcagatggcttgccacattgattcttcctttactgtcacttaaactcaggattacattgctcccaggcttcaagatgggcctagtctagacaaaaactttcagtctcatgattatgtgaaactctcaaataatgtgtaccatgaagtgtagaacattgcctgacactgagttagctctcagtaaattacttgtagcaggaattgtactgataatgcaaatcattccaatcttcttttagcttttcttgcagtgaggttggctgtgtcacttgttcaaggaaagagaatgtgaacagaaggaatacgtgtgtcctccaggactgaacttaaaaatttgccacacatactccccaagattccttctccaactgcccagtagatatatatactcaaggtgacattgaagatgagagtgatctgttggtccaggtcatgagtggctggtggagcttcagccctcatcaccctcatccttagataatatgtgagaaaggaactgctcctgtgagtcactcaaattttgagtttgtttattgaaagattaggctactttcaaagacacagtaccagctcaggaccttgcaggctgaagattgcttactgtgatgataactctgcttccatgaacatccgtcttagggcaagaagctgagtaaattatttatcccacactctatccatccaaacaggtgaggggctgtgccttgcccttgtgtgaagaaaccaggttctattcaagagtgttactctttctgtgaacaggaggcaaaggtatgagctgagactctacctgctctcaaaatcatcagaaagagggctcagaaggagtcacaattactgaactgctgactgaaagactctctggggactcaacccatggctattaatgtcagtgtcactgggccagctcagatgcaaacccaccactactgtgtcatccacaaaatacatacagctatcgcatatcagcattgtgggagaacggtattaaagtgaacttcactttatagaaactgtattcctgacagccacatacagttcttatgaccatgaatcaaatcgcatttatatgaacaaaggcaagtaatttaatagaaccctattacagaacataggattctctacaggacagactttacacagtgaacattcacacttagttttatcattaattttcatagggcaggtttgctgtactaactaggcatgcaaagaccatgggtctcagcaggtaatataaagtgatgaggcaggttgggtggatttgaggtaaaagtgagcgttggggaatgcagtagatcatgaatatttaccccacccaaatgaggcagctcccactcagggacaacggttcattgtagagacattgtgggacaagtgatgctgcatacaatgagtgatctcgatttctttgtgaaatcatatgatttaaaaatgttagcatgataacctgatcctacttggaagttaataagttagcctgctgcattttgtaatgctggcaaaaaccgtaagatttctgggtcagtgagtaaagaatgtattactcacagcaacagcaatagccagagtagccagtttcttgagtcccaagtccacagggtgatgaaaagaggtccaagtaacgcctgtccatgcagtaggctacatcacaagagaggaaccctgaacttagggaacatgaatcttttatactgaacagtaagcgtgcctgccccttgctccagaaagaaatactatctctatcttccaagacgatttgctgtacaaacaccccaggaaagctagtgcagaacaaagacactcagaacctctgctcataagacatgcataaatatgagagaccacctcccaacagtattctcccctagtttctacaccatcttgtcttctggagaattttcccttggatacatcagtccatcagcactctgattaatctgatcaggagagctgggcctaaatctgttcaatttggcttatatagcatttaactgcagttactatcaacaggacttcaagcagtagaatgaggccaacctgcaaaactgacctgtcatgccctgcctcaccatcttatccagtcctggactcaaccagctgaactaatccctaaaccaccaagtctaccttagaaaaacaggtatgtttttcctcaagtttctctagcgatctttccacttagccaaaggcattattgcagatacagcagggtgtatttgcaattacacagactccaccttggcctgcaaggggaaagtctagggtgattttattatcattcataaggaccctggccagcaagttgtgtattagctgaatgcctttggcagacaaggtggtatatttaatcccttcagctaaaagggaaaatttttaactgtgtgattcccatcacagggataacagcttgcattttgtgcataaatgattaattagttattcctcaaggaagcttctctagagttcaaggatggcttccctttgaaaacatctcatagtctcatgcaggtatcatgatctactgctgggatttccttgaagacacttgaagatctcttatccttcatatgatggttacttttatgtgtcaacttgacttggcatcagggtgcccagattaaatattatttctgggtgtgtttgtgagggtgtttctgaatgagattagcatcaaactgatggactcagtggagtgattgccctcccctgccttggtagggatcacctgatccatttgaaagcctgaatagaacaaaaggcagaggaaagaagaattcagtctttttttctccctcgttgcctgagctagaccctctgtattagtccattctcacgttgctatgaagaaatacctgagactgggtaatttataaagaaaagagatttaattgactcacagttccacaggactagggaggccttgggaaacttacagctgtggcggaaggcacctcttcacagggcagcaggagagagaatgagtgctcagcaaagggggaagagccccatataaaacccttaggtctcatgagaactcacttactatcatgagaacagtgtgaggtaatctgtccccatgatttaattatcttcacctgatcctacccttgacatgcggggattattacaattcaaggtgagattttggtagtgacacagagccaaactatatcaccatctcatctcattgtttcctgcccttggactgggatttagataaactcccctcagtctcagttcttcagatttggattgaattataccaccagctttcttgggactctggcttgccggcagacagcagattgtgggacttttcagcttccataattacctgagccagtttctcataataatacatatttttggaaaactctaatatagattttggtactgagactgcctctaaaggaacatattaaggatgagttttctgaattcattccaaggtttatggaactggctctctaatctgattagatttaaagatactggactctatttccagtagtaaagagtgcagggatagtcagtggcatgatctggcaatagagatatgcaaaatacctgcactatatgcttccagtgagccactaataagaagtaaggaggtgggagactttgtatacggtatttttgaaagtttctggaaaactaatgcatataacgacatagactggtttgtcctaatgtcattggaaaagttgcaccaaaaaagaatgagctcagggattcaaattctcagccgtataaataatcaaatagcttctatgggtaccctgaaaaagacgcttatctcctgtagccacaggactgaaactgctaaaaattaaacacaaaacctcattctgcaactggctaaattacaatgaagttgactttccagctttgcaggatttctacactaaagtgagggcatcaattggaaagaatgggattctgtaaattaaagataaagacatgagcaaagatcctgatgaagctggagacattgagtccctaaattctgatgagtcttctttgccagtggaagaagtcttccaacccccactgaaagcggcctccccagccccagtggaactaattccctaacccctggcaaagtggcctcccagccctccatgacagtggcatcctcacccccagttgtattgggctttccatccttatctgaggggttaaccctgcattacctgacaaaatggtaatagtctctctgaagcattgctatgcaagacaatgctgtttcttctcagggcccatccgaaccatccctctttccttttagacctataactagactctagttttagcaggcctctgaacgtgaggtacaacatatgacccatgaggatgtgcatcacactctaaaagaaccacttttgttttctaacttatgcagacaaaaatccagggaatatggtgagaatggatatgaagcatgtgggatgatggtagaatcagtgtacagttggatcaggctgaatttattgacatgggctcgctatgcagagattctgcatttagtgtcgcagcttaaggagttagaaaaggctctaaaagtttctttggttggttgactgaatcatggaccacaagatggcccacagagagtgaactggaaatgtcagacattccttggtttaatgtagaagaagggattcaaaggcttagggaggttgaaatgttagaatggatttgtcgtgtaagacctactcacttacactggtggtgtccagaagacatacctttcgccagtactgtgagaaataaatttgtgagagcagcactggcatctttgaagggctccgtaatttctcttttctgtaggccaggccttattttgggatctgcagtcactgaattaagaaacctacagacaatgagggtaataaaatctggagtgacagggaccaagtggcagaactcaaccaccaagagcaaagtagatgtggtaaccttaatagagagcagagtcaaagcatccatcagaatagtgtgactcatgtagatatatataacattggctacttaatcatgatgttcttataaatgaaatagataggaagcctactaaattcttacctgacatatgtaagcaaaaattttcaggttaagcaaacaaaagtttaacttgaatcttaaaaacagagaatcatgggcctcaatcaattcccagatttgagccagtttacaaagtcagagccccttgaatgaagggaatgcttggcccccttgaggatgtacttagtataccatcaaaaacatacactggtaatgtttctctcattcttccccgaaggaacctccagccttttaccagggtagttgtgcaatggggaaaaggaaatcatcagaccttctggggaaggagatattgatattgatttgagagccttgaaagatcactgtggtcctttagttagagtatgtcctaatggaagtcaggtgttcgatggagtttagctcaggtttaaatcacaatgggtccatttcgtcactgaacccatcccgtggttatttccccagttccagaatgcataattggcatatacataattaatagctggaggaagctccacattggttccctaacctatggaatgaagacctggtaagaaaggccattggaacttcctctacctaggaaaatactaaaccaaaagtaataccacatacctggagggattagaaaaattagtgccaatataaagaagaaagatacagtggatggtgattctcaccatatacctgttcaactctcctatttggcccgtgcagaagacagacggctcttgtagaatgacagtggattatcttaagcttaattaggtcattacttcaattccgctgctgtaccagatgtggtttgattgcttgagcaaactaacacatctcctggtaactggtatacagctattaatctgacaaatgacttttttctccatacctgcctataaggcccaccagaagcagtttgttttcagctggcaaggacagcaatacaccttcactctcccacttcagggttatatcaactctccagctgtgtgtcataatttagtttccagaaatcttgatcacctttcccttccacaaaatgttacactggtccattactttgatgacattatattgattgaacataatgagccaaaagtagcaaccactctctacttattggtaagacatttgtgtatcagtgggtgggaaataaattggactaaaattcagagccgttctatctcagtaagctttctaggggtccagtggagtggagcgtgtcaagatattccttccaaggtgaaggataaattattgcatttggccccttctatgaccaagaaagaggcacaaggcctagtgggtctatttggattctgcagacaacgtattgggatatgttgctctagcccatttatccagcaacccaaaaagcttctagttttgagtggggcctagaacaagaaaagacactgaaacaggtccaggctgctgttcaagctgtactaccacttgggcagcatgatccagccagtgtccttacggaacaggtcctttccattgtaatttacatgaataatacagacataatcaattgcagtttgtttccagagttcatgatccccaaaatgggtgccttcaatctaccaatatgtagtattccaaatagcagaccaaacagctaggacattagcaacaacctaagaataagtaaaaataggacacagttgggtttcaggttagctgaaccatcagggaaagaggcacggacacacaagaaaaatcttgagttgaaggtcccattgagccagcagctttgctgtaaatggcaaagtgatgaataaaggtttccccagagtgatagcagctactttttatgttaagacaaggtgctgctggcagtgggatagctgcattcttgaatatactatttccacttaccaagcagggattgtcaggccctctccatcttattaatcattgagtctgagtcaacccacccaaaatgaaaatatcaggctggagagtctcaagacttttatgggtcaggtattttgttttgaaggagcatagtagcaagccaaaaaggctttacatcaaagtgatttaacagctgtgtcaaggaggtgatgagtccagacctcaacaaagacttctgggtgaaggctgaccccctttatcagaggctccaatcaccaagactatcagacatggaaacttgtatctcagaaaggtcatgagggttgtggggcccctttggtgcttgcatttatctacttactgctgttactgatcaggagaattttccaccacttcctggataaggcagtgtgagcacctcaatttctaatgtatattcagacatggatatgacaaaaaataatacactgtattggccaaaaagaccacaaacagaaggtcacacttgtctcctttccctactctaatttctgaccaaactctattaatggaaagtttgtgtgtcccattctgacaagggccctgggtgtcagtatacaacagaggcataaaattatgattccctcccctccccaaaatctctgcccccagctatacttgggcagttgcttttggcctttgattccacttggaggcagggagagcctgacctcacctctaatcatttatctccttaaagtagctgagatcagagtagagagagagggatgggttaggtgtaagatgtagagagtagctttccaccaataagggaggagcacttacttttagttatgagcacacagaggcagctcaaccaaatgaacttgcagtgtattcaacctgcccaacactctaataggtggcagattgctcataactgacaccatctgtttcagctttgggggtcccttgaccaagcagccatagcagcattgtattccgactgtgggcatggatttctacccatcacctgacatgggattgactggcacagggataattttttaaaaagggatgcaccttaatctgagacatttggtgcctcattatcaagcaaatatacttaagttcttgtccaatttaaatgcaagtgctataggagttttccaaggctacaggtctgacctcagcaatttatctacactgtcagtctctcattctccagtgagtcgctttcatcagtgttgcatacccaattcaggggcattaaaagcccaaagattagtcaacgcctgtatttgtttgttagggtttccttgataatgaaccataatgcctgggtgacttaaataacagaaatttattgtttcttagttctggaggctgcaagtccaagatcaaggtgtcaccatcattggttgcttctgggggctgttgagaaagaacgtgttccatgcctcccacctagcatctaaaggtttgctgaacatctttagtatttctgggtatatctacgtaacactgactctctgccttcatctttacatgatgtttttcctgtgtgtctctatgttcaaatttctcctttgtataaggacaccaggtatattgcattagtggcccaccctgctctagtatgacctcatcttaacttacctcattatatttgccatgaccctattccacataaagacacactctaaggtacttgaggctatgacttctgtggggacagttgaaaacataacaatgccttatgtatgctttcctgtggaagtttgtcagctccagagaaatctctccaaaagggacaaaactcccattggagccaggacctactgaaaaacactctgagacctttaactgtcatctctgaaagaatccaaggttggcatgaaagactgcaagaagggctcagccgtaaggcttcccagctgttgccattcttcctcaacaagtgttatgcatctattgccctcatctctcaaatgaactagccaagttctaatcgcttactcagtttctgctcataggggccatatattttcttctttgtggtcagtgtaaatgtacactgtgttagtcaaaggatacaaaatttcacttaggagaaataaatttaggagatctattgtgcaatatggtgactatagttaataacaatgtacctcatattggaaaattgctacaagagcagattttaaatgttctctccaaaaaaaagtgtgtgaggcaatggatatgtaaattagcttgacttagcctttccacaatgtatacatatatcaacagatcatgttgtttatcaaaaatatgtacaatttttatttgtccatttaaaacaattacttgactaatttttgtttttaaaatggatatatctgtaactcttgtctgaagaaggtcagaaccttcctctaacccaggatggttatatgttatgacctgtaagaattttccagggctacaggtctaacctcaatcatttatctcattgacttgaggcttgaggagtagacttgaggctgacacccagtggaggaagaatctgctcacccagtgagagttaacaaccaagccactgtttaggccactgcttttgaacctactttctgatgcttgagatgcaatgttttctgaattcattctcaatagcaggtgataaaattggggaccatttctcactctgctgatttataatttggttaccacatttgccatcgattcccatgaactttttcctccaatcccattaatcagctcacaatacccttactcttctcttccaggaagccaagtttctgtcagcatcccaacctcatttccaaactgtcaataattgtgaagggtctgagattttactctacttgcaagttaaacagctagactgccacaatttcatggatgctggcagaagacgtgactcctgggtcagagaaagggaacttcactgactcacacaactatagcacctgaccaagggtatcagcgttatcttgcactggttcactgggtcacacacagcaatgtgaaaacaaataaactacacctgcacacagtgagttgcacgccagaagatacatcctgagtttaggaacacaaatcttttataatgggcagtaagcctggcggctctttgttcttgggagagacattatctctgtcttctaaggctgcttgctatacagacatctttgaaaagataatattaaacaaaggcagtaggtacccctgccctctataacattcaggagatcgatagaaaactgtctcccaacagagtgcaatggagacacatttatcccccagtttctgttataagccactacacttggataagccaagcacagttttatttctacggttggtgttttctaaaaagtttgactttatatatgatttgaaaaactgagactttttaattttgttttttataaatagaaataaatttttttttaatttaggttttaaaacaattgtgattttatgtataatatgagttttgttggaaataggagctttagaaataaatgtattttgattatgttagtacactatttaacaataactttttgaaaagggatttgaatacattgacctgtacagagcaaatgcctgacataatagttgttaatcctggttttccacactgttacgttggaagttttctctctctttagtagcaagacctacatcaaagcaaatatattcgtataacttgggacaatggtgtttttccttgatataaagttcatttttctttttaaacataactcattttaaattttaagaagatttttatttaaacacttttattacaatatttaggtggcacaataactaacaagcttctgagacaggaggtaacattctcatagactttgcaactcagccagaagtaaaactcgaaataaatatgtcatttaaagtaactatgaaggtaataataaaaggagtgttgttagtactaagaaggttttcaatgcagggtccaatagctatatttacatatacagaaaaaatgaaattagttactaaacataacaaaaaaaacttggtatacctatagcaatgcatatttaccaaaaactggtgaaaaatatattgagagatatgttaaatatttgctgaaaagaaaactacttgtctgtatgtgaaaccccatgaatcattttacacatcaggaccaaaaaactaactccttttattcctcaaaaatcaaaagtggtctcatctaacccaaaaatcacagacttgtcagttattgtcttctcattttgaacattatttactattttttctctctaaacagtaagttcttagtcaaaatttattattttgtcccatctagacaatatggtctatggtacttcactagggtggtctaggagtagattctagggagttccccaagctctggcagtacatgtgtcttgtatggggccatatgcacatttttggaggggtgatagtctatgtcttttatccatttctcaaagcaataaaagtgaagacccactagccaactgtagcagtttcccaagtaaaatataaagatgattcactggggtaggtgaagaaaatactagaatttctatttatattcatgctcatttagaaaagaaaagctgtcctaatatgtagtatatggattgagagcagcatgtatcagtctatagagttgttgtcctacaatattttattaacagattgcaggaatctaaaaggttggagaatagtgctctagggcagtgaacatcaagttctcatattttccaaaacatctttaatttacatgtgcaaggaggtgaaggaatagtaaattctcaaatactaatacattttcattcacttcaatgttataagatacaacttattcagggaagacggcagctcactgtgattaagcagtgtctggatgctgcacatccctgtatcactgggaatgattatttctaaaggcccagaattcccagaccactgttgccataactttccaaagagcttcagttttgctgctcttgttccaggtgtataatacttaacacattgggctgtccaacttctgtaatagcatttttgttttgctttgttttgttttcactttctctgaaactacctcatagctctttcctcaatacagatggatttggcagcatttcagatggaacaaacatctgagcagatacttcaagatttgggcattatgcaaagtcattttcagccaagtaattttcctcacaatctggaaggctcatttgtgatttaattttctgacattatataatttaaaaaattaaaaaacttgacaggcagccacaaccttatctaaatatcttctaccagaatatctgagattaagtcctggatatgacatttaaattttgtcctgatttctaccccaatgtttaaatccaagatattaacattttcataaaactaagtttatgatgatggaaaaaactgcactggtgataaaagaagtgtggcttttagaatatcacttttttttccttttgcttaggtttttctgattgaaatctacacattgataaaacttttgctactattttggcagatactatatatgaaaataaatggacataaataaggtaatgggatagctttggatgaaaaatgtgtaggttatgtgggctagtcaaaacagatgcaggtgtggctgtaataagcaaagctgcttcaagggaagtacagatggcaatcaaatgtacagtgattactgcagcttaaaataagaatactgaatgagtgcacatcatcaatataataaaagaaacatttaaagaggagcaacagttcatcaaagatctactagctgacgtgctaagaacagaagaaaactaaactcaacaaagatgacaaaggattaattggctccgttactgtaagcttatggctttcagtaattgattcttccccttttcactaaaggatacaaatatttcccaggtcttgaaaagtatcataacagagaccataaactctaacactcaattttggttgaaaatgacattttggttttctctgcttaataagttaagcagtcactgcttttagcctgtgttttaacaagagaactgcactttgctaccgtctctgcagcactgaatttctgttttggaggagacggtaagtccctcctcacaggaatcgcaaggcgtctctgccctcccacctctcatatatgctgtgttctccatccctgcacccttaaagacagtcttcatttaactgtccaagtacaaccacctaaagagtgaaattgcattgaagtgcaaaatgaggtgaaataaatgatccatgttttactgttcagtgaaaggtagtgatctgacaagctcctcaacaaatatgtatcaaagattcaattttacttattaaaatgatttacatgggtcaaacccatgcacctaaaaccactgcagtaattcgttaacccattctgctttgggcgactaacatttgcatcaacaagggcttgattgtggatttaaagcattatacagcactagacttcactggagctttgcatgtaaagctccatctgggcagttttccagctgggtaatctcagaggactgatgtctattcctcaggcactagttagactagacactcttgtttattcatctctttcatttgctaggcctaccacagaccaccctcttccatcctatgccttcttttaacctaacctgagacagagctgggaagtctttgccttggcgtggctggaaatatcagggagaagaaatttggaagaaatgttagtcaaaatgctgaagcaactcttctttcctccctaccaaaggatctgaaagactaattagccttgagagtgacataaaattccaatgccagacatcccagctttatgctcatacttatggtaaaatatccaggatataaaatgaatcattttcccttgatgtataaacagatgttcattcagtcacctgtgcaagtaaatatccaccctatccttaaccatgctgtttctaatagtctttggaaaacacacatctgatcataccattctcttcctttaagacttgatgcttctcttatctagtagctttaaaactgttggagagggagtcagtagccaatggatctaattttactcatgttgcttcttttttttttttcttttgtaaaatctattttgaacaagtgcctcaagtgaataataatggctcttattgtagttattaatattactattatggtaatgttgataactacttaaggtcatgtaaagctgtgtcatgtgtagttaattactatatattaatattacaatagcaatatgaataactatcatggcacacctttacatgacacacataccccttcgtgatttttgcccagatggtcttttgtccttattctcctccctcccaactcatcccaggtaactggctgtccagaatgtactatgcaccctcaagaccccatctctttgtgtgctgatttattttcctggaccatgtccttccccttctctgcttgatgaactctaagaccaaaagacttctctaagaactgagtcccctactttgtgctcccacagaatgttgtaattatgtcttacatttgtctttcactttcctttcttgatagattgggccatattttatccttaaatttcaaaaatatagcaatattcctggcacatagtaggcattcaataaattcttcatgaatgaataaatgaaaatgagtaaatgaatctattcttatttaggttttcttaaatatacgctttgcattttcctacaaagtctagatcgatatagaaaaacaaagcaaaatggaaagaaacattttaattctgtatttccctttgcttcttgatgcaaagcatctttagaaaagttcagtataactctgtgcttgcctgagaaatatgtataaggagataatttatatagtgtactcgacacaaaaaacaacaggacaagaagccatgaatggaaactatataaccagaacagaaagaatagctcctgcattttcaggagaaacagtctgttagggaagcagtcttgagacccagtcttattttttagaatagttaaatggcaacataactcagtacccgtaatccccaacctgattacaggctgtttgtccactatttttccatcttcaatacctcaaagctctgacttgggccccttatttattttaagaatctatgctccagggtctctactctggctaccatcagtggtctggatctgaaaagtttctcgctaatcgctatgcttcaaagcagataagatagagaaaaaaagatattccttaaattttttagcatctaaattgtgaatttcgtatggactctgtcatggaaatgtatgttcatcctctttgaagatccacaaattattctgtatttaatgggcctttgtaggctggtctggggaacctatcacagatgatttaacttcactgtgaaaatatgttttttggtaattttttatttagatataatgccacgtttatagaaaagttgcaggaatcgtacaaaaaactcccatacaacttttcaccaagattatatacattcccctcatttgttttgtgtatatgctaatacatcacaaacacacaaaatactttttgaattctgattgaattataaactttttgagtacagattgtaagcaaattgaggtctgctgaaatgtttgatcaagactacattccatttcatgcttttacattttctttatttctattatttccccataataagagttcggttccagaaagaaaaatgtatttacattttttttccttgtaagtagtgacttaacttcatatatttgtgaggatgtaactatactttctcaaggcctatggcactttccaataataggctgagtggtttattgagtcaaagtcagtcctgtaagatacggggttatcacttagtaaacagcatcattgtaatatctatagtagtacccttggaatactagaggccaatcagtaatgaatatccctgatgtcaccatgggctagcctgggtactaagggaggcagttaaagaagttttaaactgagttatttctcagaaccccaaataagagctcagcctatctatggctctgctaatattgattttaaaactataacgttcatctttttcttgctttaaattcaagctgtttttaaaatcaaatctttatccccaaccaaaatgtagattttttagttcaccagagaacttcaactgacctaactaaaactaaaagttcctagctgcttaaatgtttatgtaaaacaaaaagaaaaagttgaagttccggaaaaaatacttagacatttccataatgtagtcactgcctcatttgcttcatcaatactaacagtcgttttttttaagtcaagatatttcaagtagaataaaaacacttcatatatgtatatttaatgagacaaaaacttgattcttagttcatagacatacaccaaaatgttcagcacaaatactcaagaaaaatgagatatccaccaaaagaacaagtcttattttctacttccatacttagattttcttcttaaaactttaaacagctcagcatggcaatagaagatgtctgctgaagtgggtccatattgttccaagtgtgcctgcacgtgtgtttacgtgtgtaaatgttttaccaatgtcctttatggtgggtcccctggatgattactttactcccatgttgcttccagctaaattagatttgaatagtattactgcattttaatagaagatataaacctcttttattctgaatactttgctattatgatagtaaaatgaattaataggaattaaattgctaacatggattaaaatagcttttaccattgtagaattttaaaaaagtcaaacaaaagaaatatttattattataacacacgttactatggagagtatttttaacttccagaactaaaaataatatttggtatttacagaactattttatattcatattgcatctaacccctaattgcttattttctaaaaagaaaaaaagaagaaagtgcaatagagaaaaaaagtcagcctatattcaaacatatactaacagttaaatgagaaaatgcaataaaatgcagagttgaaagtttgaaagaactgagggtgtcaaagaatgtaatttctattcaaaaaccttttttttgacgacactatcacaaacacactttaataaccatggcattccaccaggatatgagattgcaggtgactccatcagtttgtacaattgactacaattatctgatatatgtacaaggtgctgaattttgagtacaggtagtaagtgctggttatataatagaaatactcccacaaaagtagctttttaaggttttttttttttttcttgaagagctgaagtactgaagtgagtacatacattttgtcatgttaaacaggagaaagattgtgacttggctcctgaaattttaatactgcttgttagccatctacaatccctaaagtgctttcagaggctctacacatcttcaatgagtcaaaccccaaagacttataaataggtatataggttgctaatctattcctgatgctttgcctaaaaaattggcattgccatcttccacatctttcttttggaagcatggttttgagtttgttccttgttggtaagcatccagaaaatggcagattccggggatttcactagggaagtttggtggaagttcctcccttgatcgaggggtaaacacgaaaccaggacagtctttgagtaatctgaaaatattaaaatataatactgacataaatgtttgtccactcaaaaaaagaacacaacataactaactgaatgttactgagaatatttataaaccagtgattttaggtgtgagtgacagaaaaagtctaacgagattaacagacatcttcccagaagtacaatgaagaatcaattcacaacaagaaaattgtgtcaattaatgtatttcacttacactggaaaaaattataaaatatttttgcaaaaataatatccacacaacagtgcaatttgcagtagtgaaaaaataatccaaagatttaatagtagcaaagtattcacacataaccataacagtattaagatttcctgagttgaaagaggtaagatgtccatttatcaattcattaattttgtaaatattatgctaattgtgggcataaaacgtaatcatgggcaagaaatagtgaaccaaaattacaatgacaaaaatgatgtgagaacaacttttgaatctttcaatagaatttttatgaattctagattagtaggactcaaatgagatttaattgtacatatcaccactctacttactaattgttattagcaatattttttctcctgaaattcatctgtttaccacattatattgtttgctattttactacttgttcattatgctaaacctatttcaaattctaatcttactgattaagctattggcatctctttccatttgcttccctagctttccatcaaatgagaacttaataagccttctctctaacctaatattcaggccatagatgaaaaatgttgcatggctgtgggttctcaaccaatttctacagaaaagcatttattatatttacccaaacagaccatggtcttctactcactagtctgtaatgtcatgtctacattaaatagaatgatgatgttgaattagattttggtactgtgacaacatctatgtattgaagaaaccaactgaatagaaatctgtctcaagaatgacagtttaattttcatcagtacatcaataaatatgtaatctgctaaacaataaaactaatgaaaatgatgccttagtcatgaatttataaatatttttgagtgactattatttgagaatacataatgaaaaggtatagaattagacagtattaaaagtataagcttcaaagtctgatagctctagcagcacagatgtccattatccagcctttaaaatggggataataatagtacatattccttaggacagttgtgaaaattgaataaatcaatttttaaaaactgactaagcatcaattagaaatcacaccacacctatcatattggtaacagtaagtctgacaatatcaccttggagaatagcacacttcatacactactggtggagcatagattggtaaaatcattttgaacagctaattaatgcttggtaaagttgaaactgggtatagcctatggtctgggaataccatttttagttatattatctaaagacatatgtataaagatacctattgaatcactttttgtaataatgaaaaattggacacactctaattacccacaggtagaagatagagtctggctcattcatactctagaatacacattatatcacatgcacatatagtaacatgctagatctggttcatactggcttgtgagagccaattgttaaaattcaggaactttgtaagacagttaagccatccatagcctgaaacctgccagagtggacatagtaacaccacagaaattggcaaatgctggaatcatggattctctacctcatcctccaaagctggtttaccagcaaaccactccattagcaatagttaaaatgaataatctagatttatgactagatttacattttatcaaggaaactatctcagaaacataatactgagtggattcaaggcaagtcttaaaaggatatggatatgttcactgggaatctattttcctctggctttatttttctatctggcaggacctaaatgaaagtgatattcctgcctctctctacccattttccttcctggaaagcagaagtgacatcttattgatgagttgtggagaagctgctgaacatgcatctgtatttgggagactgtctgcagagagtacatattctcagctcctacaagctataaagcacttaggatccattgtgtcagagcgtgtaacagggaccatgtaagagtttctatttatattttattatattaatatctatttactttacattttatttttttataaaacagaaacctgaaagaaatatggcaaaatgttgatacttaaatattagtattggctacatgagtgtcatcttgtttgctttgtttttttatatgtttaaaaattaaatacaaaaatgtatgcaactataagagtctaataaataaaaatgtataaaactataggtgattaaaaaaatttatctattattttttctatctacagcaactcaacatttcctactaaatacggtaatgacaattataatctcctatttaaatgtattatatacatatattcagcctttagaagtggctttaaagtgcatcatttgaatttattaagggcattgctctagattgcacgtctggtaagtgctgtttgtgggactctaaggcatgtcttctgaatacaacttctatgctttttaaagacaccttaacagacaaacataattagaatgctatggagtttgaaatgttcattatattgtctttttaaaaaatcagaatatataagaaaaaccatttagaaaaacaaaccatttcgatgaaagcttgatgatagcaacacccaaaaaaggcagcaacagcccagatgtcctcataggtggacatttttctctacaccttcttcaccagacaccatttttccgcaaattactgcatcaatgctccaattctctgttatctttcctatattcctttattcactttgttccttcttcttacagaagtcaattcttgcttctctagcacaatgtgctcagacttgtcctacaaataagagattctaggcctgtttaccccacaaactggacagcatccatgtatcctccccacttgccatttttgtttctaatgatccctggatcaccacataatcctctaacattgacccactgacctggaactaatgtagtagacctggatccactgactcactgatctaatgtagtctctactagtctctattagttttccttacttcaagaatgcaaacaaaatgtcgaatattatcccctaaaattaaagtaaaactcctgacaatgtttaaaaaacaggagtgtataactgtcattacactgctacattctgaatcactgattagaatttggaagtggaaatttgaagatacaggtttttttttggtataggtatgtattctgtgtcctaaatttacctgtatttcagaaatatatcatggaagctacattcaatattaaacagattattttattattaaagaaatatcattccagaatttaatgtatcatcaataaagctctccaaagaaaactgcattttgtgcttgtacaccttctttagcagccgcttcttgtactatagtttgagtaaaactaaaattatgcccctaccgttgactaaatttcccaaagcattggatgtttaattgaaatttatttatattattttaagttttcccagcatatggaagaaaagtgaagtatctctgaaagaaaggaatttaaaatgttgttaaaagtatttaacattaagtatttatattttgcctaaataagacagtattaactactgctttcccttaggcaaggcataaatatctcatatacgagaataaaaagaaagtaaactattattgaaatcaatacaaaaggaaaaaagctatatcatatttgtctataaaaagtagaaatgacttcagcaaatgagctgaagttcaatataaattaaattaaaatttcaatgtcattcatgcatgtgtatataaacgtaactgaaacaaagacagaaatgttaatagttactatctctgggtaatgagattatgacttattcctattacttactttataatttactaattttgtaaattttctgaaatgggcatgtgttaccacagtaataaaaaatttatttaaaagataacaccatctatgctataaaaacctatatgtgcatttctagtaacattgccaatacagtgaatctaagacttaagtcatgagcattgtcctgttgtcagaaatatatgtcaacatgttgtggtcctctgctgaaatacatgagatcctgaattatattttgaagaaaaataagtagctaatatcagtgccccctaacatgactcaagaccaccagactttgagatacaaaatgcaagaatttaaaaagtttagtttcatactgttaaaaacaatcaatatagaagaaaaaggaaaatatgctttttatatgtagcagttttgttttaaggtaggattcctcagtatgcacatatacacataacacatcacttaacattgaaagagaaacacaggtgtcagtgaaaggatgggcaactgactcctacttaataggatgattcctcttaatgcctacagagagagtagaatcatattatctcaaggcatggattcttatccctacctattatacccagtaggaccataggccaattaccattacaatgaaccccagggatttttccaattctcaggttatactgagttgttttcatacccaggagtcaacagttaaccagtaaatacttcttgctttccttctgtgtttcacgcgctgatctaggagctgggaatatggaatgtgtaccacaggaagctaacattctaaaaagggaggcaggcaatcaacacataaataaatactgcataagacaatttcagatacaggtgtgaactgagaagatgaaataggataatgagagggtatgtgaggagaaggttgttgctatagttatgatagttaaggagtgtctctttaaagagaggagaagaaatgatcactgtaaggtcgatggtctgagtaactaggtggatggtgatatcattttaaggtgattggtaagcttgcctgggtgggaagtggatttcatcagtctgtgtgggaaatcactcattctaattcggtccagttcattttaatatgtgaattagtcatccaagtggaaatatggaataggatatatgagtctgtagctctgcagagaatcacaggatgggcgaaagattttttttaaaaaatagttagagtcataaaagttcagatacagaagataacaggaaaagaggctctgagcacggcaacaaaggtttggtagggaaggagtaccaagagaagacactgagaaggtgtagacatggggtagtgagaaaactaggtgaatgtggtgccaaaaatttaaagaagaaaatgtttaaatgagggagttctcagcctattcaaatgcttctgagaggataaggagagaaaactgacagtgattttattttagcagggcacaatgatgaccttgataagaaccgtttgagtggagtaaacactgactggagtgagttgaagagaaaataagaggtggaaaaaatacagatagtgagtatagacaacttctttcagttttgctataaaacagagcagaaattgaaaattgtagcagaagccggacttggaaacaaggaattttttatttttcaattcagagctttaatagatatggttgtattctgttgggaataatcatttcatcaaagagaaattgatcatagagagttaatttccacagaaaagttattgagaaggtgagaggaaatagcactgagagtaaaactgtatcaaccaggggcaggaggggagctgggccaatgtaatgggaacaaagaaggaggagatgagtactggggtagctctgctgagagaacctacagcacaaagatggcaaagctcctgtctgacttctattctggtggtgaaatatgaggcaagattatcaactgagagtctgggctggaaaggaaaggatgctggtgccttaagaagggagaagaccaggtgcattggctcacgcctgtaatcctacactttgggaggccaaggtgggcagatcacttgaggtcaggagttcgagaccagcctggccaacacggcaaaacccagtatctactaaaaatacaaaaattagccagacgtggaggcgggcgcctgtaatcccacctacttgggaggctgaggcaggagaatcacttgaaactgggaggcagagcttgcagtgaggcaagactgagctgctgtactccaacctgggccatagagcaagacttagtctcaaaaaatataaataaataaataaataaataaataaataaataaataaataaatgcagaagaagaagggagaagatgctattaaagagttatctctgagaataggaaagctaacttgcttagtataatttagctactgtatagatctgaaaaactcttttgttacttaagagtttccaatataatgagacaaaatgtacatgaaagatatatttctagttaccacggattggtttattttccggttaaaggtggataagtgataacaaaatggtagattttattgactcaggctgtatactacatgttgtcaaggcgacagtgaaaataaaggttcttctgatgctagatgacacgtggctcctctgaccgtgttaacacttgctgccagtccataaaactgcaatctttgaaaagtgctcgatgaccagctagttttcctctggtaaattcttccctgtatgatagggaatttgactggctgttgtccttgattgcagggagggagcttctaaaactcttggattttttgagttataggagtatctgttttccatgaggcccttggatcacttttgagtttatattaatgagatgactcagaatgggggcagggccccagaaaaatcaatcttgtggatagaaggttggggttttgtgttagcctaacctctggggaggagagtgggggtggtgactgagttcaatcacatggccaatgattcaacgaatcatgcctacataatgaaaacaataagactccatacaccaaactcaaagctcaatcaggtggagcttcctggcttgtgaacccactgaggtggctggagggagataagttctgattctatggaagctctgcatttgggaccctcccaaaccttacccaaactgtttcttcatttggttggttctgatttgtatccttcgaaataaaactgtaatagtaagtatagcaattccctaagtattgtgagtcattttagataattatctaacttgagaggttcatgggaacccccaaaattgtagctcagtcgtctaaatgcatacttaggtcctgaagggtggctggcatctgaattaagggcaggcttgttgggttctgtgccctgtaacttgtggttatgactgtgctgagtctgggtgtttactgccagaactgcactgcagtgtactagttgatgtcagaacacctatcaacatactaagagtattaatatgtgcttctaaaagaagaatgatggaagactatgtaatatctgtcatggatttatggctgggccacaaacacctcaggcaagtcattcttaggaggctaataagcagtgtaaaaaagttaaagtgaggatgataaaagttatcacgtttcttttttccttacgactaaggaagaaaaaagttatgaagaatgaatcaaatcagtggggcaaatggcaattgtatataaataaaaacaagagattttaggtgtttaacataattactacctgtaagttgttgggctgacattgatttttcgtggtacactgcaggactcaaatttgttagccagagtgacattgtttccaaaaagacagtaacggggcattttaactccaacgacgccagcaaaaactgatccagagtgcagtccaattcgcatctgaaagcaaaaaataacatgatgttcccagtcactggtaaaacacttcctactcgtcagtgtcgtttgaataccgtctaaattattttaccattcaccctcaggacaccacccctttcctcaaaaccttcagccaacttccctctactcctcacatgcaattaaaaattactagtgcagaatcaacagtcacacttcttaactcttttccatttaggtttattacacaaaccatcttaattgtgtctgtcttcaacatctgaaacaagtttccatgcacagggatcatagtaaccacagcaacatagcaagggggatatgagctgtttatgtgaaactgccacctctcattcaagtgagatagacataatttctggactcctggaatcattttcccaaacactgacaaagaagggcaaatgaagctagctctaaggatattaaatatatcttttagacattttacctcaaaatagctttataccaaacactatcccactgttctaatgacttacaactgaggaaatgaaattgtaatatttaagcagaaaatgacagaggaagcagaaagtggaaggtacttgagtccaatgacgatgacaatgtctatatacatgaaattaaaaatccatactgatgaaagatcagatttgtgccatgcctagatataagaggcagtctgggatgtgttgtggcaaaattttaggaattctggaagtctccaatgatttggaaagccactgagacagaatgttttattttatttattttttagagacagagtcttgctctgtcactcaggctgtagtgcagcggtaccatcatagttcactgtaacctcgaacccctgggcccaaatgatcctcccatctcagcctcctgagtagctagaactacaggcatgcaccatcacacctggataagttttaaattttttgtagagacaaggtcttgctatgttgcccagctgattctcaaactcttgggctcaagtgatcctcctgcctcaacttcccaaagtgctaggattacaaatgtaagtcaccttgcctagccaaagcaggatatttaacaatggaatcttcaaaggtctgattacttgatgatcttaaaagattcacaaacacttaaattactgctactacaaaacaagctaaagtggagatattttaaatgatgatgataccatgttaattattttcaaactctttgtttactcaaccattttaaaaagttgtattagatgtatgcgttttgaatgaagaccaagaactcagtaacttggatataatttagaaatcagaagaattcagttatgtctagttttaataactaatttcagatatctgctagaaactttccagactcatttacttttaagatgtcatatgagttctacaaaattttcaaacaaaagcactagactgtgaatcaggaactttgtattctaacaagctctactagtaactggtagtctggcttccttaagtctctttacttccctccgatcatgttttcacctataaaatgcaggggtttgattatttttaaagacctttcttcatcaaagttttactttaacaagcaaaacttagatgtctgttatatatctggctttgtctcttttaggagaatattaatttacacatttagcaagtgatagatagatagatagatagatagatagatagatagatagaataaagctcttctgctcataaatatcaactatgacttcagcatatttagattgaggaatttttgctttctgtgagcactcttctggttaaaccagtttttcttcctatacttagttactccagcttattatatattaaatttaccatagcatttttatcttgtttcataattaaaatgtaaatatattctgctcgtaaaaaccgaaacgatgcagacaaggcaagtgtccctcctagtccttccatgccgtgctgccttcctccccttctccagttttgaatctctcaaccctaacattttcttacagagcttttcaaattcatcagcaagatttaaaacacaacgtgtttaagacacacatcgtgccctgagcatatctcttttcactggtaacagacttccctgtgaaataatgaaaggcaaaggcagaattctggctcccgggaaaagtttgaatgggaacctactaagggcatcttctatttgagaccacttgtggcatagcccatttctcatcataagcttctaggcttaggaaaggaggggtactagaagtttgcttccgtgttcacttgtcacctactgactttcacggttacactcagcccactggtctctgtcaccaacccacctgctgctttctaagcctgactggatggatgttagtgaggaaatcaatgggaataaatgaatagatgaatgatttgcacatgtgacgtgctaaaggaaagcatagagtttgaaaaataaaaaaagaaaatattttctattctttcacttcaattctataccaagaggtagcagggcaagaagcagaaaaagcctgggctttgaagtcacatggacatgactccaaatactgacacataggagacttgactgctcgtgtctgccagtgtagatgcctctttaaagtgttattataaggattaaatgagacgatgttaagtcacaatatgctcctttctgatacatgttaaagtttccctactaaaaatactctccactctgcctctgtgatgtttcaccggtatcttttagggacgtcagatatttcccagggctgcaggttagtatgcataaaaaggtatgattaatgattccctttataggtcttttaaactaatctatcattacccaggtaactatctttgaaactagctataaataattcttccaacttagaaaaatctaaaaatacatttctgcaaataacaaaaatgccataaattcccccaccccaaacatacacatcttaattataaaatgtatttgtgtgaaaatcacttaaaatgttgttaatagtctagatatttttaacatttacattgatgctacaagccccctcaaacaaattagaaagagaaagaaatatatagtcctcctctaaaaaaatgcaattatcgaaaataaatctgaaaaagtcagatattgtgatgcttttaaaacatacttgcaaattattttacactcctcctattgattcttgggctctgagccctctctcttctgggcagacttgtgattgctctggctgatagagtgcagtggaagtgttgcggtgtgtcttccaagactaagtcatgcagcttccaccagtcttgttcagtgggacatgaaccacctcataaaaaatacatattagggcatcacatgtaacgctaacaatcccagctgaacccaacctttcagccatccccaccaaggaatctgacatgtaagtgaagcagcagtcttggaatgaatcctccagcttcaatcgttgcaatcccagctattccaggcaccctcacccatttcggtattcccagctgaggtcccatagattgtagagcacagacgaggtattcctgctatgacttatccaaatttttgatgcacagaatccacgcataataaaagtttgtgattttataccattaaatttagaaaagtatgttgcacagaaacatataaatgggatgacaatagacttagaaaatagttacgaaggcttaaacatatggcttctattaaggttaaagagagttaagggtaaatctccaaatatagttttgtttttaaatttcttctttcttctctatatgttttatagcataccggataagattttacaatacctcaaaggcttatttctgaaggtagtcaactgaggactgtcctataaaaattaaggggcagattcactgtgagacaaaggtaatctgcttatattctatctgttaaaccaatgcctaccatgttaactttatttcctcatgtgtggtgagatattagtctggaatctggctcatctagaaattttgaaatgtcacgtgccttggaaaatattggtgaattaacagtcaacatccattcaaatgtaatatcctctcctccaggggctggaaaactaaaactacattttctaaactaacttgcatcttctaaatgcagttaggcttggtgggggggatgcacagttgcatgagattcggcaggcacaatctccactataactcaagaacaattgtggaagcctttgtgttttctatagcatgcttagtagagagtccattttcccatcactcattttgtaagtcatgatgcaggattgccatcttgctgattattatagtagatgtggttttgaagaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaataaaataggatggaaatatgccaaatataatgttggttaaatctgagttgtgttaaccatgtccaaaaatttcataggtttttttgtgataattctttaccacttaaactacttatttccttccttatccattatttgcctgcctttgagtaggttttgctagggatatttaactgcaggtacaacttttccaaacttaattttgttttttccttggcattcaatctccatggatatgacatcaacaagattttcaggtacttccttccacaaacacatcttcattctaggattattgtctactcctttcatttatatttaagtctataagtaaagtcataaatcttttattataacatataacttcagtggagagattaaatttttattttatataccatacctcagttgatttgttaacatgaacatgttatttctcaaaccttccggcacagattttctatataacatgcatttcctgctactatggtagttaccagaggcttatgaaacttcaggttccatctgcaatagggggagcataaccataactcagccttcaagaaataaattttttcaacaacataagatttcaggtgtgttagtccgttttcacgctgctgataaagacatagccaagactgtgaagaaaaagagatttaactggacttacagtttcacatggctggggagggctcagaatcatggtgggaggcaaaagacacttcatacatactgtcagcaagaaaaaaaatgagggagatgcaaaagcaaaaagccctgataaaaccatcagatctcatgagacttattcactatcatgagaacagtatgggggaaacggcccccatgattcaaattatctcccacaacacatgggaattatgggagtacaattcaagatgagatatgggtgggacacagagacaaaccatattactaggtatcattacgaagcactgaccatctacttcccaaccaattgaatgtacaatgggaaaatcccttgttcattcgcagcactctggccaggaaatagcaatgttacatgagtgttcttaccagagaatcattgacattggcttagtccgtacataatagtcctacaaatgtactcctctagcctgaatctctatcatcttgagccactgcctcccaacttttaaatacactttttttttttttttgagacagagtctcgctctgttgcccaggctggaaggcaggggtgcgatctcagctcactgcaacctccacctcccaggtttaaacaattctcctgcctcagcctcccgagtagctgaaattgcagctactggctaatttgcacccctggctaattttttgtatttttagtagagatggggtttcaccatgttagtgaggctagtctcgatctcctgacctcaggtgatccacctgcctcggcctcccaaagtactgggattacaggcgtgagccaccctgcccagactaaatacacttttaaaagcagttttaggtttacacacacacacacacacacacacacacacacacaaaactgagtgaaaaatacagagctcccatatacccccttaacctaccacccccagtttcccctattattaacatcttgtatgattgtggtacttttgttgcaatcgatgagccaatattgatgccttattactaactaaagtccatagtttatgttaggattcactctttgtgttgtacattctatgggttttgacaaatgcataattacatgtgtctaacattacagtatcatacagactagtttcactgccctaaaaatcccgtgtgctccatctattcatttttccctccttccctcaagctcctgtcaatcactgatatttcaaaattctttatggtttcaatagttttgtcttccctggcatgctatatcggtggaatcctatggtatgtagccttttcacattggtatctttcacttagcaatatgcctttaatgttctttcatgtcttttcatggcttgatagctcatttatttttatgttgctgctctattattgcaatcaagatcttatcaaatccttcctgactgaggatatgtgctctttccaaggatgtttgggaaggggatgctccattagtgtagaaacatctattgtcccttgtatctctacattatcttttcaggcagcttccagtaatgccaggtcccctgggtcctttatgaatattctgtgggtgaatcgccagtgataaatggatatacagaagtatgaatatgatcaacataacttgagggaaaaaaaacccaaattgtgattccaacctgctcttcttttaggcaggtcatcaagggatactgattgatacactttctttgaataactcaaacagagttgttgggttaacttcagagtttgccttcaggtcacttctccacttttggggaaaagcaaatccaggggttatctcagcatggatcctgacccataccccaccttgagagaattaagagtcttaactatcttgctctgaggctctctctcaggaaaagaataatagcatcctttcccatttgtcattgaggtgctctcttcccatctctcacagtttctccctttccattttcacctgtctcatctcagacttgtcctcttcaggtgagatttttctcattcagtcattctaccatctacaggcaagtagcagaaacagccttccttgaacatccccttttgtccattttcaagaaggagtactgttagaaaggtaaagattactggactattcctaccagaggcactgggccagcatttccagccagtcctccattagccacatgatgtggagtcctcactcgatctatctgttcccttggtaatatttatactttctgaagaatcagcataatgtttgcatggtgattcttcccaggatgcagggcacacaagtgcaatcccagagctggtacaactctatgaagtaaaataagaaaaatgtttttgaggcgagaaagtcagagagtatccttggaccttcatcctcatccccatatcccagaaactgtttcaaaggtgttcctgctccactatccctcatattcagataaggtatttactcaggagtcagctcagctcttcatgcctctagcacattcttcccatattttagtaaaggtgttttcattccactttgaaggtggtaggggatataataacaaacaatgttaagcttcttacctcatgtaggtacatactaatagcagtggcatgtcaccttgtaaataaaatgtagttggtgggaaccaaactgatcgacaatctgtttttccaaacctttaaggaggcttaaatttaataatctccattctcagctagttagctcttccatgacatcaaccccattttatctctttcaaaatacagttgccctttgaacaacatgggtttgaaccgcactacatggcttttcttccacctctgccacccctgagatggcaagacgaacccttgccctgtctcctcctcctcagcctactcaacatgaagaagacaggatggagggcttcataatgatccacttccacttaatgagtagtaaacgtgttttcttttcttcatgattgtcttaataacattttcttttctctagctaactttattttaagaatacagtgtataggctgggcacggtggctcatgcctgtaatcccagcactttgggaggctgaggcgggaggatcacgaggtcaggagatcaagaccatcctggcgaacacgatgaaacctcatctctactaaattagccgtgtgtggtggtgggcgcctgtaacccagctactcgggaggctgaggcaggagaatggtgtgaactcaggaggcagagtttgcagtgagccgagatcccaccactgcactccagcctgggcaaaagagtgagactttgtctccaaaaaaaaaaaaaaaaaaaaaaaaaaaaaaagaatacagtgtgtaatacatataacatataaaatatgtgttaattgactgttggtgttattggtaaggcttccggtcaacagtaggctactagtagttaacttttggatgagtccgtaatgatatgtggactttgcactgaacaggggcttggtgctcccaacccttgtattgttcaagggtcaactgcatatgtatttgtcgttttcttatcccaggaatattggaagataaattcatgagccatatgtgatacttaatatttcttaaaatacagacttcctcaagttcaaaaaataggagcgttttctcaattatattctgctacttgacccattttatgttagacacaacaaaatttgtggagtctttctatgaatcattcaaaatacaaattgtttcaagatttttcaatatattgaaaaaaaggggtaagaatggtatcttctagctgagtttattaaggggtgtccaaccttttggcttccctgggcatcactgaaagaagaattttcttgggccacacataaaatacactaacactaatgatagctaatgagatggaaaaaaaaaacatccatacatcattttcataatctccgccactacagataagcaaaaaagttatttcattcaaagggttacacatggctataattagaatcaatatttggataagtaggccgggcctggaggctcacgcctataatcccagcatttggggaggctgaggtgggtgaatcacttgaggtcaggagttcgagaccagcctagccaatgtggtgaaaccccatctctactacaaatacaaaaattagccaggtgtggtggcaggtatctgtaatcccagctacttgggaggctgaggtaggagaatcgtttgaaccctggaggcagaggttgcagcgtaggttgcagtggctgagatcacaccactacactctagcctgggcgaaagagcaagactccatctcaaaaaagaaaagataaatagaggaattgcaataaagtcagaagctgcatgccacatctatgggagaatttctccagagtatttacccaagattgagatttctgagtcatgagatatttgtattcctaatgtcagtaccatgaaattgcactggcctcattcccacagaactagcacttgcttatcattaagctggaaaacaatatcaagacttttaggagttattctcttacatctgggagaaattgcaataccaccaagaataaattaggggatagattctttgacttgattattatttcttaaatatgtttgagaaatcttctaaaagtcacctttaattgaatattctgagacaccgaaagtacatctattacccgagtgggagagagaacactgaaactcctgagtttcctctctggactaaatatgtaaatagaagtacttgggaaaaaaatgagaaaatgccatgtattttcaagatagtacagatttaactttattttcatttagattgatgttaagtaccacctgtatgggaaacagatttctgtccctccgaaagtcatttacaatagaacactccagataaaagggaaaaaaaagtatgttgaatcttaatgaaaataaaaattctttaaaactttataaagcataaattatacatatcatttcttgaataaagaaatattttgtttcctcatgtatagtttatttattgggtctagtttacatatttagtcataaaaaatgtctcttttccactacaccaatattcattcattgagaaacatttatgagatgtctattacataatcttgactgttctaggtggagggaacacaaaggtgaaaaagataattcctatttctttatttcatatatatatatatatatatacacatatatacacatatatatacatacacatatatatatacacatatatatacacacagacaaagacaaatagaaagaaatatatatgaagagatacatatctttaatggcttttatcgaactctcaaaactatagaaaagtgacgtaacaatgaattagtcaagataaatctgaaggtatcatgaaaaggaaactattttattctaagtgaccattaacactcctctatcctatttttatttcccagatgattctgaggtggaggagtccactgggtcatttcagatatcttcagcaaaaagaaaatgcatatccagtagaaattggtgaatagatattcaataatttctatgatcgtgatcagactggccccacaaaatagacccagctgaccaccaagatctgctgtaataaaaccaataaagattgaatgtcattatttttcataaaaaacaaatattattagaagttaaattagttgaagcaaaatagagtcataactgagatgaagattggttaattttattatgttaacatttgatatgaataaggtaatagcaattggtaatatcaaagtggtattcatacaaatcatcataaaaagcagagaagaaaagcaaacataaaatctgattccaaacatataactctaagactctggaggatataaaatctcggtaaattcattagagattctagataattacaaactcatttctaggtgttaaatgaagagcagaagagtgccatccagaaggcactactggatgatgatgttttccagaaaattaatttccaagcaagttgtgacatcaagtgtcaaaactgtatttctttttatgcattcataatatttcctttaaccaaaaattaacataacattgcctttttgatattttaaaatttgtacttatatttactgtttgtgcttttttattgtggtaaaatatatataccctaaaatttaccatgttaaccatttttaagtatacagtactgtggcattaagtacgttcacattgttttgcagccattccataatctttatctatttgcaggatgttttcatcttccctaactgaaactctgtacgcattaaactctaactcctcatccttcctctcctcggcccatgacaaccactcttttactttctgtctctgaatctgattactccagcagtgctttcataaacagattacattattaatcattatctcattgccctaggccattcttaggaccattccgtttttgtaaagtgtgataatgaagtataggcttctccaagagctttggcaaatttacatatttcctccatattcagggtctcagctgtgatgtctgactgctatcacttttgcctgagtctaaggccttcttctgtctccgacacagcttcctctaagatgcggtgagatctaggaaattagccattatgtttattagggactgtggagaaggcaagtgagcaggcagtggttgcctaattttgcttcctcaggttcttctttttaagccactctagggtagagccctgtgtggaggtatgaggtggctgacatttacccaggtgtagcattgctgaaacattctacaaagtcacttgatgagggatctaatggtaaagtttgatgtgagtgtgagtaggagatgaaacaccaccagccttttctttataaaagtccatgtgaggttgtacagtgttttctagggagtggaaatatgtcagatttaaatagcagctgaatgacaagttcaattgattcttgtcctccactggtaagctaaaccttaaatatcaaagaaaaaaaactgataaaaatttttaaatataaggagcaattaggccgggcacggtggctcatgcctgtaatcccagcaccttgggaggccaatgtgggcggatcacggggtcaggagatcgagaccatcctggccaacacggtgaaaccccgtctctactaaaaatacaaaaaattagccaggcgtggtggcgggtgcctgtagtcccagctacttgggaggctgaggcaggagaatggtgtgaaccccctgggaagcggagcttgcagtaagccaagatggcgccactgcactccagcctgggctacagagcgagattctgtctcaaaataaataagtaaataaataaaataaggagcaattaaataacacttaccaagtaactcagacacactcaccgccttttgctgctgggttatcttatagtttaggtcactatagttaatttcaatttttacaagattctccctagggataaaaaagagagttaatatagcttaagattgtcagcattacttttaattcccgtcgaataaaggtaatattcatttttgttttatatgctatttaatgttaaactgacacagaattgttaatattaatcacaattagcaggatgtagaaggcattttgtcatgttggtttttaagaaaaaataattttccccaataattttctgcacctcattcctgagaaactagaaaaagacattaaatgtaaatatctactgaataaaatatttacaaacatctaaatgtgtgtttgattttcattcaaatcttggttctgtcttttgttacttacatgaccttcaagaaatcacattggtttttttaagtcatggtttcctcaagagtagtcaggattttagagctgagctcacagagttgcagtgaggcaaaattaaattatgtatatgcaaaatggacacagagcctgacgtatattgggtgttcgatgaatactaattattaactatgtgtcattaatatacctaggcaaagactctgttttcttaatgtctgtttgaaatctttatacatgaccagagtggctaaacatcttttaaaatgatactaatagctcgttacacggtcttgaaagccctcactttctctatcaccagcctagacccctaccttgaacttcaataatatattcagttcattcttatatttctacttggatgtctaatagatgttccaactttaatacgttccaagtcaaatccttcacatttcccttatttatagtgtaaattttcacactccatagccagtccatcaggaaagcccattttttatgttaccttcaaaatatatctggactcctatgacctctcaccccattcaaagctaccacctttgcccaagccttcatgacctctcacctggatttttgcaatagcttgctaactggtctttctgtttccaactttagtgcaccccacttcaacaaatattgtcaggaaggactttcctgacaataatccacaaccctgaaaacaaccctccctgactctggcttccccacactgcttttctgctgccactctctcacagcacttaatgccatctgacatgactatatacagtggtcaccaatttccatcccagtggacaggagtgcagcaaggcaggatttttgtcttttatacactgaggtatctctaatacctagacgactcctgaatcctaacaaataataggttgtcccttagatatatgtaaaatggacaaataagggagcaaattaaagatgaaaccaccaaatgaatggatcaatattaccctgtcagtgttataagatgaatctgaaaccaagtacctcatgattttaaaatggcaataataataatagtattcattaagagtggatactattagccgagtttcctagaattatgtggtgatctttaatacacctaaagaacatggctgtaaatcactctccttaatgtttacaatttttgaaatcaataattccaaatatacttaattcaaaatatatcattaatatgtttacttaatttccattggtttaaccctgagtaacaggagaatgtactatgtttaagtagataatcagggattacagtaatatttacaaatgataaaacagttcccaaagtaatggaaaaataaagttgtcttggaagtccagaaattttagtaatattccaatctctttgtcccccattgtgctcctaatttgcatgtgcactgataatattgtttaatttcccaagcacaacattcataatcatatccaagcattatccacctgtgattttgagagaagataaattcttcccctgcaaaaatattgtgatatttgctcaacttctctcaggcagttgttttgtctaatcatttgggtctcactgaatctgtgcctcatggcaatagtgaaattgtccaggatttaacaggatttgagtccaaaaatatttgtgtcaggagaggcacaattataaagtaaaaactaaaaactgaatttttttattgtattacaaaatagtttctaaaaaagcttccagatgcttgaacaggagtgctgcattaggggaatgaattaatttggcataaagaaaacaattttatacaatttttatttcaaaatttgttaagttaaaacagaacaatttttaaaattcacttgttttactaatgaagttggcctaacgtcagacacaatttacccttatcttcaagggtaaactccacactgcctgctacagtcaagctaagcctctcaacattttgaggtacagcagcagccccatgaggccgtggattgaccatgaagtagcatttatttctgtcccaggaagacaaaaacatacacctccatggttaagttaggggtcgcttcttttaaaatgactgctgctaacatttcacatttgccgtcctgacaaactgcacttctgttttctgatttaccactctctaatttccagtacctttgcacaaaccagttgcttaattatatgtgtgacttctaactgtctctgtgtgggagttgaacaacccctgagtccagccacctttgttcagctgctcaccttagtcttcctggtaatggcctccactatttaaaaccatgtaaggtccctacatgtacattatgtgataggattaattggataataattaataattatatggacaggaaacagaaaaatactgggtagaagagggcggttccctggcaaaggcccaccctcagacctggatacccgtggccctaaatgagaacaggcatttctgtttttgcatccaaaaagttgccttttggcccactacaccccctatcctgcctccatatgaaacccaaaccccaagctccagaagagacgagaagaccaacagatcaacaaaccagtgatggcaaaatgatgtggcagagaaagagagaagagaaggcacatctgaacaccaaggggagctcggccgggggtgttcagagaagaatctagtcacaggctgcctgactccacgcaaagatcacaatcccactccatacccccttctggctcctgatccatctcactgagagccacctccaccactcaataaaaccttgcacccatccttcgagcccgtgtgtaatccagttcttctgggacactgggaaagaccttgggatacagaaggctgtcacactggccctctgcccttgcgataaggcagagggtccattgagctgattaaccctccagccatctgtagacagcaaagctgaaagagctttgtaaccctagggttgcaggcacccaatgctagacactaccatggggtaggagcccaaagcgctccccctggcctctgcacctgcccgtctgcatgctctccataggagtttgagctgcggggataccgaacaggcgagcaacaccctgttgcacattttgcaaggggaatcagagaattctcccgtttcaagaataatctgagttttggaaaaaaaaaaaaaaagaaaaaaaaacagcattccctaagatcctcttgtttcatacatggctttcttttctgattcaccagtatgtctccaaaatcatgcctactttctctgtatattaagtaattatgaaactcaggagatttctatcaaccaaaagacacaggaagtgctcttctaagtcatatttaaatacatcaggaacaatattaaaagcaaatcagtttcccacattaaaacaaaatatataaatagatgtctgtgctggagtcggaagtgtaaacaaacataattgtaaattttacagcctgtttatcactttatctgctgtttgaaatcactccattagccctgttttgatgaaaatgctttttctttctgtaaagatcactttgttacctcagcgtttacatgggaatttttttttattttattaaagtaagcacaaggctaacattggaatctaagcatgctttccaaattatgcacacatctgtgaaaatgtacgtcaaccttaagttagttaacatatttttattttaatagatataactacaaataccaatatactgtatatattgctgcttcatgtcctagtcctaaaaatacaaagaagaaacaaagagaaatgctaatgttgcttccctgttaaaaaacgaagagttatttatgaagcttgatttgtaatttttaagaggctgtatggtatagcagaaagaggactaatttaattctcagttaatcaggggacgtggaatcatgtatctgagttgcaaagacatggggaccagcgtcaaagttcttgggcataacttctttctgggcccctgactaccttcacagaaccatttcttaaaatacgatgtccttcagtttcctcttctgtaaaattagaatattaatttctgtttcatctaccttgtacagttattagaagactcanagtgtataatgtacaggtaggaaattttcatattttaaggcatccattattatcagtcaacattattattttccccagtttgggcaataaatgtttgatcatgagcaagttactcaagcaagcatcattttccttttcacacaattacagtaaggagttgaaagatatgatcttatttacttttaaaattctatggtttcattgtcagttcagtgaaaaaagccattgcctcccttgattgattagaatttcatttcctgagtctttgagggttttcaataaagaaaaaaaaatctatgttaattatcaataatttaaaaggctagtaaggttacctgatgtatttccggctttgattcaacttcttggaaagatatttcaaagctttttgacttggaaaagaggaataagaaatagtggccgggtattctatttcttcacaagaaacggggcagctagagttatgtgttcctactgtacataaatccttaaattcaatgtggtctgaaatgaaaatcaggacagggaattcagagaagagaaatatttcatcatgggtaggatagttttataaagtttatcatttcactttcaatattaaaaaaaggtggttaccaacaacaaatggtgtttactctgaaacaaatatgttactctgaatatagcagaggagatacaatataaggcctggaagttccaatctttgaattcattgagtcactggattgggatctttattggatttaagaaattattttcaggaagtatctgggtctatttctgtaaaatatcagatatttggacagaagaatatctgggtctatttctgtaaaatatcagatatttggacagaagaatatctgggtctatttctgtaaaatatcagatatttggacagaagaaagatggaaagtgtgtaaacatcttcagatttgtctcaacatacaatggtaagaaatgtgctcctgagtatttgcagttccttatttctgttggcccagtcacttccaaatcaaccactgaggttctctgttcagtgatctctcttggctccttcagagcaaccaggctgtccatgcaaatgcatactagaaaaaagtccctgtagcaggtagtgtttgtctccagcttccctttggaccacctcccagtgctcattctcactgctcctcattcatggtagatactcaggttcttgtttttcctttggttttgatactgaaaccctttgttctcgtgaatcagttgtttaccaagacctcctggttactgaccaactttaggtttgcttttcagactctaactttgacttctctaggagaagtcaaagcatgcatctgctagttctttagcattgctggcaaagaccaccactcttctcctacactcacatgcactcatacactcccatacacacataggctcacatgcacatactctcacacacacacttacatacatatactctcatgtacataatactctctcacacacacaaacacacacactcttctgacctccttatcccacaacagtccccgctaaactgcatgcatggtttttttccccaagctctccctccagcctccaacaaggataagaaatctgaagaaatctggcaacatcatttcaagtctattggttagtaatacggcttttgtatattcagcctgtgatctacataaaaaaagattccttctacctatagccataccttgaaatattttacaaccattgaaaagaaccttttaaatctattcctgctaccctggagatatttccacaatgggttagaaaagcaagttgcatagaagggtatatattgggattctgttttaataaaacaaccaatgacttcacgtgataaatatggagatgtatatgtggagaaagttacagtaggatatactaaagtgttagcgattattgcaggttaaggtaagaggtgtgaaagcaaaaaaaagagggggctctttaataagaacttgtccccaataaatatggcatgtacagttataattccatttatgaaaaaattttatatgccaagtgcatgcaataaagtgaatgaatgaaggactattcccaatcagtgagaggagttctcagtaataaaacttctatttatttttacatgatttcctttctattctgtagtattttaactattcacaataaacttgttttatttgtgaaacaataaaaagcagctaggatattttgattataaaaaagaacaaaatttaaaaatttagatgtgtcttaatatcagagggtttttgtttgtgtatactgcctgtgtttgttagctactatttaagaacacttacaaaaagatcttctcttatatactatattctgtttaatttctgggctatccatagtttgacttaatgtcttatattactttgagcaactttaatataaatcctgaaaataaaagtaaattaagcacttaccaagtacaggagaaacacagctgaagtacttttgtaggtcacattctatcccatatccttaaaaataataagtgtaacatatagagactttacattttgtaaaataagtgatctaatgacaaaaatacctttcttacctacatctatccatccattcatccacccacccattcttctatatgaacatctattctttcataagcaaaactatggaatcaagttagtatccagtgaaaatatgtgttgggaggaagaataatgcttttgtttctttccatacctctttccatctgtctatatcactctaaaatctttaagcgggaataataattttcaaatgtgagtagttttcaaatttaaaattactttcacagttatcatctcatttgatatggttcacaatcctgtcatatgagaattttcattttacagataaagaaactaagcctcagaaagagtggcttgctcaagaccacacagttaggggaaacttggcacattctgtacatcttccatcctgctccctctatccatttacacacacacacacacacacacacacacacacacacgttcctgacagactgccattccagaagtaggatggggagggggcaggtaagtaccaatatttgttaagtacccactatagattgtgagataagtagaaaatcactatgtatcagagctgggtttactcctacggctgattctaaagacttagtcttttagccttaatacaatatttgtctattgctatagcccatgtgactcagcagttagccacaatgtttagtcagtaatcttgaaaataaccaaaggagattgtccacttacaacgtgcggacaatttgtggaatgtttggctaactgtaaggaagtgctctttatgaataaaatacaataagtaaaaaaaaaaaactacctccaacactgagttaggaaaaaacctcatttttctgaattatttcttgctctggcaataatatttcttccattataaagagcaattcagaaaaaaaagatggtagatggaacacagaaaacaaaaataaaaacaaacaaacaaaagaacaatgaaattttggaaataaccaatatctgaaaagctgttttggagattgcttcctcaaattgttagtggaattacctcagcgaatttgtttaacgtttagaatcttccgtttattcatatatcagtacacttaaagggatgtgtgagaatctaaccaaataagatcatattcctgaatgcatttgatatatatgctaaagttcatgcagatatcattctttgtgttttatatagcaccttgcctagtttctggtccacattagccagtaggttttggtttaataatcataacttttaaaattgtcaacattattctgcatgaaacaattcagacttctttccagcaactttgccaataaattaggaatgagaaatcagagccagagccagcagttttatatagaacagcagtttttgcagtggccagagatcaataatctggatactaaaaagaaggatgtgatgattatggaaacctctggaaaataatgaaaccacttttgcaaaatttatatcttttttacttttattattttttatattatatataatataatatatattttttatattttatatatattttatatttatacatattttatatttatattttatatattttttacttttattattacattgaaagagatttgacctaactgactccatcttgtttctaacctccaagctgtccttgttcattcttgggcgttcgctgaactaactttgggaggaacttagtttatggtttagctctgaaacaaagacaataacagccctttcccaaaataaactcccttcctgcctggggactagattgcctatatagggctaacaaattagccacaagattagaaattatgtttaggggtcatgcagctggagggtgcaagattctaaaccgccccaaattgcttatagtgataacattactattgtaaaagctaagatcagtgcttgagatatgttgcagaccctgcctccaatgatcagctggcaccacccagaacaaaaatctggcccatctggttttgtgacccctacccaggaattgactcagcgcaaaaagacagcttcaactccacatgatttcatctctgatctcaccaatcagaacccctgattcactggttcactggtcccctacccaccaaattatccttaaaaactcttatctccgaatactcagggagactgatttgagtcataataaaactccaatctcccgcacagccagctctgcatgaattagtttttctctattgcaattcctttgtcttgataaatcagctctgtctaggcagcaggcatggtgagcccgttgggcagttacaatattttggcaggtagattgtgttacaatatatctttctagatatgctgtgggagggggatgtaagagacctgaatacaaatatctgacatttacatagactatattctgtgccaggcactatttgaaccacttacatacccctttcattcttacaacaaccctataagatgggattattatcccctcctccatttgacagcagaggagagaggttaggtcacttgttcaagatcacctaggggagaaattgtagagccgacatgagcagcctggttgagatccagctcttattcaccctgctccattgcttccttgaatgcttgcctctcacccatggttctggaaaagttgcaaattgtatgaaaaggaggcctgaagttaattttacagtcccaaagaaaataacttttcaacagtgaaatcttcaggcttggtaagacatatccatgatctaatttttctttcaaaattaagaaaatcttggtgtgctgttttagatatttgagaccatcaaattttagcctttgggagtaagtattggggccccaccctgatatcgcaattttctcttccagaaatctctgcgatgattttataatgtaaaatttgtcagtcgaagttgtacctggcaaatggataattttccagtgtagaatttcagaagaatggaaaacaatttagtgatatgttgatttgctatgtttcaaattaagtaaatataaacataaaattcaaagaaatttaggactaaacttctggtgactttgttattagttgtcattttagtctgcaaagagaaggaacaggcttaagaatataataatttttctgtgaatgggtcagcaacatctgtaattacacttaataatacttaatattccatttgtatttcaatactctatttctcccagaccttttgtaaccctctaagaaacacttaactgtctagttaagtaagggggcagttagtctttatttaccaggaagaagaaaaggcacacatccacattgcttttttatgtgctgggctttgcattccttcaagcaaccagaagtgctgtagctgctaaaattctgcagcttgatgttaggattgcattctccccaagggtattcttgatgaactgtcttaagataagataaatactgggttcaggagatgtgtttacatcaattcacttattttccatcaagaagctggtacacatttttattttcctctcagagctctttgattttcaaaaccaaaattggttacatttgtctattttgcctactctattgacctgaaattccctgaagctaagtataatagtgatccttaaacatatatcccaaatgtgaattaataaagtcagacacattgagtattctaaaaatcaactcatataaggaaaaaatatcatttatgtaaatgttatattagtgtagtaatcagctcacctacaaaacagtgaagaaaatcacattttaaaaagatcctttaataagcaactattatgtgccaggcaacatggtgttgcagaactttctccttagttcagctaaaactgggctcttgtcacatgaccaggaaaaatgaggctcgcgggcaaatagaagggtgaggaaaatggaatttattgggtgaaaagaaaaaaagaaaaatgactctcagctaagtgagagagagtcctgctggtaggtttcccacctcacagattgaatctcaggccaccacacaggaacatgagaggccaggctcctccccactgcaaacagtgcaaacttcccaaggctccaccccttcctcccaatgtgcaggtgggcatttttcagaaagaatcaggtgggaaaggacagccttcatctgggacaagcagtccagtttttcagccttcaggctgttttaggtttgaaggcagggtttcaccaggggcccttggctgtctcctgtgtctatcaatggtaagtgctttctttatggtacttcacttaaacttccttactatgaaagaaaaatattaccccttccctatttgtgcagagaggagatgcaaattcaggaagctaattaaagaagttaccgggcaagtaagtggctggcaaacagaaacctaccagaccagcctgactccaagattcctgcttttcccactaattttctccttcccagatagtctcattgaagaatcatagctcagcaacttaggtacctctttatacttaatgaccctttccatttctaatgaaatgtaaagtttggatttgataggcttcccagctttcacttacatagtttctgtttccactaatcatgtaccttcagataaaggaaaataatctgaaatgaaattttggaatttctaatggtgttttacaggcatgggagaaaggaatttctagtgaaaagcattacttatgtgtttgatgcattcactaccccacctaatttcctcagactcgagtatttaccttcacttggcggatggttacccttgcgtgcattcccacaggtgacaacaagcctaacccatcaaactgtggcaccttctttggtgaatggataacaaagatgatcccagcatcaacgaaaccaagggctgggttatcagtgaatgcctcctgaaacaaaaatatgtttttgcccaaattgcaaattgactatatcagttctaaacaagtttaggatttagttttatcatctcatgatagcgtttgactaatcctaaatattttatgttatatttctatctctttttgtctaaagtgtatgaaaattaaagacatactttagtggttgaatcggtgaaaggaaactatcttgggtcccttcaaggtggggtctactcaggacagtcctgaaataattgaggcttgtctcatcatttttcttgattgatgtctggtaaccacaaagggatcctgagtgaagctgacctggcctgcagcagctagcctatcagtgcttggtaccagcttgggcaccttatagcccaaaccaataggacgattgctgaactccaggaactctttcctccagggatccctgatcttccattgtttttcatttgggggtctgaggttcattcgctattaaaaaaacaaaaaacaaacaaacaaaaaaaactcctttttttgtgggagtttccactgcatccaccaaggaatgtgaacctacctgcttctgcatcggcagagagcagttttcagcttgggccccatcactaggtaagaaaactggtttgggattctttcttgcaaattctttttaaagaactaaagttagcattaacaaccagctgatgttaatttctgcttacacttagagcgctcagaaatcatataatttgtgtgatcactgttagttttgcttaactgttttgttgtttgtttctctcttgtggggttgtgtgtgtgtgtgttttggttctttctctcattggatttgaccaactcagaaccctctagctcatgagtatagaattttccactccaaagaaataaagcaccttgctcccctaagccttttggggcattctcatgtgactgagaatcacatgggggtgtctgggaggaatgctccctaaaatgtgcagtggctctaaataagtatccccctcagaagaatatacttagggtctaatctcagctggcaggtgcatgttaggagccaacccctgctgcatcttgagcacctaacacactgtgccaggtagctgcaacacaggacgaccaatcttgttcagggataacagccctgaaaagctaagtctgctagcagcacattttgggtccaacacgtgtcccaacttggtcaaatccaaaggggaactctaaactatggggaacaaggcctctgaagtggaaagaaaacagcaatcaagaggaaaaaaaaaggaaagattttttattttgactactaaaggggctttatttacataacaaagccacctttttatcagccagaccaaactgaaagagcaatggctgcacttctgaaatatggtaatgaggtctaaaaagaatttttttaaagaagctcagtgtttcaaagtcaacttaattaaaagattaacatccaagatgtgtgtgtgtatgtgtgcatgtgtgcatgtttgtatttaaaaggccttcaggtttttgtgggttttttttttgtttttctctcctaagactttgtcttttttttgagcaaaagttttttttttccttcagttgactgaattccgttttcacctgatcttttgactaaaatagttattgcaacagaggctaatcttgggtttttaaggaagagtgtagttaagacactcagaaatatctttgttaaaaaaaaatttaagtgcactctgaaagcatcacagggtctaacctcaaaataattctaccgttttttggagacccaggattcaatgtgagctctgcccagagcttagagatccagttaaaatataggtagtccctatctaaataagattggtctccttatacaatattatgatagagttctataattttatgttagatttggctcaaagaaaaataaaagcatctccctctagcaccaacagactttttctctctgtaccttatgatataaagtttgctattttattttcacctgagttgtttcctataataagcaaatttaaggctatttagctaacaactgcctagggttgtaaaacaggttatcaagaatctgaatgtctaagataggaaaaaaataataaaagggtctttatgaatctataaaatgtacctttattggcatacctaatatgtctatgtatttatatgtcatatacacaatatttcactacagaaaatatataaaagggctctaattaattggcttaaagaaaaataaaagtgtttaaatcatatattttatcaggaaaaaagaaaagacagttcaaattctttttcaagtttatgtaacttaagtaaaatctttaatagaaaagctagctttaaaattactagtaaagtaatatcagaaatgtcttaagaattgccagcatactttttttgtttatgtttattaatcaggctatttcaacttatccctgccaaacactataaaatgtcaaaatttggcatagagattacaaaactgtaaacccagccccaaacagaatgatcattacttgtgtagtttttaataaataagacattgatattggtttaatgaaaatagctgcatcttaaattttcaaaattaccataatttctaatcttgtggctttaggcagcctagtccacaggcagtaaggaggtttgtttgggaaaggactgctattgtctttgtttcaaacctaaactataaactcagttcctcccaaagtccaggaatgaacaaggacagcttggaggttagaagcaagatggagtcaattaggtcgtatctttttcactgcctcagttttatttttgcaatggcagtttcataactttaaatcatgactatcgtagttttcctaaataatctaggtgaacaattaaaataaaatagttaggtaagggataaatacttgtagacaaacatgtcgtaacttagaatataaagttatattcagttaaataatagatatttcattatgtgggtattttccaataaatatatattatagaaaaacattcttgctaaaaaaaagtgtgtcctttataaaaaaacataaacaaattttgtctaattcaaagcttatctaaaggttatgtataaaacaaggtaagaagaacaagcaaacaaaaagagatgtaaagaaagctataaaaataaggaggtttttttgtggtaagacagcttaaagagaaataatatggtaaatttagtcctaaaataaaatgactggttgtttaagaaaggagaagtgttcaggtcaaaccagaaagttcaagcatgtcattaatagtcagtgtaagtcacaataaggatttattttttaaaaaaccaaaaactttaatatgatcaagttgtcacattattattaagtgttggtttgcttaggaaaaaaactgagataaaaatttttgttttcaaattaaggttattacatccatgtatcttcctgtatgtgcttttaaagtccttgtgacattaagttacagggctttgactccagggtctaaaaaggataccaagtcctactaaatcttaaacactaacagcaattaaatcctcatcttcaggccccacagcagattccaataaaaataaaatgcattcctggccaggcacaggaattcacacctgtaatcccagcattttgggaggctgagcaggtggatcacctgaggtcaggagttccagaccagcctggccaatatggtgaaaccccatctctactaaaaatacaaaaaatcagctgggcatggtggcgcatgcctgtagtcccagctacctgggaggctgaggtatgagaatcacttgaacccagtcagcggaagttgcagtgagccaagatcatgtcactgcactccagcttaggtgacagagtgagactctgtctcagtaaaaaaaataaataaataaataaaatgcatttttgagatgtggggccagaaattaaagccattcaactcctcgaggcctagggactattgaggaagaggtgggcatgtgagattgcaatgggcgatattaaaagacaaaataagttcagtttctctataaattaatcacgactgtcaaaggcacaatgatgcaagaccagcatatggactcctgtgtcagattaacaaggttttcttgaagcattaactaactccttaataaagatcataaaggttataaaaggcttatggaagttatattttatggtcaagattaaattttatagattgtttacaaaattttggaaaacaaatttaattggcttcatgctgtttttattagggcttcttatttggaaaattaagtctcctctctcaaagaatgaagtttttttctttttttaaaaaaaaatccttgagttatcactttggttaaatgaatgactttacaataacctgtaatcctatttcataatatcaagtattttacacctttgatatttgaagatctttctaaaatcaaattataaattatgtctttttctgacctaattaatcctttaagatattagtttccctaaagtccaaaaatgacataatttggcttacttggtataaaattatacaggaagcattgtcaaatatgaaatggtgtttggttttatttgggctgtatttatgtaaatgttattggtaagtgttccagaattaatggaaaggcctgtaattctgatatgacttagtgtacattatcaataataataataattgttatgttaaaattattgtgtaccactgaggtaacaaatttccttgtcaattgtgtctttgactatgtctgccctaaaaccttttttcatccaaggacaattgtgctcatgttttggtcctctttagaaggtgtttttataatcagctacaaaactctaacaggtgcccttaaatgcaggtttctgataactttggagattgtaacatcagaaaagaggaaaaactttcaggactcatggagagctaaaatgttcatgagtattaaacagaacaggaattaactgcatggactcaaataatcttttttactttttacttaaaatgtttgctgatcctttgttttgtttttcagagtcttaaaacttttattttgagctacaatttagaatactcctatgaacaaaacgtggagcatactttatcctgtctgcctgatttctccagaatttggaaactatttgtggatattcttaacttgtggcaatacagttatttgcataagtgcaataagaatctgttttcacttgtaacaggacacaattggagaaactggttattttaccaaggcttttactggaatggtgtgctttcctttaaggaatcaaacttggcttatgaaaccaataatgtccttggaaaaactgacctcatattttgtgtacagagtccctgtacagggtttctgacctgtggtaagtaaagaatgtcactttctgacaggcccagaagctccaagtttatcttggaacctcgagtggcgaggagattcacccaactcataggtacttgatggcacaaatctacggctgggctcggcttttaaaaagtcttatctgacattccttctatggaacaaagttccaccaaaggcaatttaaaagcctatgtaaaaaataactattcttggtgcactgtatacaaataatttggcaaagtaaaataaagcaaactagtcctaacatgatttgtctttagcaaaaatgggaaattttatgtcctaattaatcctttagttaggattagagaagagagaaaaattatgtttccaaaactagggtacacctgttgttagattctagtcttgcccagtgtttttcaatttttattattttctacagtttggaccaaattctattttttcttggctacaagccttcaaaataatgttttcaatttttttcttttttttcccccatatttcctaatttggagtcactgaaaactaagctgtgctttcttaaagtccggcaaactgaagccagtcaacttaaactttagaagaaagtaactgcagcctatttacatacataagccacttttcatacctgcctactgatgtacggacttcaaagtaacatggcctatatgaatatttccagtattgttcttttttttgctgttgtttttctcccttcctcccactattttctcttcatagaacatgagactttgcaatctgctaaaaataagcttttgggacctacccatctagtaataaaccatcctaaccatgagaaatcagatgaaaactgagaccagagactcatattctcctaaaatgctttctcctaaagattttttttttaaagggagcaggggaatgggaaaggaaattatcttgggctctgtcaaactgggagctgcctcccattctatttaaagttattcctttgctcactgagatgaatgcctattctgattgcctcctttggaaaggtcaatcagaaactcaaaaaaaatgcaaccatttgtctctcacctacctatgaccttgaagcctcctccctgcttcaagttgtccccacctttctggataaaaccaatgtatgtcttagatatattaattgatgtctcatgtcttcctaaaatgtataaaaccaagttgtgccctgactaccccggactacctcaggacttcctgaggctgtgtcatgggtgcctgttcttaactttggcaaataaactttctaaaatgattgagacttgtctcaccgtttttctcaattgacattcattgcttaagacctctatagttttccatggatcacatttcaccttagataggtgcaggtaagtatttacaattactgttaatgatcttgttccctctggccttctcttcacctagttgccttttagttgcttccctcttggctgttttctttgactcatttgcttagaaaatcatttggccctcttcattttgcaattaaaatccctgcccttatgttaaattcaaattcattttcccataagagtgaagatttacactatttctttaacactaatgttctttttaataaatcttttgtcagatgaagaaatttaagagtaaacttctggtggctgtcattagttgtaattttaggctataagaagaaggcacagtcttaagaatataaatataaagaatatcgtctatgaatggatcaactgcctatgtaattacatttaacaaacatactaaatattctatttgtaattcaaaactctatttctcctggcccttttgtaaccctctaagaaacacttaactgtctagtctggagaaatatctaaatgttatttacctatggaacattgcattgaatttgtagatgttcattgcattctaaaaatatttggaaaattctagtttgtaaaaattgataatataatgttcgaactgaccatagggctaatatttgaatgactcgaaagtattaatcaaatacattacaagtatatgtaggtacatgcattaaagtttgcagtgttatgtttggacgtaaagaaaaatatcttgaatgggatacaaaaggaagctatgaagagtacattactggaaattctttaaaagtagccaatggcatgattcacttggattgggaggtgaatgcaggaacatcaattttacttttcatctttgtcattaattgtatgattttggatacttaatccatcagcattttacaatcctcttttataaaaccataataataaagtagaatgccttaaaatgtgtaatactagaagtagttatttaaacaaaaggtcagaattaactaatcttgggtgggagctatactaggaacccttgaagaaaacaccactgcatttcttaggtgtttgaacatctgcctcccaggactaaacacttagaacttattaggtcaatttcagctttccaaaagctgggcttggacaaaatctatttatataatattctttatataaccattatgttttccatcacttttccagatatgcattctatagtttgtcaaatgtgtatgtagagtttctttctaaaacgtgatatttacataaacgtatttctaaaattattgttcaatgcaaagagttctcttaaaatagctacatacattgcaacttagaatctctgggttttcttattttgtggcatgaacattccctggagtcaataatttaagtcagtaggatagctaaaaatctccagtttatgcctcaaaattttgcctgtttaatttctaagccagtagttatataaaatccataggagtctagactaaattaacaaaaactgaaataatatccaatgtgtgggaactatctgatatggcccaatagtctgatcaagtttttgaaacccccacacattctcgtttgtcttgaacctggagaaaattcagtacctgattcacattgaagagtaagctcaaacctcttccagagacactcacttttctctttgcttggagagtttcaccatgattaaaagtaaaacaatttccatattcagtgaagacatgtgcaaaatcctccaatatgtaaatgaaccaaaaaaaaccatcaatttgatatttttgtcacttctcaagtttgccataacctttttttttaaagcacaaatacctaacaagactgcagaaatatcagagagaatagtctgatttatatgtgtattcagtgatagttttttttttttggtaatatgcattcgtcaatttgcatgtgattatttaaaggagacattttcatataaaattaagcatgtatttaaaacatttaaaaaaattacttgtttaatgttttcctgaagatgtatggaaacatgtttttatctgtggagcactacttcagcatcctttcttggaggagctatgcctaatgtaacctgtatggtcacagtgtggcagtcaatcacatgacccttttactctccttttaccctccttttccaaccactacagaggtgtgcatgggacacaagctgggccaagcttaaacctcaccttcttgcctcagtggttggttcaagaattgatatgtgtcccaagtggagctaattagagtctttggtgagattattaagtggatactggagtgctaaactctgataatgagtctagggggagtcagcagccattttggtgaggtgtggggagaactgatacagaaagaagccaaccagaattatgagatggaaacaaagtcagaatcttaaagacagtcttggagtccgttggttcagcattttttgaagccagacctactgttggagttcctgcttaattaagccaattcattacttttagtcttcagtttactagaacagaaatgggtttctgtaatatgcaactgaaaaaattctctaacatgaaattttttgttggtaagttaaacttgttggtgaataaaaacagcataagaaagcttactttccaagttaagagtttaggccagaacctcacctctgtgaaatgtctctcaggcatttttttctgcttactgtgtgctaaggcggcctaccaaaaacataagaaattgtattgaagaacaaagaatttgtgtttacttaggttcccctaacaacacaggctacagaattgctgatacgggaaatacggcatcaaattgcctgtatagttctttattaatagattacagatagatctagtactttatcttaaatgtagattaattttataaacaataataacaggattttgtatttgttatgtatttgttttgtatttgttatgagtgaatgtttattatatataatatatactagttattatatataatatataatatatactagttattatatataatatataatatataataataatatataaatttttcatagttaaatgctgggatcctcttattttctatagtctctaatgcaataccataagcatatatgcagagtgatggatatttggtgattataaaaatactatgttggaataggttgtttgttgatgaatgaaaaaaattaaaatatcctcttgtgttaaattgtttttcttttggaaggactatactacaagaaaggcaatcaagttttctcttttctatctttccttatctaactgcttgaaagaggaaatttgtctcatgtgtattccatacccctgatatcataaggctgagctaatgaaatagaatctgtaagcaaatttggattgattcaccatgggagtaaatgccgtgggagctcaatgtgaaacagtgcagtaacggaacttattactttgattatatttgaagaatagaaaatcgttacctttgggctacatggctttccaaaaaactcacagtccaacaaagtgctattgttgagataaaaacctttgttcctgataaattccacaatgctgaagttttggtgacttgcagcaaaatcagtagcctctctagagccagtggaattggcagtaatttcttgaagatggaggactttggatacaatgtgccataagaaaaaaataacaccaaatttggctacagcatctgtttggaacctattagcaggaatgaaggcaatagttagaataatgaaaacttataattatctggaagacagataccaacatctagattctattagcttcttcttatctcccacactctcccttatttgcaagacatttaagcaaaatttgatgcctaagaagtctttgacccattgtgcaattaatcttgtatttgcacatgtagtttcatttacctagaatattccccagaccttgatctggatgacacctaatggatcttcaagattcaatttaaaggaagaggagggaatatatagttgttaaaaatgcatttaggagccagatttcctgtgtgtggccttgtcctgtgtcagcttagctaaattagaactgcatttcccagcattcccatccttgtatatatgtgagtttcactgtcgtgcaggagctgtggtagctcacccacactgcaatccatctgctgactcaacttgctagtatcgggcagcagccaggaccacagctcctccagctcctttgtgaccttttccttcagcttcttcaaatactgggttaggtaagagtgcgacctggtaaagatgacagctatcctgcaggtcattcacaccatcaagattggaggtagtgaaaaagagacctacattttgagcttgtatttgtttccccgacttcatatccattttcccttcctgctggccctgcttacttcagccccagcacaaagaacaggcgaaagacttacaaagactgcttaaccagctcctgcaacgaagtggggcttaatccctagaaaaaaagccctttattctgtcattcatggtgattctgtttctccagttaggttctaattgttacagccacttgatggtatgacttagcaaatttttttttcatttccctctgacttaagtttcctctcttgtaaaatctgtataacctacctcaaagtgttcttttaatgattacattaattgtttttcttaaaatttttagacacctgcctggaatatagtgagtgttaggtaagtacttattaaataaatgattcattctggccaccaaagcagaagtaatctatttaacttaacgactgtatcactttctaattctcttatcttgccgggcaattttgttctgcattccatatggttcttcatttgcatgccttccattcctttacaaacatgatgaggactttcatattttagatattataaattttgaaactgaacaaatgttctctctttaaattgactgctaaaggcatagaacggaatgcatattccactcttggtaagtatatggtgtatgtggtttgtatgtcatacctgaacctcctttgtgtcccagattttatttctattttgctttatattatattatcttatttaatatataaatatatatctatatgtgtgtgtatatatatacatatatataaagagagaagaaaataaaaatatttactctaaaagacattttttgacatattttgaaatggctgctgcagggtcagctaacggaagtggccttgcaaagctgtcttttatgtggaaaatttgcatctgtagaaaacgtccattaatgcagccgggcctcctcttgctaggcatttgctaggtataggagagattgagattctgacacctttaaaagtctaaaaataaacatttgccatcttgtctctctgaagaagtcttcatctatataacaaggtcacctttgctagtcaagcctcttcctttctccctactataacctgtataacctgtcttggtgttaaaacctgctttcagtaatgttctgagcccacattctttctataatctcaagatgataaataagcctctgtatccggttgggatgttgggctttattctgaaggctcatgtgtatacacattaaatatcattgtaagccttttcttctattaataaatctgcctcatgtcactaatttttcagcctatctttagggggccaacacccatggcccccacaatgtatacatataatttttctttcaaattcaagccatattaaatcctttctcagtcaaggtagggcatacacaaaacacaaaaatggagaaacaacctcaagccaacattacatactctttaagagatgaaaaactgttttcacagtcaataactaatattgtagcttgcttaactagctctgatacttgcaaagctagcccaggtcctaaaactctgtaaatatcctcctcggacttcctgattccaaggcactattggtcatctgtcatggtggtattctgtctaactacagtacatctaataaccttgcatttcttggtcaaaagtatttcttggatcattaaggaattaagagtttacatcaggaacagattttggcagaagtacagtgtaaaccctatattgagagatggacctccaagctacttcaggtttccaactcccagtgacagacaggttcccaacaatctgctccaaaaccctacaactgtgaaatcctgcacagtcagagccagtattccctgaatatattcattggattatattcaaaatgattatgcagagaaataaaggaattactatacatatgggagcaatgaatacaatctacaaaaaagttaaataggagggcatcttaaaatatttttaagctttttttattatactaataatatttgagaccatgagagtaatttatatagattaattaaatttaataaataaagcctaacaatattaagccaaggaagaaattttttgtgaggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgagactgttctcacaacaaaatatgcacatcttaaaataccatctggcttctcaggtccacttgagactggtgacctcacttttgaccttgccctaaaagtcaactggagtccatatacctcaggtacaaacagctttaataagctgccacttcctgttcctctcatcttctctctatggcaggactcagagcattaactaacctaagggcctggtagaatgtaagctggaggtagagtagttaaagtctgtgcattttgtactggtcattttccagttcgtctgttgctaagaaacagcaacagaggcccgctctagagggttacaaactccctggaattatggctcccggacatggtgtagggaaaggtgtcccacagggatcaagcctagaatatcgctggatcttgtacagtttagtaaagggcaggactggatttaacataacgtccaccaaactacattactgaaaatttttggtgcttcactcaactagtctcattgactagaagtgaaaatatgtttttctcaatattaccagctttaaatttgccataaactgttgtgggaccaagccacagaggaaatgtttttccgctagacaaagataagcatgaggatgccagcaacagaggatattccttttaaggtctgtccattgcaaagtacacacgtacacgctcacacaacaaacacgccctgttgccttttgcttgtgttaaccactgagggcagatctctgaataataatacatttagtgggtccacaagagctgcatttcctctgctccagaattcatgcaaggagcacggctggggcttctccactgtcattgttatggcctcaaatgaaggccttgtcaccaggcgggggcgggaaggcactggagcgcagcagcattgagagcatccgaattccctcctctgtccctgattggttgaagaactggggagcaccgaccagccccctgcggctcgactcgccattggctgggaagaggtggcaatcagggcgggccaaggcggctgttctcgctccagctcgatgctgcctccccggcccggttgcgctgtagccgctgccgcctctgcctgggtcccttcggccgtacctctgcgtgggggctgcctccccggctcccggtgcagacaccatggtaagtgctctcagccgggtgcggcccgaacctcacccctcctcggccggcctggcagcgagggaactggccgcgcagccggagcttggctcgggggccctgggcgctcactgccggccacgggagcagcctcactccttgccctcgcccagtcaggggaggtgggaacgccgcgagtcgtggcgggggcgatcggtgcctttgcttcccaggcgggtttgccgctccacaccgcagcttcctgagctcgggaaggggaggtgcggcggaggcgtggggtcttcccggctctggccggccccaccagtgtgggaggctgagcgccagcggaagggaaggctccgggttcgcgtccccgcgctgcccccgatgcccagcctctcccggagcggtgacaggtgaggagggtgggaagatagcgtgggggtggggcggatggtgacggggagaatccaccagggattggggggttgcgcccccacgatccgactcctttagtgcatagtacccctaagggagaggagtcaggggcggggtgaaaggttaagccagcgccatgggagcagaggcagcagcctcgcccccaagccgcttctcaggtacagcgggtccctgacgctcaagtctctccctgtccccgcagtacggatttgtgaatcacgccctggagttgctggtgatccgcaattacggccccgaggtgtgggaagacatcaagtaagtggccggctaccctggctgtggcccaggtcggcgcccagtgtgggaggccccccgcgcctcgcctggtcctcagcctgctggccgggtcgcgggcgcgcatccttggaggtgcctccgcgcctcgctcccggctcgctgcagctgcgctcccacgctccgggactggaaccaggaggggaggggcggctggggcggggctgcgagggcgagaaccgcggaaaggagcccctaggggtcaccacggcttcccgacccggcctgagcgtgggaacgcgctgccggcgctcccaggctcagcaggccgggcagctcccacgcgtgcagagccccgagggaccacacgggcctccgcgaggttcctgcgcccggaggccttagaaacaaacggaagactttggctcctttatgatttacacacctggcgtaagttaggcctttttggtatgctgaaacacgcgggagattatttcttgagcaacagtgaattaagcgcatttattcatttttaaataaagtgaaaattggaagttttttgttttccccaaatcccactgcccacccccccccgccccaacccctatcccccgtttttttctactgtgttgaagacttacagttctaggaggcattctactttcagtcctggagctttgagcccagtattggggatgtgaacggaaataccacacccacctgagagtttaaacctgttggaatgactgatgggtcagcatgtctgcaggcaggttgactatttcactctaataaagttgtctctcctttttcctctcactctcaaaaaatttgcatttaacttatggtacagctgaccaccaccaactagctactccaaaaccctatttatggattttcatattagagcaacagtcagactatcacactgtaacaaaggtggaaggaagggtttcctgggtgggcagatgggaatacactgttattattcactctgatttcctgtacttagtgatttcattttgttgtctcaggtaggtgaggcttccatgctgtttttccctaagcacattacaattattagcaatcaattctaacaggccttattatgcagttacatgatggtgatttctcataacgatctgtttccatagaaatgaccagggttatatattaagcacgaaaatgtaacttttctttcccctggatctgtatcactgagcgtttctttcatgtttcagaaaccatttgtattatccacagggcatattacctacatattataaacagaagccgagatttggcaagtatgtggttgaatccaatgagaacataggcaccaaaagttactttaggatttgaattttctggccatcttaataccattataatttttattttgcttagtagacagatatcaacaaggcagacatcaaaaaaagattgttaacagtataacttaactaccgaattatttttaagccacttcctttgtaaatttataaatgctatatatatatatattctccatatgatgcttttgcatctgtaagatagaaattcccaaatttgctggtagatatgctatgacaatgaatatgcttagaatttttagagcagaataaaagatggctattatgattaaacagtcattatgtaatttaaccagatttagattgtatatcctagccattatcagtttagatcactgttaaatacagagatgaaaaataatactttatagagtaatgtatcagacattttaaaaagttaaaattatccagagatttgtttatattgcttatattttcccagaggaagtccaaatatgtaaagattcaattgacgagaatttaactctgcaaaatcaagctgagaatattgttccattctactttaaaaaattatttattaacttactaatttatctttaagtagcttccaacagtattcataatttgtctaaaggataaaaattccaatcatccacaacacaatttttctggtaaaaatgaaacaagttatattggtcttttcttaaataaaattgcctcaaggttatgcccctgcaagggaaaaactgttagtcacactggtgttggtaatccaggtgttagtgttgattctttatttgtctaagcatcttttggtggaccccagctgattttgggtgtggtttagtgtctgggctagagaacatggttgaactctctgcactgagggcattaaagctgctccttggtttccaacccaatacccctgagtaattactaaatgctccctgggagagcatttgctttccacagaaaagaatcttgctgcctggggagaggggaggagaacctggaggagtgtaattgaaaaatgctccttgggtgattctggttcctcctcccctccctaccctctgcttcagggaatattaaagcagtcattggtccaagatgctttcagttttaacttttataagaaattgaatataagaggaaaaacatttttttttgtctcagtaagtaccaaactcttattttttcttttggtctttgagaaactatctaaattgatgtttatacaaatttacattatacttttttgcttcactaggaaatggccatccatccttcatgagaaaggatgggaaaagatgccaaacatttaatgacaaataaaaccctaagtaacagattccagacagcagacagaaaattctaagtggataagggccaaaaatgcttcattgtaaactcagtatccacttacactcttaccaacaatataaccatttcacaaaagctttgaattcctcttaagaagaagaaaaattgtctagaccaagggccagcaaactttctcttaaagggccagatagtaaatgttttaggtgttacaggccaaggtaaaactgaggatattatgtaggaatcaatataactattaaaaatataaccacttaaaaatattaaaaccattgttagctcttggaccaggaaaacgcaaacaaacaaccaaaccagacaaagtgggctacatgtgatccatgggctatagtttgatgacctctgatttagaccataacaaatgtgaaaaagcaaaagagacctctgaatccatttcaaagttttttttgttgatttcatttcaatattcaaatagtccttttaagaggctatgatttctaagtaatgacaaaaaaattatcagggtagaaaataaataaagttaggagtgatacatcctagaaaagcacctaaatacttgatttcccatctctgttatccttccatcagccctgactaaactcatcattgcgtcaaattactgggtttaattttagattttttctctgctttctttccatattttttctgggtagcaacggagggaagcagttagagacaggtcttgctgtgtgcccaagctggtctcaaactcctgtcatcaagtgatcctcttgactcggcctcccaaagtattggattatatgcaccaccacgcccagcccaactttagattttttaacctcactgaaacatacaaattaggaagatagatccaaaacactgaaaaatcccaaatgattaaaagtctgtaaagaaagtacactgtcctgacttaaataaatcagataaaacgtttatctttaatgagtatagtcaagtgttttaggcactgaggatagaaaagcaatgatgtaatccttaccttcaaaaaaaaaattctagagatttcatgatattttagtcttattattttgtttttattttcctgcctataactcaaatacggtaaccttttgtctgttggatagcatatacttccagttttgaaattaatgtgtcttgttaaatttgctaatcacaaattaatcattatggttcaaataaggaaacactttgctataaactagttatgggactttccaagttgcctaaactttccaatcctccattgcatagaggaaaaaataatacttcacagagctgatgtaatgcattgaataatgtctgcaaaatgcttaacatagttcttgacaatagcttaataaatgatgatttttttttattttactctagaatcagagtttttaaactttccgttccccaatttacttgaaagatattaagccctcctttaaaaaatattattgcagtctatattctatcagtctaaatttttttatcactctaggataagtaatcatcatcatcatcatttttcatcattttgttttgaaatgcaacaaatgctcttaatatccattgtatcaaagcaaattaaaacaaacaaaagagcacatcaccatgttttgccgtgtttattcagattttcatatttttgaaggtgtgatttaaacttctatacctttctgtagatctgcttaagtagtaatgagcctatgtatgttttaaagtagtaaggatactttatattataaagcaatttcaaatttccaaagttttgttctctctataattttggttgactgctacaacatccatttgaaataggaaaggactttacagctcaatttttttaataacaaagttgattttccaagaggttaattcccatatgtaattacttattgtcccagagaattacatataggaaactgaaaagtcagaacttcagcccttgttcttctgttgccaggtttaatgcttcttttaagatataactgtgaaataggaactaatgtagtatacatacaaaaatgaaaccacatttattttcttacccagaaacttcctcagtacaaataaacacattaatatacagactaaggaaacaagtaaaccagatcattgttaggatcaggatccttttctgtagaaagatctgccagttagcctgaaatacagaggaaggcgttgcattgtattgtgtgttggctgttaagcttactaatgtttgaagttatcttttgcagctggaactggatgcccaagtgtatactccatcacatacctgctattgagctctttgcatagggctcttccagaacctggaccatgtctgtgagcagcatggcaggcacctaggtgataaggcgggtcatcacatgatctcagggagtttgtgggtgtttcttttgagttttattttggttctgatgatctgaggtatcccccagaaatcaagctgggtcttggagtctttattttccttgcattctgtgatttttgagaattaaaaaaaaaaatcagaggagtacatcacttaatttggcgtgtcggaaggatctttgcacattacggtgagcatcagaaatcagaaaatctgtattctcactcttaatgtacccagatctagttttggcaaacatttactaagcattgttatgatttgcaagaacaaagtcctttctactttcttgcctttctccagaataaggggctctagcaaatgtttctctttaatgtgttggaaaactatagttatgggttgatgctctgcagacatcaataggaaaacacacttaaggaaatgaatgctataaagaaatctaactggctagatccgagggtgacagctcagcagacgtgagtacttgcagctttttcattctctcctttgcctccttgttcgttaaagaggaaatggtgggagaaaaaatgtctttattgtttatgtctagcaaaatgaagcatctttttctgtgtcaaactatatcaaaagatagtgaagttaaagtggattttattccaggtaaaagtagacattctgaagtatttattggacctcctcgttactgtggctttggttctgttgcatattctggtctacttccccttcattgttgaatcatgaaatatgaaattgttaaatttattgaaattctttgtggtcaatcttcagataacccaaacatggaaacctttcactcaacttgtcctacatacccttagcattccagccccatgggcctgtttttagtttctcagccataccgagccttaaatgtgtccgtttaacacctgtgcttgctatggtctccttttcttcaccctcccaaccctcctgtaaatgtaaacacctctcagctttgagatgtcaaagcaaatgtcacttcttaagggaaccttctctcacatccaaacttttccctttccttcatagtgctgtaatgcaagagtttgtacttagtatttgtactactgattaatgtctatcctccatgacaatagcctccataactctttctagagtgctctctctctccctctctctatgtattcaataaaatatcaggctctctttctgtaataaaaatatggatgtttatatgtgaatgaagtatctttcacctttttcatttccaaagaatagagcacacacatatttttatcaaagattcttctctgcaaccagaatttatgtcagcatggtgacatattcaactactatttaaatataagctgttttcaaatagacatttctttttctccaagatttattgagatataattgacaaataaaagttatatatatttaaggtgcacaacttcatgttttgatgtacatatacattgtgaaataatcactgtaatcaagccaattgccatattcatcacctcacataggcaccctctttttttgtagtgagaacacttaagacctcccctcttaacaaatttcaagtattcattatagtattgttaactgtaattgttcattgtaattgttaatagtgacattgctgtgccaaataaacatttcttatatcagcaaaggcagaagatagagcaatatagcaatacataatagcaattccttttggaagtgtcagagttcttttatgttttctcatgtcatgtggatgtattggaaggactaagaactctattgttagaatgacaatttcatatgtcaaaagacaattactagattggattcaggacatgattgaatcctttagcaagtttctaaaaggcagggatgtgtggtgctgtttaaagtcagtgtcatagactgtaactcagtgtttagaatactgaaattaaatttgaaatccagctgagctctcaccatgtcatgctatttcgaatgtaatatgtcacgtatattaaacacataccatatggttctgcagtcacagtcctagatgctttatactcattatctcagttaatcctcatcaccaccacagaaaatagattccttcattatcctcattttgcaaatgagagaatagaggcgtaaaagaaagcctgcctcaaacactcagctattaattattagagctgggatttgaacacagataaagttgtaattatcatgttatgtgttttcctccaaagagaaagacctataaccttcttctttccaaagacaaccaatttagcaaggacatgtgccatgtttatgtgtattagtactgcctgctgctttttcttttttcttggaatgttcaagcatgcgtacaggtgatttttgacaacaatatttttaaaggcgaattgacatgaagcatatacacaagtgtctaagtaaatgcatttgtctgttaaagctgctgaaaatggctcagcagctcaaatgaagatttgtaagttttgtggatatgttttcaaaattggagaaattaccattaagggtctcttgtgactcaaaattggagaaattaccattaagggtctcttcctgacaattgcatgtcaggaagaaacatgcaattatgtatcaagactgttgttgtttcccctaaaactaaggtatgggtgtcactcaggagagcatttattgttactgtttacagtgattggatgagtgagtgaacacattgacatgaaaggactgacctttactaactaaaagggggttcaggcaggaagtcgttactggatttaaagggcctgtctctccacttggcagctccctcaagccagagtctctcctttggggttcaggcttactgagaatcattagattaggctattgaaaacttagaggctattcatgctccacagccacttagaaaaaagcaatataaagttttacttttttagatgttatatatagttactatccacagtttatagtgggagattttagttttaataatggttggggacaatgcaacattatggtaccaattcagctcactacaatggaccattctaggatttattatcaaaacgacacctacgtactgttttttaatacagcatgtgttctgataatgttgtttatgggatcaatagtatcttggagctgtcattggtcagtttgattgtttgatcctgattgaccttttctgcccaagagcagaccttgtgttgagattatgttttcaacatgcctgtgttcaggctctcccatttcaaaatactccatggaaaacttctttaactgaaatatagcagtcttgtcatattcttaacttactctaaaaatgtacagtagcaggagctcagggtcagaatatgaattcagtttttctgtgttcacatattgtagttaaggaatatctccttgtatgtgaagttataagaaatgttacctggatattttggacaaattgtatacctagttttaaaaaataaataataaaaaatattctttagtttgaaagcaaatgatggacttttgttagtaatgtttgattacttgtttgtttgtttttttttttttgtagggagcttggaaatgtgtggggaaattgatttcattaattcattcactaatttttaaacatgtatacctttattaagcacatgtgcacctttattaagcacatgtgcaattccaggcacctgggttaggcaccagtaattacattaaagaaaaaaatcccaacttaccaaacaccaccactataaaaataccaactacctctttgtgaaaaggtaattagtataaataaaggatctgcttaaattccaacaaataagagtaagtaggaagttgattgctactgggaaactatttcacttatacgtattcaacttctaaacagttactgtggctacagatacaaagaatgtgattatttctgattttgttgccaggtgagaattcgatctttatttttaaagcccttaggggcgacagagcttgtttgtttcttggctattttagtttgtaaattctatcagcaaatctgcatactaagtgcaccttaattatttgattctgactaaaaatgactaagattttgttgtcattcttcccttctcttcttccacttgccatttcctctttctcctttttcccttcttccttcatcttctccaactactataacttaccagttagaggaagtatcaggcattaagtgtgcaagaagtactaccctgagatttttgcataggttaattctgctaaccaacacaatgaccggaggtaactgctgctgttagcctgttttacagatgaggaaatgaaacaccaagagttgaatctgcccaaactgcacatgggattggcttagttgtttattttccatactgtgtgaggaaagactttgacttatgacatagatgaaatcaagggagtttctctttatctaggtttacttgttagccaatgaaagtgcttttaaacttgaacctctctaaatatttattattgtctgctgacctctttcattgagtaattcactgaatagaaaaatttaacgcttaagccaagcagagtcattgggtatgtaaatatggcaagtcatagtatttgaaaatagtgaaagactatgttcacttgggatttggtaggacttttaaaaataaaaataatagtagtaagcagcaacagactgtaatacaaactaggatgcatgaagattgtagtgctgttggttattatttgatagatgagcactcttgaacacagtttaatccaatttgcttagagtttatatgatattttttgtcatctaagaagcattgaaaaaaatgacagtatgtccaaaattttcagaaaaatcttaaattgtggttctcagggaaacctgtggtttttctcacatgtaaaataagtacaatttaaataataaggtaattgcataatttccctattatgtaattatataattacataataataatgtaacaatgtatacataatgcaacaatttaaacaaaattggagctcaaaaaataattctgaaagaatgaatgagtctataaaatttggtgaatgtacttggctgaatttggataatttaagcccatataaattctacttgtcacaaaaataaattttaaattactagaagtgttattcttgcctttgctcttgaggaaagaacttaaaagttatgggatagcaaagtgggagatgcagaattatgagataatattacttgaattatgtaaattgtttatgtaatctgaatattagcttacttcctaacttttcttaaatttactggtcgccctctgtgacttcaggtagcagataaaaaaacagccctaaagctaaggagagaaggaagcctgcagtgtgggaagggaagccattaaaactttcctcaaagaacagtggcagctatattttgcagagtgctgggaaatggttattatcacatctctgtgctttctggaggttaaaacatttgacagcatgaagtccgattatatataaatttacctaaaagttagaagaaaagttgtattgagaattgagagacaacagagaaggcttggttgattcaaaatttctactagcttcaaaggccatattatctgaaattaattctcttagttattattccttcattgtatattctccatctcagtttagtgtattaaactgcttcatactaaagagtttgcgatacctccaattaacttttgcagtatttccaattagcttttcccctgaaaggaaagaagatgaagaataggctagagaaggaaaggaagtggccccagaaaggttaggtataaggtggtaaggatggaggtttcctgctcaagtccttgctccggcgtcactgtccccatgaagcctaccatgaacacctatgtatattatcctcatatgtatactgcatccagccccttcctcttgcatttccactctcatgtctttatccagagctacctcttcctgtagtatctattgcttcataacatattatatcattttgttatatatcatgtttattgcttattacttgtatctgtcagctaaaatataagctttatgaggacagggatctttacctcttttgttcattgatatattaaaagtgcttagaaaagtgcttggacataataggcatccaaatatttgtggtaaaactgttccataagaaaaaaagaaaaagaaaaagggtagccccacattcccaaagatgtaaatagagtcctaagtactttgaataattcaaaggaattcagagtgaaatttttaaaagaagattattatttccttccatgtactactgtttaatttctatttcagtccccactggggagcattgacactggtcatttcaggaagtaaagctttgatgataagggagcttattttaataaagagagagaaaggaaaggaccaatgcattgacccatggttagggggtttctgaggaaatggctgttcccctgttttatgcagagggactgtggattctgcttgtaccttgtttatttatgggttactagagattggtgagtgtttcaaataaacagagtttattacagaaatttatgtgatttgatttattccaattggaagaaacagaatggtgaaacatgaataaatttctacttattgtatctttaattagaatattctgtcagagtctccaaaattttaatctatagatcagtgttgtttcaaaataaaatgttaatagcaaaatgacaaaacataaggtatccatatccgtcaaactatttatcccattcctaggaatttgttctgtttttacatagaagtgtaagaagctataaagatattcataacattttttatacttgtggaaaaaataaaaacatctaactacctaccaagacttcaatatgtctacaatgcaattttatgcagccatggtatagaagtataattatgatatgaaaagatatccatcaaatattcactcaaatgggttttaaagcagtatgttctatatataagtatattcaagcatgaacaacttgaaaattataatagcagtgattatctcaggatagtggaggcttattttctctttttagtttttcctttcctgtactttatatatatttttacaattagaaattagaatgcagaaaatatatagtagcatttatcagaggataagacaatgtttgtatattacgttttcataattttttgatatctcactgacttttggtattaaaaagacttgaggtcttcgaagagttgtgttgcttttgtttaacagctgtttatgtttggaaaaagggagaagaatttaatattttatgtacagtgctataattgtttgagtagacatggggagtgctatattcactgggaatatgtatagtactttaactgcttagaatatacttttgtacactttatctctttactgttgtgaaaggtgaattatctgttgcttccacttcagaaaagagaggttttgtttgtttgtttgttcgcttgtttgttagctgagtcaggatctggctctgttgcccaggctgaaatgcagtggcaagatcacgcctcactgaggcctcaactcccgaggctcaagcaatattctcccacctcagcctcccaagtagctgggactacacacttgcgccaccacacctgcctaattttttttttatttctaggagagaagaggtcttggtatgtcgcccaggctgttctcaaattcctgagctcaacggatcctcccacctgggcttcccaaagtgctgggataacaggcatgagccaacctgcccagccagaaaagagagattttaatgcttatgtgctttatgtgatttgctccggttcatttagctggtaaagcctgggctctgaaatgcagatctctgtgcttacagaaaacatgcttttaaaaaaaaagttatgaataaatgcaggcaaataaattataagctctgaaggacagaggatgtttttaagactatctgcatactaaggtatggatgtaagacataccaccatttggaatttttcccttgagcatacacagatatagtcatgagtggcagctgtgatagcacagcttctgaaggagagaaagcaactaaggactgccttgaaaatatttaaggagttatttgagctgctgtgttgcaagttcgcctaattagagtggctactttcctttgtattctcttggcaatgtacagggattccgatgaattggattgagagtaagaggttgacatccaagctccatgtatttttgtgaagttaacattggcaatgatatctggaacttagttcttttctacagttggccatgtgggtttttttatttacatacatttatgaggtacatgtgcaatttgttacatgcgtagtaggtggcagtgttttaaacaatggatttcacctctcctttgaatgcttgtttgagaaaagtaattcaaatcctatctaaataaatacatttatataatcatgacaacttttaaaaacaattataattttctttcactagtactgtgtatgttttgagctactgtgtttaataagctaaggatatatttgaagattttatcagtccacttggcatctcaaacttaatatgtttaaaaacatctcaaacttaatatgttgaaaacatagctcttgaccttgaccccttgtccgcctccaacaagtctttcccaattcagctactggtgacaacatccttttagttgcttagaccaaaaacctcagtgtcctccttgagtcctctcttttcacaccctgtgtccaatccatagcaaatcatgctgaatttacctttagaatatatatgagtttgaggcatttcttacctgctccacgggttcacgccattctcctgcctcagcctcccgagtagctgggactacaggcacccgccaccacgccaggctaattttttgtatttttagcagagagacggtttcaccgtgttagccaggatggtcttgatctcctgacctcgtgatctgtcagtccccgcctcccaaagtgctgggattacaggcatgagccaccgcgcccagcccaccaacacttcttacctgaatgttggcagcagcctcctaactggtctccctcctctgtcttgtacccctttagtctattttcaatgcagtagccaggtgaatccctttaacctagagcagtttatatcaccactcatgtcacaagtgctcaagcccttcacctctttcaagtaaatggcagttatcccaacgtccaaccaggccaagcactgtagcctcatgttacctctcctacccgttccctgatagctacttttccccctcctctgttccagccacattggcctctgctgtcctctgacaccccaggcatgctcctgccttgccctttgcacttgctcttccctctgcccagaacacaatctctctaaattgctcgtggcttactctcggagctccttcagactttcttcaaatgccagcttctcagtgaggccttcctggctgttgtccttgagctctccactgctctactctgctttatttttctccaaagtataatacttaccattatatgttatttattaattttatgttgtgtctgttttcccagaatatatgtattgcaagaagtcagggatttttgtctgttttactaaagattgtacccctagtacccagaacaatgtctggcacctaatagggactcaacaaaacttgctaaacatctattaacccaaaagttatttaatttattatatttaatggcaggtattgtattgagcagtagagtactatgtactgttaatcttgctaaaatatacttaaatatatcattttaatttagggagggggatatgacatcatttctgctctctcactagaattttaggtacatgatgatgtttaaacaaatttgcccaaaaaaaccaaattctatttcagagataaagccattgtttatacaattattatacttatagtagctatttttttaacttcaacttttctcttctgtctttcttgtttttgttttccagaaaagaggcacagttagatgaagaaggacagtttcttgtcagaataatatatgatgactccaaaacttatgatttggttgctgctgcaagcaaagtcctcagtaagttgaatgcaactttccttctttggccaagttacacgtagaagctcacagaatgcatggttcaagatcacaacgcagggttacagaagtggtgcagagcatttgtacaacctgcatagttgtgtggtgggcatccacatatcatgttaggctcaggctatgccaagtcttatttttccttttgcaaattctttagagaatagaaaagagtaaatgtgctcttcttgttgttttttgagacagggtctggctctgtcacccaggctgcactgcagtagtggcccaactccggctcactgcatcctctgcctgccaggctcaagtgatcctcccatctcagcctcccaagtaactgggactacaggcacgcaccaccacgcccagctaattttttgtatctttggtagagacgaggtttcatcatgttgtccaggttggtcttgaactcctgagctcaagtgatctacctggctcagcctccgaaagtgctgtgattgcaggtgtgagcctccacgccaggcccagtcctttgtctcccccaacagaaagccatgtattcaggaaggaaagagaattattgatacttaatgtaacaacatcagttgtggtgtcaagaaaacggtagttattcaagtataatatgtgtttctccaaatctcttttgtagtcacaagttatattaactattggtaaaagaaacatgaaactgagatctaccacagataaatatctttctgaagaaggcaaagttttaaaaatttcatggagattctcaaagtagtatctattattcccacagtgcttaatttttaactgggctatagtttggatacaatagaatgaagcttttagctttgccacatataactaggaaagcaaacactatctatagtagaataaaaattgttttaaaagattggtaaatgtttaatattaacaagaaaaatatctagcctccagcaatcaaaattgaaacaaacatgaaataactttttttattatcaatttggcagtcttaaaaacggaaatatctagtgtaatgatggagtacagttaatgggaaatcacatacattttaaatgtgatggtagttaagacaacttttttgagaacaattttgcctcgtgaaagccttttaaaaaaatattaaaaccttcagtaatttttcctaaataaattattagatctctttccattaagatttatgtatgtgttttcatgtcagagttccatatattatcaaaaactgaaaaaaagcctacattttcaataagaagggattggttaaataaattattgcatccatatgttgaataagatagcaattaaaactgatacacagcactttgggaggccgaggtgggtggatcacttgaggccaagagttcgagaccatcctggccaacatggtgaaaccccctctctactaaaaatacaaaaattatccaggtgtggtggtgggttcctgtggtcccagctactcaggaggctgaggcaggagaatggcctgaacccgggaggtggaggttgcagcgagccgagatcctaccagtgcactacagcctgggtgacaaagtgagactctgtctcaaaaaactaaaaaaaatgtgatacagaaaatttaagttttaggaaatattaatgattgtttattaagaaaaaagcaggacaccagggtacatgttatatgtaatcaagttattaaatatacatatttatataaagacttttgtatcaaaatattgtcatgtgcattcttgatgattttaaaaatctgtgttttttggaaaaaagaagtagtatatcaaatatatgctttaaaatggtcaaaattaagtacagggtgagtatctcttgtctaaaactcttgagaccagaagtgtttaggctttcagattttttttggattttggaatacttgcatatccataagaagatatcttgaggatgggattcaagtcgaaacataaaattcacttatgtttcatatacaccttatagacataggctgaaggcaattttgcacaatattttaaatgattttctgcatgaaacaacattttgactgcaatccattacacgagttcaggtgtagaattttccacttgtagtgtcacgttggtgaccaaaaagtttcaggttcttgagtatttagaatttcagattttcagattaggtatgctccacttataatttgtaatagaagctgaaaaactggttgctttgcttgtaaatgcaaatgtacaccattaacatgtttatatgttgtcttctctgatttcattgtattgtcctggcgggatttttttcatatgatctatgcttgccatgcacaaaaattgaagacattttacattaacttaataaacacttatcttcattttttctattaacaatatttcacctattatatgctttttttcccctcttgaatttgtaaaatagatctcaatgctggagaaatcctccaaatgtttgggaagatgtttttcgtcttttgccaagaatctggttatgatacaatcttgcgtgtcctgggctctaatgtcagagaatttctacaggtaagcaatttgaggtcctatagttaaaagttctgtgtttataactctcaaatatagactctagaatacttttgttcactcagctgtacaagagtgtcttgcttctgaaatggaatcgtagtcacctttttctaattcacataaaatcatcgttattggagcgtctcgaatacattttagcattatattttttctttaacttctggttgctgatactgaatatgcaagttgtaaattaatgttccctgaaaaactccaggtggtcttttgtgatctgttcaactgccctttttttggggtaatggctctatatcctagtgattgtggttttatgtgttagacatttaatatggccttattttctatttacaaaacattcctagtttgtagatataatcgagcaatgtatcagtatttatgaagttctacctggagctcaatgtttaggaaaaagtagtagggttttcaggtgttctgtcaccaaaacatacttaagatccattatcattagttaaatattaattaatctactgtacgagggaaaataggaaacagtccaaagtcagtttcaggtgaaaaagttaaataatgcaatcacaggcctttgtcttacattcttgttaaattacccttaaaagattgtactagtaaattacacattagatgagagactatttctggattgactatactattttaagcattgacatttgtgagaccctcttcttgaaatgtgttaacattgaaaattatatagttgtttgcaaaggatatttctaaactggctgctttcagcagccagatcttgctggaaaatgatgtcagtgcaatactagctgaaccgaagcccagttttgagggaatgtaaactactttcaatccagctgcattattgatttaacagccaaaataagaactttagtgtttaatcaaaatgtctgttgtttacatagtgacatttttcttataaacttaaaacagcattaaatcaagttggaaactggtttgcaatctcctgccatggatttactaaactactggtgtcaaaaaaagctgccacttccaaatgaagtagctgccaagagatatttaaataaaattgaacaggcagcatttaatgggattattttaaatggattaaaatttgatacttaaatgttttgggggattttgggtttttgaatacatatttcactttttagtacttttgttggccactatcaacttatagcaaagtttcaactttcagttgttctgacttacaactctctctctcaatgcccatgtttacataacaaaacaaagattcaagtggttttattaattccttttttttttttttaacctctactcattgtgaccatctgctaagcttgttatggcacagtaagagagcaagcggccttttaggtgtgtttctctgtattccagagctgctttatccaataggacagccactagtgcatgaggctactcagcatttaaaatgagatgtgcaaactggggagtggctcacacctgtagtcccagctacttggaggctgagatgggaggatcacttgagcctgggagatggaggctgcagtgagtgagctgtgatcacatcactgcactccagcctgggtgacagagtgagaccctgtctctacatacatacatacatgtgtacatacatacatacacacaacaaagtgcaatgtgctgcaagtgtaaaatatacaccagatgtcaaagacagtacaaaaagaataaaagatggctcattcacaattttatattgatggatttcatgttaaaatgttaatattttggatatatttggttatataaaatgtagttaacatagctttaaattatacacaattttattttatttctctttcaacgtgagagtgaaaaatgaagaaactagaaagttagaatactgtagttttcatcaatattttacattatgattcagttcttttattttctcctggttttgaaattatacattttaagggcaaattgaatatgttaaaatactgcttttctaaaaagtagatatttatgttatcattaggctgattacacagaaattaaatggactttttctgtgtgactataaaacaaaatcattaactttcatacattgttttaacatgccagcacacatatattgacatttttattacgtaaattagtaaaatattctttatggtgacttcaactcttcactaaaaatttaaagactttctttctgtttcagtcctggttttaatcagtcacagcatctttagattctatttcccaaatactgagctttgggaattctgccacttttctctatttccccccacacttctttgggagagtctaccattatctagaggttgaatggattccctgcttctcttcatgactcctccaatctcttctccatcctataactctagtgctttttctgaaatgcaaatgtctctcgtcgtatcgttttcttgcttcaactctggagtaattttttattgctcactggaaactgtcaaaatatttaaaacaattgaaacctacatcctttctgcatctttcctctccctctcccccagtccagccaaacttaacctctttaagaaccttgtatttatcttgttcccgctatcctctcagcttttccactgctcttctctttgcctggagcatgtttccagcactttccccatagataaattctattaattctgcaggtaaaattacttctctgggaagacgccactcctctctccaagctctgccctatcctagtcacggtaactaacctctctcttattttcataggacactctatttccccagccacagtagttttcatactgcattatcattcccatttgatggactggaatgtaaacttcaggaagccagagatcatgtataatctcttaatgtttgtaccttcacagatgcaatctgtatatctgagaataaatgaatatcttttttaaaaaagtgaatatttgttgtttgtgcaaaagcagaaaacacacaataggaagcaaacacaccaatagatcaagctgaattatttgcctcaggataacccatttcgagtgagatcctgtaatttttttttacggatgaatcatagattgaaatataaaagaagagacatacactagaccaatttccttttcaggtgaacatcctaaattactgtttattatttaatttacagggtacattgcttgtatctgttgacacttttaaaatggtcgttctgacatttccattttaagcttttagggaacactaaaatagctactgaaatcataacatgggagactatttgattatgccttagctgagcaactctgactcactcatcatagcaagcagaatttccttggaagttgcctgtccacagcctctcagttagatacattattaagaataggtctgtcttatatatgtatatacataccataccattgtttttacttttcttgaaataatgaaagaaaatttctaagtactagacacagcatataataggcaattaatgaaacagtctttaaataatagaattaattaattctgaagtatatgtgtctttataattatttggtccagcctgttttatcaggataatacctcttttgcttaaattttttattcgaggatactgtatagataatatctgtagcttagctccagaaaaatatttgttttctcacatggaggttatcatttccattcacagttgacttaacgaatactaacatatctttccccttgaaaggctttggtgccaatatccaattcatgaccctgcttatgcagtctctcctgtgcttacaaattcaagtttaaaaaatttgcattatgcgtattttaagccacactgagactttaacaaaaaaatagctagaagtcctaaatggataaataaaatataatgggatatatttaggataaataaaatttaggataaataatatttaggatatatttacgataaataaaatttatcctaaatggataaataaaatataaaatttaatggataataaaatataagaaatatttttagagaaaactttgtagaaatactcctcttctctaggcatttcacagtttagaattgattttttcttaaatcaccagtgaaagcatcaacttgtgggctgatgaaaaaagtaataatctttggcatttattctttggttgatttaataattgaaaatataatgtatcaaaaagagaaaaagaagacatttttaaaatttgatttttggatcttatatattttacctgggtatattgttttaaagacaagaccatatttcagtaagaattttgtctagtttttccacttttttattcataaattgacaataaaatgtgtggttccatgctattgctatagtcaatgtctttttttgcgttttttaatgttttattattattattattattattgagacggagtcttgctctgtcgcccaggctggagtgcagtggcgcagtctcggctcactgcaaactccgcctcccgggttcacgccattctactgcctcagccgcccgagtagctgggactacaggcgcccgccaccacacccagctgatttctttttcttttattttgtattttttagtagagagggggtttcaccgtgttagccagaatggtctcgatctcctgacctcgtgatccgcccgcctcggcctcccaaagtgctgggattacaggcatgggccagagcgcctggctgctatagtcaatttctatctgaagcttctagtgtactgatgaaaagactgcaaagaatacatgggatttgaaagcttcataagaaaatagctgataaggaatgttcagtttaatgttatttctaccttaatccacgctcacctctactctctaatgtcctattgcattatggcacattctattttccctaaattactgcaacagtctctcccatctcacatgcatttctgtagtatgattttgcctcttccttgcgggaggtaaaattctcttttccttgaatctgggttggctttagtgactatttaaccaatcgaatggggcagaagtgagatatttaaacttcctaggctagagtacaggaagtcttggtttccttcagggtatcttggaacagttgctgttgcgatggtacctctagaaacccagacacatgaaatgagaagcacaagccacatggagaggccacgtgtagactggcagatagcagttctagctgggctctcagcaaagagctggaaataactaccagccatttgactgatccatcttggaaaaccagcctgctccggcattctgataatttcagcccagtttccatctgattccgtcccaagggtagaactttctaatttagccagacaacccacagaaccatgaaaaataacaaataaattattaaagttgtttgacatacagctatggataactagaccactactctgtgcattatttttccctttatattttatgtctgtgttaatatgtgtgtatatgcttgtacaatgagtcaagagctttgagaaagctgtatttgataagataatctattacccctgggaataggagccatgtcagtcccaaacagggctaccaattcaatttcgatggtattacattattattacattatattgtttttacctcccctacaaatttacagagttaaaatgcaattaaaatgagcagcgaaggctgccaggttgactatgaaaatatacgtgggcaaatgcaattttagagaacagttaggatttaaattatgacctaatttaaattagaactcaggtgaatagttagatcagcgttctttcattatttgctggagacactggcttcttcaactaaccttgatatgttgacagtttcagtcattcactcaactcagatatatttgagtttatcaactagcgcctctgttcagcctgccatgggattttcttcaattcctgttggctccaggttttgacttattaaatacaacattgtgatttggccaccaggaacaatctgagattgatgactgatctaattcttaaatgtttgtgcaaagggttattttaggaaatatgaggattttcctgaacagctgtaaaattctaatacttccctaaattatttatatttcttaagaaaaaagagcaccattcactttatttttaactttggaattttaaaaaagagctgttttctcagcatccacaacgccaatttctataatccaataataaaaatagttttattttttaaaaacttatgaaggaagataagaagtattccagaattgtaaggcttttcagtttgaataactggtatatattttcataatttttcaaaatattgattaattgttacatgtagtattatatagactggatcttttttaacatgagtttaaaagcaatagaataagagaaatattttaactgtttttctttaattaaaatatttccttggagaggatggtaatgataaattgatatagctttaaagctatttccatttaaataattttagtgactctggtacagtttctttttaaatagagattttcaatgtttcttttagaaaagaatctctatatcttttcagttgaaaagctatttaattctcaagaagcagctccaggaagaaaaggcaggaaaagtattatgaaagcattcacttcctactccaatctcatgctatttactttggtgtttctcatcatctagggtttgatgttccattttacttaggtcgactttgtttaaatgttttatcttcaaattccaaaatgacttttttcttataaaacttaacaaaatattttttaaattacatgactctcattaaagaatgttggtgttggtcctatttttcagaggtgtgagggatagaaagaaaggaaatactatacctcatcctccttcattgaaaaacaaaaagaggtttgcaatgactggttttaaagatttcttgcagatctaaactgcagtaatattctcaacacagacaactcccttctccccatccactaaaacaaggattcttggatactgaaaatgtttttctttaaaacgttcaaggcaaggtcttacccatttctgtggatcctttaaaaaatttaattagatgtccttgaaacaggaaaatcaggtagttgacttctggggactttgcccctgtaattatgtaagaacttatttttggcatgcatattacaaaaagaaaatgtgcatttattttgaaacatcccaagatctttaaaaatgtgacactttattactcaaaggaatgataaattgtcttttgttttctaaatctgtgaagtgtactatgtctgaaattccagtagagtaccccagaataccagaactaatttttcttttataaatgataaatgcaaggtgataaaataaactagactgacagggagcagaattcctgagtagaaaaaccacattcatttgcttgtagaaaaacaataaaatgatattagaacattaagaaatgaaattattaaattaaaacctttaggtatctcaagatatcaaaatagatgaatggacctactctctatatttttcctgttttttgattttatgaagcctagaagaatgaatcaaagcatatttcagatttacatcattgtttcctgggaatctacttgtgctgcaatataagagtttacattttttcagtgtcttttttactttcatacatatggacatacttctttgcaattggccacacagctctttactttctgtaagtgcttctgtgggtatatttagaaataacacagaatatcagttttgatatgaaactcttattccccagaatacattgttagagcaatgtggatgaactatcatctttgtttagatagcattatcaaatgtagaaagggttttctcttggccattgtaagaatctttttgtagcaaaaaaaaaaaattgtagcctgtatatgaggatgacacatttattagcaatataagtgtacttaaatgaaaataacttttaatcattaatctcatttcttgtaatagtctggctgtggtaattactttaaattttgaaagagtaaagccaagtaaggaaggttgtaactcaagaaatcccaagagtattttttatattagatactatctaaagaaaatcaatattacattaagtggtgttcaatcttttatgattcaggagagcactttacttgttcaattaactaatagtaaattcaataacacagtaaatctctggagagttggttactgcaaaaagacagaaaaggtgaacattctcatttggtgtttttggtatcctttggtgagggtgcagtgcctatctttagtgactcaggaagtactgacttggatgattgtgtccactctctgacttgccccagcataagtggtgccagtgacataagtggtgcaagtcgttattgccttatgccatctgcttgagtgaaagtagaaatgttttaataatccagtaaaaaagatgagtatgaaaatgatataagcacattggtacattgatttatgaaatatgtaagcattctgtatagaaaaaatattaaaatgcctttttgaagttattgtttggttcaatttcctttttttttttttttttttttgaggcagaatctcactgtgtcgcccaggctggagtgcagtggtgagatctgggctcactgcaacctccacctcctgggttcaagcgattctcatgcctcagcctcccaagtagctgggactacaggcatgccccaccacgcctggctaatttttggattttctttctttcttttttttttttttttttttttttgagatggagtctcactctgtcgcccaggctggagtgcagtggcgagatttcggctcactgcaagctccacctcccgggttcatgccattctcctgcctcagcctcctgagtggctgggattacaggcacccaccgccacaccctgctaatttctttttttttctcttagtagagacggggtttcaccgtgttagccaggatggtcttgatctcctgaccttgtgatccgcccgccttggcctcccaaagtgctgggattacaggtgtgaggcaccgcgcctggccaatttttgtattttcagtagatgtggggtttcaccatgttggccaggctggttctcgaactcctgacctcaagtaatctgcctgcctccgcctcccaaagtgctgggattacaggtgtgagagcacgcctggcctcaatttcttttttaagtgagccaacaacacagtacctgggcctgactctattagaaacattttctaaatgcagccacaagctcaattttgactttgtgagaaatgcagataaaaataaaactctctcagtgattttttttttctcacccgattttctttcacaaaatgtttcacaacataagtagaagtataaggggtctgagagggctatgaagcagttctcaggtcgtcccttctaagggaagagtcctaatgagaatgattcatcattggaagaacctctcatactattcaaccaagtaaaacagttttaggactccggggtttgtaggttatatatacctcttcaatacttagtggctgccatattggattacttttacatggcaatagcagcctctagaggcaatccggatgaactgatgtttttgctaagaattagcttgactccatcaggtataattgtggggaaggaaacagtagatgcagacggttcaaaaagtcaccagatttatcgagttgaggaggtcattgaatcatttatctggattagattgtcaaacttcatgacctagctcaaatcacatcttcagtttgaagtatttttaactccattctttcagtctgaattgttggcttcctcatttgctcatcaaaagccactctacacatccatttgtgatataaaccatgagctgcttaatcctcatcatgcagatctgcatccaaacatctaccctgcacccaagcccacatgacacagtgtatgatatataatgcacatacaataacattatagagtgaatgcatgattgagtggatgtgtggaagggacagaaaattaatgctgtaagtgtcctgttagagtattgctcaaatgtggacacccgcggaatcatctcattctcaccgtagtttcctcccagtaatctcactggatttttctgtttttctaaattgacctctttgagacatttggaatgaaagccatagtttaattttggcatatctccagtactttcattttaggtatgttcttttatgcccatgacattttgaaactgttttgttgtttttcttttcttctctttaaaaataatcaccagttgtgaatagattactacttgcttggtgtatttgttttttattgctaccataacaaatgaccacaaacttaagtgcttaacacaaatttattaccctatggtttcataagttggaagtccattgcaggtcttactggactatattcagcgtgctgtgctggtgggacgggactgtgtttctttatgttggcttttggggataattgttttcttcctcattcaagtattgacagaattcagttccatgcagttatagggctgaggacccagtttccttgactgtcagcaggcagctgccctaaagtcctggagacctctctccagtccttcaacagtctcctacatctcagaaccagtcgcatggtgttgaatccttctcatactcccatctctttgaccttttgtcattgcacgtctctctgtcaccatgtagcagggaaagcttcttgacttttaagtgcacagtcacccatgtgactaggttgggctcacctggataatccaggataatcttcccaactcaaggtccataaccctcatcacatctgcaaaatccctttcaccatgcgatacaacatattcacagggattaaggcaagggaatctttcagagacagttgttttttcagcctcgtctgaaatgtgctaagtgctttatatacatgtttaaagttatctccattttgccagaagaaataaagtcttagaaaaattgggcgaattggctaccttcctatgaaatgacagtcattatttaaacccatgtctacttgatcctaagtcatattgctcttaattcctataccatgctgtgctgtgtgtatatcctactatatatgtgcagcacacacacacacaccccaacatgttatgaactgagtgttatttgaaaaatcatcttgggccacataaaatgtatagcttacaccgtttcttctagtactctttacaccaattagttttgtatattagtaatctgagactcagggaaacgtggcatcttccaaactaatggcaaggctacaaaaacagaaaatatttattggccaatttattagttttcctagactttcaaaatgtagttatttcacagattagctatgaaactccaccttgatgatagcatactaaagaacatgaagaaattacattgttttagtttctaaaatagatttttaatttctaagatattagaatagttaaatagaaatcacaaaataatatgcttatgttttatatgcttataatttgggcatagttttttgagttgacaattaagttttagaattgcagtataaacagaagggaaatgtgtgagtcctttggccattccctgttgttgatcacaacacgtagaaacagctgccccaagctcttgtaaatgtatctttgccttagcccttaactatgtttttttctgttttagccatcctttttaatcccacttgtttatcttttcttaaaatgtactaactattcatagctgcagatatttttcttaattctactatcttagtttcattttctctgtggcacttgctttacagatatctcagtggagtgaaggatgtgctacatgtgctgcttcgctgatctgcccactagactccgatttgactcgttttcatatcttgctttcccagagtctgctgtcattgcgaaagcattgtttattggtctccctttcttctttgctgcagaaccttgatgctctgcacgaccaccttgctaccatctacccaggaatgcgtgcaccttcctttaggtgcactgatgcagaaaagggcaaaggactcattttgcactactactcagagagagaaggacttcaggatattgtcattggaatcatcaaaacagtggcacaacaaatccatggcactgaaatagacatgaaggtaacaaacagcaatggagacttctgaacacagatgacatctaaaaatattttaaatgacactctctaaatttacctgcagttatcactgttagtgctcttccctggaagtatatataagccaaaacagtggaaagaccagcaatcttctatatttggcctgggagtgcattatacaggatatttttttccttctggcagcagatttttctttctaattatatagtgttcaaggctacaagaaatttactttgcattccctgtgaacacacaccagctgggaagcagaaggatatgtgacgactgagctgtgggtttcagcatgcaaaatatccataatatggatagtcaaattctaaggcaatataaaattaagtattattttatagatcattttctctttgtcgatttttgtctgccctgaaatatagttggaatgtctaattttgtgaatgaaagtgtttgtttttgtttttgtttttaatcaagttacttggtgaaagaagccaggtctggtactttaaaaattgtgctgttattgataatacacactcttagttttgagtgatgatttcaaggtactgatagggcaaccgtacaacaaagagaaccactgtttctctttacctctggcccttacggtttctcccagtcaggctgctttttatgtgttcagcatgttcatacccaagagtcaattctcagtagctttaaacttgaggccaaagtggggagatagatacctttttccaacctttctttgtttatagaaaacctattagacagttctttcttatagatcatagatatattgtaacacacttgggaggtctaaaacccttaagtggagtattatccaaaattaaaatagtaacatttaaataaattagaaagatattccacatatttaatctagtaaagttatgttagagttcatggttttctttctttcagaagttggtgttagttcttacgtggcaccttttggtgatttccaggtgcatcgttagttttctacaaccattagtaatggcggaaaccacgattactgttgcaccaacctaataga

SEQ ID NO: 7 - DNA, 21 bp, Mus musculus
catgatgcgatcacaggaggc

SEQ ID NO: 8 - DNA, 21 bp, Mus musculus
cgcccggagcctaggaagcag

SEQ ID NO: 9 - DNA, 21 bp, Mus musculus
ctctctgtgtgtgagagagag

SEQ ID NO: 10 - DNA, 25 bp, Mus musculus
gtcagtgtcagacctgaagatgctg

SEQ ID NO: 11 - DNA, 21 bp, Mus musculus
cccttccttgcttctcagtac

SEQ ID NO: 12 - DNA, 27 bp, Mus musculus
ctgctacaagcattgcctagacggacg

SEQ ID NO: 13 - DNA, 21 bp, Mus musculus
gacaccatgtacggtttcgtg

SEQ ID NO: 14 - DNA, 20 bp, Mus musculus
ctccaccttgtagacatcca

SEQ ID NO: 15 - DNA, 19 bp, Mus musculus
tgcacttcagagaaccttg